(19) United States
(12) Patent Application Publication
(10) Pub. No.: US 2025/0340554 A1
(43) Pub. Date: Nov. 6, 2025

(54) 5-Amino-1H-Pyrrolo[3,2-b]Pyridine-2-Carboxamide Derivatives as MTA-Cooperative Inhibitors of PRMT5

(71) Applicant: BeOne Medicines I GmbH, Basel (CH)

(72) Inventors: Jing Li, Beijing (CN); Sanjia Xu, Beijing (CN); Zhiwei Wang, Beijing (CN)

(73) Assignee: BeOne Medicines I GmbH, Basel (CH)

(21) Appl. No.: 19/269,655

(22) Filed: Jul. 15, 2025

Related U.S. Application Data
(63) Continuation of application No. PCT/CN2024/073247, filed on Jan. 19, 2024.

(30) Foreign Application Priority Data
Jan. 19, 2023 (WO) PCT/CN2023/073190
Jan. 19, 2023 (WO) PCT/CN2023/073254

(51) Int. Cl. C07D 471/04; A61K 31/437; A61K 31/444; A61K 31/4545; A61K 31/4709; A61K 31/501; A61K 31/502; A61K 31/506; A61K 31/517; C07D 519/00

(52) U.S. Cl. CPC C07D 471/04; A61K 31/437; A61K 31/444; A61K 31/4545; A61K 31/4709; A61K 31/501; A61K 31/502; A61K 31/506; A61K 31/517; C07D 519/00

(57) ABSTRACT

This disclosure provides compounds containing 5-AMINO-1H-PYRROLO[3,2-b]PYRIDINE-2-CARBOXAMIDE derivatives as MTA-cooperative inhibitors of PRMT5, the use thereof for selectively inhibiting the activity of PRMT5 in cooperative with MTA in tumors bearing MTAP^{DEL} mutation, and pharmaceutical compositions comprising the compounds as treatment of various diseases including cancer.

### CROSS-REFERENCE TO RELATED APPLICATIONS

This application is a continuation of International Application No. PCT/CN2024/073247, filed on Jan. 19, 2024, which claims priority to International Application No. PCT/CN2023/073190, filed on Jan. 19, 2023, and to International Application No. PCT/CN2023/073254, filed on Jan. 19, 2023, the disclosures of each of which are hereby incorporated by reference in their entireties.

### FIELD OF THE INVENTION

This disclosure provides compounds containing 5-AMINO-1H-PYRROLO[3,2-b]PYRIDINE-2-CARBOXAMIDE derivatives as MTA-cooperative inhibitors of PRMT5, the use thereof for selectively inhibiting the activity of PRMT5 in cooperative with MTA in tumors bearing MTAP^{DEL} mutation, and pharmaceutical compositions comprising the compounds as treatment of various diseases including cancer.

### BACKGROUND OF THE INVENTION

Epigenetic modification is a process that can modify genetic output changing the primary DNA sequence. Epigenetic modification plays an important role in gene expression and regulation, protein production and cell differentiation in multiple dimensions. Typically, this process is reversible and selective, on DNA, its regulatory proteins such as histones and other proteins such as transcription factors [Bradbury, E. M., BioEssays, 1992, 14 (1): pp. 9-16]. PMTs (Protein Methyltransferases) are central players on epigenetic modifications, consisting of two sub-families named PKMTs (Protein Lysine Methyltransferases) and PRMTs (Protein Arginine Methyltransferases) [Copeland, R. A., et al., Oncogene, 2012. 32 (8): pp. 939-46]. PMTs are associated with various human diseases and considered as potential therapeutic targets [Copeland, R. A., et al., Oncogene, 2012, 32 (8): pp. 939-46].

As the name implies, PRMTs catalyze the methylation of the arginine residues of proteins. Besides their primary functions of methylating the histone tails, PRMTs also target on other cellular proteins such as NAB2p, FOXO1, PABP1, Sm D1, etc. [Bedford, M. T., et al., Molecular Cell, 2005, 18 (3): pp. 263-72]. Divided by the products, the 9 mammalian PRMTs can be classified into 3 subtypes: type I (PRMT1, PRMT2, PRMT3, PRMT4, PRMT6 and PRMT8) catalyzes aDMA (asymmetrical dimethylated arginine) formation; type II (PRMT5, PRMT9) catalyzes sDMA (symmetrical dimethylated arginine); and type III (PRMT7) catalyzes MMA (monomethylated arginine) formation [Yang, Y., et al., Nature Reviews Cancer, 2012, 13 (1): pp. 37-50]. In addition, type I/II PRMTs can also catalyze MMA formation as an intermediate to aDMA and sDMA. The PRMTs comprise a pocket to interact with its cofactor SAM (S-adenosyl methionine), and an adjacent pocket to interact with the arginine residue on a protein, namely SAM-pocket and substrate-pocket. The methylation process involves an SN2-like mechanism of transferring an activated methyl group from cofactor SAM to the guanidino group on the arginine residue. [Bedford, M. T., et al., Molecular Cell, 2005, 18 (3): pp. 263-72]. The side product of the process is SAH (S-adenosyl-L-homocysteine).

The overall arginine level in cells is roughly 1500:3:2:1 for Arg:aDMA:MMA:sDMA, and PRMT5 accounts for the vast majority of sDMA formation [Dhar, S., et al., Scientific Reports, 2013, 3:1311]. In contrast with PRMT1, the major type I PRMT which functions on its own in cells, PRMT5 binds to MEP50 (Methylosome Protein 50) to form a heterocomplex that is often elevated in cancer cells and correlates to poor patient survival [Gao, G., et al., Nucleic Acids Research, 2019, 47 (10): pp. 5038-48]. PRMT5 promotes tumerigenesis in varied mechanisms. PRMT5 is a strong repressor of numerous genes; when PRMT5 methylates histones H2a and H4 on Arg3 and histone H3 on Arg8, it represses gene transcripts that involved in differentiation, transformation, cell-cycle progression and tumor suppression [Karkhanis, V., et al., Trends in Biochemical Sciences, 2011, 36 (12): pp. 633-41]. Besides its epigenetic roles, PRMT5 may also regulates RNA-binding proteins such as splicing factors. For instance, a reproducible event was observed in PRMT5 knockout mice, in which exon 6 skipping of MDM4 (Murine Double Minute 4) occurred and p53 was released to upregulate p53 pathway [Gerhart, S. V., et al., Scientific Reports, 2018, 8:9711]. In addition, PRMT5 could directly influence key proliferation pathways by direct methylation of p53 [Jansson, M., et al., Nature Cell Biology, 2008, 10 (12): pp. 1431-9], EGFR [Hsu, J.-M., et al., Nature Cell Biology, 2011, 13 (2): pp. 174-81], PI3K [Wei, T.-Y. W., et al., Cellular Signaling, 2014, 26 (12): pp. 2940-50], etc., Thus, PRMT5 has a good potential to become a clinically relevant target.

On the other hand, PRMT5 is an essential gene in normal tissues, and the systemic inhibition of PRMT5 may result in significant liabilities, especially hematologic toxicity [Ahnert, J. R., et al., Journal of Clinical Oncology, 2021, 39 (15-suppl): p. 3019]. Therefore, strategies to selectively block the PRMT5 activities in tumor cells are required for a safer therapy.

Homozygous deletion of tumor depressor CDKN2A (Cyclin Dependent Kinase Inhibitor 2A) occurs in about 15% of all tumor types. Interestingly, the mutation frequently involves the co-deletion of proximate genes existing in 9p21, including the gene that encodes MTAP (Methylthioadenosine Phosphorylase) [Firestone, R. S., et al., Journal of American Chemical Society, 2017, 139 (39): p. 13754-60]. As a result of MTAP deletion, MTA (methylthioadenosine), the substrate of MTAP, accumulates. MTA is structurally related to SAM, and is a weak ligand/inhibitor of PRMT5 that occupies the same pocket with SAM. The formation of MTA-PRMT5 complex provides chances for further PRMT5 inhibition by formation of a tertiary complex. In such way, a correlation of MTAP null status and dependency of PRMT5 is established through MTA concentration level, to provide a precise oncological therapy.

Currently, most of the clinical-stage PRMT5 inhibitors are unable to differentiate normal cells and cancer cells, based on a SAM/MTA competitive mechanism (JNJ64619178, PF06939999, PRT543, and PRT811) or a non-MTA cooperative mechanism (GSK3326595). There thus remain unmet and continuous medical needs for potent and selective MTA-cooperative PRMT5 inhibitors.

### SUMMARY OF THE INVENTION

One objective of the present invention is to provide compounds and derivatives which function to act as PRMT5 inhibitors, and methods of preparation and uses thereof.

Aspect 1. A compound of Formula (X):

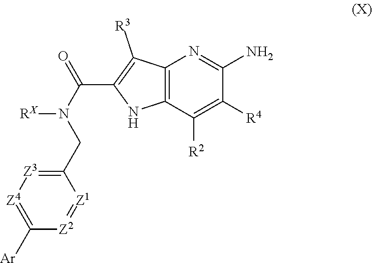

or a N-oxide thereof, or a pharmaceutically acceptable salt thereof, or a stereoisomer thereof, or a tautomer thereof, or a deuterated analog thereof, wherein:
Z¹, Z², Z³ and Z⁴ are each independently selected from N or CR^{Z};
at each of its occurrences, R^{Z} is independently selected from hydrogen, halogen, —C₁₋₄alkyl or —C₁₋₄alkoxy; wherein each of said —C₁₋₄alkyl or —C₁₋₄alkoxy is optionally substituted with at least one substituent selected from halogen;
Ar is selected from phenyl or 5- to 6-membered heteroaryl, each of said phenyl or 5- to 6-membered heteroaryl is optionally substituted with at least one substituent R^{Ar},
R^{Ar} is each independently selected from hydrogen, halogen, —C₁₋₄alkyl, —C₁₋₄alkoxy, —C₃-C₆cycloalkyl, —C₃-C₆cycloalkoxy or —CN; wherein each of said —C₁₋₄alkyl, —C₁₋₄alkoxy, —C₃-C₆cycloalkyl or —C₃-C₆cycloalkoxy is optionally substituted with at least one substituent selected from halogen;
R^{X} is R¹ or

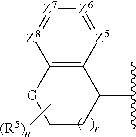

R¹ is each independently selected from —C₁₋₈alkyl, —C₃-C₈cycloalkyl (preferably, mono, bridged, fused or spiral cycloalkyl) or 3- to 8-membered saturated heterocyclyl (preferably, mono, bridged, fused or spiral heterocyclyl); wherein each of said —C₁₋₈alkyl, —C₃-C₈cycloalkyl (preferably, mono, bridged, fused or spiral cycloalkyl) or 3- to 8-membered saturated heterocyclyl (preferably, mono, bridged, fused or spiral heterocyclyl) is optionally substituted with at least one substituent R^{1a};
R^{1a} is each independently selected from halogen, —C₁₋₄alkyl, —C₃-C₆cycloalkyl, 3- to 6-membered saturated heterocyclyl, —C₁₋₄alkoxy, —C₃-C₆cycloalkoxy, —CN, —OH, —NH₂ or oxo; wherein each of said —C₁₋₄alkyl, —C₃-C₆cycloalkyl, 3- to 6-membered saturated heterocyclyl, —C₁₋₄alkoxy or —C₃-C₆cycloalkoxy is optionally substituted with at least one substituent selected from halogen, —C₁₋₄alkyl, —C₃-C₆cycloalkyl, 3- to 6-membered saturated heterocyclyl, —C₁₋₄alkoxy, —C₃-C₆cycloalkoxy, —CN, —OH, —NH₂ or oxo;
G is selected from CH₂, O, or NH, provided that when G is NH and NH is substituted with R⁵, R⁵ is not halogen;
Z⁵, Z⁶, Z⁷ and Z⁸ are each independently selected from N or CR¹, provided that at least one of Z⁵, Z⁶, Z⁷ and Z⁸ is N, and others are CR^{Z1}, and Z⁵ and Z⁸ are not N at the same time;
at each of its occurrences, R^{Z1} is independently selected from hydrogen, halogen, —C₁₋₄alkyl, —C₁₋₄alkoxy, —C₃-C₆cycloalkyl or —C₃-C₆cycloalkoxy; wherein each of said —C₁₋₄alkyl, —C₁₋₄alkoxy, —C₃-C₆cycloalkyl or —C₃-C₆cycloalkoxy is optionally substituted with at least one substituent select ed from halogen, —C₁₋₄alkoxy, —CN, —OH, —NH₂ or oxo;
at each of its occurrences, R⁵ is independently selected from hydrogen, halogen, —C₁₋₄alkyl or —C₃-C₆cycloalkyl, wherein —C₁₋₄alkyl or —C₃-C₆cycloalkyl is optionally substituted with at least one substituent selected from halogen, —C₁₋₄alkoxy, —CN, —OH, —NH₂ or oxo; or
two geminal R⁵ and the carbon atom which they attach to form a 3- to 5-membered carbocyclic ring; wherein said ring is optionally substituted with at least one substituent selected from halogen, —C₁₋₄alkoxy, —CN, —OH, —NH₂ or oxo;
n is 0, 1, 2, 3 or 4;
r is 0 or 1;
R² is selected from hydrogen, halogen, —C₁₋₄alkyl, —C₁₋₄alkoxy, —C₃-C₆cycloalkyl, —C₃-C₆cycloalkoxy or —CN; wherein each of said —C₁₋₄alkyl, —C₁₋₄alkoxy, —C₃-C₆cycloalkyl or —C₃-C₆cycloalkoxy is optionally substituted with at least one substituent selected from halogen, —C₁₋₄alkoxy or —C₃-C₆cycloalkoxy;
R³ is selected from hydrogen, halogen, —C₁₋₄alkyl or —CN, wherein —C₁₋₄alkyl is optionally substituted with at least one substituent selected from hydrogen, halogen, —C₁₋₄alkoxy, —C₃-C₆cycloalkyl, —CN, —OH, —NH₂ or oxo;
R⁴ is selected from hydrogen, halogen, —C₁₋₄alkyl, —C₁₋₄alkoxy, —C₃-C₆cycloalkyl or —C₃-C₆cycloalkoxy, wherein each of said —C₁₋₄alkyl, —C₁₋₄alkoxy, —C₃-C₆cycloalkyl or —C₃-C₆cycloalkoxy is optionally substituted with at least one substituent selected from halogen, —C₁₋₄alkoxy, —C₃-C₆cycloalkoxy, —CN, —OH, —NH₂ or oxo.

Aspect A1, the compound is Formula (AI):

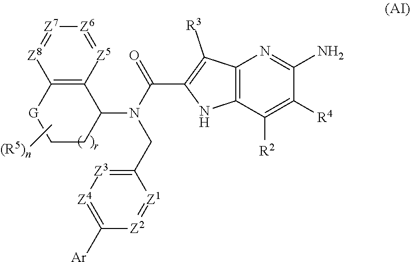

or a N-oxide thereof, or a pharmaceutically acceptable salt thereof, or a stereoisomer thereof, or a tautomer thereof, or a deuterated analog thereof, wherein:
Z¹, Z², Z³ and Z⁴ are each independently selected from N or CR^{Z};
at each of its occurrences, R^{Z} is independently selected from hydrogen, halogen, —C₁₋₄alkyl or —C₁₋₄alkoxy; wherein each of said —C₁₋₄alkyl or —C₁₋₄alkoxy is optionally substituted with at least one substituent selected from halogen;
Ar is selected from phenyl or 5- to 6-membered heteroaryl, each of said phenyl or 5- to 6-membered heteroaryl is optionally substituted with at least one substituent R^{Ar},
R^{Ar} is each independently selected from hydrogen, halogen, —C₁₋₄alkyl, —C₁₋₄alkoxy, —C₃-C₆cycloalkyl, —C₃-C₆cycloalkoxy or —CN; wherein each of said —C₁₋₄alkyl, —C₁₋₄alkoxy, —C₃-C₆cycloalkyl or —C₃-C₆cycloalkoxy is optionally substituted with at least one substituent selected from halogen;
G is selected from CH₂, O, or NH, provided that when G is NH and NH is substituted with R⁵, R⁵ is not halogen;
Z⁵, Z⁶, Z⁷ and Z⁸ are each independently selected from N or CR¹, provided that at least one of Z⁵, Z⁶, Z⁷ and Z⁸ is N, and others are CR^{Z1}, and Z⁵ and Z⁸ are not N at the same time;
at each of its occurrences, R^{Z1} is independently selected from hydrogen, halogen, —C₁₋₄alkyl, —C₁₋₄alkoxy, —C₃-C₆cycloalkyl or —C₃-C₆cycloalkoxy; wherein each of said —C₁₋₄alkyl, —C₁₋₄alkoxy, —C₃-C₆cycloalkyl or —C₃-C₆cycloalkoxy is optionally substituted with at least one substituent selected from halogen, —C₁₋₄alkoxy, —CN, —OH, —NH₂ or oxo;
R² is selected from hydrogen, halogen, —C₁₋₄alkyl, —C₁₋₄alkoxy, —C₃-C₆cycloalkyl, —C₃-C₆cycloalkoxy or —CN; wherein each of said —C₁₋₄alkyl, —C₁₋₄alkoxy, —C₃-C₆cycloalkyl or —C₃-C₆cycloalkoxy is optionally substituted with at least one substituent selected from halogen, —C₁₋₄alkoxy or —C₃-C₆cycloalkoxy;
R³ is selected from hydrogen, halogen, —C₁₋₄alkyl or —CN, wherein —C₁₋₄alkyl is optionally substituted with at least one substituent selected from hydrogen, halogen, —C₁₋₄alkoxy, —C₃-C₆cycloalkyl, —CN, —OH, —NH₂ or oxo;
R⁴ is selected from hydrogen, halogen, —C₁₋₄alkyl, —C₁₋₄alkoxy, —C₃-C₆cycloalkyl or —C₃-C₆cycloalkoxy, wherein each of said —C₁₋₄alkyl, —C₁₋₄alkoxy, —C₃-C₆cycloalkyl or —C₃-C₆cycloalkoxy is optionally substituted with at least one substituent selected from halogen, —C₁₋₄alkoxy, —C₃-C₆cycloalkoxy, —CN, —OH, —NH₂ or oxo;
at each of its occurrences, R⁵ is independently selected from hydrogen, halogen, —C₁₋₄alkyl or —C₃-C₆cycloalkyl, wherein —C₁₋₄alkyl or —C₃-C₆cycloalkyl is optionally substituted with at least one substituent selected from halogen, —C₁₋₄alkoxy, —CN, —OH, —NH₂ or oxo; or
two geminal R⁵ and the carbon atom which they attach to form a 3- to 5-membered carbocyclic ring; wherein said ring is optionally substituted with at least one substituent selected from halogen, —C₁₋₄alkoxy, —CN, —OH, —NH₂ or oxo;
n is 0, 1, 2, 3 or 4;
r is 0 or 1.

Aspect A2. The compound of Aspect 1, wherein the compound is selected from formula (AIIa), (AIIb), (AIIc) or (AIId):

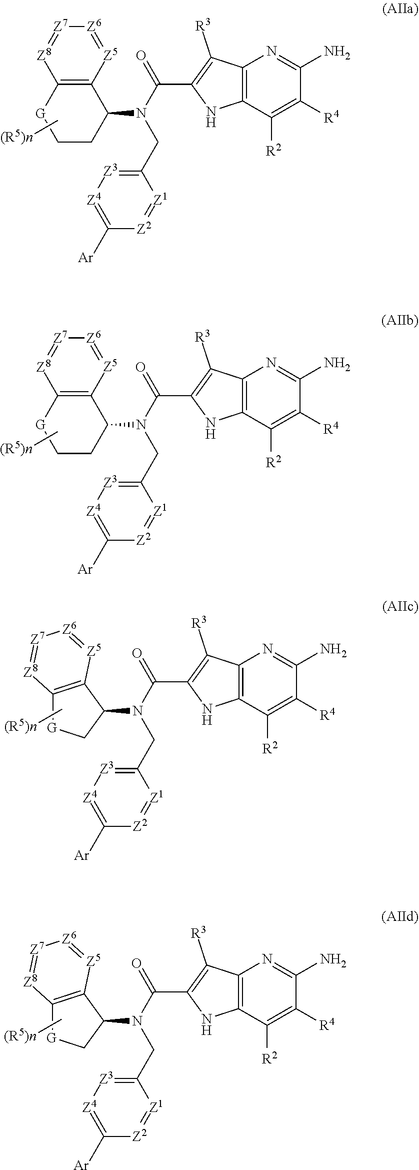

wherein, R², R³, R⁴, R⁵, Z¹, Z², Z³, Z⁴, Z³, Z⁶, Z⁷, Z⁸, Ar, G and n are as defined as Aspect 1.

In some embodiments, the compound is selected from formula (AIIe), (AIIf), (AIIg), (AIIh), (AIIi), (AIIj), (AIIk), (AIIl), (AIIm) or (AIIn):

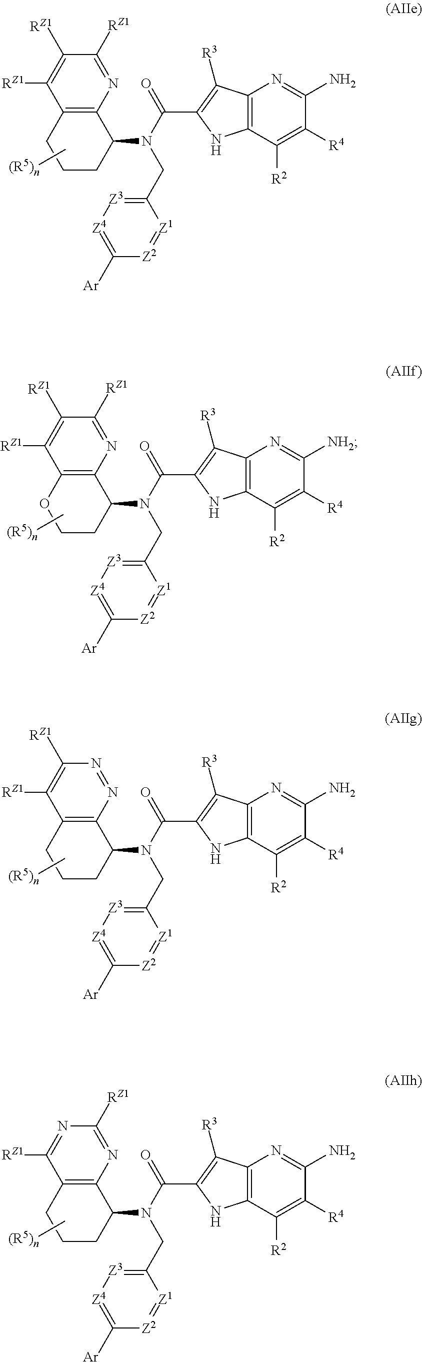

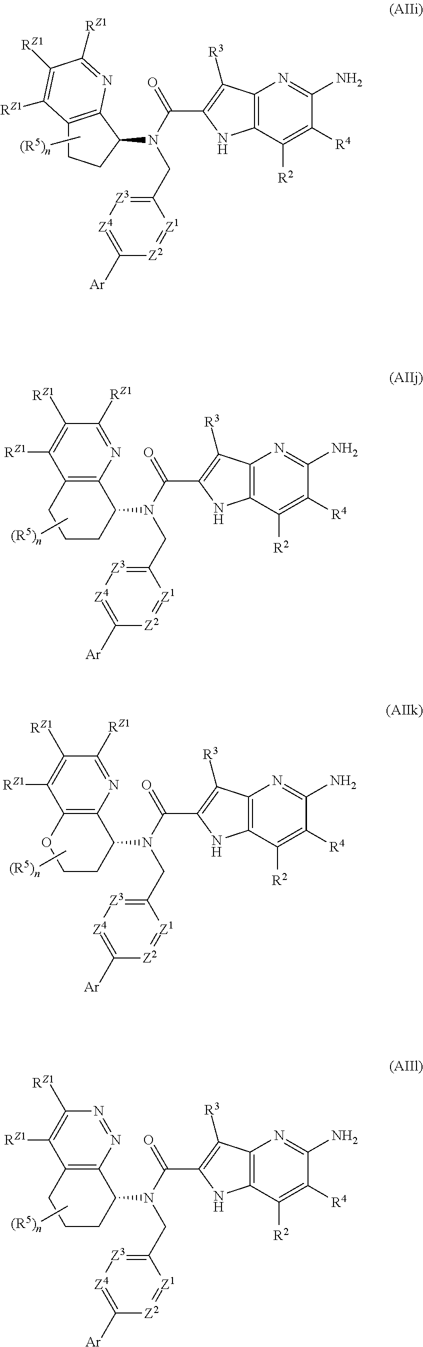

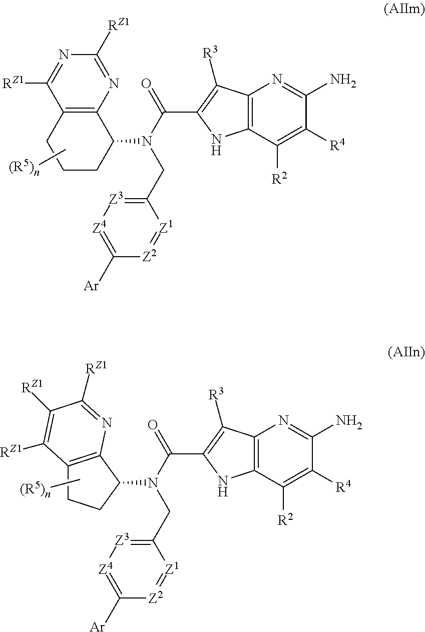

wherein, R^{Z1}, R², R³, R⁴, R⁵, Z¹, Z², Z³, Z⁴, Ar and n are as defined as Aspect 1.

Aspect A3. The compound of Aspect 1, wherein the compound is formula (AIIIa), (AIIIb), (AIIIc), (AIIId), (AIIIe), (AIIIf) or (AIIIg):

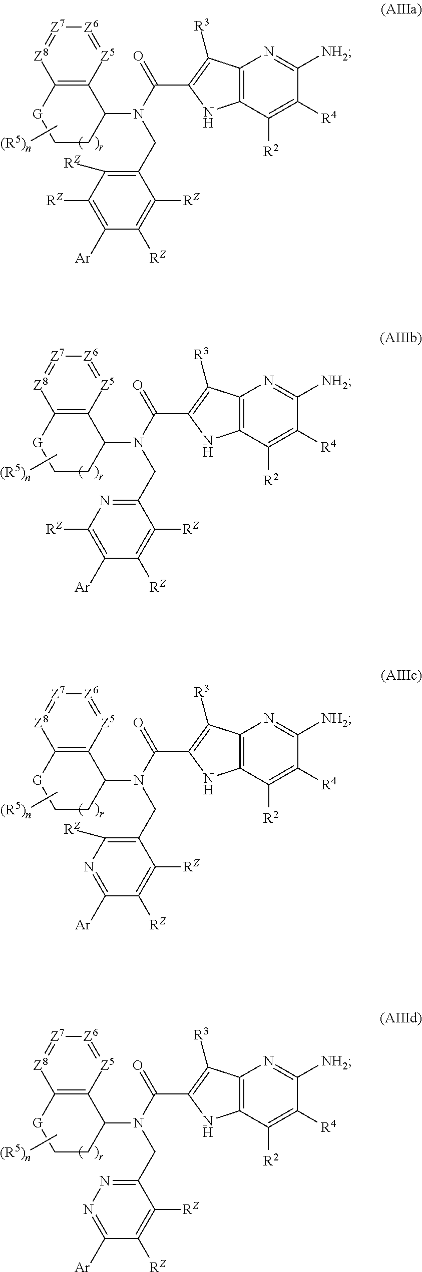

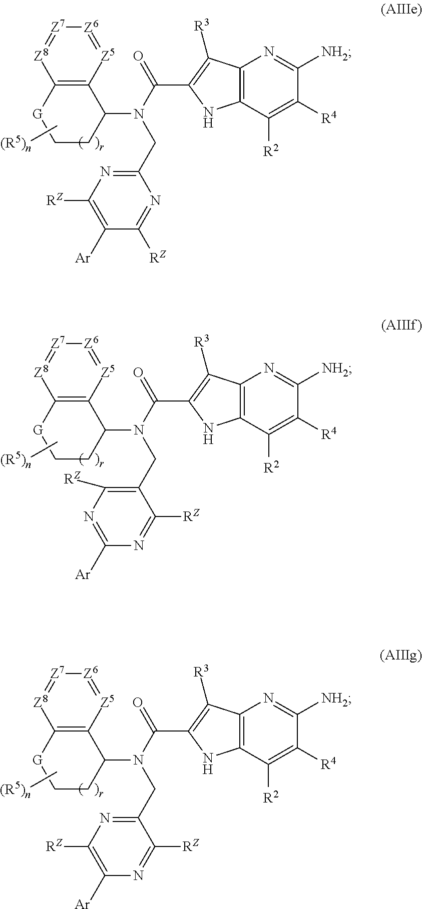

wherein, R², R³, R⁴, R⁵, R^{Z}, Z³, Z⁶, Z⁷, Z⁸, G, n and r are as defined as Aspect 1.

Aspect A4. The compound of Aspect 1, wherein the compound is formula (AIVa), (AIVb), (AIVc):

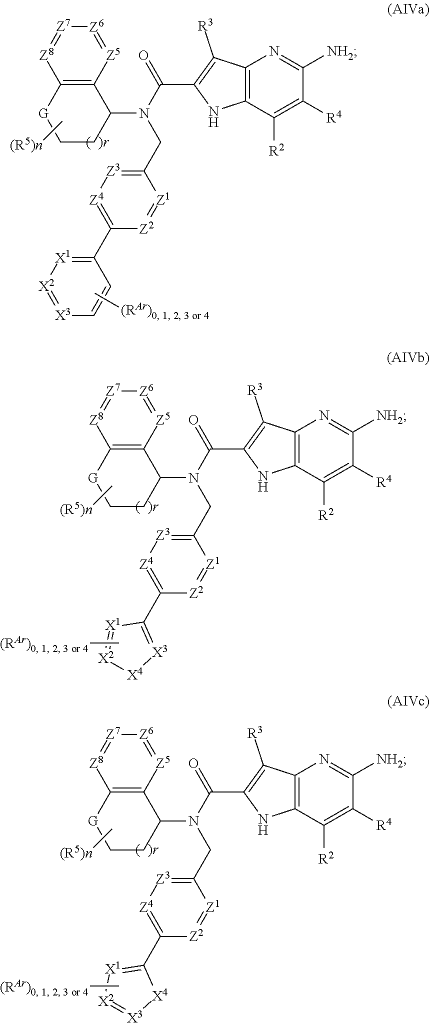

wherein, X¹, X² and X³ are each independently selected from N or CH, provided that when anyone of X¹, X² or X³ is CH, CH is optionally substituted with R^{Ar};
X⁴ is each independently selected from N, O or S;
R², R³, R⁴, R⁵, R^{Ar}, Z¹, Z², Z³, Z⁴, Z⁵, Z⁶, Z⁷, Z⁸, G and n and r are as defined as Aspect 1.

Aspect A5. The compound of any anyone of preceding Aspects, wherein R^{Z1} is selected from hydrogen, —F, —Cl, —Br, —I, methyl, ethyl, propyl (iso-propyl or n-propyl), butyl (n-butyl, sec-butyl, iso-butyl or tert-butyl), methoxy, ethoxy, propoxy, butoxy, cyclopropyl, cyclobutyl, cyclopentyl, cyclohexyl, cyclopropoxy, cyclobutoxy, cyclopentoxy or cyclohexoxy; wherein each of said methyl, ethyl, propyl (iso-propyl or n-propyl), butyl (n-butyl, sec-butyl, iso-butyl or tert-butyl), methoxy, ethoxy, propoxy, butoxy, cyclopropyl, cyclobutyl, cyclopentyl, cyclohexyl, cyclopropoxy, cyclobutoxy, cyclopentoxy or cyclohexoxy is optionally substituted with at least one substituent selected from —F, —Cl, —Br, —I, methoxy, ethoxy, propoxy, butoxy, —CN, —OH, —NH₂ or oxo.

Aspect A6. The compound of any anyone of preceding Aspects, wherein R^{Z1} is selected from hydrogen, —F, —Cl, —Br, —I, methyl, ethyl, propyl (iso-propyl or n-propyl), butyl (n-butyl, sec-butyl, iso-butyl or tert-butyl), methoxy, ethoxy, propoxy, butoxy, cyclopropyl, cyclobutyl, cyclopentyl, cyclohexyl, cyclopropoxy, cyclobutoxy, cyclopentoxy, cyclohexoxy, hydroxymethyl, hydroxyethyl, hydroxypropyl, hydroxybutyl.

In some embodiments, R^{Z1} is selected from hydrogen, —F, —Cl, —Br, —I, methyl, ethyl, propyl (iso-propyl or n-propyl), methoxy, ethoxy, cyclopropyl, cyclobutyl, cyclopropoxy or hydroxypropyl

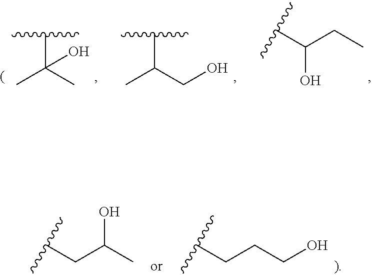

Aspect A7. The compound of anyone of preceding Aspects, wherein R² is selected from hydrogen, —F, —Cl, —Br, —I, methyl, ethyl, propyl (iso-propyl or n-propyl), butyl (n-butyl, sec-butyl, iso-butyl or tert-butyl), methoxy, ethoxy, propoxy, butoxy, cyclopropyl, cyclobutyl, cyclopentyl, cyclohexyl, cyclopropoxy, cyclobutoxy, cyclopentoxy, cyclohexoxy or —CN; wherein methyl, ethyl, propyl (iso-propyl or n-propyl), butyl (n-butyl, sec-butyl, iso-butyl or tert-butyl), methoxy, ethoxy, propoxy, butoxy, cyclopropyl, cyclobutyl, cyclopentyl, cyclohexyl, cyclopropoxy, cyclobutoxy, cyclopentoxy or cyclohexoxy is optionally substituted with at least one substituent selected from —F, —Cl, —Br, —I, methoxy, ethoxy, propoxy, butoxy, cyclopropoxy, cyclobutoxy, cyclopentoxy, cyclohexoxy;

In some embodiments, R² is selected from hydrogen, methyl, ethyl, cyclopropyl, cyclobutyl, cyclopropoxy, cyclobutoxy or —CN; wherein methyl, ethyl, is optionally substituted with at least one substituent selected from —F, —Cl, —Br, —I, methoxy, ethoxy, propoxy, cyclopropoxy or cyclobutoxy.

In some embodiments, R² is selected from hydrogen, —F, —Cl, —Br, —I, methyl, ethyl, propyl (iso-propyl or n-propyl), butyl (n-butyl, sec-butyl, iso-butyl or tert-butyl), methoxy, ethoxy, propoxy, butoxy, cyclopropyl, cyclobutyl, cyclopentyl, cyclohexyl, cyclopropoxy, cyclobutoxy, cyclopentoxy, cyclohexoxy or —CN.

Aspect A8. The compound of anyone of preceding Aspects, wherein R² is selected from hydrogen, methyl, ethyl or —CN.

Aspect A9. The compound of anyone of preceding Aspects, wherein R³ is selected from hydrogen, —F, —Cl, —Br, —I, methyl, ethyl, propyl (iso-propyl or n-propyl), butyl (n-butyl, sec-butyl, iso-butyl or tert-butyl) or —CN, wherein methyl, ethyl, propyl (iso-propyl or n-propyl) or butyl (n-butyl, sec-butyl, iso-butyl or tert-butyl) is optionally substituted with at least one substituent selected from hydrogen, —F, —Cl, —Br, —I, methoxy, ethoxy, propoxy, butoxy, cyclopropyl, cyclobutyl, cyclopentyl, cyclohexyl, —CN, —OH, —NH₂ or oxo.

In some embodiments, R³ is selected from hydrogen, —F, —Cl, —Br, —I, methyl, ethyl, propyl (iso-propyl or n-propyl), butyl (n-butyl, sec-butyl, iso-butyl or tert-butyl) or —CN.

Aspect A10. The compound of anyone of preceding Aspects, wherein R³ is selected from hydrogen, —F, —Cl, —Br, —I or —CN.

Aspect A11. The compound of anyone of preceding Aspects, wherein R⁴ is selected from hydrogen, —F, —Cl, —Br, —I, methyl, ethyl, propyl (iso-propyl or n-propyl), butyl (n-butyl, sec-butyl, iso-butyl or tert-butyl), methoxy, ethoxy, propoxy, butoxy, cyclopropyl, cyclobutyl, cyclopentyl, cyclohexyl, cyclopropoxy, cyclobutoxy, cyclopentoxy or cyclohexoxy, wherein methyl, ethyl, propyl (iso-propyl or n-propyl) or butyl (n-butyl, sec-butyl, iso-butyl or tert-butyl), methoxy, ethoxy, propoxy, butoxy, cyclopropyl, cyclobutyl, cyclopentyl, cyclohexyl, cyclopropoxy, cyclobutoxy, cyclopentoxy or cyclohexoxy is optionally substituted with at least one substituent selected from —F, —Cl, —Br, —I, methoxy, ethoxy, propoxy, butoxy, cyclopropoxy, cyclobutoxy, cyclopentoxy, cyclohexoxy, —CN, —OH, —NH₂ or oxo.

In some embodiments, R⁴ is selected from hydrogen, —F, —Cl, —Br, methyl, ethyl, propyl (iso-propyl or n-propyl), methoxy, ethoxy, propoxy, cyclopropyl, cyclobutyl, cyclopropoxy or cyclobutoxy, wherein methyl, ethyl, propyl (iso-propyl or n-propyl), methoxy, ethoxy, propoxy, cyclopropyl, cyclobutyl, cyclopropoxy or cyclobutoxy is optionally substituted with at least one substituent selected from —F, —Cl, —Br, —I, methoxy, ethoxy, propoxy, cyclopropoxy, cyclobutoxy, —CN, —OH, —NH₂ or oxo.

In some embodiments, R⁴ is selected from hydrogen, —F, —Cl, —Br, —I, methyl, ethyl, propyl (iso-propyl or n-propyl) or butyl (n-butyl, sec-butyl, iso-butyl or tert-butyl).

Aspect A12. The compound of anyone of preceding Aspects, wherein R⁴ is selected from hydrogen, —F, —Cl, —Br, —I, methyl, ethyl.

Aspect A13. The compound of anyone of preceding Aspects, wherein R⁵ is selected from hydrogen, —F, —Cl, —Br, —I, methyl, ethyl, propyl (iso-propyl or n-propyl), butyl (n-butyl, sec-butyl, iso-butyl or tert-butyl), cyclopropyl, cyclobutyl, cyclopentyl or cyclohexyl, wherein methyl, ethyl, propyl (iso-propyl or n-propyl), butyl (n-butyl, sec-butyl, iso-butyl or tert-butyl), cyclopropyl, cyclobutyl, cyclopentyl or cyclohexyl is optionally substituted with at least one substituent selected from —F, —Cl, —Br, —I, methoxy, ethoxy, propoxy, butoxy, —CN, —OH, —NH₂ or oxo.

In some embodiments, R⁵ is selected from hydrogen, —F, —Cl, —Br, —I, methyl, ethyl, propyl (iso-propyl or n-propyl) or butyl (n-butyl, sec-butyl, iso-butyl or tert-butyl).

In some embodiments, R⁵ is selected from hydrogen, —F, —Cl, —Br, methyl, ethyl.

Aspect A14. The compound of anyone of preceding Aspects, wherein two geminal R⁵ and the carbon atom which they attach to form a 3-, 4- or 5-membered carbocyclic ring; wherein said ring is optionally substituted with at least one substituent selected from —F, —Cl, —Br, —I, methoxy, ethoxy, propoxy, butoxy, —CN, —OH, —NH₂ or oxo.

In some embodiments, two geminal R⁵ and the carbon atom which they attach to form a 3-, 4- or 5-membered carbocyclic ring.

In some embodiments, two geminal R⁵ and the carbon atom which they attach to form a 3-membered carbocyclic ring.

Aspect A15. The compound of anyone of preceding Aspects, wherein at most two of Z¹, Z², Z³ and Z⁴ are N; R^{Z} is each independently selected from hydrogen, —F, —Cl, —Br, —I, methyl, ethyl, propyl (iso-propyl or n-propyl) or butyl (n-butyl, sec-butyl, iso-butyl or tert-butyl), methoxy, ethoxy, propoxy or butoxy; wherein each of said ethyl, propyl (iso-propyl or n-propyl) or butyl (n-butyl, sec-butyl, iso-butyl or tert-butyl), methoxy, ethoxy, propoxy or butoxy is optionally substituted with at least one substituent selected from —F, —Cl, —Br, —I.

In some embodiments, R^{Z} is each independently selected from hydrogen, —F, —Cl, —Br, —I, methyl, ethyl, propyl (iso-propyl or n-propyl), butyl (n-butyl, sec-butyl, iso-butyl or tert-butyl), methoxy, ethoxy, propoxy or butoxy.

In some embodiments, R^{Z} is each independently selected from hydrogen, —F, —Cl, —Br, methyl or ethyl.

Aspect A16. The compound of anyone of preceding Aspects, wherein Ar is independently selected from phenyl, pyridinyl, pyridazinyl, pyrimidinyl, pyrazinyl, pyrrolyl, pyrazolyl, imidazolyl, 1,2,4-triazolyl, 1,2,3-triazolyl, oxazolyl, furanyl, thiazolyl or thiophenyl; each of said phenyl, pyridinyl, pyridazinyl, pyrimidinyl, pyrazinyl, pyrrolyl, pyrazolyl, imidazolyl, 1,2,4-triazolyl, 1,2,3-triazolyl, oxazolyl, furanyl, thiazolyl or thiophenyl is optionally substituted with at least one substituent R^{Ar};
R^{Ar} is each independently selected from hydrogen, —F, —Cl, —Br, —I, methyl, ethyl, propyl (iso-propyl or n-propyl), butyl (n-butyl, sec-butyl, iso-butyl or tert-butyl), methoxy, ethoxy, propoxy, butoxy, cyclopropyl, cyclobutyl, cyclopentyl, cyclohexyl, cyclopropoxy, cyclobutoxy, cyclopentoxy, cyclohexoxy or —CN; wherein each of said methyl, ethyl, propyl (iso-propyl or n-propyl), butyl (n-butyl, sec-butyl, iso-butyl or tert-butyl), methoxy, ethoxy, propoxy, butoxy, cyclopropyl, cyclobutyl, cyclopentyl, cyclohexyl, cyclopropoxy, cyclobutoxy, cyclopentoxy, cyclohexoxy is optionally substituted with at least one substituent selected from —F, —Cl, —Br or —I;

Aspect A17. The compound of anyone of preceding Aspects, wherein Ar is independently selected from phenyl, pyridinyl, pyrimidinyl, pyrrolyl, pyrazolyl or 1,2,4-triazolyl; each of said phenyl, pyridinyl, pyrimidinyl, pyrrolyl, pyrazolyl or 1,2,4-triazolyl is optionally substituted with at least one substituent R^{Ar};
R^{Ar} is each independently selected from hydrogen, —F, —Cl, —Br, —I, methyl, ethyl, propyl (iso-propyl or n-propyl), butyl (n-butyl, sec-butyl, iso-butyl or tert-butyl), methoxy, ethoxy, propoxy, butoxy, cyclopropyl, cyclobutyl, cyclopentyl, cyclohexyl, cyclopropoxy, cyclobutoxy, cyclopentoxy, cyclohexoxy, —CF₃ or —CN.

In some embodiments, Ar is independently selected from phenyl, pyridinyl, pyrimidinyl, pyrrolyl, pyrazolyl or 1,2,4-triazolyl; each of said phenyl, pyridinyl, pyrimidinyl, pyrrolyl, pyrazolyl or 1,2,4-triazolyl is optionally substituted with at least one substituent R^{Ar};
R^{Ar} is each independently selected from hydrogen, —F, —Cl, —Br, methyl, ethyl, methoxy, ethoxy, —CF₃ or —CN.

Aspect A18. The compound of anyone of preceding Aspects, wherein G is selected from CH₂, O, NH.

In some embodiments, G is selected from CH₂ or O.

Aspect A19. The compound of any one of the preceding Aspects, wherein the compound is selected from

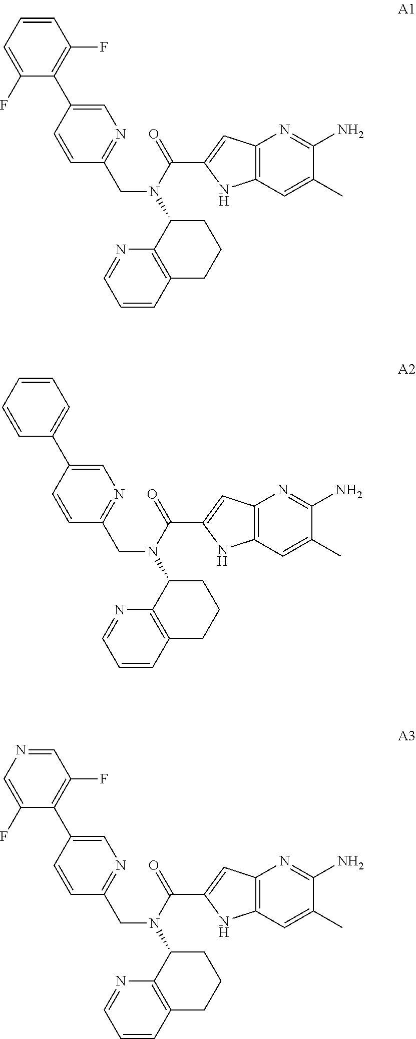 [SMILES: Cc1cc2[nH]c(C(=O)N(Cc3ccc(-c4c(F)cccc4F)cn3)[C@@H]3CCCc4cccnc43)cc2nc1N ; Cc1cc2[nH]c(C(=O)N(Cc3ccc(-c4ccccc4)cn3)[C@@H]3CCCc4cccnc43)cc2nc1N ; Cc1cc2[nH]c(C(=O)N(Cc3ccc(-c4c(F)cncc4F)cn3)[C@@H]3CCCc4cccnc43)cc2nc1N]

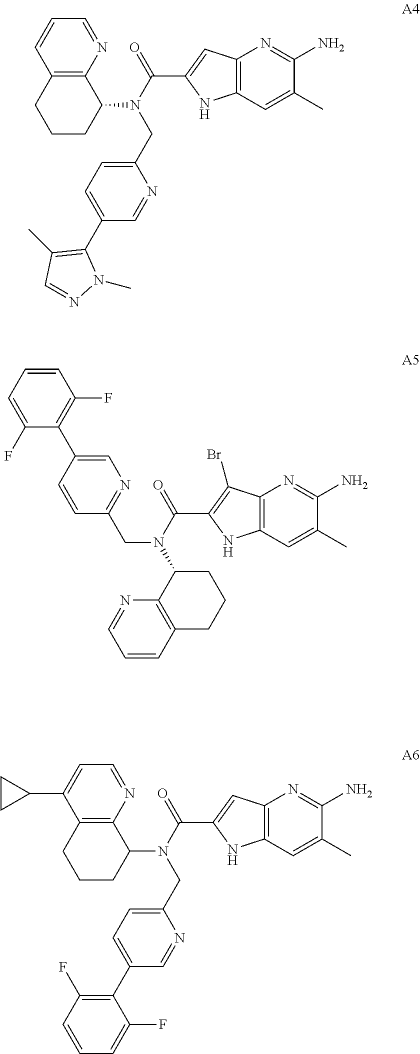

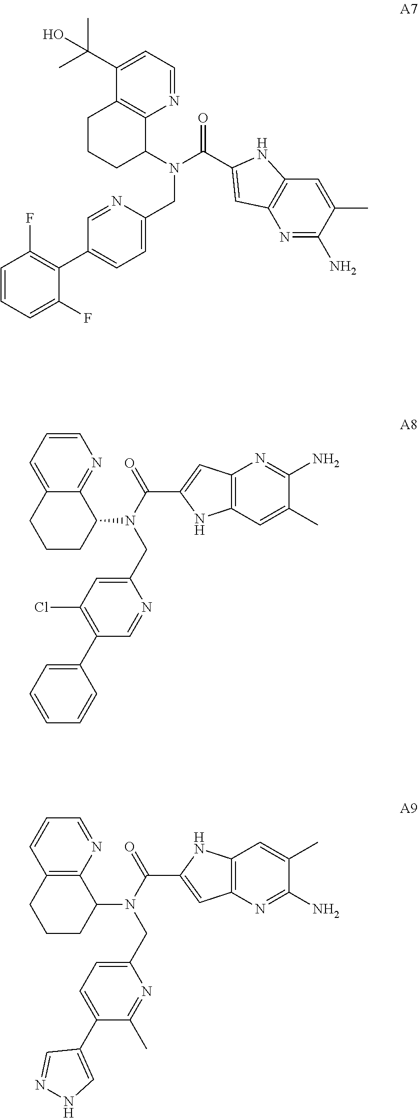 [SMILES: Cc1cc2[nH]c(C(=O)N(Cc3ccc(-c4c(F)cccc4F)cn3)C3CCCc4c(C(C)(C)O)ccnc43)cc2nc1N ; Cc1cc2[nH]c(C(=O)N(Cc3cc(Cl)c(-c4ccccc4)cn3)[C@@H]3CCCc4cccnc43)cc2nc1N ; Cc1cc2[nH]c(C(=O)N(Cc3ccc(-c4cn[nH]c4)c(C)n3)C3CCCc4cccnc43)cc2nc1N]

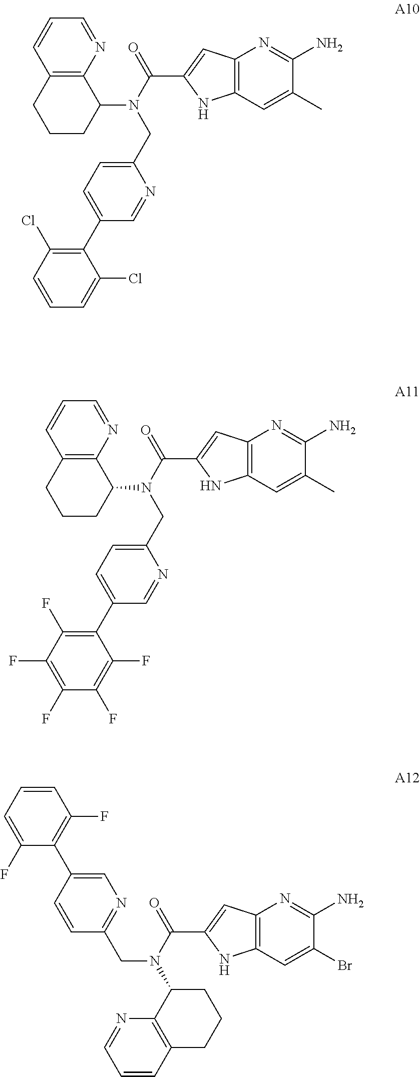 [SMILES: Cc1cc2[nH]c(C(=O)N(Cc3ccc(-c4c(Cl)cccc4Cl)cn3)C3CCCc4cccnc43)cc2nc1N ; Cc1cc2[nH]c(C(=O)N(Cc3ccc(-c4c(F)c(F)c(F)c(F)c4F)cn3)[C@@H]3CCCc4cccnc43)cc2nc1N ; Nc1nc2cc(C(=O)N(Cc3ccc(-c4c(F)cccc4F)cn3)[C@@H]3CCCc4cccnc43)[nH]c2cc1Br]

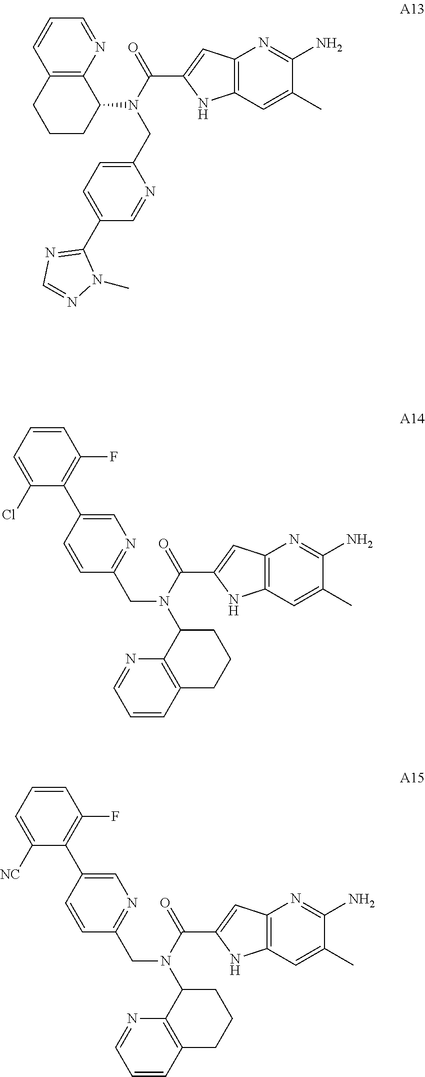 [SMILES: Cc1cc2[nH]c(C(=O)N(Cc3ccc(-c4ncnn4C)cn3)[C@@H]3CCCc4cccnc43)cc2nc1N ; Cc1cc2[nH]c(C(=O)N(Cc3ccc(-c4c(F)cccc4Cl)cn3)C3CCCc4cccnc43)cc2nc1N ; Cc1cc2[nH]c(C(=O)N(Cc3ccc(-c4c(F)cccc4C#N)cn3)C3CCCc4cccnc43)cc2nc1N]

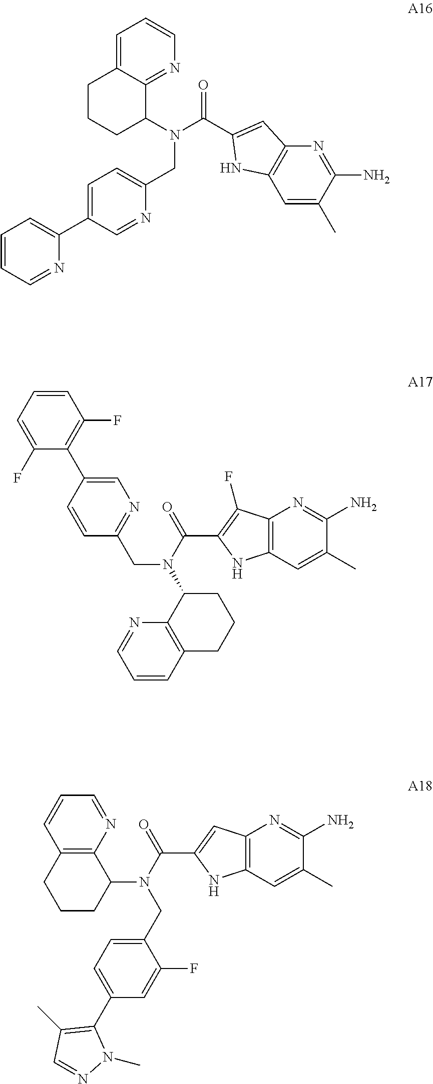

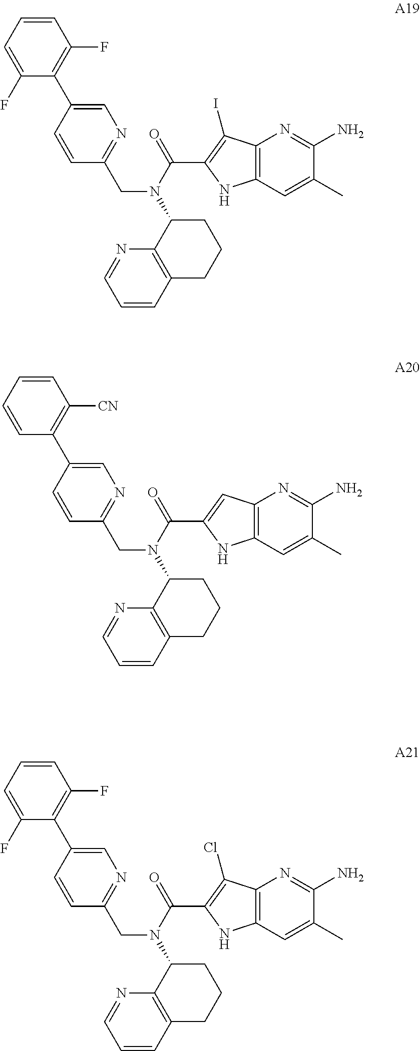 [SMILES: Cc1cc2[nH]c(C(=O)N(Cc3ccc(-c4c(F)cccc4F)cn3)[C@@H]3CCCc4cccnc43)c(I)c2nc1N ; Cc1cc2[nH]c(C(=O)N(Cc3ccc(-c4ccccc4C#N)cn3)[C@@H]3CCCc4cccnc43)cc2nc1N ; Cc1cc2[nH]c(C(=O)N(Cc3ccc(-c4c(F)cccc4F)cn3)[C@@H]3CCCc4cccnc43)c(Cl)c2nc1N]

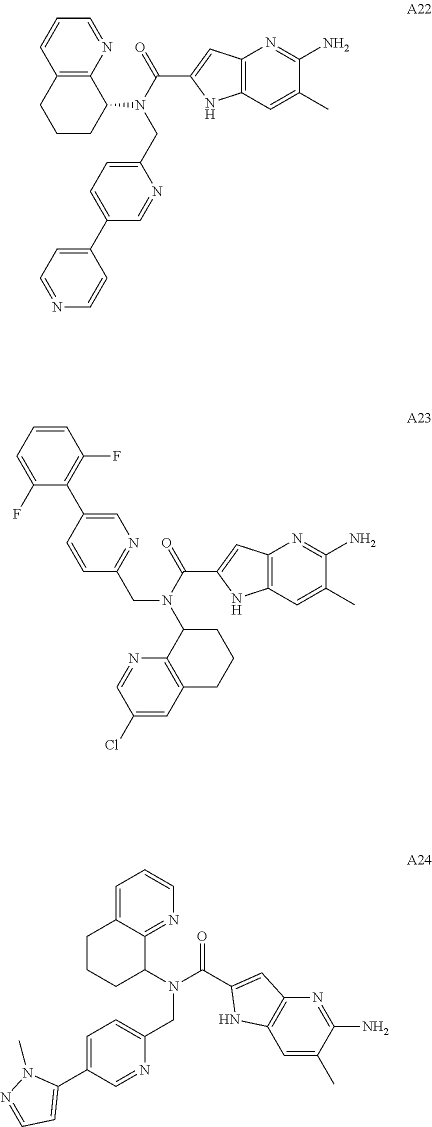 [SMILES: Cc1cc2[nH]c(C(=O)N(Cc3ccc(-c4ccncc4)cn3)[C@@H]3CCCc4cccnc43)cc2nc1N ; Cc1cc2[nH]c(C(=O)N(Cc3ccc(-c4c(F)cccc4F)cn3)C3CCCc4cc(Cl)cnc43)cc2nc1N ; Cc1cc2[nH]c(C(=O)N(Cc3ccc(-c4ccnn4C)cn3)C3CCCc4cccnc43)cc2nc1N]

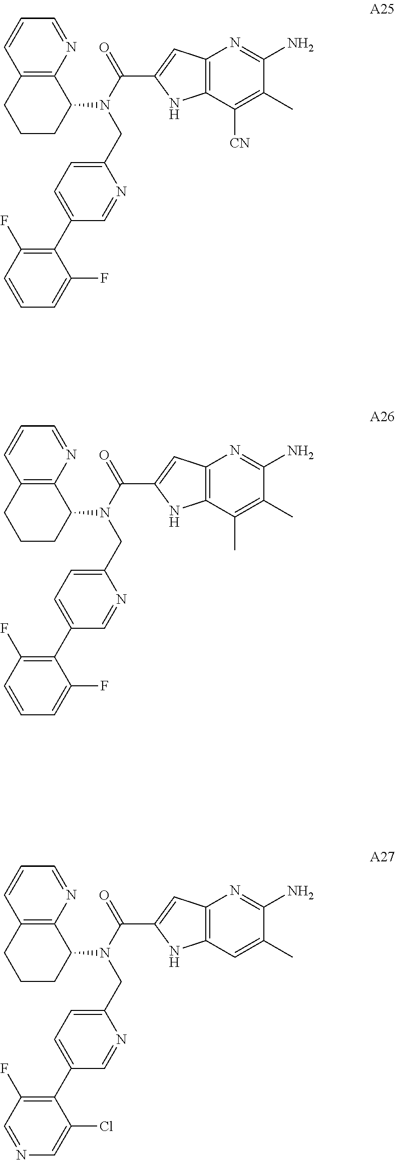 [SMILES: Cc1c(N)nc2cc(C(=O)N(Cc3ccc(-c4c(F)cccc4F)cn3)[C@@H]3CCCc4cccnc43)[nH]c2c1C#N ; Cc1c(N)nc2cc(C(=O)N(Cc3ccc(-c4c(F)cccc4F)cn3)[C@@H]3CCCc4cccnc43)[nH]c2c1C ; Cc1cc2[nH]c(C(=O)N(Cc3ccc(-c4c(F)cncc4Cl)cn3)[C@@H]3CCCc4cccnc43)cc2nc1N]

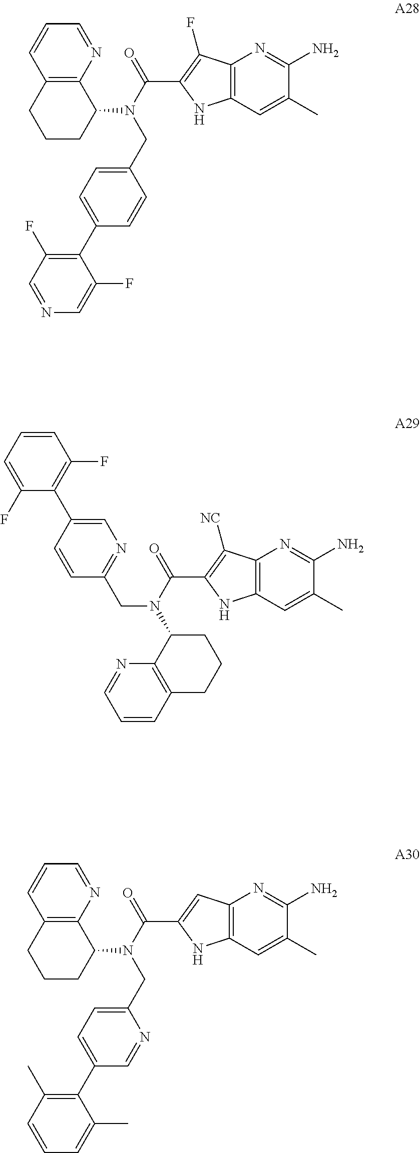 [SMILES: Cc1cc2[nH]c(C(=O)N(Cc3ccc(-c4c(F)cncc4F)cc3)[C@@H]3CCCc4cccnc43)c(F)c2nc1N ; Cc1cc2[nH]c(C(=O)N(Cc3ccc(-c4c(F)cccc4F)cn3)[C@@H]3CCCc4cccnc43)c(C#N)c2nc1N ; Cc1cc2[nH]c(C(=O)N(Cc3ccc(-c4c(C)cccc4C)cn3)[C@@H]3CCCc4cccnc43)cc2nc1N]

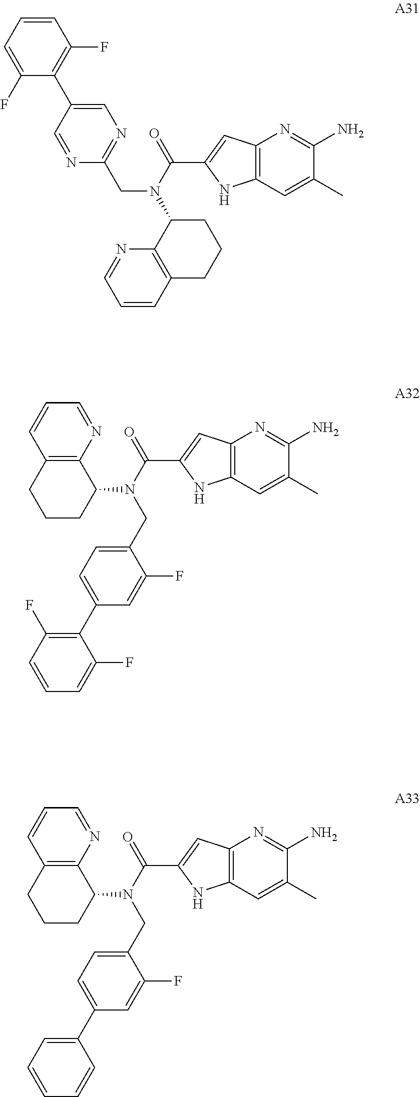 [SMILES: Cc1cc2[nH]c(C(=O)N(Cc3ncc(-c4c(F)cccc4F)cn3)[C@@H]3CCCc4cccnc43)cc2nc1N ; Cc1cc2[nH]c(C(=O)N(Cc3ccc(-c4c(F)cccc4F)cc3F)[C@@H]3CCCc4cccnc43)cc2nc1N ; Cc1cc2[nH]c(C(=O)N(Cc3ccc(-c4ccccc4)cc3F)[C@@H]3CCCc4cccnc43)cc2nc1N]

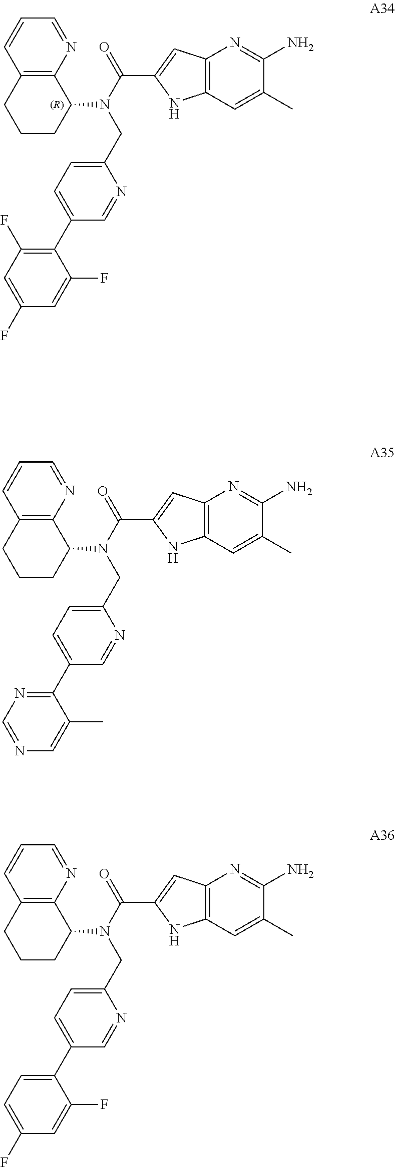

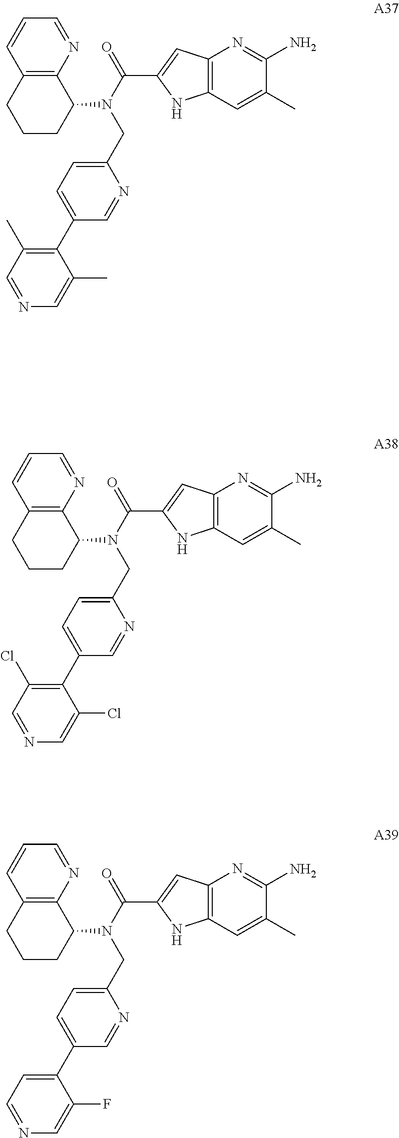 [SMILES: Cc1cc2[nH]c(C(=O)N(Cc3ccc(-c4c(C)cncc4C)cn3)[C@@H]3CCCc4cccnc43)cc2nc1N ; Cc1cc2[nH]c(C(=O)N(Cc3ccc(-c4c(Cl)cncc4Cl)cn3)[C@@H]3CCCc4cccnc43)cc2nc1N ; Cc1cc2[nH]c(C(=O)N(Cc3ccc(-c4ccncc4F)cn3)[C@@H]3CCCc4cccnc43)cc2nc1N]

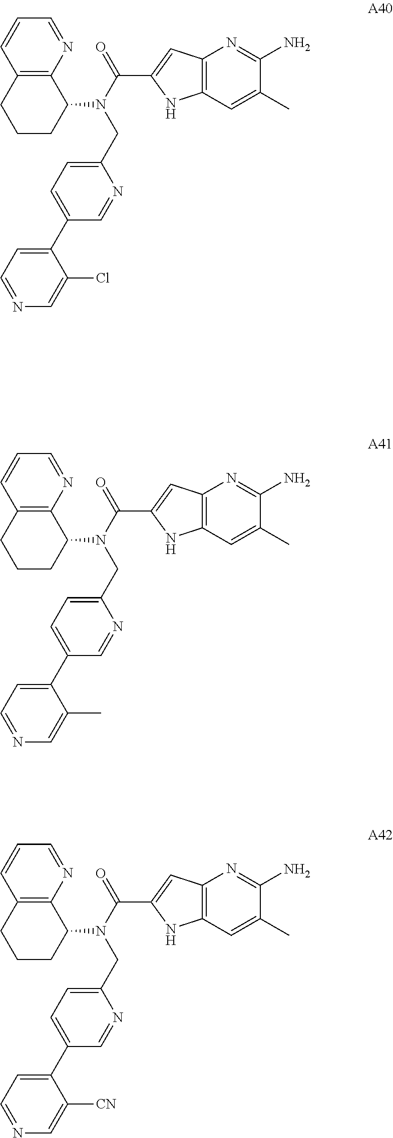 [SMILES: Cc1cc2[nH]c(C(=O)N(Cc3ccc(-c4ccncc4Cl)cn3)[C@@H]3CCCc4cccnc43)cc2nc1N ; Cc1cnccc1-c1ccc(CN(C(=O)c2cc3nc(N)c(C)cc3[nH]2)[C@@H]2CCCc3cccnc32)nc1 ; Cc1cc2[nH]c(C(=O)N(Cc3ccc(-c4ccncc4C#N)cn3)[C@@H]3CCCc4cccnc43)cc2nc1N]

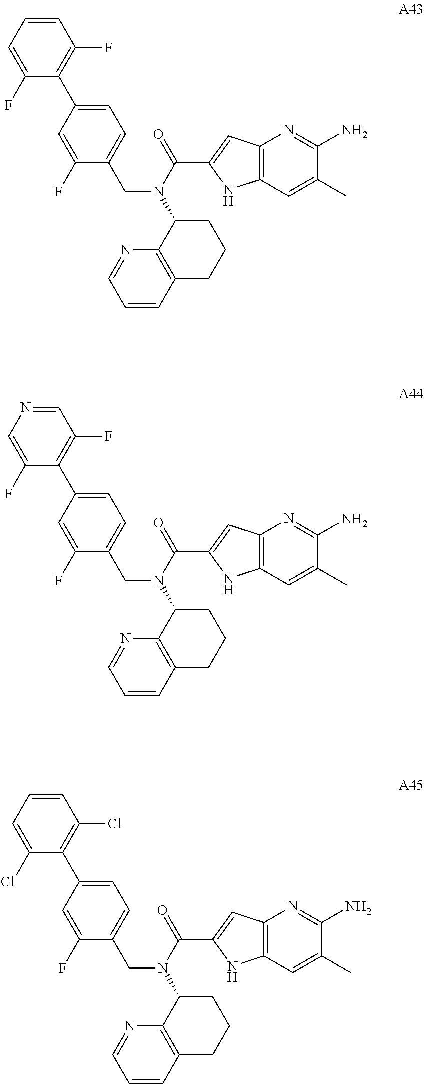 [SMILES: Cc1cc2[nH]c(C(=O)N(Cc3ccc(-c4c(F)cccc4F)cc3F)[C@@H]3CCCc4cccnc43)cc2nc1N ; Cc1cc2[nH]c(C(=O)N(Cc3ccc(-c4c(F)cncc4F)cc3F)[C@@H]3CCCc4cccnc43)cc2nc1N ; Cc1cc2[nH]c(C(=O)N(Cc3ccc(-c4c(Cl)cccc4Cl)cc3F)[C@@H]3CCCc4cccnc43)cc2nc1N]

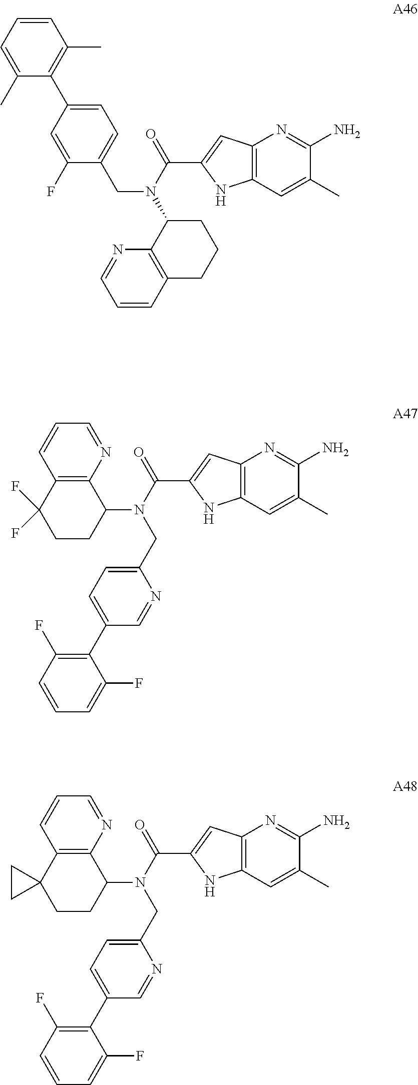 [SMILES: Cc1cc2[nH]c(C(=O)N(Cc3ccc(-c4c(C)cccc4C)cc3F)[C@@H]3CCCc4cccnc43)cc2nc1N ; Cc1cc2[nH]c(C(=O)N(Cc3ccc(-c4c(F)cccc4F)cn3)C3CCC(F)(F)c4cccnc43)cc2nc1N ; Cc1cc2[nH]c(C(=O)N(Cc3ccc(-c4c(F)cccc4F)cn3)C3CCC4(CC4)c4cccnc43)cc2nc1N]

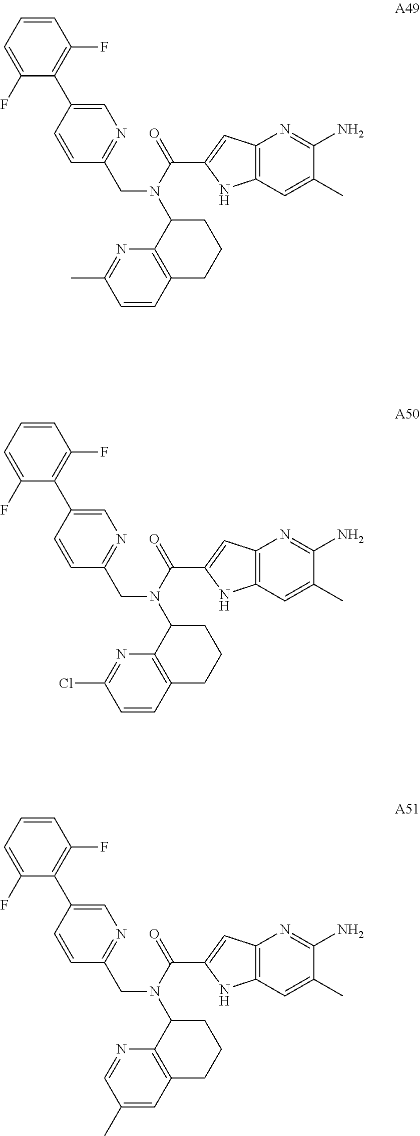 [SMILES: Cc1ccc2c(n1)C(N(Cc1ccc(-c3c(F)cccc3F)cn1)C(=O)c1cc3nc(N)c(C)cc3[nH]1)CCC2 ; Cc1cc2[nH]c(C(=O)N(Cc3ccc(-c4c(F)cccc4F)cn3)C3CCCc4ccc(Cl)nc43)cc2nc1N ; Cc1cnc2c(c1)CCCC2N(Cc1ccc(-c2c(F)cccc2F)cn1)C(=O)c1cc2nc(N)c(C)cc2[nH]1]

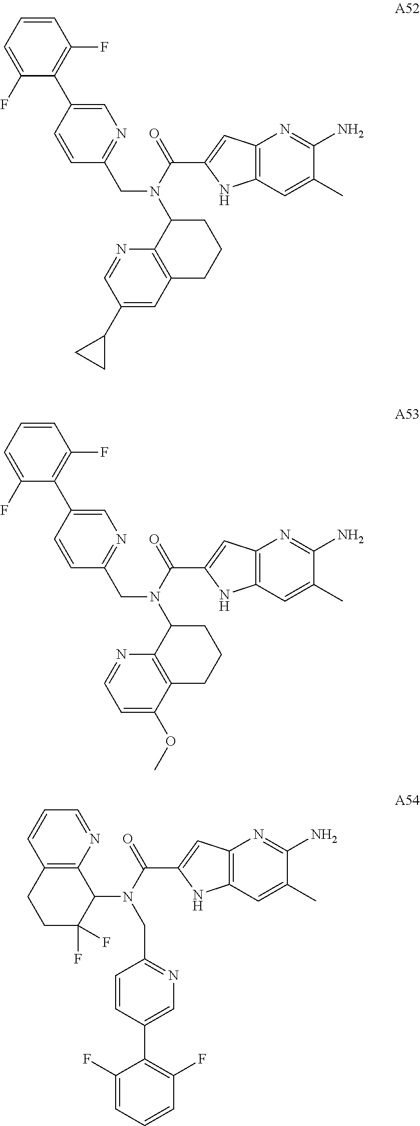 [SMILES: Cc1cc2[nH]c(C(=O)N(Cc3ccc(-c4c(F)cccc4F)cn3)C3CCCc4cc(C5CC5)cnc43)cc2nc1N ; COc1ccnc2c1CCCC2N(Cc1ccc(-c2c(F)cccc2F)cn1)C(=O)c1cc2nc(N)c(C)cc2[nH]1 ; Cc1cc2[nH]c(C(=O)N(Cc3ccc(-c4c(F)cccc4F)cn3)C3c4ncccc4CCC3(F)F)cc2nc1N]

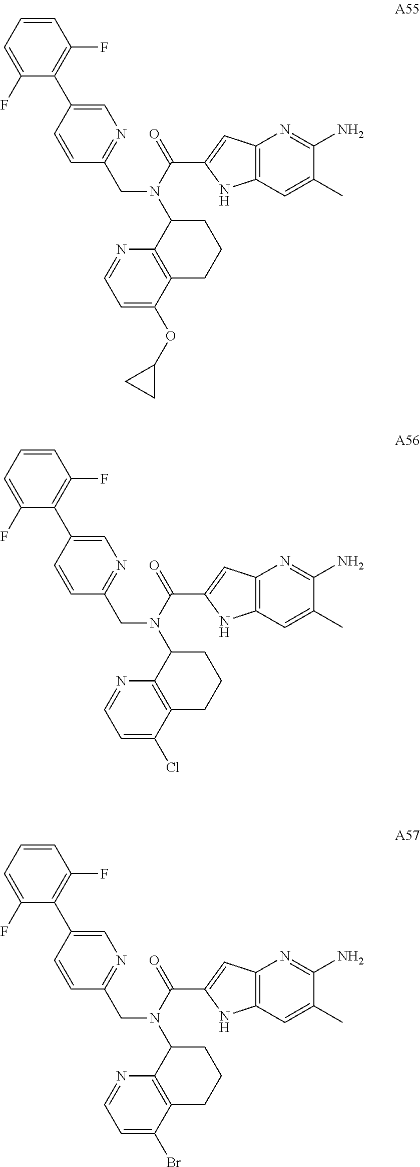 [SMILES: Cc1cc2[nH]c(C(=O)N(Cc3ccc(-c4c(F)cccc4F)cn3)C3CCCc4c(OC5CC5)ccnc43)cc2nc1N ; Cc1cc2[nH]c(C(=O)N(Cc3ccc(-c4c(F)cccc4F)cn3)C3CCCc4c(Cl)ccnc43)cc2nc1N ; Cc1cc2[nH]c(C(=O)N(Cc3ccc(-c4c(F)cccc4F)cn3)C3CCCc4c(Br)ccnc43)cc2nc1N]

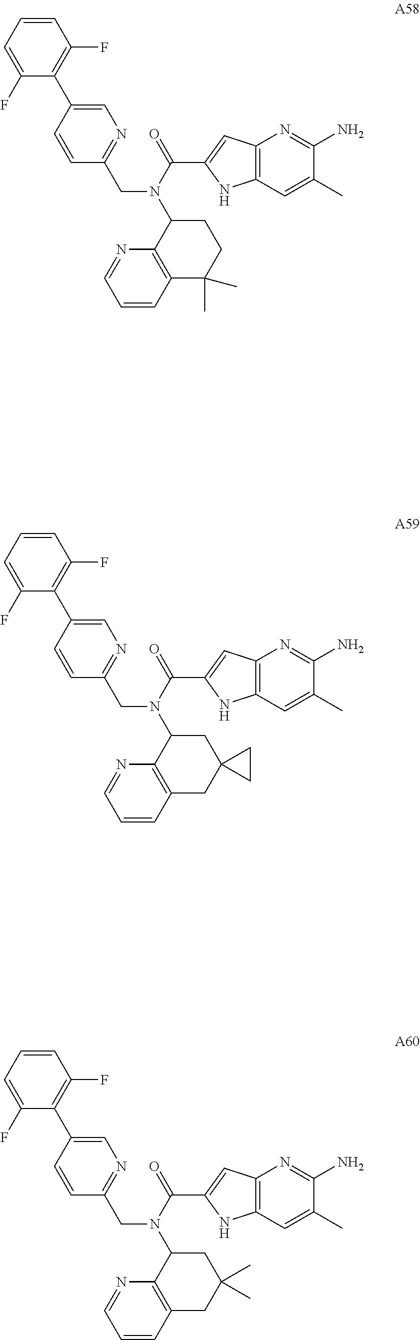 [SMILES: Cc1cc2[nH]c(C(=O)N(Cc3ccc(-c4c(F)cccc4F)cn3)C3CCC(C)(C)c4cccnc43)cc2nc1N ; Cc1cc2[nH]c(C(=O)N(Cc3ccc(-c4c(F)cccc4F)cn3)C3CC4(CC4)Cc4cccnc43)cc2nc1N ; Cc1cc2[nH]c(C(=O)N(Cc3ccc(-c4c(F)cccc4F)cn3)C3CC(C)(C)Cc4cccnc43)cc2nc1N]

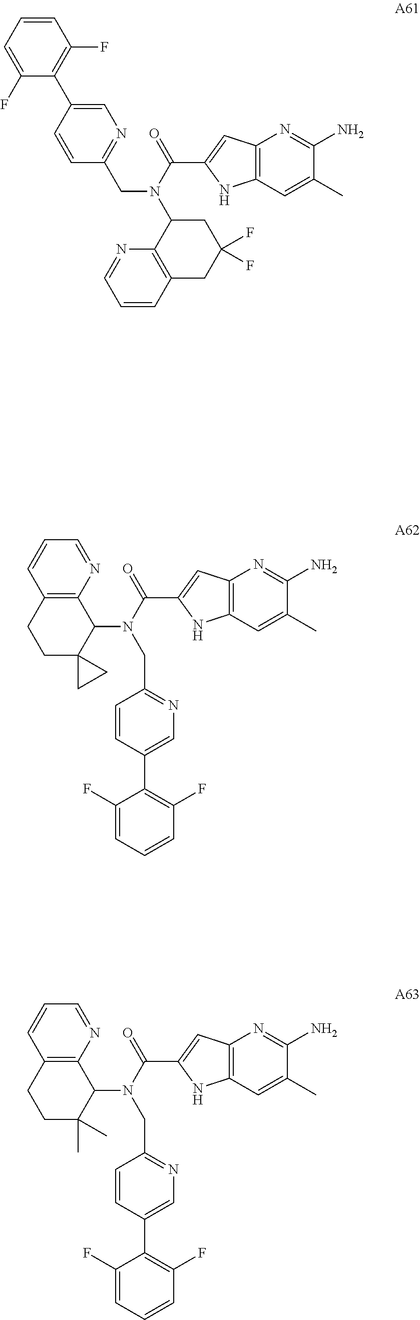 [SMILES: Cc1cc2[nH]c(C(=O)N(Cc3ccc(-c4c(F)cccc4F)cn3)C3CC(F)(F)Cc4cccnc43)cc2nc1N ; Cc1cc2[nH]c(C(=O)N(Cc3ccc(-c4c(F)cccc4F)cn3)C3c4ncccc4CCC34CC4)cc2nc1N ; Cc1cc2[nH]c(C(=O)N(Cc3ccc(-c4c(F)cccc4F)cn3)C3c4ncccc4CCC3(C)C)cc2nc1N]

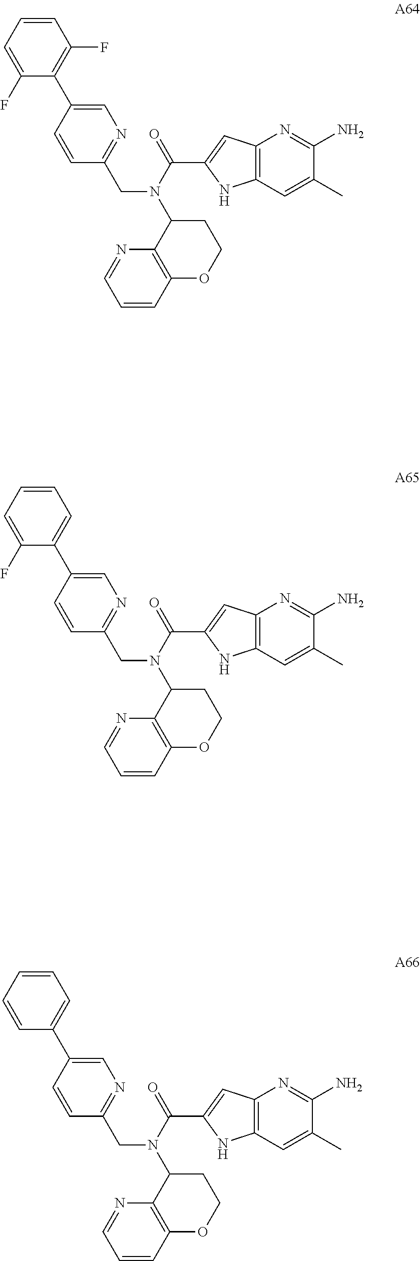 [SMILES: Cc1cc2[nH]c(C(=O)N(Cc3ccc(-c4c(F)cccc4F)cn3)C3CCOc4cccnc43)cc2nc1N ; Cc1cc2[nH]c(C(=O)N(Cc3ccc(-c4ccccc4F)cn3)C3CCOc4cccnc43)cc2nc1N ; Cc1cc2[nH]c(C(=O)N(Cc3ccc(-c4ccccc4)cn3)C3CCOc4cccnc43)cc2nc1N]

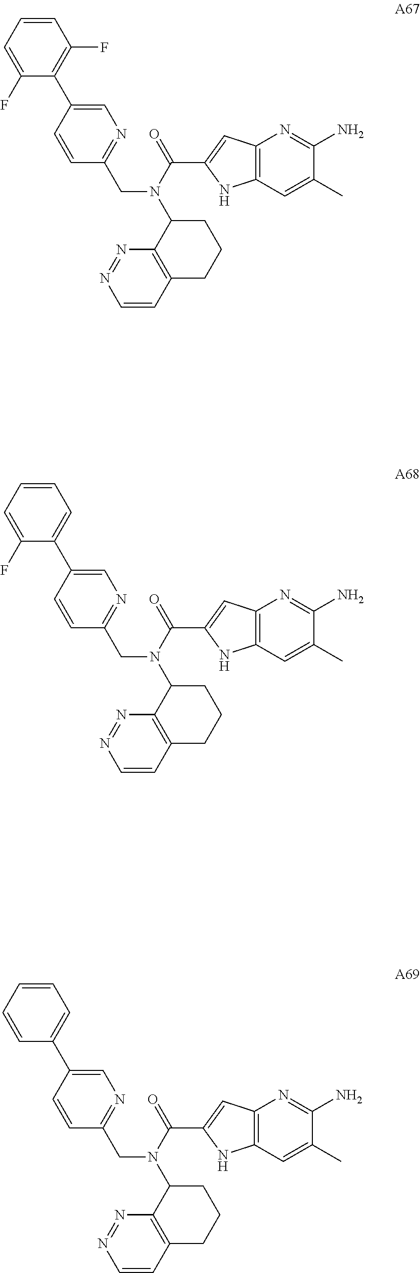 [SMILES: Cc1cc2[nH]c(C(=O)N(Cc3ccc(-c4c(F)cccc4F)cn3)C3CCCc4ccnnc43)cc2nc1N ; Cc1cc2[nH]c(C(=O)N(Cc3ccc(-c4ccccc4F)cn3)C3CCCc4ccnnc43)cc2nc1N ; Cc1cc2[nH]c(C(=O)N(Cc3ccc(-c4ccccc4)cn3)C3CCCc4ccnnc43)cc2nc1N]

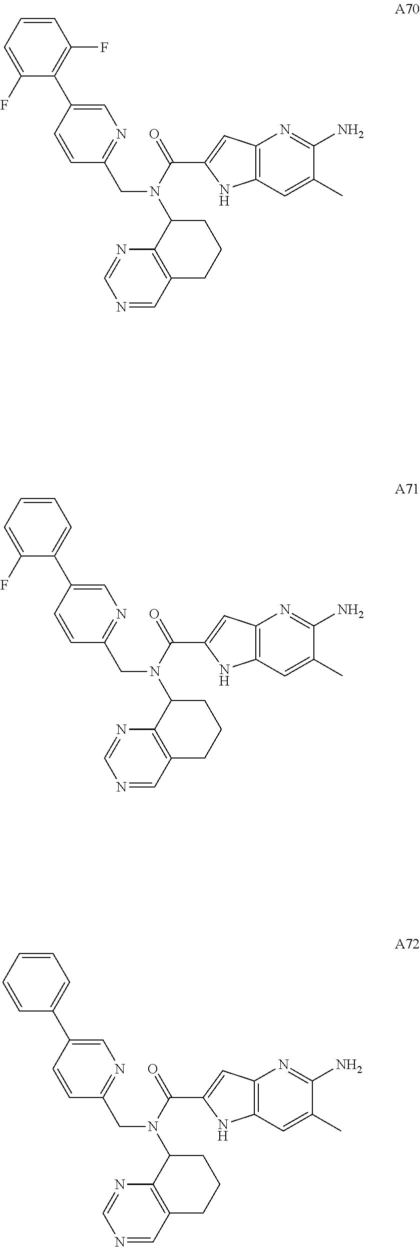 [SMILES: Cc1cc2[nH]c(C(=O)N(Cc3ccc(-c4c(F)cccc4F)cn3)C3CCCc4cncnc43)cc2nc1N ; Cc1cc2[nH]c(C(=O)N(Cc3ccc(-c4ccccc4F)cn3)C3CCCc4cncnc43)cc2nc1N ; Cc1cc2[nH]c(C(=O)N(Cc3ccc(-c4ccccc4)cn3)C3CCCc4cncnc43)cc2nc1N]

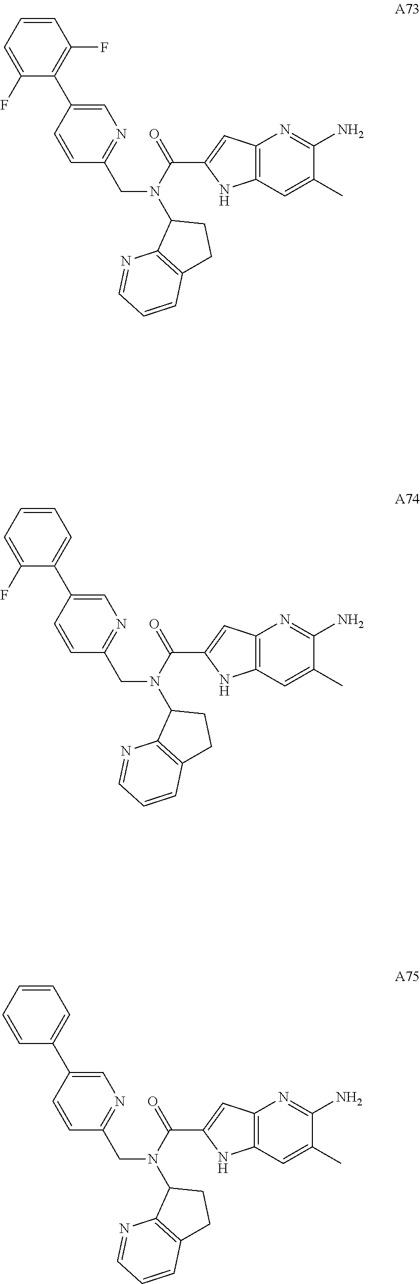 [SMILES: Cc1cc2[nH]c(C(=O)N(Cc3ccc(-c4c(F)cccc4F)cn3)C3CCc4cccnc43)cc2nc1N ; Cc1cc2[nH]c(C(=O)N(Cc3ccc(-c4ccccc4F)cn3)C3CCc4cccnc43)cc2nc1N ; Cc1cc2[nH]c(C(=O)N(Cc3ccc(-c4ccccc4)cn3)C3CCc4cccnc43)cc2nc1N]

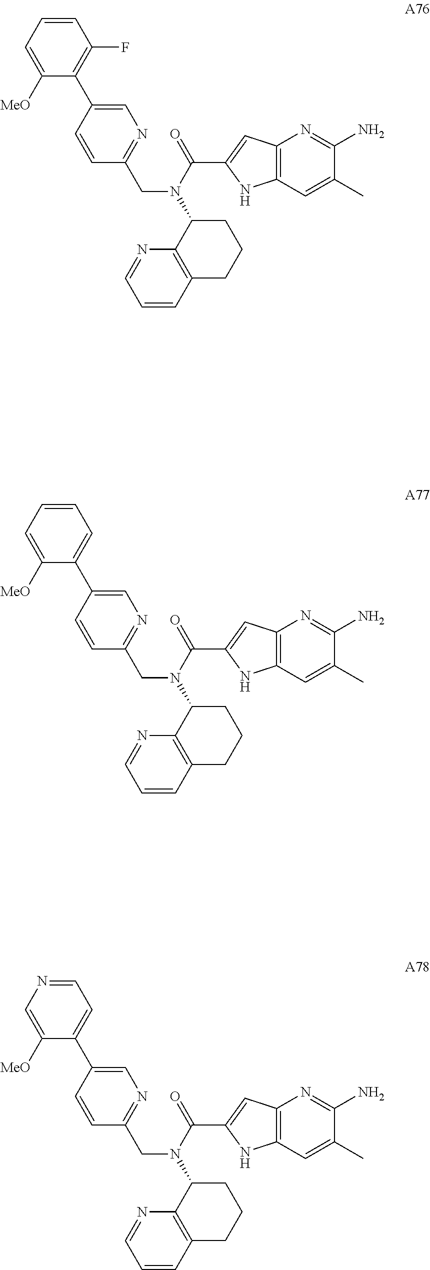 [SMILES: COc1cccc(F)c1-c1ccc(CN(C(=O)c2cc3nc(N)c(C)cc3[nH]2)[C@@H]2CCCc3cccnc32)nc1 ; COc1ccccc1-c1ccc(CN(C(=O)c2cc3nc(N)c(C)cc3[nH]2)[C@@H]2CCCc3cccnc32)nc1 ; COc1cnccc1-c1ccc(CN(C(=O)c2cc3nc(N)c(C)cc3[nH]2)[C@@H]2CCCc3cccnc32)nc1]

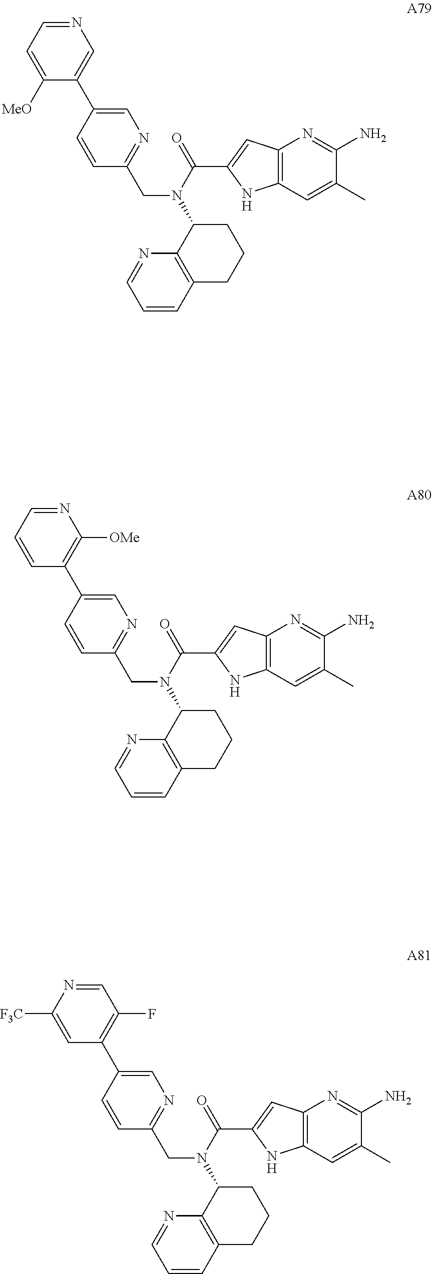 [SMILES: COc1ccncc1-c1ccc(CN(C(=O)c2cc3nc(N)c(C)cc3[nH]2)[C@@H]2CCCc3cccnc32)nc1 ; COc1ncccc1-c1ccc(CN(C(=O)c2cc3nc(N)c(C)cc3[nH]2)[C@@H]2CCCc3cccnc32)nc1 ; Cc1cc2[nH]c(C(=O)N(Cc3ccc(-c4cc(C(F)(F)F)ncc4F)cn3)[C@@H]3CCCc4cccnc43)cc2nc1N]

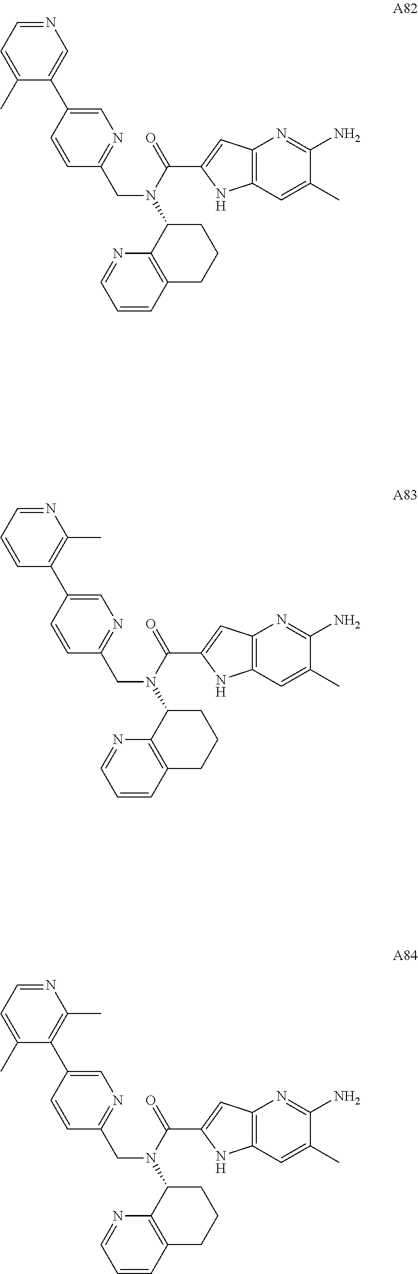 [SMILES: Cc1ccncc1-c1ccc(CN(C(=O)c2cc3nc(N)c(C)cc3[nH]2)[C@@H]2CCCc3cccnc32)nc1 ; Cc1cc2[nH]c(C(=O)N(Cc3ccc(-c4cccnc4C)cn3)[C@@H]3CCCc4cccnc43)cc2nc1N ; Cc1cc2[nH]c(C(=O)N(Cc3ccc(-c4c(C)ccnc4C)cn3)[C@@H]3CCCc4cccnc43)cc2nc1N]

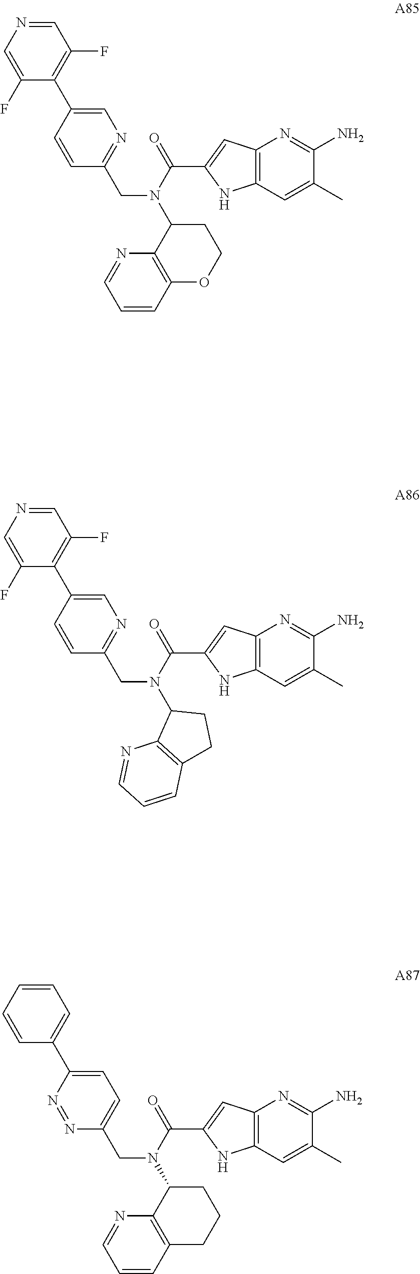 [SMILES: Cc1cc2[nH]c(C(=O)N(Cc3ccc(-c4c(F)cncc4F)cn3)C3CCOc4cccnc43)cc2nc1N ; Cc1cc2[nH]c(C(=O)N(Cc3ccc(-c4c(F)cncc4F)cn3)C3CCc4cccnc43)cc2nc1N ; Cc1cc2[nH]c(C(=O)N(Cc3ccc(-c4ccccc4)nn3)[C@@H]3CCCc4cccnc43)cc2nc1N]

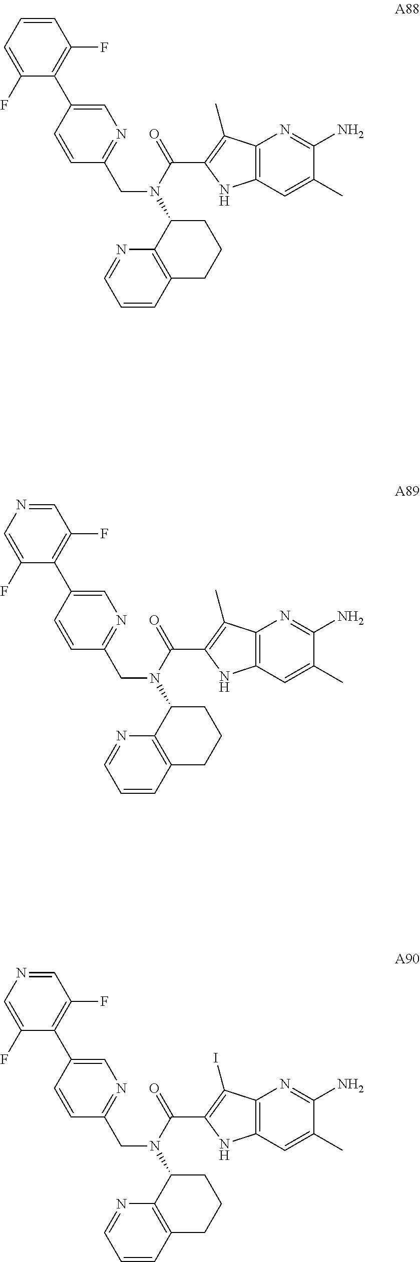 [SMILES: Cc1cc2[nH]c(C(=O)N(Cc3ccc(-c4c(F)cccc4F)cn3)[C@@H]3CCCc4cccnc43)c(C)c2nc1N ; Cc1cc2[nH]c(C(=O)N(Cc3ccc(-c4c(F)cncc4F)cn3)[C@@H]3CCCc4cccnc43)c(C)c2nc1N ; Cc1cc2[nH]c(C(=O)N(Cc3ccc(-c4c(F)cncc4F)cn3)[C@@H]3CCCc4cccnc43)c(I)c2nc1N]

Aspect A20. A pharmaceutical composition comprising a compound of any one of Aspects A1-A19 or a pharmaceutically acceptable salt, stereoisomer, tautomer or prodrug thereof, together with a pharmaceutically acceptable excipient.

Aspect A21. A method of decreasing PRMT5 activity by inhibition, which comprises administering to an individual the compound according to any one of Aspects A1-A19, or a pharmaceutically acceptable salt thereof, including the compound of formula (I) or the specific compounds exemplified herein.

Aspect A22. The method of Aspect A21, wherein the disease is selected from cancer.

Aspect A23. Use of a compound of any one of Aspects A1-A19 or a pharmaceutically acceptable salt, stereoisomer, tautomer or prodrug thereof in the preparation of a medicament for treating a disease that is modulated by PRMT5.

Aspect A24. The use of Aspect A23, wherein the disease is cancer.

Aspect A25. The use of Aspect A24, wherein the disease is MTAP-null solid tumor, including but not limited to lung cancer, bladder cancer, melanoma, pancreatic cancer, esophageal cancer, gastric adenocarcinoma, breast cancer or glioblastoma.

Aspect B1, the compound is Formula (BI):

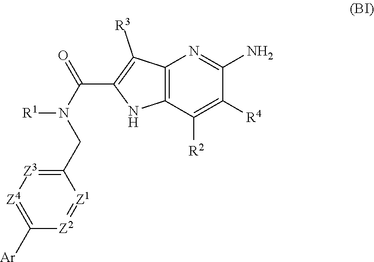

or a N-oxide thereof, or a pharmaceutically acceptable salt thereof, or a stereoisomer thereof, or a tautomer thereof, or a deuterated analog thereof, wherein:
Z¹, Z², Z³ and Z⁴ are each independently selected from N or CR^{Z};
at each of its occurrences, R^{Z} is each independently selected from hydrogen, halogen, —C₁₋₄alkyl or —C₁₋₄alkoxy; wherein each of said —C₁₋₄alkyl or —C₁₋₄alkoxy is optionally substituted with at least one substituent selected from halogen;
Ar is independently selected from phenyl or 5- to 6-membered heteroaryl, wherein each of said phenyl or 5- to 6-membered heteroaryl is optionally substituted with at least one substituent R^{Ar},
R^{Ar} is each independently selected from hydrogen, halogen, —C₁₋₄alkyl, —C₁₋₄alkoxy, —C₃-C₆cycloalkyl, —C₃-C₆cycloalkoxy or —CN; wherein each of said —C₁₋₄alkyl, —C₁₋₄alkoxy, —C₃-C₆cycloalkyl or —C₃-C₆cycloalkoxy is optionally substituted with at least one substituent selected from halogen;
R¹ is each independently selected from —C₁₋₈alkyl, —C₃-C₈cycloalkyl (preferably, mono, bridged, fused or spiral cycloalkyl) or 3- to 8-membered saturated heterocyclyl (preferably, mono, bridged, fused or spiral heterocyclyl); wherein each of said —C₁₋₈alkyl, —C₃-C₈cycloalkyl (preferably, mono, bridged, fused or spiral cycloalkyl) or 3- to 8-membered saturated heterocyclyl (preferably, mono, bridged, fused or spiral heterocyclyl) is optionally substituted with at least one substituent R^{1a};
R^{1a} is each independently selected from halogen, —C₁₋₄alkyl, —C₃-C₆cycloalkyl, 3- to 6-membered saturated heterocyclyl, —C₁₋₄alkoxy, —C₃-C₆cycloalkoxy, —CN, —OH, —NH₂ or oxo; wherein each of said —C₁₋₄alkyl, —C₃-C₆cycloalkyl, 3- to 6-membered saturated heterocyclyl, —C₁₋₄alkoxy or —C₃-C₆cycloalkoxy is optionally substituted with at least one substituent selected from halogen, —C₁₋₄alkyl, —C₃-C₆cycloalkyl, 3- to 6-membered saturated heterocyclyl, —C₁₋₄alkoxy, —C₃-C₆cycloalkoxy, —CN, —OH, —NH₂ or oxo;
R² is selected from hydrogen, halogen, —C₁₋₄alkyl, —C₁₋₄alkoxy, —C₃-C₆cycloalkyl, —C₃-C₆cycloalkoxy or —CN; wherein each of said —C₁₋₄alkyl, —C₁₋₄alkoxy, —C₃-C₆cycloalkyl or —C₃-C₆cycloalkoxy is optionally substituted with at least one substituent selected from halogen, —C₁₋₄alkoxy or —C₃-C₆cycloalkoxy;
R³ is selected from hydrogen, halogen, —C₁₋₄alkyl or —CN, wherein —C₁₋₄alkyl is optionally substituted with at least one substituent selected from hydrogen, halogen, —C₁₋₄alkoxy, —C₃-C₆cycloalkyl, —CN, —OH, —NH₂ or oxo;
R⁴ is selected from hydrogen, halogen, —C₁₋₄alkyl, —C₁₋₄alkoxy, —C₃-C₆cycloalkyl or —C₃-C₆cycloalkoxy, wherein each of said —C₁₋₄alkyl, —C₁₋₄alkoxy, —C₃-C₆cycloalkyl or —C₃-C₆cycloalkoxy is optionally substituted with at least one substituent selected from halogen, —C₁₋₄alkoxy, —C₃-C₆cycloalkoxy, —CN, —OH, —NH₂ or oxo.

Aspect B2. The compound of Aspect B1, wherein the compound is formula (BIIa), (BIIb), (BIIc), (BIId), (BIIe), (BIIf) or (BIIg):

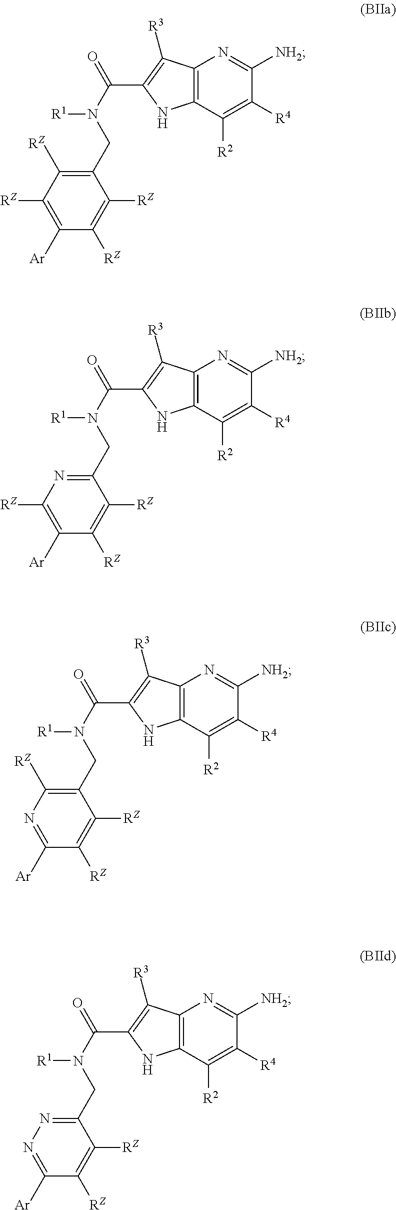

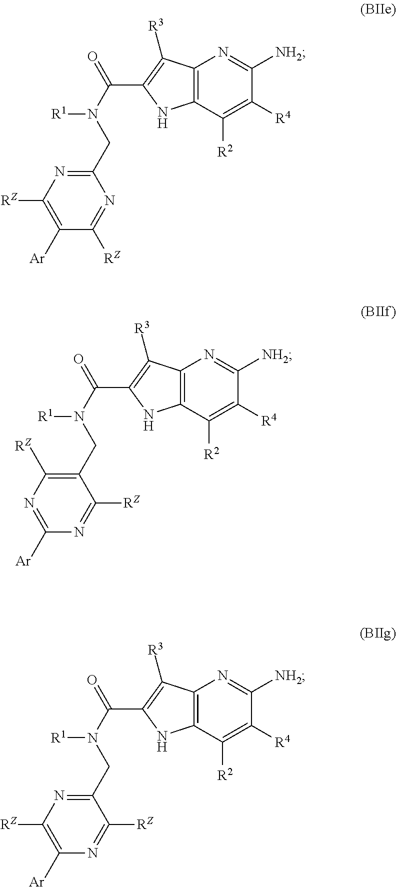

wherein, R¹, R², R³, R⁴, R^{Z}, Ar are as defined as Aspect B1.

Aspect B3. The compound of Aspect B1, wherein the compound is formula (BIIIa), (BIIIb), (BIIIc):

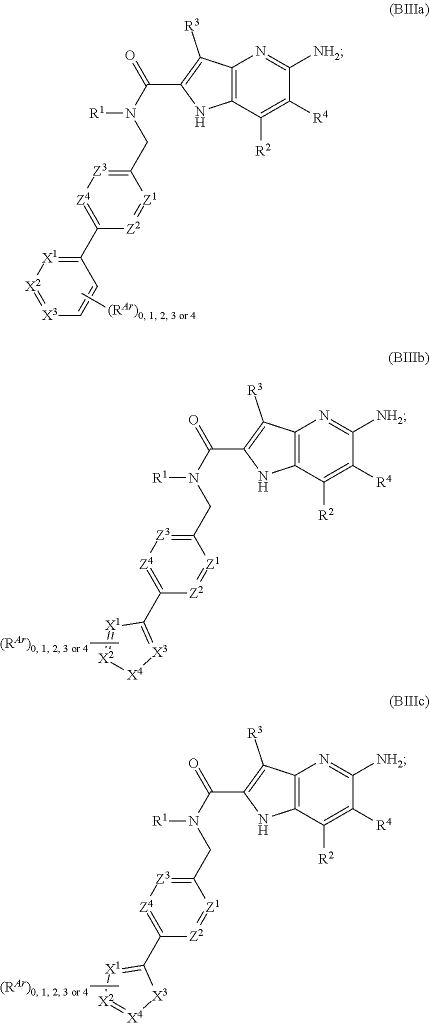

wherein, X¹, X² and X³ are each independently selected from N or CH; provided that when anyone of X¹, X² or X³ is CH, CH is optionally substituted with R^{Ar};
X⁴ is each independently selected from NH, O or S; provided that when anyone of X⁴ is NH, NH is optionally substituted with R^{Ar};
R¹, R², R³, R⁴, R^{Ar}, Z¹, Z², Z³ and Z⁴ are as defined as Aspect B1.

In some embodiments, the compound is formula (BIIId), (BIIIe), (BIIIf), (BIIIg), (BIIIh) or (BIIIi):

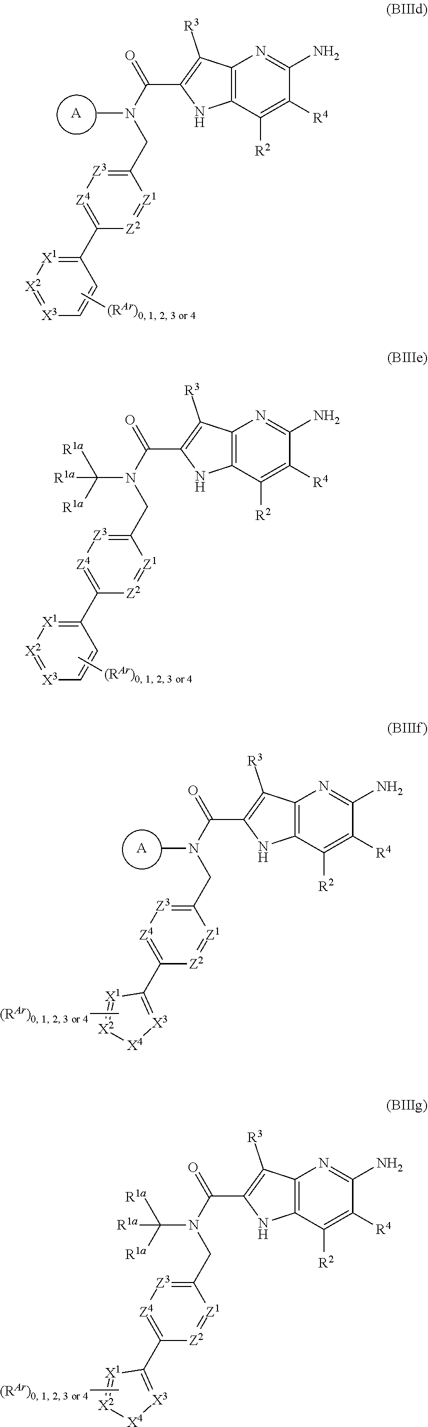

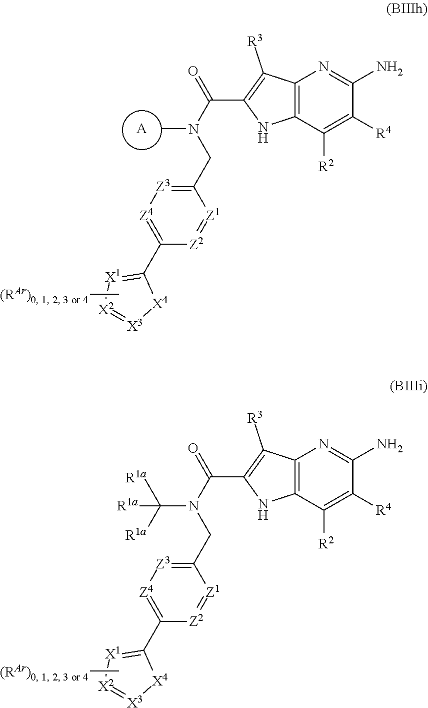

wherein, ring A is —C₃-C₈cycloalkyl (preferably, mono, bridged, fused or spiral cycloalkyl) or 3- to 8-membered saturated heterocyclyl (preferably, mono, bridged, fused or spiral heterocyclyl); wherein each of said —C₃-C₈cycloalkyl (preferably, mono, bridged, fused or spiral cycloalkyl) or 3- to 8-membered saturated heterocyclyl (preferably, mono, bridged, fused or spiral heterocyclyl) is optionally substituted with at least one substituent R^{1a};
X¹, X² and X³ are each independently selected from N or CH; provided that when anyone of X¹, X² or X³ is CH, CH is optionally substituted with R^{Ar};
X⁴ is each independently selected from NH, O or S; provided that when anyone of X⁴ is NH, NH is optionally substituted with R^{Ar};
R^{1a}, R², R³, R⁴, R^{Ar}, Z¹, Z², Z³ and Z⁴ are as defined as Aspect B1.

Aspect B4. The compound of any anyone of preceding Aspects, wherein R¹ is selected from methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cyclopentyl, cyclohexyl, cycloheptyl, cyclooctyl, 3-, 4-, 5-, 6-, 7- or 8-membered saturated mono-heterocyclyl, 5-, 6-, 7- or 8-membered saturated bridged-heterocyclyl, 4-, 5-, 6-, 7- or 8-membered saturated fused-heterocyclyl, or 5-, 6-, 7- or 8-membered saturated spiral-heterocyclyl; wherein each of said methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cyclopentyl, cyclohexyl, cycloheptyl, cyclooctyl, 3-, 4-, 5-, 6-, 7- or 8-membered saturated mono-heterocyclyl, 5-, 6-, 7- or 8-membered saturated bridged-heterocyclyl, 4-, 5-, 6-, 7- or 8-membered saturated fused-heterocyclyl, or 5-, 6-, 7- or 8-membered saturated spiral-heterocyclyl is optionally substituted with at least one substituent R^{1a};
R^{1a} is each independently selected from —F, —Cl, —Br, —I, methyl, ethyl, propyl (iso-propyl or n-propyl), butyl (n-butyl, sec-butyl, iso-butyl or tert-butyl), cyclopropyl, cyclobutyl, cyclopentyl, cyclohexyl, 3-, 4-, 5- or 6-membered saturated heterocyclyl, methoxy, ethoxy, propoxy, butoxy, cyclopropoxy, cyclobutoxy, cyclopentoxy, cyclohexoxy, —CN, —OH, —NH₂ or oxo; wherein each of said methyl, ethyl, propyl (iso-propyl or n-propyl), butyl (n-butyl, sec-butyl, iso-butyl or tert-butyl), cyclopropyl, cyclobutyl, cyclopentyl, cyclohexyl, 3-, 4-, 5- or 6-membered saturated heterocyclyl, methoxy, ethoxy, propoxy, butoxy, cyclopropoxy, cyclobutoxy, cyclopentoxy or cyclohexoxy is optionally substituted with at least one substituent selected from —F, —Cl, —Br, —I, methyl, ethyl, propyl (iso-propyl or n-propyl), butyl (n-butyl, sec-butyl, iso-butyl or tert-butyl), cyclopropyl, cyclobutyl, cyclopentyl, cyclohexyl, 3-, 4-, 5- or 6-membered saturated heterocyclyl, methoxy, ethoxy, propoxy, butoxy, cyclopropoxy, cyclobutoxy, cyclopentoxy, cyclohexoxy, —CN, —OH, —NH₂ or oxo.

Aspect B5. The compound of any anyone of preceding Aspects, wherein R¹ is selected from methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cyclopentyl, cyclohexyl, cycloheptyl, cyclooctyl, 3-, 4-, 5-, 6-, 7- or 8-membered saturated mono-heterocyclyl, 5-, 6-, 7- or 8-membered saturated bridged-heterocyclyl, 4-, 5-, 6-, 7- or 8-membered saturated fused-heterocyclyl, or 5-, 6-, 7- or 8-membered saturated spiral-heterocyclyl each of said is optionally substituted with at least one substituent R^{1a};
R^{1a} is each independently selected from —F, —Cl, —Br, —I, methyl, ethyl, propyl (iso-propyl or n-propyl), butyl (n-butyl, sec-butyl, iso-butyl or tert-butyl), cyclopropyl, cyclobutyl, cyclopentyl, cyclohexyl, 3-, 4-, 5- or 6-membered saturated heterocyclyl, methoxy, ethoxy, propoxy, butoxy, cyclopropoxy, cyclobutoxy, cyclopentoxy, cyclohexoxy, —CN, —OH, —NH₂ or oxo.

In some embodiments, R¹ is selected from methyl, ethyl, propyl (iso-propyl or n-propyl), cyclopropyl, butyl (n-butyl, sec-butyl, iso-butyl or tert-butyl), cyclobutyl, cyclopentyl, cyclohexyl, tetrahydropyranyl, tetrahydrofuranyl, piperidinyl or pyrrolidinyl; wherein each of said methyl, ethyl, propyl (iso-propyl or n-propyl), cyclopropyl, butyl (n-butyl, sec-butyl, iso-butyl or tert-butyl), pentyl, hexyl, cyclobutyl, cyclopentyl, cyclohexyl, tetrahydropyranyl, tetrahydrofuranyl, piperidinyl or pyrrolidinyl is optionally substituted with at least one substituent R^{1a};
R^{1a} is each independently selected from —F, —Cl, —Br, —I, methyl, ethyl, propyl (iso-propyl or n-propyl), cyclopropyl, butyl (n-butyl, sec-butyl, iso-butyl or tert-butyl), pentyl, hexyl, cyclobutyl, cyclopentyl, cyclohexyl, tetrahydropyranyl, tetrahydrofuranyl, piperidinyl, pyrrolidinyl, methoxy, ethoxy, propoxy, butoxy, cyclopropoxy, cyclobutoxy, cyclopentoxy, cyclohexoxy, —CN, —OH, —NH₂ or oxo.

Aspect B6. The compound of any anyone of preceding Aspects, wherein R¹ is selected from

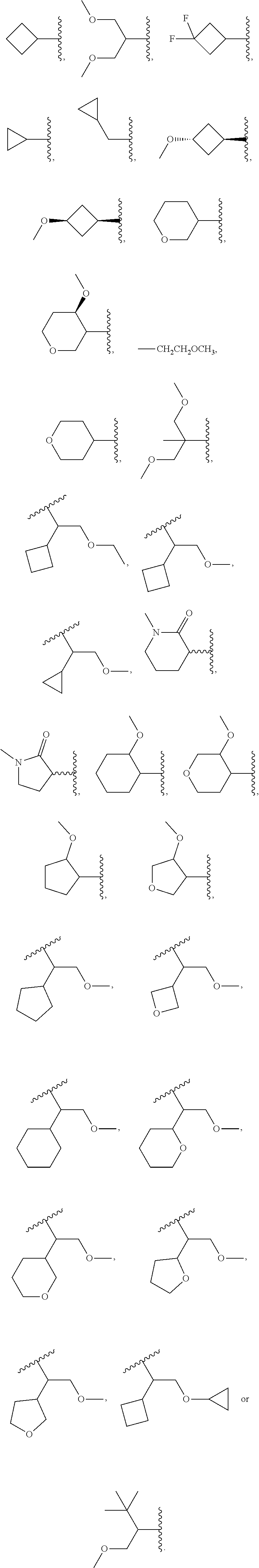

Aspect B7. The compound of anyone of preceding Aspects, wherein R² is selected from hydrogen, —F, —Cl, —Br, —I, methyl, ethyl, propyl (iso-propyl or n-propyl), butyl (n-butyl, sec-butyl, iso-butyl or tert-butyl), methoxy, ethoxy, propoxy, butoxy, cyclopropyl, cyclobutyl, cyclopentyl, cyclohexyl, cyclopropoxy, cyclobutoxy, cyclopentoxy, cyclohexoxy or —CN; wherein methyl, ethyl, propyl (iso-propyl or n-propyl), butyl (n-butyl, sec-butyl, iso-butyl or tert-butyl), methoxy, ethoxy, propoxy, butoxy, cyclopropyl, cyclobutyl, cyclopentyl, cyclohexyl, cyclopropoxy, cyclobutoxy, cyclopentoxy or cyclohexoxy is optionally substituted with at least one substituent selected from —F, —Cl, —Br, —I, methoxy, ethoxy, propoxy, butoxy, cyclopropoxy, cyclobutoxy, cyclopentoxy, cyclohexoxy.

In some embodiments, R² is selected from hydrogen, methyl, ethyl, cyclopropyl, cyclobutyl, cyclopropoxy, cyclobutoxy or —CN; wherein methyl, ethyl, is optionally substituted with at least one substituent selected from —F, —Cl, —Br, —I, methoxy, ethoxy, propoxy, cyclopropoxy or cyclobutoxy.

In some embodiments, R² is selected from hydrogen, —F, —Cl, —Br, —I, methyl, ethyl, propyl (iso-propyl or n-propyl), butyl (n-butyl, sec-butyl, iso-butyl or tert-butyl), methoxy, ethoxy, propoxy, butoxy, cyclopropyl, cyclobutyl, cyclopentyl, cyclohexyl, cyclopropoxy, cyclobutoxy, cyclopentoxy, cyclohexoxy or —CN.

Aspect B8. The compound of anyone of preceding Aspects, wherein R² is selected from hydrogen, methyl, ethyl or —CN.

Aspect B9. The compound of anyone of preceding Aspects, wherein R³ is selected from hydrogen, —F, —Cl, —Br, —I, methyl, ethyl, propyl (iso-propyl or n-propyl), butyl (n-butyl, sec-butyl, iso-butyl or tert-butyl) or —CN, wherein methyl, ethyl, propyl (iso-propyl or n-propyl) or butyl (n-butyl, sec-butyl, iso-butyl or tert-butyl) is optionally substituted with at least one substituent selected from hydrogen, —F, —Cl, —Br, —I, methoxy, ethoxy, propoxy, butoxy, cyclopropyl, cyclobutyl, cyclopentyl, cyclohexyl, —CN, —OH, —NH₂ or oxo.

In some embodiments, R³ is selected from hydrogen, —F, —Cl, —Br, —I, methyl, ethyl, propyl (iso-propyl or n-propyl), butyl (n-butyl, sec-butyl, iso-butyl or tert-butyl) or —CN.

Aspect B10. The compound of anyone of preceding Aspects, wherein R³ is selected from hydrogen, —F, —Cl, —Br, —I or —CN.

Aspect B11. The compound of anyone of preceding Aspects, wherein R⁴ is selected from hydrogen, —F, —Cl, —Br, —I, methyl, ethyl, propyl (iso-propyl or n-propyl), butyl (n-butyl, sec-butyl, iso-butyl or tert-butyl), methoxy, ethoxy, propoxy, butoxy, cyclopropyl, cyclobutyl, cyclopentyl, cyclohexyl, cyclopropoxy, cyclobutoxy, cyclopentoxy or cyclohexoxy, wherein methyl, ethyl, propyl (iso-propyl or n-propyl) or butyl (n-butyl, sec-butyl, iso-butyl or tert-butyl), methoxy, ethoxy, propoxy, butoxy, cyclopropyl, cyclobutyl, cyclopentyl, cyclohexyl, cyclopropoxy, cyclobutoxy, cyclopentoxy or cyclohexoxy is optionally substituted with at least one substituent selected from —F, —Cl, —Br, —I, methoxy, ethoxy, propoxy, butoxy, cyclopropoxy, cyclobutoxy, cyclopentoxy, cyclohexoxy, —CN, —OH, —NH₂ or oxo.

In some embodiments, R⁴ is selected from hydrogen, —F, —Cl, —Br, methyl, ethyl, propyl (iso-propyl or n-propyl), methoxy, ethoxy, propoxy, cyclopropyl, cyclobutyl, cyclopropoxy or cyclobutoxy, wherein methyl, ethyl, propyl (iso-propyl or n-propyl), methoxy, ethoxy, propoxy, cyclopropyl, cyclobutyl, cyclopropoxy or cyclobutoxy is optionally substituted with at least one substituent selected from —F, —Cl, —Br, —I, methoxy, ethoxy, propoxy, cyclopropoxy, cyclobutoxy, —CN, —OH, —NH₂ or oxo.

In some embodiments, R⁴ is selected from hydrogen, —F, —Cl, —Br, —I, methyl, ethyl, propyl (iso-propyl or n-propyl) or butyl (n-butyl, sec-butyl, iso-butyl or tert-butyl).

Aspect B12. The compound of anyone of preceding Aspects, wherein R⁴ is selected from hydrogen, —F, —Cl, —Br, —I, methyl, ethyl.

Aspect B13. The compound of anyone of preceding Aspects, wherein at most two of Z¹, Z², Z³ and Z⁴ are N; R^{Z} is each independently selected from hydrogen, —F, —Cl, —Br, —I, methyl, ethyl, propyl (iso-propyl or n-propyl) or butyl (n-butyl, sec-butyl, iso-butyl or tert-butyl), methoxy, ethoxy, propoxy or butoxy; wherein each of said ethyl, propyl (iso-propyl or n-propyl) or butyl (n-butyl, sec-butyl, iso-butyl or tert-butyl), methoxy, ethoxy, propoxy or butoxy is optionally substituted with at least one substituent selected from —F, —Cl, —Br, —I.

In some embodiments, R^{Z} is each independently selected from hydrogen, —F, —Cl, —Br, —I, methyl, ethyl, propyl (iso-propyl or n-propyl), butyl (n-butyl, sec-butyl, iso-butyl or tert-butyl), methoxy, ethoxy, propoxy or butoxy.

In some embodiments, R^{Z} is each independently selected from hydrogen, —F, —Cl, —Br, methyl or ethyl.

Aspect B14. The compound of anyone of preceding Aspects, wherein Ar is independently selected from phenyl, pyridinyl, pyridazinyl, pyrimidinyl, pyrazinyl, pyrrolyl, pyrazolyl, imidazolyl, 1,2,4-triazolyl, 1,2,3-triazolyl, oxazolyl, furanyl, thiazolyl or thiophenyl; each of said phenyl, pyridinyl, pyridazinyl, pyrimidinyl, pyrazinyl, pyrrolyl, pyrazolyl, imidazolyl, 1,2,4-triazolyl, 1,2,3-triazolyl, oxazolyl, furanyl, thiazolyl or thiophenyl is optionally substituted with at least one substituent R^{Ar};
R^{Ar} is each independently selected from hydrogen, —F, —Cl, —Br, —I, methyl, ethyl, propyl (iso-propyl or n-propyl), butyl (n-butyl, sec-butyl, iso-butyl or tert-butyl), methoxy, ethoxy, propoxy, butoxy, cyclopropyl, cyclobutyl, cyclopentyl, cyclohexyl, cyclopropoxy, cyclobutoxy, cyclopentoxy, cyclohexoxy or —CN; wherein each of said methyl, ethyl, propyl (iso-propyl or n-propyl), butyl (n-butyl, sec-butyl, iso-butyl or tert-butyl), methoxy, ethoxy, propoxy, butoxy, cyclopropyl, cyclobutyl, cyclopentyl, cyclohexyl, cyclopropoxy, cyclobutoxy, cyclopentoxy, cyclohexoxy is optionally substituted with at least one substituent selected from —F, —Cl, —Br or —I;

Aspect B15. The compound of anyone of preceding Aspects, wherein Ar is independently selected from phenyl, pyridinyl, pyrimidinyl, pyrrolyl, pyrazolyl or 1,2,4-triazolyl; each of said phenyl, pyridinyl, pyrimidinyl, pyrrolyl, pyrazolyl or 1,2,4-triazolyl is optionally substituted with at least one substituent R^{Ar};
R^{Ar} is each independently selected from hydrogen, —F, —Cl, —Br, —I, methyl, ethyl, propyl (iso-propyl or n-propyl), butyl (n-butyl, sec-butyl, iso-butyl or tert-butyl), methoxy, ethoxy, propoxy, butoxy, cyclopropyl, cyclobutyl, cyclopentyl, cyclohexyl, cyclopropoxy, cyclobutoxy, cyclopentoxy, cyclohexoxy, —CF₃ or —CN.

In some embodiments, Ar is independently selected from phenyl, pyridinyl, pyrimidinyl, pyrrolyl, pyrazolyl or 1,2,4-triazolyl; each of said phenyl, pyridinyl, pyrimidinyl, pyrrolyl, pyrazolyl or 1,2,4-triazolyl is optionally substituted with at least one substituent R^{Ar}; wherein R^{Ar} is each independently selected from hydrogen, —F, —Cl, —Br, methyl, ethyl, methoxy, ethoxy, —CF₃ or —CN.

Aspect B16. The compound of any one of the preceding aspects, wherein the compound is selected from

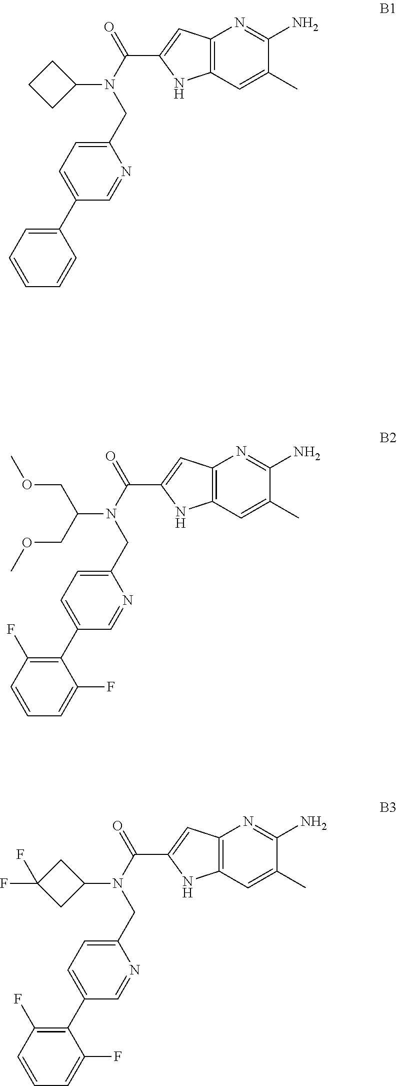 [SMILES: Cc1cc2[nH]c(C(=O)N(Cc3ccc(-c4ccccc4)cn3)C3CCC3)cc2nc1N ; COCC(COC)N(Cc1ccc(-c2c(F)cccc2F)cn1)C(=O)c1cc2nc(N)c(C)cc2[nH]1 ; Cc1cc2[nH]c(C(=O)N(Cc3ccc(-c4c(F)cccc4F)cn3)C3CC(F)(F)C3)cc2nc1N]

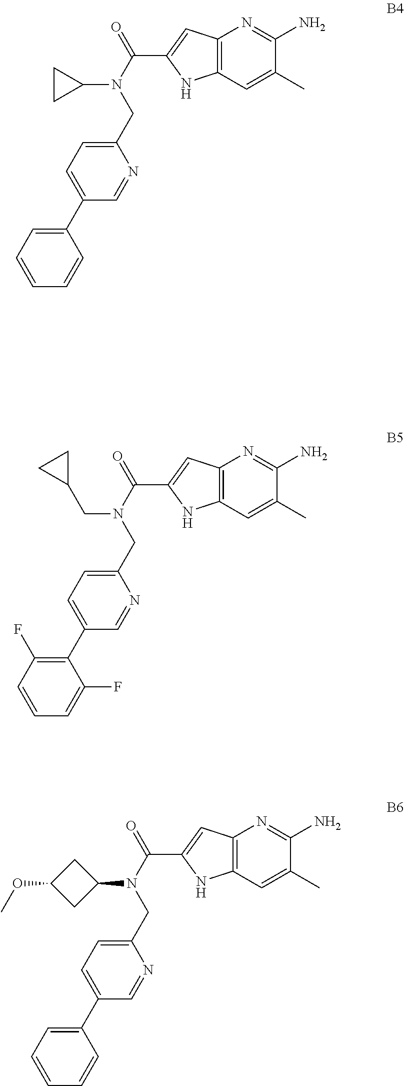

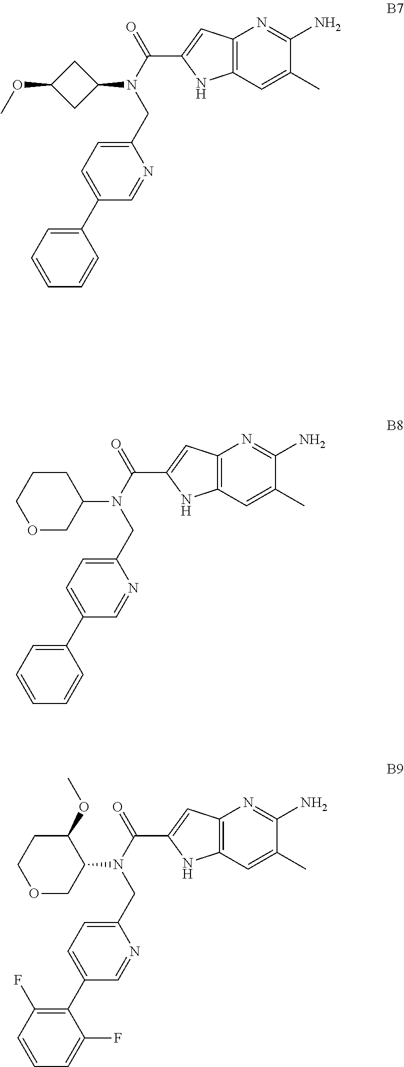

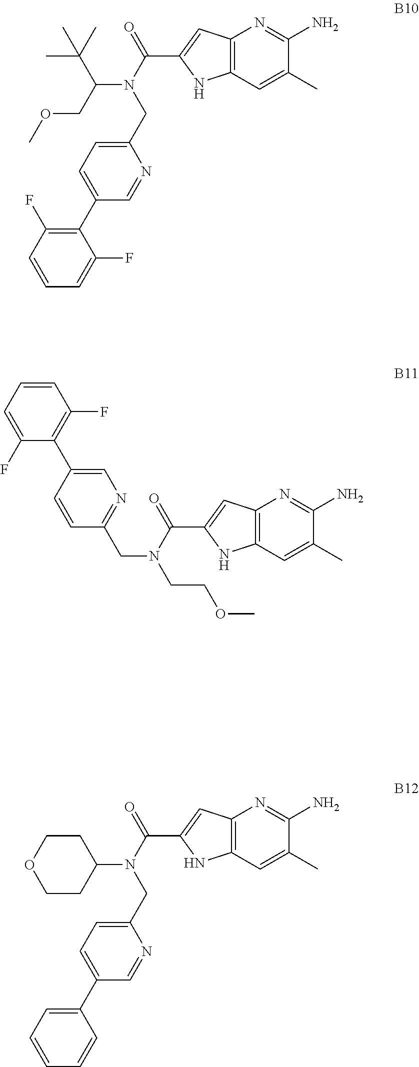 [SMILES: COCC(N(Cc1ccc(-c2c(F)cccc2F)cn1)C(=O)c1cc2nc(N)c(C)cc2[nH]1)C(C)(C)C ; COCCN(Cc1ccc(-c2c(F)cccc2F)cn1)C(=O)c1cc2nc(N)c(C)cc2[nH]1 ; Cc1cc2[nH]c(C(=O)N(Cc3ccc(-c4ccccc4)cn3)C3CCOCC3)cc2nc1N]

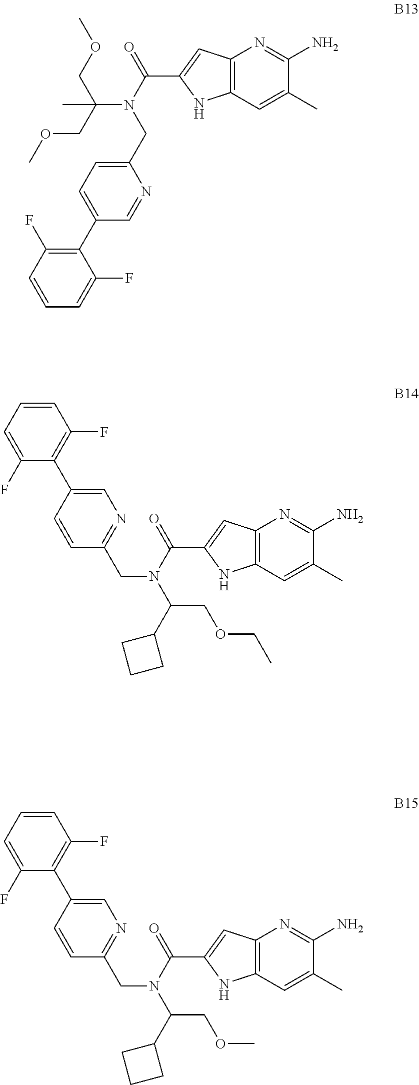 [SMILES: COCC(C)(COC)N(Cc1ccc(-c2c(F)cccc2F)cn1)C(=O)c1cc2nc(N)c(C)cc2[nH]1 ; CCOCC(C1CCC1)N(Cc1ccc(-c2c(F)cccc2F)cn1)C(=O)c1cc2nc(N)c(C)cc2[nH]1 ; COCC(C1CCC1)N(Cc1ccc(-c2c(F)cccc2F)cn1)C(=O)c1cc2nc(N)c(C)cc2[nH]1]

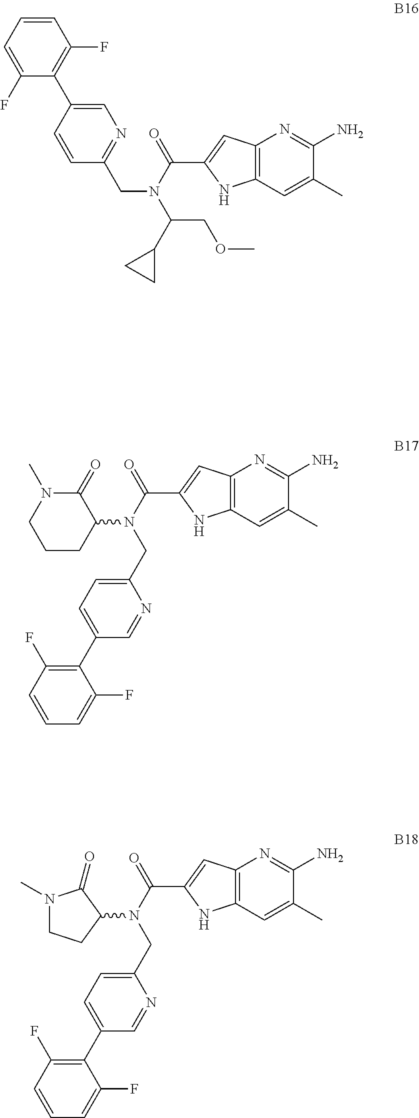

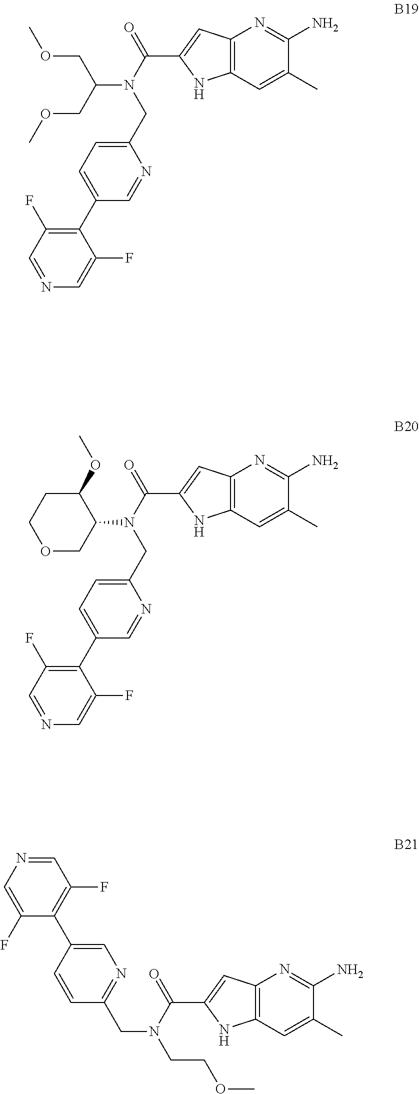 [SMILES: COCC(COC)N(Cc1ccc(-c2c(F)cncc2F)cn1)C(=O)c1cc2nc(N)c(C)cc2[nH]1 ; CO[C@@H]1CCOC[C@H]1N(Cc1ccc(-c2c(F)cncc2F)cn1)C(=O)c1cc2nc(N)c(C)cc2[nH]1 ; COCCN(Cc1ccc(-c2c(F)cncc2F)cn1)C(=O)c1cc2nc(N)c(C)cc2[nH]1]

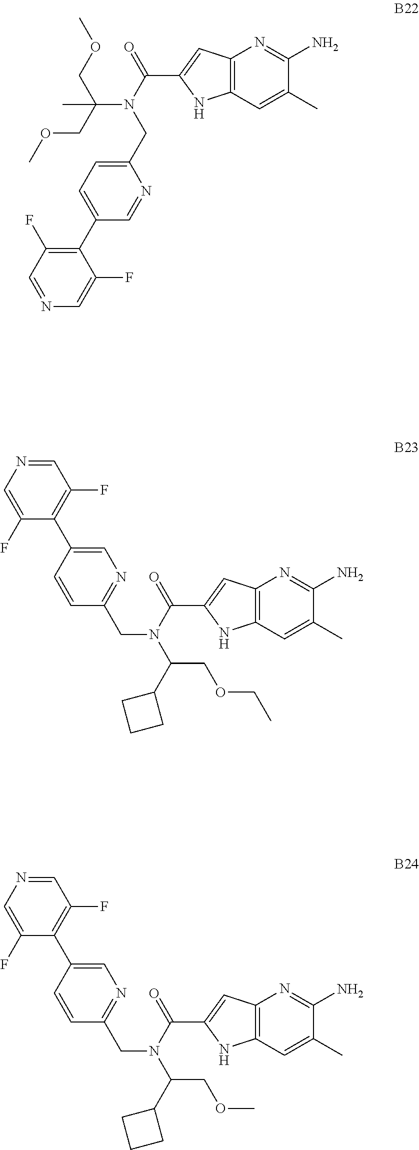 [SMILES: COCC(C)(COC)N(Cc1ccc(-c2c(F)cncc2F)cn1)C(=O)c1cc2nc(N)c(C)cc2[nH]1 ; CCOCC(C1CCC1)N(Cc1ccc(-c2c(F)cncc2F)cn1)C(=O)c1cc2nc(N)c(C)cc2[nH]1 ; COCC(C1CCC1)N(Cc1ccc(-c2c(F)cncc2F)cn1)C(=O)c1cc2nc(N)c(C)cc2[nH]1]

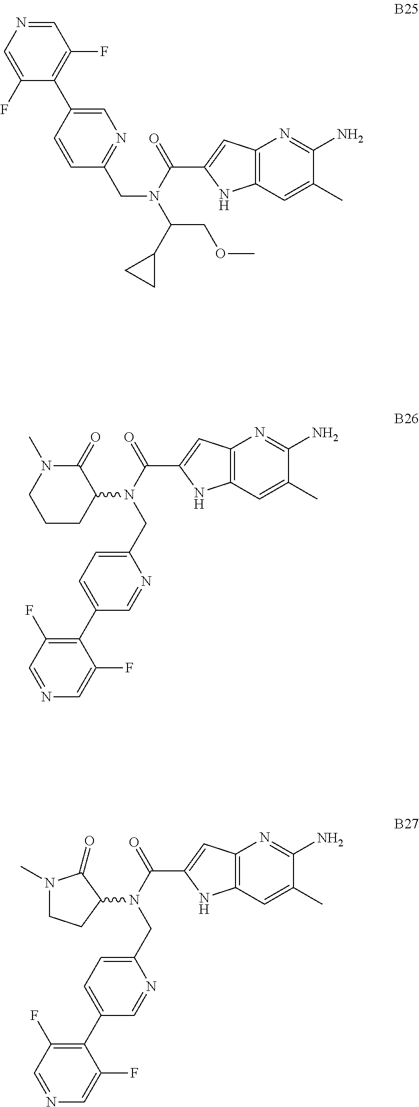

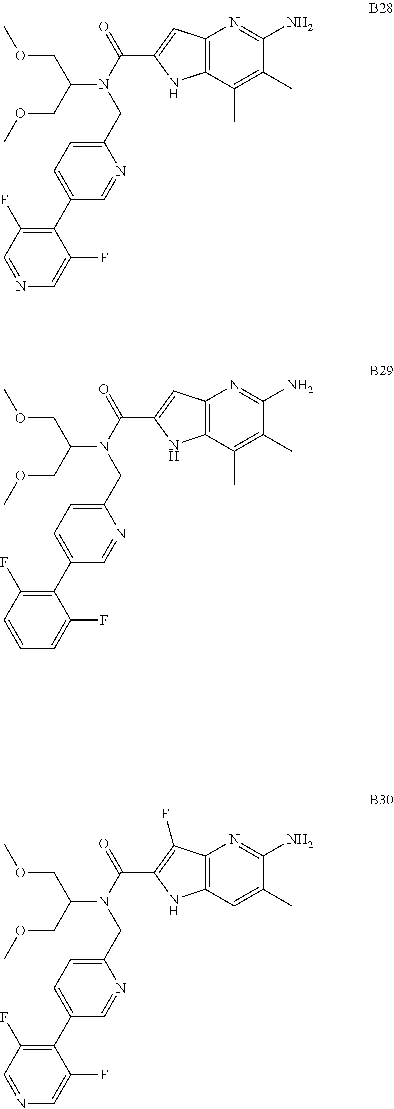 [SMILES: COCC(COC)N(Cc1ccc(-c2c(F)cncc2F)cn1)C(=O)c1cc2nc(N)c(C)c(C)c2[nH]1 ; COCC(COC)N(Cc1ccc(-c2c(F)cccc2F)cn1)C(=O)c1cc2nc(N)c(C)c(C)c2[nH]1 ; COCC(COC)N(Cc1ccc(-c2c(F)cncc2F)cn1)C(=O)c1[nH]c2cc(C)c(N)nc2c1F]

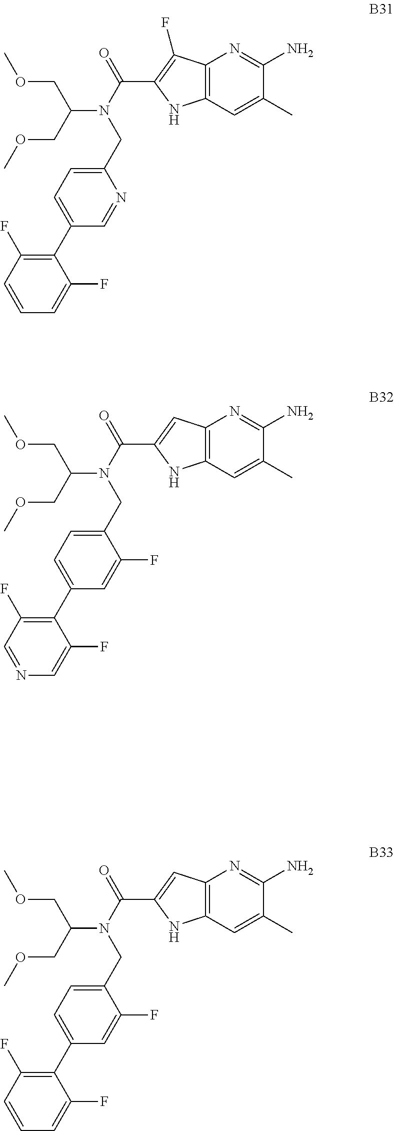 [SMILES: COCC(COC)N(Cc1ccc(-c2c(F)cccc2F)cn1)C(=O)c1[nH]c2cc(C)c(N)nc2c1F ; COCC(COC)N(Cc1ccc(-c2c(F)cncc2F)cc1F)C(=O)c1cc2nc(N)c(C)cc2[nH]1 ; COCC(COC)N(Cc1ccc(-c2c(F)cccc2F)cc1F)C(=O)c1cc2nc(N)c(C)cc2[nH]1]

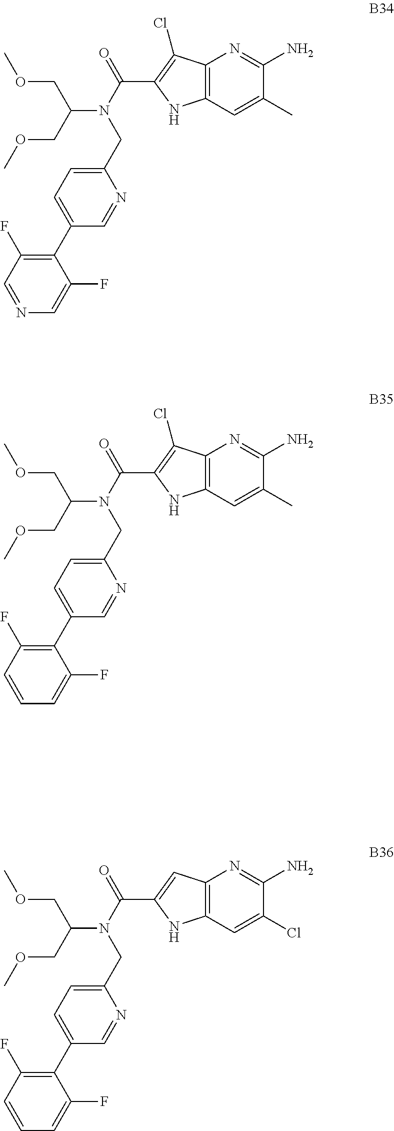 [SMILES: COCC(COC)N(Cc1ccc(-c2c(F)cncc2F)cn1)C(=O)c1[nH]c2cc(C)c(N)nc2c1Cl ; COCC(COC)N(Cc1ccc(-c2c(F)cccc2F)cn1)C(=O)c1[nH]c2cc(C)c(N)nc2c1Cl ; COCC(COC)N(Cc1ccc(-c2c(F)cccc2F)cn1)C(=O)c1cc2nc(N)c(Cl)cc2[nH]1]

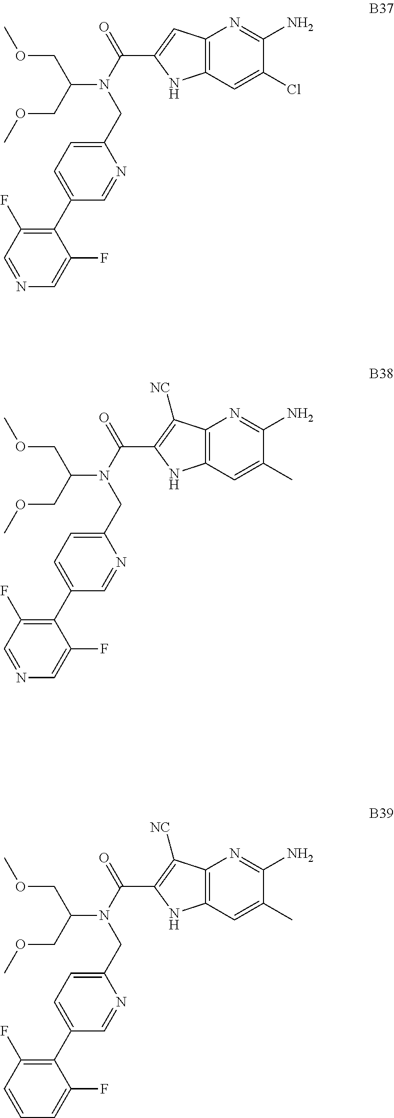 [SMILES: COCC(COC)N(Cc1ccc(-c2c(F)cncc2F)cn1)C(=O)c1cc2nc(N)c(Cl)cc2[nH]1 ; COCC(COC)N(Cc1ccc(-c2c(F)cncc2F)cn1)C(=O)c1[nH]c2cc(C)c(N)nc2c1C#N ; COCC(COC)N(Cc1ccc(-c2c(F)cccc2F)cn1)C(=O)c1[nH]c2cc(C)c(N)nc2c1C#N]

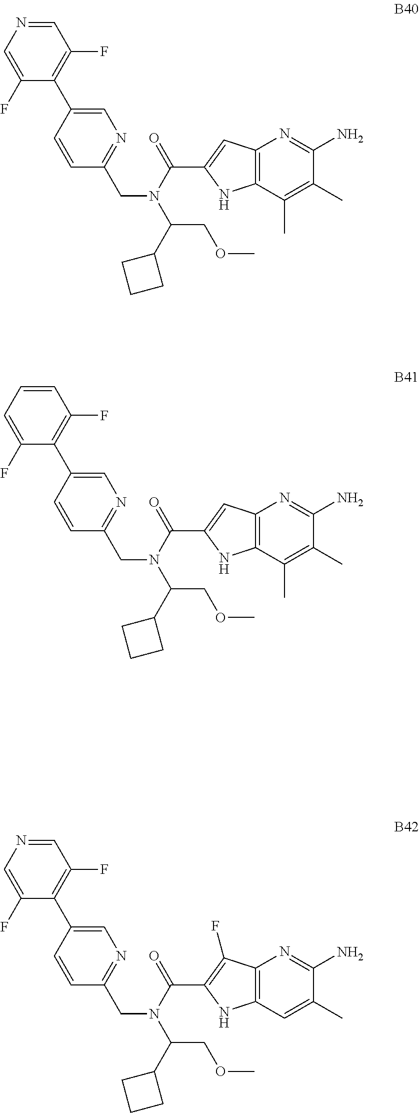 [SMILES: COCC(C1CCC1)N(Cc1ccc(-c2c(F)cncc2F)cn1)C(=O)c1cc2nc(N)c(C)c(C)c2[nH]1 ; COCC(C1CCC1)N(Cc1ccc(-c2c(F)cccc2F)cn1)C(=O)c1cc2nc(N)c(C)c(C)c2[nH]1 ; COCC(C1CCC1)N(Cc1ccc(-c2c(F)cncc2F)cn1)C(=O)c1[nH]c2cc(C)c(N)nc2c1F]

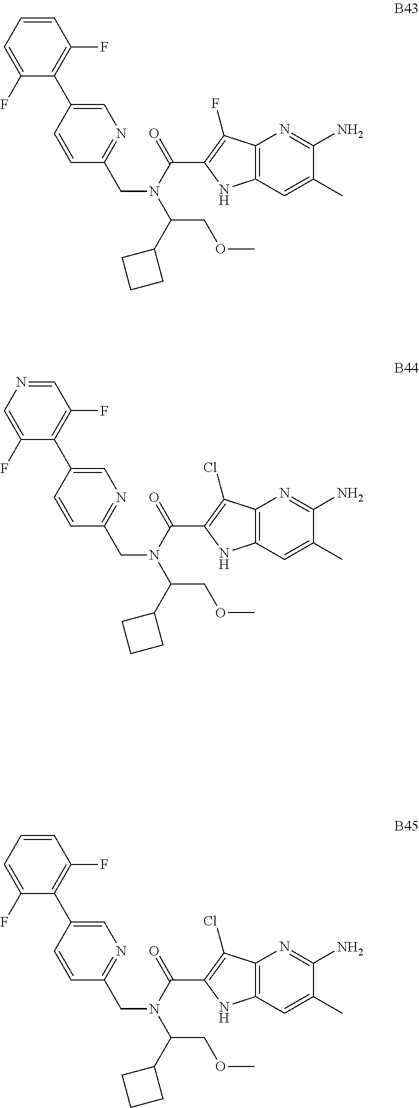 [SMILES: COCC(C1CCC1)N(Cc1ccc(-c2c(F)cccc2F)cn1)C(=O)c1[nH]c2cc(C)c(N)nc2c1F ; COCC(C1CCC1)N(Cc1ccc(-c2c(F)cncc2F)cn1)C(=O)c1[nH]c2cc(C)c(N)nc2c1Cl ; COCC(C1CCC1)N(Cc1ccc(-c2c(F)cccc2F)cn1)C(=O)c1[nH]c2cc(C)c(N)nc2c1Cl]

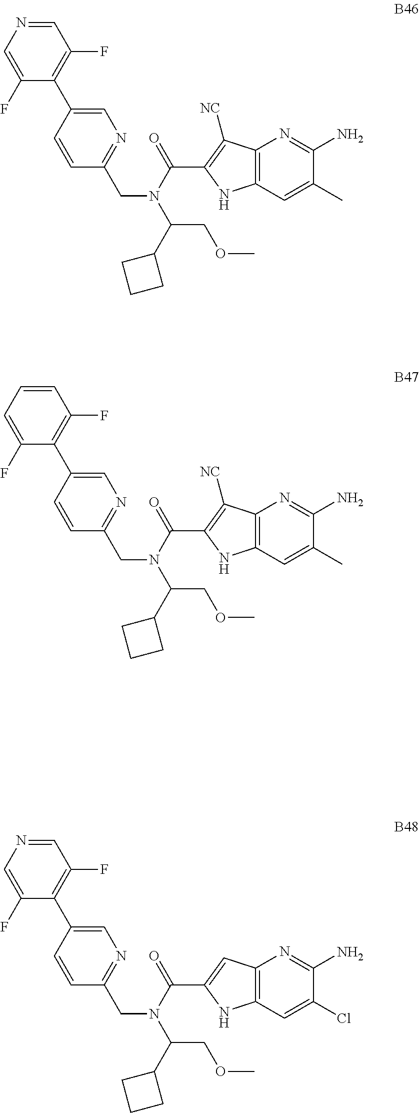 [SMILES: COCC(C1CCC1)N(Cc1ccc(-c2c(F)cncc2F)cn1)C(=O)c1[nH]c2cc(C)c(N)nc2c1C#N ; COCC(C1CCC1)N(Cc1ccc(-c2c(F)cccc2F)cn1)C(=O)c1[nH]c2cc(C)c(N)nc2c1C#N ; COCC(C1CCC1)N(Cc1ccc(-c2c(F)cncc2F)cn1)C(=O)c1cc2nc(N)c(Cl)cc2[nH]1]

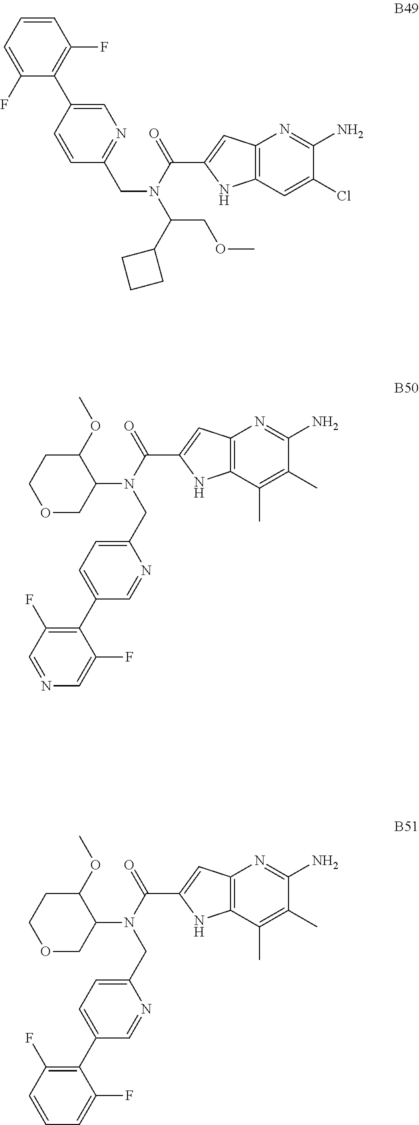 [SMILES: COCC(C1CCC1)N(Cc1ccc(-c2c(F)cccc2F)cn1)C(=O)c1cc2nc(N)c(Cl)cc2[nH]1 ; COC1CCOCC1N(Cc1ccc(-c2c(F)cncc2F)cn1)C(=O)c1cc2nc(N)c(C)c(C)c2[nH]1 ; COC1CCOCC1N(Cc1ccc(-c2c(F)cccc2F)cn1)C(=O)c1cc2nc(N)c(C)c(C)c2[nH]1]

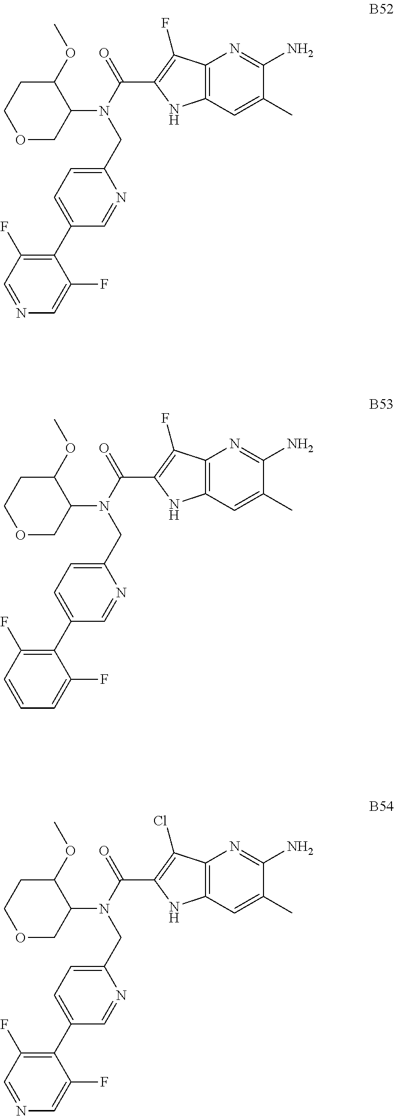 [SMILES: COC1CCOCC1N(Cc1ccc(-c2c(F)cncc2F)cn1)C(=O)c1[nH]c2cc(C)c(N)nc2c1F ; COC1CCOCC1N(Cc1ccc(-c2c(F)cccc2F)cn1)C(=O)c1[nH]c2cc(C)c(N)nc2c1F ; COC1CCOCC1N(Cc1ccc(-c2c(F)cncc2F)cn1)C(=O)c1[nH]c2cc(C)c(N)nc2c1Cl]

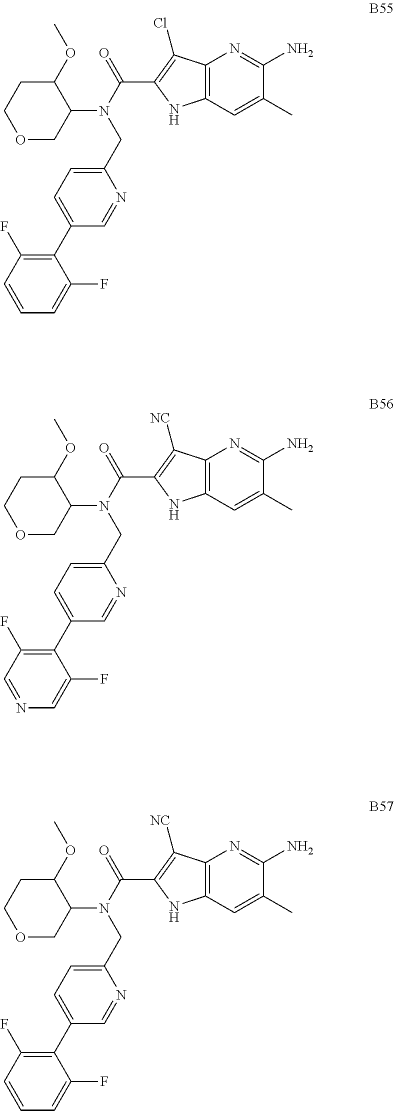 [SMILES: COC1CCOCC1N(Cc1ccc(-c2c(F)cccc2F)cn1)C(=O)c1[nH]c2cc(C)c(N)nc2c1Cl ; COC1CCOCC1N(Cc1ccc(-c2c(F)cncc2F)cn1)C(=O)c1[nH]c2cc(C)c(N)nc2c1C#N ; COC1CCOCC1N(Cc1ccc(-c2c(F)cccc2F)cn1)C(=O)c1[nH]c2cc(C)c(N)nc2c1C#N]

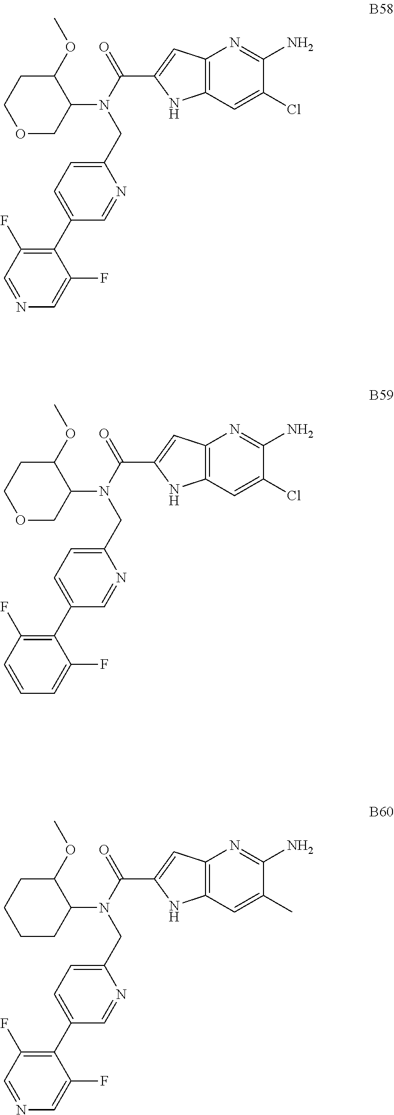 [SMILES: COC1CCOCC1N(Cc1ccc(-c2c(F)cncc2F)cn1)C(=O)c1cc2nc(N)c(Cl)cc2[nH]1 ; COC1CCOCC1N(Cc1ccc(-c2c(F)cccc2F)cn1)C(=O)c1cc2nc(N)c(Cl)cc2[nH]1 ; COC1CCCCC1N(Cc1ccc(-c2c(F)cncc2F)cn1)C(=O)c1cc2nc(N)c(C)cc2[nH]1]

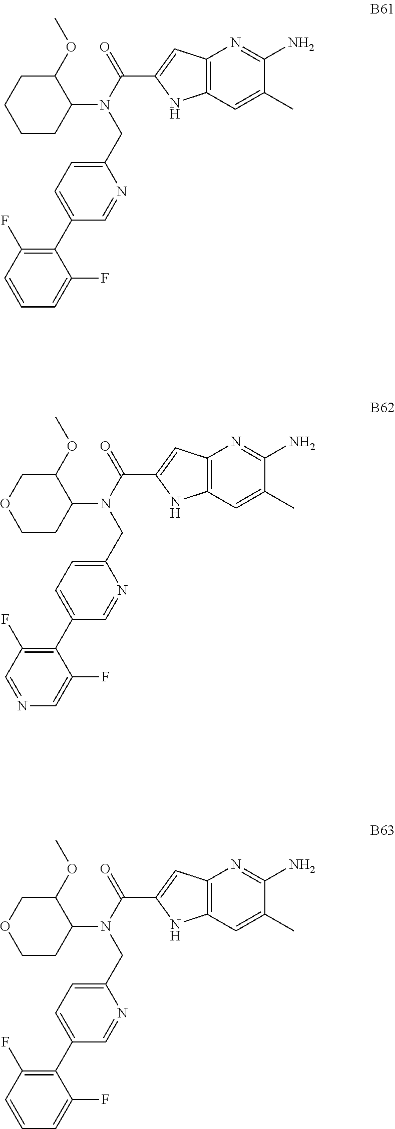 [SMILES: COC1CCCCC1N(Cc1ccc(-c2c(F)cccc2F)cn1)C(=O)c1cc2nc(N)c(C)cc2[nH]1 ; COC1COCCC1N(Cc1ccc(-c2c(F)cncc2F)cn1)C(=O)c1cc2nc(N)c(C)cc2[nH]1 ; COC1COCCC1N(Cc1ccc(-c2c(F)cccc2F)cn1)C(=O)c1cc2nc(N)c(C)cc2[nH]1]

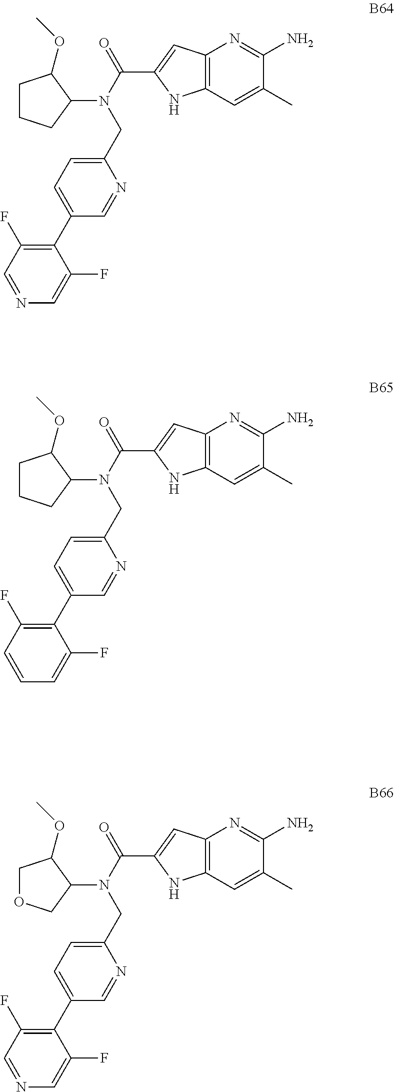 [SMILES: COC1CCCC1N(Cc1ccc(-c2c(F)cncc2F)cn1)C(=O)c1cc2nc(N)c(C)cc2[nH]1 ; COC1CCCC1N(Cc1ccc(-c2c(F)cccc2F)cn1)C(=O)c1cc2nc(N)c(C)cc2[nH]1 ; COC1COCC1N(Cc1ccc(-c2c(F)cncc2F)cn1)C(=O)c1cc2nc(N)c(C)cc2[nH]1]

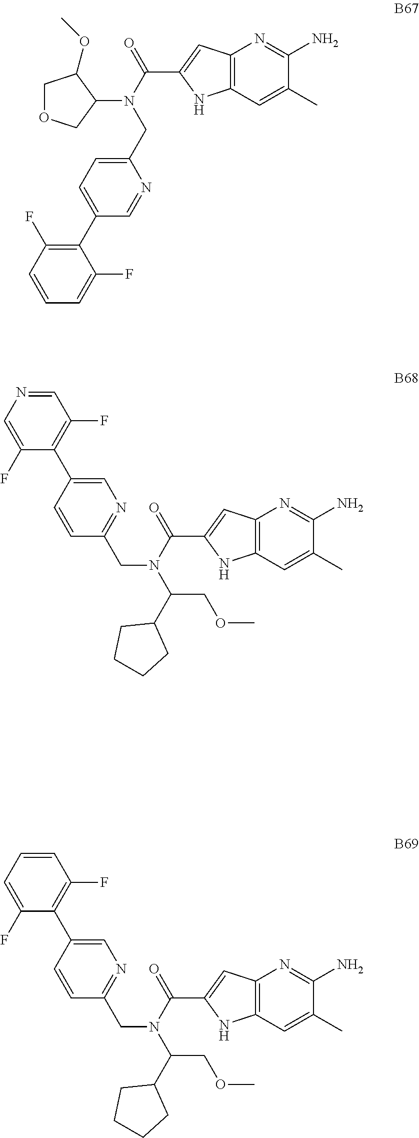 [SMILES: COC1COCC1N(Cc1ccc(-c2c(F)cccc2F)cn1)C(=O)c1cc2nc(N)c(C)cc2[nH]1 ; COCC(C1CCCC1)N(Cc1ccc(-c2c(F)cncc2F)cn1)C(=O)c1cc2nc(N)c(C)cc2[nH]1 ; COCC(C1CCCC1)N(Cc1ccc(-c2c(F)cccc2F)cn1)C(=O)c1cc2nc(N)c(C)cc2[nH]1]

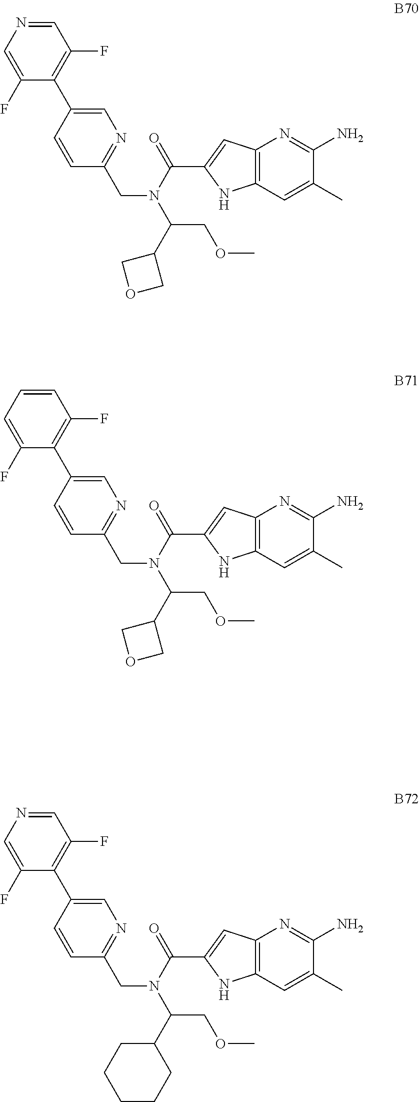 [SMILES: COCC(C1COC1)N(Cc1ccc(-c2c(F)cncc2F)cn1)C(=O)c1cc2nc(N)c(C)cc2[nH]1 ; COCC(C1COC1)N(Cc1ccc(-c2c(F)cccc2F)cn1)C(=O)c1cc2nc(N)c(C)cc2[nH]1 ; COCC(C1CCCCC1)N(Cc1ccc(-c2c(F)cncc2F)cn1)C(=O)c1cc2nc(N)c(C)cc2[nH]1]

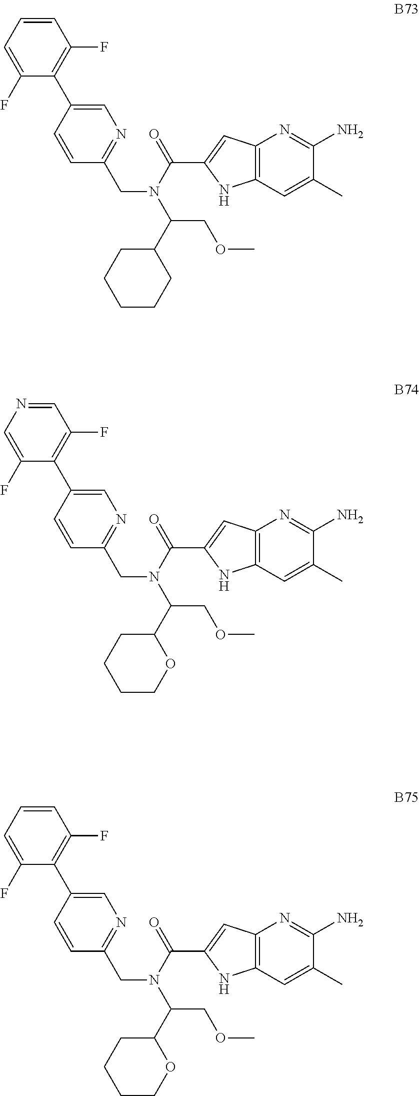 [SMILES: COCC(C1CCCCC1)N(Cc1ccc(-c2c(F)cccc2F)cn1)C(=O)c1cc2nc(N)c(C)cc2[nH]1 ; COCC(C1CCCCO1)N(Cc1ccc(-c2c(F)cncc2F)cn1)C(=O)c1cc2nc(N)c(C)cc2[nH]1 ; COCC(C1CCCCO1)N(Cc1ccc(-c2c(F)cccc2F)cn1)C(=O)c1cc2nc(N)c(C)cc2[nH]1]

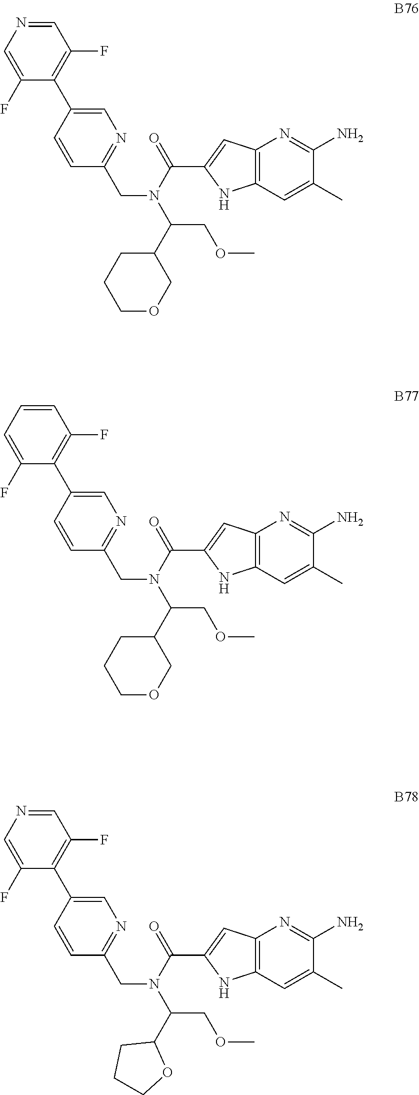 [SMILES: COCC(C1CCCOC1)N(Cc1ccc(-c2c(F)cncc2F)cn1)C(=O)c1cc2nc(N)c(C)cc2[nH]1 ; COCC(C1CCCOC1)N(Cc1ccc(-c2c(F)cccc2F)cn1)C(=O)c1cc2nc(N)c(C)cc2[nH]1 ; COCC(C1CCCO1)N(Cc1ccc(-c2c(F)cncc2F)cn1)C(=O)c1cc2nc(N)c(C)cc2[nH]1]

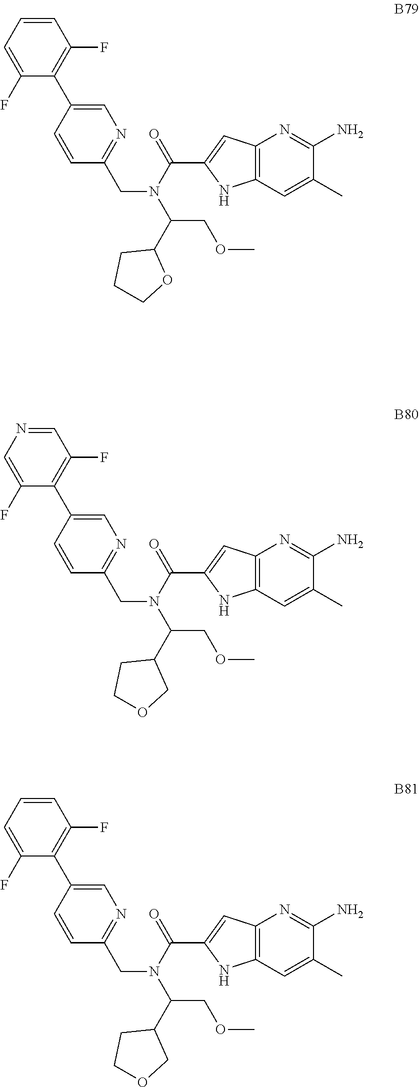 [SMILES: COCC(C1CCCO1)N(Cc1ccc(-c2c(F)cccc2F)cn1)C(=O)c1cc2nc(N)c(C)cc2[nH]1 ; COCC(C1CCOC1)N(Cc1ccc(-c2c(F)cncc2F)cn1)C(=O)c1cc2nc(N)c(C)cc2[nH]1 ; COCC(C1CCOC1)N(Cc1ccc(-c2c(F)cccc2F)cn1)C(=O)c1cc2nc(N)c(C)cc2[nH]1]

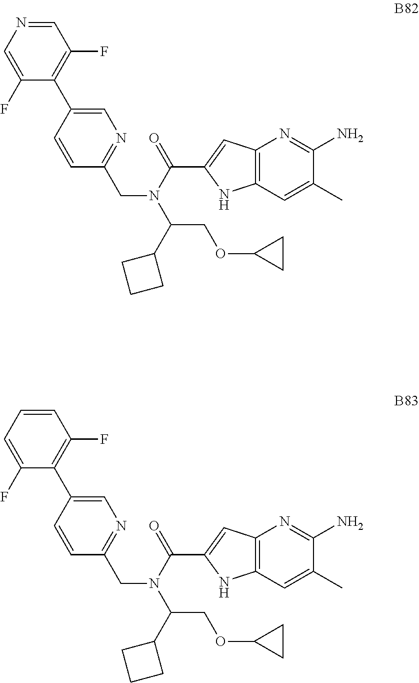 [SMILES: Cc1cc2[nH]c(C(=O)N(Cc3ccc(-c4c(F)cncc4F)cn3)C(COC3CC3)C3CCC3)cc2nc1N ; Cc1cc2[nH]c(C(=O)N(Cc3ccc(-c4c(F)cccc4F)cn3)C(COC3CC3)C3CCC3)cc2nc1N]

Aspect B17. A pharmaceutical composition comprising a compound of any one of Aspects B1-B16 or a pharmaceutically acceptable salt, stereoisomer, tautomer or prodrug thereof, together with a pharmaceutically acceptable excipient.

Aspect B18. A method of decreasing PRMT5 activity by inhibition, which comprises administering to an individual the compound according to any one of Aspects B1-B16, or a pharmaceutically acceptable salt thereof, including the compound of formula (I) or the specific compounds exemplified herein.

Aspect B19. The method of Aspect B18, wherein the disease is selected from cancer.

Aspect B20. Use of a compound of any one of Aspects B1-B16 or a pharmaceutically acceptable salt, stereoisomer, tautomer or prodrug thereof in the preparation of a medicament for treating a disease that is modulated by PRMT5.

Aspect B21. The use of Aspect B20, wherein the disease is cancer.

Aspect B22. The use of Aspect B21, wherein the disease is MTAP-null solid tumor, including but not limited to lung cancer, bladder cancer, melanoma, pancreatic cancer, esophageal cancer, gastric adenocarcinoma, breast cancer or glioblastoma.

### DETAILED DESCRIPTION OF THE INVENTION

The following terms have the indicated meanings throughout the specification:

Unless specifically defined elsewhere in this document, all other technical and scientific terms used herein have the meaning commonly understood by one of ordinary skill in the art to which this invention belongs.

The following terms have the indicated meanings throughout the specification:

As used herein, including the appended claims, the singular forms of words such as “a”, “an”, and “the”, include their corresponding plural references unless the context clearly indicates otherwise.

The term “or” is used to mean, and is used interchangeably with, the term “and/or” unless the context clearly dictates otherwise.

The term “alkyl” includes a hydrocarbon group selected from linear and branched, saturated hydrocarbon groups comprising from 1 to 18, such as from 1 to 12, further such as from 1 to 10, more further such as from 1 to 8, or from 1 to 6, or from 1 to 4, carbon atoms. Examples of alkyl groups comprising from 1 to 6 carbon atoms (i.e., C₁₋₆ alkyl) include, but not limited to, methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1,1-dimethylethyl or t-butyl (“t-Bu”), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl groups.

The term “propyl” includes 1-propyl or n-propyl (“n-Pr”), 2-propyl or isopropyl (“i-Pr”).

The term “butyl” includes 1-butyl or n-butyl (“n-Bu”), 2-methyl-1-propyl or isobutyl (“i-Bu”), 1-methylpropyl or s-butyl (“s-Bu”), 1,1-dimethylethyl or t-butyl (“t-Bu”).

The term “pentyl” includes 1-pentyl, 2-pentyl, 3-pentyl, 2-methyl-2-butyl, 3-methyl-2-butyl, 3-methyl-1-butyl, 2-methyl-1-butyl.

The term “hexyl” includes 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl.

The term “alkylene” refers to a divalent alkyl group by removing two hydrogen from alkane. Alkylene includes but not limited to methylene, ethylene, propylene, and so on.

The term “halogen” includes fluoro (F), chloro (Cl), bromo (Br) and iodo (I).

The term “alkenyl” includes a hydrocarbon group selected from linear and branched hydrocarbon groups comprising at least one C═C double bond and from 2 to 18, such as from 2 to 8, further such as from 2 to 6, carbon atoms. Examples of the alkenyl group, e.g., C₂₋₆ alkenyl, include, but not limited to ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-dienyl, hex-1-enyl, hex-2-enyl, hex-3-enyl, hex-4-enyl, and hexa-1,3-dienyl groups.

The term “alkenylene” refers to a divalent alkenyl group by removing two hydrogen from alkene. Alkenylene includes but not limited to, vinylidene, butenylene, and so on.

The term “alkynyl” includes a hydrocarbon group selected from linear and branched hydrocarbon group, comprising at least one C═C triple bond and from 2 to 18, such as 2 to 8, further such as from 2 to 6, carbon atoms. Examples of the alkynyl group, e.g., C₂₋₆ alkynyl, include, but not limited to ethynyl, 1-propynyl, 2-propynyl (propargyl), 1-butynyl, 2-butynyl, and 3-butynyl groups.

The term “alkynylene” refers to a divalent alkynyl group by removing two hydrogen from alkyne. Alkenylene includes but not limited to ethynylene and so on.

The term “cycloalkyl” includes a hydrocarbon group selected from saturated cyclic hydrocarbon groups, comprising monocyclic and polycyclic (e.g., bicyclic and tricyclic) groups including fused, bridged or spiro cycloalkyl.

For example, the cycloalkyl group may comprise from 3 to 12, such as from 3 to 10, further such as 3 to 8, further such as 3 to 6, 3 to 5, or 3 to 4 carbon atoms. Even further for example, the cycloalkyl group may be selected from monocyclic group comprising from 3 to 12, such as from 3 to 10, further such as 3 to 8, 3 to 6 carbon atoms. Examples of the monocyclic cycloalkyl group include cyclopropyl, cyclobutyl, cyclopentyl, cyclohexyl, cycloheptyl, cyclooctyl, cyclononyl, cyclodecyl, cycloundecyl, and cyclododecyl groups. In particular, examples of the saturated monocyclic cycloalkyl group, e.g., C₃₋₈cycloalkyl, include, but not limited to cyclopropyl, cyclobutyl, cyclopentyl, cyclohexyl, cycloheptyl, and cyclooctyl groups. In a preferred embodiment, the cycloalkyl is a monocyclic ring comprising 3 to 6 carbon atoms (abbreviated as C₃₋₆ cycloalkyl), including but not limited to, cyclopropyl, cyclobutyl, cyclopentyl, and cyclohexyl. Examples of the bicyclic cycloalkyl groups include those having from 7 to 12 ring atoms arranged as a fused bicyclic ring selected from [4,4], [4,5], [5,5], [5,6] and [6,6] ring systems, or as a bridged bicyclic ring selected from bicyclo[2.2.1]heptane, bicyclo[2.2.2]octane, and bicyclo[3.2.2]nonane. Further Examples of the bicyclic cycloalkyl groups include those arranged as a bicyclic ring selected from [5,6] and [6,6] ring systems.

The term “spiro cycloalkyl” includes a cyclic structure which contains carbon atoms and is formed by at least two rings sharing one atom.

The term “fused cycloalkyl” includes a bicyclic cycloalkyl group as defined herein which is saturated and is formed by two or more rings sharing two adjacent atoms.

The term “bridged cycloalkyl” includes a cyclic structure which contains carbon atoms and is formed by two rings sharing two atoms which are not adjacent to each other. The term “7 to 10 membered bridged cycloalkyl” includes a cyclic structure which contains 7 to 12 carbon atoms and is formed by two rings sharing two atoms which are not adjacent to each other.

Examples of fused cycloalkyl, fused cycloalkenyl, or fused cycloalkynyl include but are not limited to bicyclo[1.1.0]butyl, bicyclo[2.1.0]pentyl, bicyclo[3.1.0]hexyl, bicyclo[4.1.0]heptyl, bicyclo[3.3.0]octyl, bicyclo[4.2.0]octyl, decalin, as well as benzo 3 to 8 membered cycloalkyl, benzo C₄₋₆ cycloalkenyl, 2,3-dihydro-1H-indenyl, 1H-indenyl, 1, 2, 3,4-tetralyl, 1,4-dihydronaphthyl, etc. Preferred embodiments are 8 to 9 membered fused rings, which refer to cyclic structures containing 8 to 9 ring atoms within the above examples.

The term “aryl” used alone or in combination with other terms includes a group selected from:
5- and 6-membered carbocyclic aromatic rings, e.g., phenyl;
bicyclic ring systems such as 7 to 12 membered bicyclic ring systems, wherein at least one ring is carbocyclic and aromatic, e.g., naphthyl and indanyl; and,
tricyclic ring systems such as 10 to 15 membered tricyclic ring systems wherein at least one ring is carbocyclic and aromatic, e.g., fluorenyl.

The terms “aromatic hydrocarbon ring” and “aryl” are used interchangeably throughout the disclosure herein. In some embodiments, a monocyclic or bicyclic aromatic hydrocarbon ring has 5 to 10 ring-forming carbon atoms (i.e., C₅₋₁₀ aryl). Examples of a monocyclic or bicyclic aromatic hydrocarbon ring includes, but not limited to, phenyl, naphth-1-yl, naphth-2-yl, anthracenyl, phenanthrenyl, and the like. In some embodiments, the aromatic hydrocarbon ring is a naphthalene ring (naphth-1-yl or naphth-2-yl) or phenyl ring. In some embodiments, the aromatic hydrocarbon ring is a phenyl ring.

Specifically, the term “bicyclic fused aryl” includes a bicyclic aryl ring as defined herein. The typical bicyclic fused aryl is naphthalene.

The term “heteroaryl” includes a group selected from:
5-, 6- or 7-membered aromatic, monocyclic rings comprising at least one heteroatom, for example, from 1 to 4, or, in some embodiments, from 1 to 3, in some embodiments, from 1 to 2, heteroatoms, selected from nitrogen (N), sulfur(S) and oxygen (O), with the remaining ring atoms being carbon;
7- to 12-membered bicyclic rings comprising at least one heteroatom, for example, from 1 to 4, or, in some embodiments, from 1 to 3, or, in other embodiments, 1 or 2, heteroatoms, selected from N, O, and S, with the remaining ring atoms being carbon and wherein at least one ring is aromatic and at least one heteroatom is present in the aromatic ring; and
11- to 14-membered tricyclic rings comprising at least one heteroatom, for example, from 1 to 4, or in some embodiments, from 1 to 3, or, in other embodiments, 1 or 2, heteroatoms, selected from N, O, and S, with the remaining ring atoms being carbon and wherein at least one ring is aromatic and at least one heteroatom is present in an aromatic ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1. When the heteroaryl group contains more than one heteroatom ring member, the heteroatoms may be the same or different. The nitrogen atoms in the ring(s) of the heteroaryl group can be oxidized to form N-oxides.

Specifically, the term “bicyclic fused heteroaryl” includes a 7- to 12-membered, preferably 7- to 10-membered, more preferably 9- or 10-membered fused bicyclic heteroaryl ring as defined herein. Typically, a bicyclic fused heteroaryl is 5-membered/5-membered, 5-membered/6-membered, 6-membered/6-membered, or 6-membered/7-membered bicyclic. The group can be attached to the remainder of the molecule through either ring.

“Heterocyclyl”, “heterocycle” or “heterocyclic” are interchangeable and include a non-aromatic heterocyclyl group comprising one or more heteroatoms selected from nitrogen, oxygen or optionally oxidized sulfur as ring members, with the remaining ring members being carbon, including monocyclic, fused, bridged, and spiro ring, i.e., containing monocyclic heterocyclyl, bridged heterocyclyl, spiro heterocyclyl, and fused heterocyclic groups.

The term “at least one substituent” disclosed herein includes, for example, from 1 to 4, such as from 1 to 3, further as 1 or 2, substituents, provided that the theory of valence is met. For example, “at least one substituent F” disclosed herein includes from 1 to 4, such as from 1 to 3, further as 1 or 2, substituents F.

The term “divalent” refers to a linking group capable of forming covalent bonds with two other moieties. For example, “a divalent cycloalkyl group” refers to a cycloalkyl group obtained by removing two hydrogen from the corresponding cycloalkane to form a linking group. the term “divalent aryl group”, “divalent heterocyclyl group” or “divalent heteroaryl group” should be understood in a similar manner.

Compounds disclosed herein may contain an asymmetric center and may thus exist as enantiomers. “Enantiomers” refer to two stereoisomers of a compound which are non-superimposable mirror images of one another. Where the compounds disclosed herein possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds disclosed herein and/or pharmaceutically acceptable salts thereof are intended to be included. Unless specifically mentioned otherwise, reference to one isomer applies to any of the possible isomers. Whenever the isomeric composition is unspecified, all possible isomers are included.

When compounds disclosed herein contain olefinic double bonds, unless specified otherwise, such double bonds are meant to include both E and Z geometric isomers.

When compounds disclosed herein contain a di-substituted cyclic ring system, substituents found on such ring system may adopt cis and trans formations. Cis formation means that both substituents are found on the upper side of the 2 substituent placements on the carbon, while trans would mean that they were on opposing sides. For example, the di-substituted cyclic ring system may be cyclohexyl or cyclobutyl ring.

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art could select and apply the techniques most likely to achieve the desired separation.

“Diastereomers” refer to stereoisomers of a compound with two or more chiral centers but which are not mirror images of one another. Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., a substantially pure enantiomer, may be obtained by resolution of the racemic mixture using a method such as formation of diastereomers using optically active resolving agents (Eliel, E. and Wilen, S. Stereochemistry of Organic Compounds. New York: John Wiley & Sons, Inc., 1994; Lochmuller, C. H., et al. “Chromatographic resolution of enantiomers: Selective review.” J. Chromatogr., 113 (3) (1975): pp. 283-302). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W., Ed. Drug Stereochemistry: Analytical Methods and Pharmacology. New York: Marcel Dekker, Inc., 1993.

Some of the compounds disclosed herein may exist with different points of attachment of hydrogen, referred to as tautomers. For example, compounds including carbonyl —CH₂C(O)— groups (keto forms) may undergo tautomerism to form hydroxyl —CH═C(OH)— groups (enol forms). Both keto and enol forms, individually as well as mixtures thereof, are also intended to be included where applicable.

“Prodrug” refers to a derivative of an active agent that requires a transformation within the body to release the active agent. In some embodiments, the transformation is an enzymatic transformation. Prodrugs are frequently, although not necessarily, pharmacologically inactive until converted to the active agent.

“Pharmaceutically acceptable salts” refer to those salts which are, within the scope of sound medical judgment, suitable for use in contact with the tissues of humans and lower animals without undue toxicity, irritation, allergic response and the like, and are commensurate with a reasonable benefit/risk ratio. A pharmaceutically acceptable salt may be prepared in situ during the final isolation and purification of the compounds disclosed herein, or separately by reacting the free base function with a suitable organic acid or by reacting the acidic group with a suitable base. The term also includes salts of the stereoisomers (such as enantiomers and/or diastereomers), tautomers and prodrugs of the compound of the invention.

In addition, if a compound disclosed herein is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

The terms “administration”, “administering”, “treating” and “treatment” herein, when applied to an animal, human, experimental subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, and rabbit) and most preferably a human.

The term “effective amount” or “therapeutically effective amount” refers to an amount of the active ingredient, such as compound that, when administered to a subject for treating a disease, or at least one of the clinical symptoms of a disease or disorder, is sufficient to affect such treatment for the disease, disorder, or symptom. The term “therapeutically effective amount” can vary with the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In some embodiments, “therapeutically effective amount” is an amount of at least one compound and/or at least one stereoisomer, tautomer or prodrug thereof, and/or at least one pharmaceutically acceptable salt thereof disclosed herein effective to “treat” as defined herein, a disease or disorder in a subject. In the case of combination therapy, the term “therapeutically effective amount” refers to the total amount of the combination objects for the effective treatment of a disease, a disorder or a condition.

The term “disease” refers to any disease, discomfort, illness, symptoms or indications, and can be interchangeable with the term “disorder” or “condition”.

Throughout this specification and the claims which follow, unless the context requires otherwise, the term “comprise”, and variations such as “comprises” and “comprising” are intended to specify the presence of the features thereafter, but do not exclude the presence or addition of one or more other features. When used herein the term “comprising” can be substituted with the term “containing”, “including” or sometimes “having”.

Throughout this specification and the claims which follow, the term “Cn-m” indicates a range which includes the endpoints, wherein n and m are integers and indicate the number of carbons. Examples include C₁₋₈, C₁₋₆, and the like.

Unless specifically defined elsewhere in this document, all other technical and scientific terms used herein have the meaning commonly understood by one of ordinary skill in the art to which this invention belongs.

### EXAMPLES

### General Synthesis

Compounds disclosed herein, including salts thereof, can be prepared using known organic synthesis techniques and can be synthesized according to any of numerous possible synthetic routes.

The reaction for preparing compounds disclosed herein can be carried out in suitable solvents which can be readily selected by one of skill in the art of organic synthesis. Suitable solvents can be substantially non-reactive with the starting materials, the intermediates, or products at the temperatures at which the reactions are carried out, e.g., temperatures which can range from the solvent's boiling temperature. A given reaction can be carried out in one solvent or mixture of solvents.

The selection of appropriate protecting group, can be readily determined by one skilled in the art. In the synthesis schemes, some protection/deprotection steps are not shown and can be incorporated before, after or in between any steps. The protecting group shown in the synthesis schemes may or may not be used based on reaction conditions. The sequences of reactions may vary and provide similar results.

Reactions can be monitored according to any suitable method known in the art, such as NMR, UV, HPLC, LC-MS and TLC. Compounds can be purified by a variety of methods, including prep-HPLC and silica gel chromatography. Unless specified, prep-HPLC uses a buffered acetonitrile/water systems and silica gel chromatography (including column chromatography and prep-TLC) uses PE/EtOAc or DCM/MeOH systems as mobile phases. NMR spectra are recorded using a Bruker or Varian instrument with preset pulse sequences.

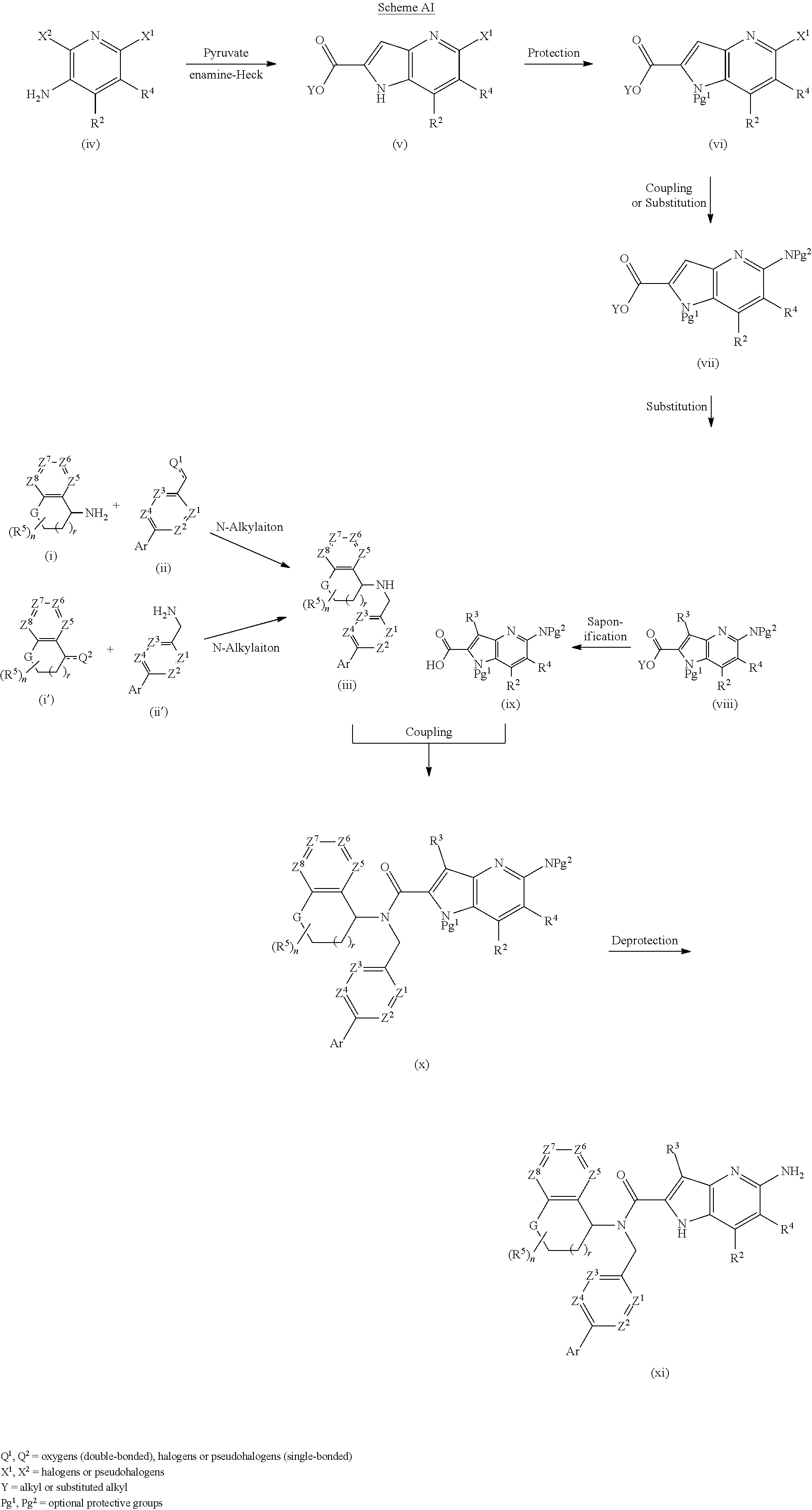

For example, compounds of Formula (I) can be formed as shown in Scheme I. Compound (iii) can be formed by amine alkylation between compound (i) and compound (ii), or compound (i′) and compound (ii′). Compound (iv) can go through an enamine-Heck reaction with pyruvates to give compound (v). Compound (v) can be protected to give compound (vi). Compound (vi) can react with amines via transition-metal catalyzed reactions or substitution reactions to give compound (vii). Compound (vii) can be substituted to give compound (viii). Compound (viii) can be saponified to give compound (ix). Compound (ix) can be couple with compound (iii) under amide coupling conditions to give compound (x). Compound (x) can be unmasked to give compound (xi) [i.e., Formula (I)].

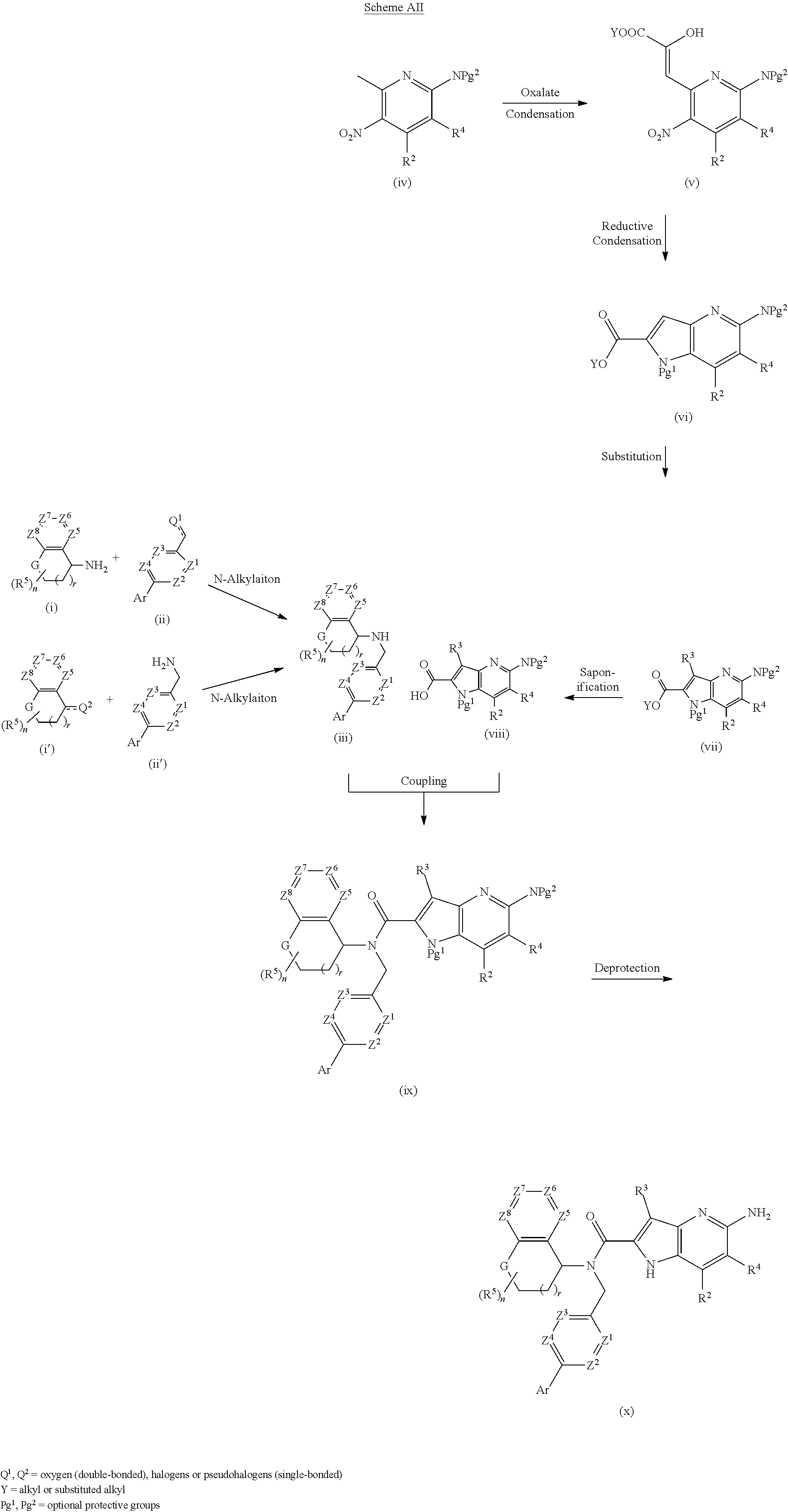

For example, compounds of Formula (I) can be formed as shown in Scheme II. Compound (iii) can be formed by amine alkylation between compound (i) and compound (ii), or compound (i′) and compound (ii′). Compound (iv) can go through a Claisen-type condensation with oxalates to give compound (v). Compound (v) can be reduced and cyclized to give compound (vi). Compound (vi) can be substituted to give compound (vii). Compound (vii) can be saponified to give compound (viii). Compound (viii) can be couple with compound (iii) under amide coupling conditions to give compound (ix). Compound (ix) can be unmasked to give compound (x) [i.e., Formula (I)].

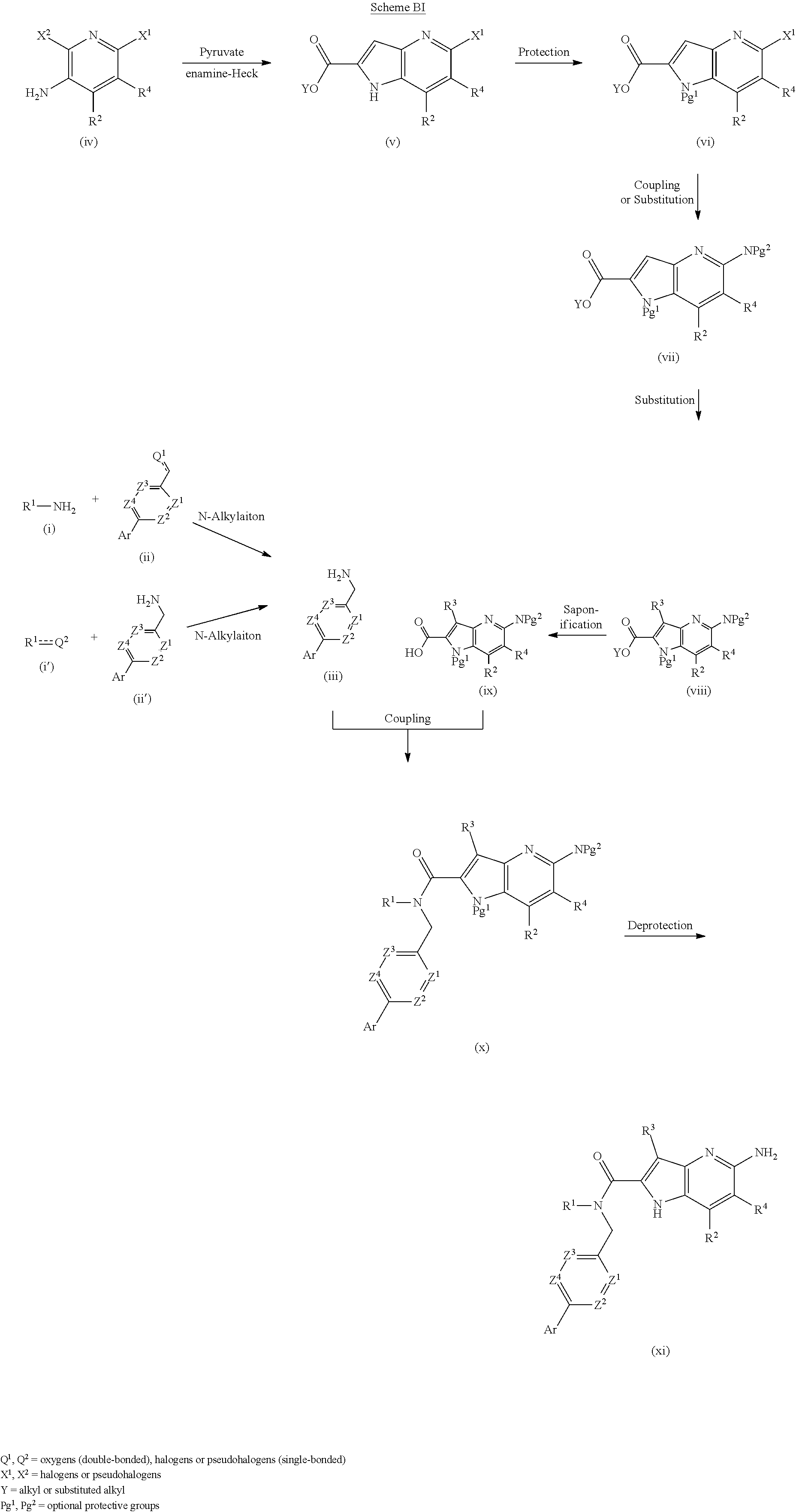

For example, compounds of Formula (I) can be formed as shown in Scheme I. Compound (iii) can be formed by amine alkylation between compound (i) and compound (ii), or compound (i′) and compound (ii′). Compound (iv) can go through an enamine-Heck reaction with pyruvates to give compound (v). Compound (v) can be protected to give compound (vi). Compound (vi) can react with amines via transition-metal catalyzed reactions or substitution reactions to give compound (vii). Compound (vii) can be substituted to give compound (viii). Compound (viii) can be saponified to give compound (ix). Compound (ix) can be couple with compound (iii) under amide coupling conditions to give compound (x). Compound (x) can be unmasked to give compound (xi) [i.e., Formula (I)].

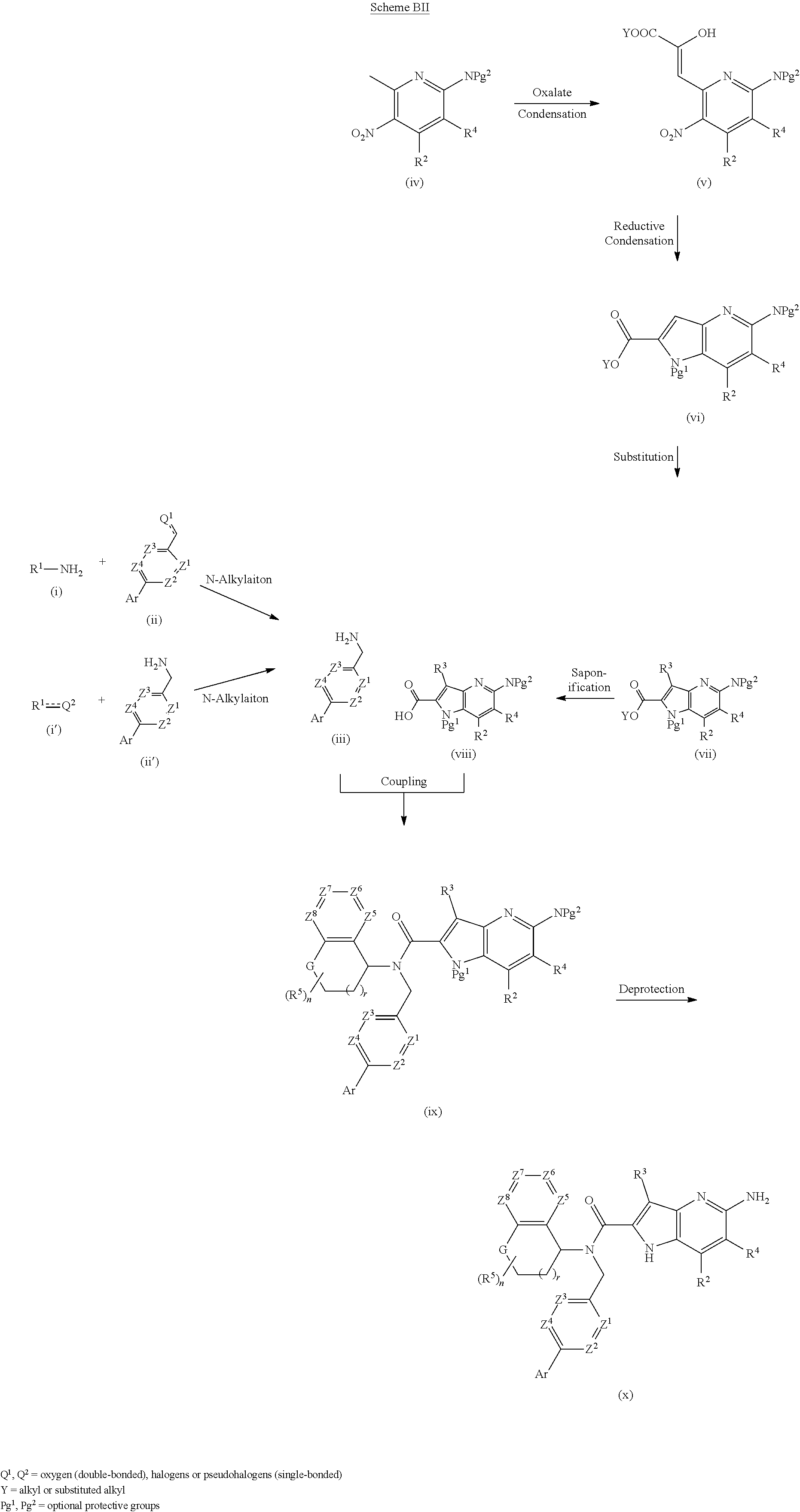

For example, compounds of Formula (I) can be formed as shown in Scheme II. Compound (iii) can be formed by amine alkylation between compound (i) and compound (ii), or compound (i′) and compound (ii′). Compound (iv) can go through a Claisen-type condensation with oxalates to give compound (v). Compound (v) can be reduced and cyclized to give compound (vi). Compound (vi) can be substituted to give compound (vii). Compound (vii) can be saponified to give compound (viii). Compound (viii) can be couple with compound (iii) under amide coupling conditions to give compound (ix). Compound (ix) can be unmasked to give compound (x) [i.e., Formula (I)].

### ABBREVIATIONS

NMR nuclear magnetic resonance
UV ultraviolet
HPLC high performance liquid chromatography
prep preparation/preparative
LC-MS liquid chromatograph mass spectrometer
TLC thin layer chromatography
PE petroleum ether
Et ethyl
Ac acetyl
Ph phenyl
DMF N,N-dimethylformamide
MTBE methyl tert-butyl ether
DCM dichloromethane
Me methyl
DMSO dimethyl sulfoxide
Boc tert-butyloxycarbonyl
SEM 2-(trimethylsilyl)ethoxymethyl
DIPEA diisopropylethylamine
THF tetrahydrofuran
dba dibenzylideneacetone
BINAP 2,2′-bis(diphenylphosphino)-1,1′-binaphthyl
dppf 1,1′-bis(diphenylphosphino) ferrocene
HATU 1-[bis(dimethylamino)methylene]-1H-1,2,3-triazolo[4,5-b]pyridinium
3-oxid hexafluorophosphate
TFA Trifluoroacetic acid
NBS N-bromosuccinimide
BPD bis(pinacolato)diboron
m-CPBA meta-chloroperbenzoic acid
DMP Dess-Martin periodinane
SPhos 2-dicyclohexylphosphino-2′,6′-dimethoxybiphenyl
DIBAL-H diisobutylaluminum hydride
DMAP 4-dimethylaminopyridine

### Example A1: (R)-5-amino-N-((5-(2,6-difluorophenyl)pyridin-2-yl)methyl)-6-methyl-N-(5,6,7,8-tetrahydroquinolin-8-yl)-1H-pyrrolo[3,2-b]pyridine-2-carboxamide

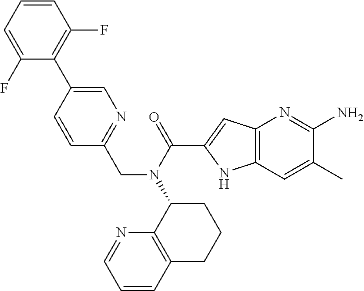 [SMILES: Cc1cc2[nH]c(C(=O)N(Cc3ccc(-c4c(F)cccc4F)cn3)[C@@H]3CCCc4cccnc43)cc2nc1N]

Step 1: 5-chloro-6-methyl-1H-pyrrolo[3,2-b]pyridine-2-carboxylic acid

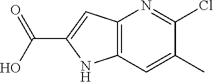

A mixture of 2,6-dichloro-5-methylpyridin-3-amine (10.0 g, 56.5 mmol), pyruvic acid (14.9 g, 169 mmol), Pd(OAc)₂ (0.94 g, 5.65 mmol), PPh₃ (11.8 g, 45.2 mmol) in DMF (100 mL) was stirred at 110° C. for 24 h. The mixture was cooled to room temperature and volatiles were removed in vacuo. Aqueous K₂CO₃ (24 g in 200 mL of water) and MTBE (300 mL) was added, and the mixture was stirred for 10 min. Solid was removed by filtration. The aqueous layer was separated, and the pH was adjusted to 3-4 with aqueous HCl (6 M). The mixture was extracted with EtOAc (500 mL×2). The combined organic layer was washed with brine (300 mL), dried over Na₂SO₄, filtered and concentrated under reduced pressure. The residue was purified by C18 chromatograph (MeCN: 0.03% aqueous formic acid, 0:1 to 1:1) to give the title compound (4.0 g, 34%). LC-MS (M+H)⁺=211.

### Step 2: methyl 5-chloro-6-methyl-1H-pyrrolo[3,2-b]pyridine-2-carboxylate

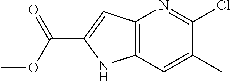

To a mixture of 5-chloro-6-methyl-1H-pyrrolo[3,2-b]pyridine-2-carboxylic acid (8.5 g, 40.3 mmol) in MeOH (100 mL) was added concentrated H₂SO₄ (10 mL) dropwise at 0° C. The mixture was stirred at reflux for overnight. The mixture was cooled to room temperature and most volatiles were removed in vacuo. The mixture was quenched with iced water (100 mL) and extracted with EtOAc (100 mL×2). The combined organic layer was washed with brine (100 mL), dried over Na₂SO₄, filtered and concentrated under reduced pressure. The residue was purified by silica gel chromatograph (PE/EtOAc=8/1) to give the title compound (7.6 g, 84%). LC-MS (M+H)⁺=225.0.

### Step 3: methyl 5-chloro-6-methyl-1-((2-(trimethylsilyl)ethoxy)methyl)-1H-pyrrolo[3,2-b]pyridine-2-carboxylate

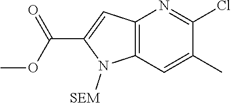

A mixture of methyl 5-chloro-6-methyl-1H-pyrrolo[3,2-b]pyridine-2-carboxylate (7.6 g, 33.9 mmol), SEMCl (11.3 g, 67.8 mmol), DIPEA (13.1 g, 101.7 mmol) in THF (120 mL) was stirred at 70° C. overnight under nitrogen. The mixture was cooled to room temperature and concentrated under reduced pressure. The crude was purified by silica gel chromatograph (PE/EtOAc=10/1) to give the title compound (6.8 g, 71%). LC-MS (M+H)⁺=355.1.

### Step 4: methyl 5-((diphenylmethylene)amino)-6-methyl-1-((2-(trimethylsilyl)ethoxy)methyl)-1H-pyrrolo[3,2-b]pyridine-2-carboxylate

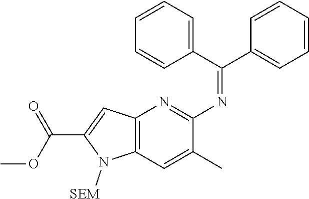 [SMILES: COC(=O)c1cc2nc(N=C(c3ccccc3)c3ccccc3)c(C)cc2n1COCC[Si](C)(C)C]

A mixture of methyl 5-chloro-6-methyl-1-((2-(trimethylsilyl)ethoxy)methyl)-1H-pyrrolo[3,2-b]pyridine-2-carboxylate (2.8 g, 7.89 mmol), diphenylmethanimine (2.14 g, 11.8 mmol), Pd₂(dba)₃ (361 mg, 0.39 mmol), BINAP (0.492 g, 0.79 mmol) and K₃PO₄ (5.0 g, 23.7 mmol) in dioxane (50 mL) was stirred at 100° C. under N₂ overnight. The mixture was cooled to room temperature, diluted with water (100 mL) and extracted with EtOAc (100 mL×2). The combined organic layer was washed with brine (100 mL) and concentrated under reduced pressure. The residue was purified by silica gel chromatograph (PE/EtOAc=4/1) to give the title compound (3.0 g, 76%). LC-MS (M+H)⁺=500.2.

### Step 5: 5-((diphenylmethylene)amino)-6-methyl-1-((2-(trimethylsilyl)ethoxy)methyl)-1H-pyrrolo[3,2-b]pyridine-2-carboxylic Acid

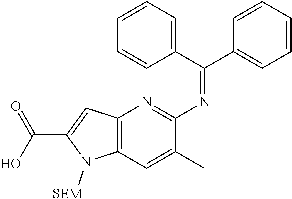 [SMILES: Cc1cc2c(cc(C(=O)O)n2COCC[Si](C)(C)C)nc1N=C(c1ccccc1)c1ccccc1]

To a mixture of methyl 5-((diphenylmethylene)amino)-6-methyl-1-((2-(trimethylsilyl)ethoxy)methyl)-1H-pyrrolo[3,2-b]pyridine-2-carboxylate (3.0 g, 6.0 mmol) in THF (50 mL) and water (50 mL) was added LiOH·H₂O (0.75 g, 18 mol). The mixture was stirred at 60° C. for 4 h. The mixture was cooled to room temperature and the pH was adjusted to 7-8 with aqueous HCl (1 M). The mixture was extracted with EtOAc (50 mL×3). The combined organic layer was washed with brine (50 mL), dried over Na₂SO₄, filtered and concentrated under reduced pressure to give the title compound (2.5 g, 86%). LC-MS (M+H)⁺=486.2.

### Step 6: 5-(2,6-difluorophenyl)picolinaldehyde

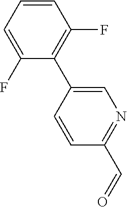 [SMILES: O=Cc1ccc(-c2c(F)cccc2F)cn1]

To a mixture of 5-(4,4,5,5-tetramethyl-1,3,2-dioxaborolan-2-yl)picolinaldehyde (62 g, 266 mmol) and 1,3-difluoro-2-iodobenzene (95.8 g, 399 mmol) in dioxane (600 mL), toluene (300 mL) and water (300 mL) was added Pd(dppf)Cl₂ (9.73 g, 13.3 mmol) and K₃PO₄ (141 g, 665 mmol). The mixture was stirred at 85° C. for 16 h, cooled to room temperature and poured into water (1000 mL). The organic layer was separated. The aqueous phase was extracted with EtOAc (800 mL×2). The combined organic layer was washed with brine (500 mL), dried with Na₂SO₄, filtered and concentrated under vacuum. The residue was purified by silica gel chromatography (PE:EtOAc=50:1 to 5:1) to give the title compound (30 g, 51%). LC-MS (M+H)⁺=220.2.

### Step 7: (R)—N-((5-(2,6-difluorophenyl)pyridin-2-yl)methyl)-5,6,7,8-tetrahydroquinolin-8-amine

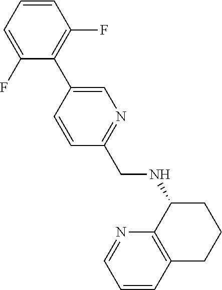 [SMILES: Fc1cccc(F)c1-c1ccc(CN[C@@H]2CCCc3cccnc32)nc1]

To a solution (R)-5,6,7,8-tetrahydroquinolin-8-amine (1.35 g, 9.12 mmol) in DCM (20 mL) was added 5-(2,6-difluorophenyl)picolinaldehyde (2.0 g, 9.12 mmol), MeOH (2 mL) and NaBH(OAc)₃ (5.80 g, 27.4 mmol). The mixture was stirred at room temperature for 1 h and quenched with saturated NaHCO₃ (50 mL). The organic layer was separated, and the aqueous layer was extracted with DCM (50 mL×3). The combined organic layer was washed with brine (50 mL), dried with Na₂SO₄, filtered, and concentrated under vacuum. The residue was purified by silica gel chromatography (PE:EtOAc=1:0 to 0:1) to give the title compound (582 mg, 18%). LC-MS (M+H)⁺=352.1.

### Step 8: (R)—N-((5-(2,6-difluorophenyl)pyridin-2-yl)methyl)-5-((diphenylmethylene)amino)-6-methyl-N-(5,6,7,8-tetrahydroquinolin-8-yl)-1-((2-(trimethylsilyl)ethoxy)methyl)-1H-pyrrolo[3,2-b]pyridine-2-carboxamide

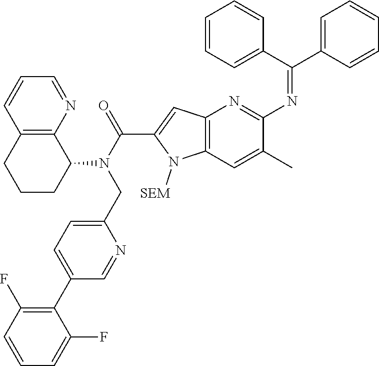 [SMILES: Cc1cc2c(cc(C(=O)N(Cc3ccc(-c4c(F)cccc4F)cn3)[C@@H]3CCCc4cccnc43)n2COCC[Si](C)(C)C)nc1N=C(c1ccccc1)c1ccccc1]

To a solution of 5-((diphenylmethylene)amino)-6-methyl-1-((2-(trimethylsilyl)ethoxy)methyl)-1H-pyrrolo[3,2-b]pyridine-2-carboxylic acid (40 mg, 0.08 mmol) in DMF (3 mL) was added HATU (34.5 mg, 0.09 mmol), (R)—N-((5-(2,6-difluorophenyl)pyridin-2-yl)methyl)-5,6,7,8-tetrahydroquinolin-8-amine (35 mg, 0.1 mmol) and DIPEA (21 mg, 0.16 mmol). The mixture was stirred for 16 h at 55° C. The mixture was cooled to room temperature, diluted with water (50 mL) and then extracted with EtOAc (50 mL). The organic layer was separated and washed with brine (50 mL×2), dried over Na₂SO₄, filtered and concentrated under vacuum. The residue was purified by silica gel chromatograph (DCM:MeOH=20:1) to give the title compound (55 mg, 82%). LC-MS (M+H)⁺=819.1.

### Step 9: (R)-5-amino-N-((5-(2,6-difluorophenyl)pyridin-2-yl)methyl)-6-methyl-N-(5,6,7,8-tetrahydroquinolin-8-yl)-1H-pyrrolo[3,2-b]pyridine-2-carboxamide

To a solution of (R)—N-((5-(2,6-difluorophenyl)pyridin-2-yl)methyl)-5-((diphenylmethylene)amino)-6-methyl-N-(5,6,7,8-tetrahydroquinolin-8-yl)-1-((2-(trimethylsilyl)ethoxy)methyl)-1H-pyrrolo[3,2-b]pyridine-2-carboxamide (55 mg, 0.07 mmol) in DCM (6 mL) was added TFA (6 mL). The mixture was stirred for 2 h at room temperature then concentrated under vacuum. HCl (4 M in MeOH, 4 mL) was added, and the mixture was stirred for 2 h at room temperature. The mixture was concentrated under vacuum, then K₂CO₃ (37 mg, 0.27 mmol), water (2 mL) and MeOH (10 mL) was added. The mixture was stirred for 2 h at 50° C. The mixture was concentrated under vacuum and purified by prep-HPLC to give Example 2 (21 mg, 60%). ¹H NMR (500 MHz, DMSO-d6) δ 11.65-11.16 (m, 1H), 8.77-8.49 (m, 1H), 8.49-8.30 (m, 1H), 8.07-7.47 (m, 4H), 7.39-7.10 (m, 4H), 6.75-6.08 (m, 1H), 6.01-5.37 (m, 1H), 5.29 (s, 2H), 5.25-4.70 (m, 1H), 3.97-3.41 (m, 1H), 2.96-2.68 (m, 2H), 2.46-1.69 (m, 7H). LC-MS (M+H)⁺=525.3.

### Example A2: (R)-5-amino-6-methyl-N-((5-phenylpyridin-2-yl)methyl)-N-(5,6,7,8-tetrahydroquinolin-8-yl)-1H-pyrrolo[3,2-b]pyridine-2-carboxamide

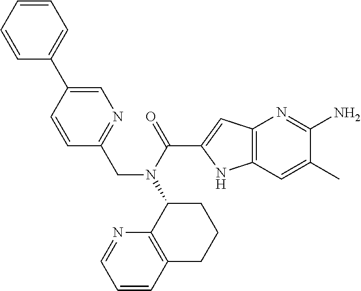 [SMILES: Cc1cc2[nH]c(C(=O)N(Cc3ccc(-c4ccccc4)cn3)[C@@H]3CCCc4cccnc43)cc2nc1N]

### Step 1: (R)—N-((5-phenylpyridin-2-yl)methyl)-5,6,7,8-tetrahydroquinolin-8-amine

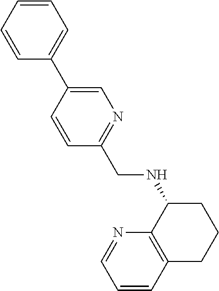 [SMILES: c1ccc(-c2ccc(CN[C@@H]3CCCc4cccnc43)nc2)cc1]

The title compound (300 mg, 44%) was prepared in a manner similar to that in Example 1 step 7 from (R)-5,6,7,8-tetrahydroquinolin-8-amine and 5-phenylpicolinaldehyde. LC-MS (M+H)⁺=316.3.

### Step 2: (R)-5-((diphenylmethylene)amino)-6-methyl-N-((5-phenylpyridin-2-yl)methyl)-N-(5,6,7,8-tetrahydroquinolin-8-yl)-1-((2-(trimethylsilyl)ethoxy)methyl)-1H-pyrrolo[3,2-b]pyridine-2-carboxamide

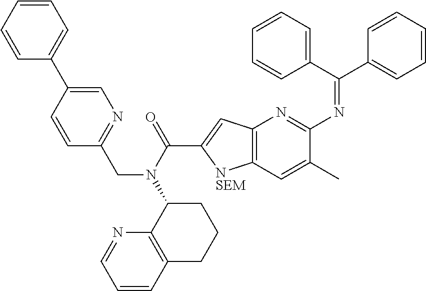 [SMILES: Cc1cc2c(cc(C(=O)N(Cc3ccc(-c4ccccc4)cn3)[C@@H]3CCCc4cccnc43)n2COCC[Si](C)(C)C)nc1N=C(c1ccccc1)c1ccccc1]

The title compound (30 mg, 47%) was prepared in a manner similar to that in Example 1 step 8 from 5-((diphenylmethylene)amino)-6-methyl-1-((2-(trimethylsilyl)ethoxy)methyl)-1H-pyrrolo[3,2-b]pyridine-2-carboxylic acid and (R)—N-((5-phenylpyridin-2-yl)methyl)-5,6,7,8-tetrahydroquinolin-8-amine. LC-MS (M+H)⁺=783.6.

### Step 3: (R)-5-amino-6-methyl-N-((5-phenylpyridin-2-yl)methyl)-N-(5,6,7,8-tetrahydroquinolin-8-yl)-1H-pyrrolo[3,2-b]pyridine-2-carboxamide

Example 2 (3 mg, 16%) was prepared in a manner similar to that in Example 1 step 9 from (R)-5-((diphenylmethylene)amino)-6-methyl-N-((5-phenylpyridin-2-yl)methyl)-N-(5,6,7,8-tetrahydroquinolin-8-yl)-1-((2-(trimethylsilyl)ethoxy)methyl)-1H-pyrrolo[3,2-b]pyridine-2-carboxamide. ¹H NMR (500 MHz, DMSO-d6) δ 11.60-11.26 (m, 1H), 9.07-8.76 (m, 1H), 8.56-8.35 (m, 1H), 8.24-8.02 (m, 1H), 7.97-7.34 (m, 8H), 7.33-7.17 (m, 1H), 6.77-6.19 (m, 1H), 6.06-5.44 (m, 1H), 5.44-5.28 (m, 2H), 5.28-4.87 (m, 1H), 4.86-3.50 (m, 1H), 2.98-2.72 (m, 2H), 2.54-2.39 (m, 1H), 2.36-1.73 (m, 6H). LC-MS (M+H)⁺=489.4.

### Example A3: (R)-5-amino-N-((3′,5′-difluoro-[3,4′-bipyridin]-6-yl)methyl)-6-methyl-N-(5,6,7,8-tetrahydroquinolin-8-yl)-1H-pyrrolo[3,2-b]pyridine-2-carboxamide

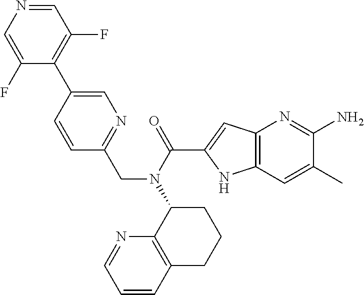 [SMILES: Cc1cc2[nH]c(C(=O)N(Cc3ccc(-c4c(F)cncc4F)cn3)[C@@H]3CCCc4cccnc43)cc2nc1N]

### Step 1: 3′,5′-difluoro-[3,4′-bipyridine]-6-carbaldehyde

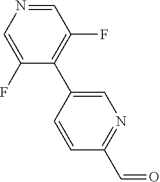 [SMILES: O=Cc1ccc(-c2c(F)cncc2F)cn1]

To a mixture of 3,5-difluoro-4-iodopyridine (0.85 g, 3.53 mmol) in dioxane (17 mL), toluene (8.5 mL) and water (8.5 mL) was added 5-(4,4,5,5-tetramethyl-1,3,2-dioxaborolan-2-yl)picolinaldehyde (1.64 g, 7.05 mmol), K₃PO₄ (2.25 g, 10.6 mmol) and Pd(dppf)Cl₂ (258 mg, 0.35 mmol) at 20° C. The mixture was stirred at 85° C. for 3 h. The mixture was cooled to room temperature and filtered. The filtrate was concentrated under reduced pressure. The residue was purified by silica gel chromatograph (PE:EtOAc=1:0 to 1:1) to give the title compound (0.70 g, 90%). LC-MS (M+H)⁺=221.2.

### Step 2: (R)—N-((3′,5′-difluoro-[3,4′-bipyridin]-6-yl)methyl)-5,6,7,8-tetrahydroquinolin-8-amine

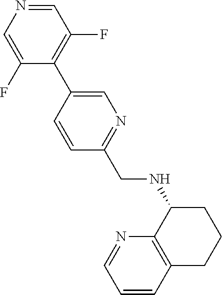 [SMILES: Fc1cncc(F)c1-c1ccc(CN[C@@H]2CCCc3cccnc32)nc1]

To a solution of (R)-5,6,7,8-tetrahydroquinolin-8-amine (525 mg, 3.54 mmol) in DCM (13 mL) was added NaBH(OAc)₃ (939 mg, 4.43 mmol), MeOH (0.13 mL) and 3′,5′-difluoro-[3,4′-bipyridine]-6-carbaldehyde (0.65 g, 2.95 mmol). The mixture was stirred at 20° C. for 1 h then poured into saturated NaHCO₃ (50 mL). The mixture was extracted with DCM (50 mL×3). The combined organic layer was washed with brine (50 mL), dried over Na₂SO₄, filtered and concentrated under vacuum. The residue was purified by prep-HPLC to give the title compound (216 mg, 21%). LC-MS (M+H)⁺=353.2.

### Step 3:5-amino-6-methyl-1-((2-(trimethylsilyl)ethoxy)methyl)-1H-pyrrolo[3,2-b]pyridine-2-carboxylic Acid

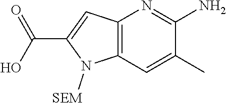 [SMILES: Cc1cc2c(cc(C(=O)O)n2COCC[Si](C)(C)C)nc1N]

To a solution of ethyl 5-((diphenylmethylene)amino)-6-methyl-1-((2-(trimethylsilyl)ethoxy)methyl)-1H-pyrrolo[3,2-b]pyridine-2-carboxylate (900 mg, 1.75 mmol) in THF (20 mL) and water (5 mL) was added LiOH (126 mg, 5.30 mmol). The mixture was stirred at 60° C. for 3 h. The mixture was cooled to room temperature and the pH was adjusted to 2 with 1 M HCl. The mixture was stirred at 80° C. for 2 h, cooled to room temperature then concentrated under reduced pressure. The residue was purified by C18 chromatography (MeCN:water=0:1 to 3:2) to give the title compound (380 mg, 70%). LC-MS (M+H)⁺=322.15.

### Step 4: (R)-5-amino-N-((3′,5′-difluoro-[3,4′-bipyridin]-6-yl)methyl)-6-methyl-N-(5,6,7,8-tetrahydroquinolin-8-yl)-1-((2-(trimethylsilyl)ethoxy)methyl)-1H-pyrrolo[3,2-b]pyridine-2-carboxamide

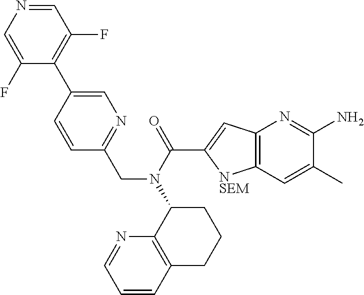 [SMILES: Cc1cc2c(cc(C(=O)N(Cc3ccc(-c4c(F)cncc4F)cn3)[C@@H]3CCCc4cccnc43)n2COCC[Si](C)(C)C)nc1N]

To a solution of 5-amino-6-methyl-1-((2-(trimethylsilyl)ethoxy)methyl)-1H-pyrrolo[3,2-b]pyridine-2-carboxylic acid (36 mg, 0.11 mmol) in DMF (1 mL) was added (R)—N-((3′,5′-difluoro-[3,4′-bipyridin]-6-yl)methyl)-5,6,7,8-tetrahydroquinolin-8-amine (47 mg, 0.13 mmol), HATU (47 mg, 0.12 mmol) and DIPEA (0.08 mL, 0.45 mmol). The mixture was stirred at 50° C. for 2 h. The mixture was cooled to room temperature and diluted with water (10 mL) then extracted with EtOAc (20 mL). The organic layer was dried over Na₂SO₄, filtered and concentrated in vacuo. The residue was purified by prep-TLC (DCM/MeOH=20:1) to give the title compound (34 mg, 47%). LC-MS (M+H)⁺=656.3.

### Step 5: (R)-5-amino-N-((3′,5′-difluoro-[3,4′-bipyridin]-6-yl)methyl)-6-methyl-N-(5,6,7,8-tetrahydroquinolin-8-yl)-1H-pyrrolo[3,2-b]pyridine-2-carboxamide

A mixture of (R)-5-amino-N-((3′,5′-difluoro-[3,4′-bipyridin]-6-yl)methyl)-6-methyl-N-(5,6,7,8-tetrahydroquinolin-8-yl)-1-((2-(trimethylsilyl)ethoxy)methyl)-1H-pyrrolo[3,2-b]pyridine-2-carboxamide (34 mg, 0.05 mmol) in DCM (3 mL) and TFA (1 mL) was stirred at room temperature for 1 h. The mixture was concentrated in vacuo, then re-dissolved in MeOH (5 mL) followed by addition of K₂CO₃ (14 mg). The reaction mixture was stirred at room temperature for 1 h. The mixture was concentrated under reduced pressure. The residue was purified by prep-HPLC to give the title compound (10 mg, 37%). ¹H NMR (500 MHz, DMSO-d6) δ 11.48-11.16 (m, 1H), 8.85-7.46 (m, 7H), 7.37-7.27 (m, 1H), 7.27-7.13 (m, 1H), 6.73-6.07 (m, 1H), 6.02-5.39 (m, 1H), 5.37-4.72 (m, 3H), 3.95-3.50 (m, 1H), 2.92-2.66 (m, 2H), 2.44-1.69 (m, 7H). LC-MS (M+H)⁺=526.4.

### Example A4: (R)-5-amino-N-((5-(1,4-dimethyl-1H-pyrazol-5-yl) pyridin-2-yl)methyl)-6-methyl-N-(5,6,7,8-tetrahydroquinolin-8-yl)-1H-pyrrolo[3,2-b]pyridine-2-carboxamide

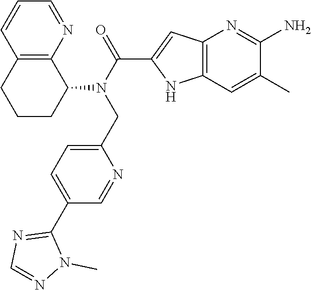 [SMILES: Cc1cc2[nH]c(C(=O)N(Cc3ccc(-c4ncnn4C)cn3)[C@@H]3CCCc4cccnc43)cc2nc1N]

### Step 1: 5-(1,4-dimethyl-1H-pyrazol-5-yl)picolinaldehyde

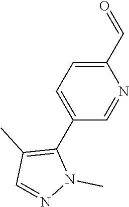

To a mixture of 5-bromopicolinaldehyde (2.0 g, 10.8 mmol) in dioxane (20 mL), water (10 mL) and toluene (10 mL) was added 1,4-dimethyl-5-(4,4,5,5-tetramethyl-1,3,2-dioxaborolan-2-yl)-1H-pyrazole (2.9 g, 12.9 mmol), Pd(dppf)Cl₂ (787 mg, 1.1 mmol) and K₃PO₄ (6.85 g, 32.3 mmol). The mixture was stirred at 80° C. for 12 h. The reaction mixture was cooled to room temperature and filtered. The filtrate was concentrated under reduced pressure. The residue was purified by silica gel chromatograph (PE:EtOAc=1:0 to 0:1). To give the title compound (0.98 g, 45%). LC-MS (M+H)⁺=202.3.

### Step 2: (R)—N-((5-(1,4-dimethyl-1H-pyrazol-5-yl) pyridin-2-yl)methyl)-5,6,7,8-tetrahydroquinolin-8-amine

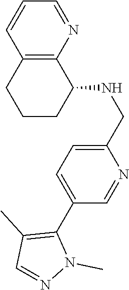 [SMILES: Cc1cnn(C)c1-c1ccc(CN[C@@H]2CCCc3cccnc32)nc1]

The title compound (220 mg, 33%) was prepared in a manner similar to that in Example 1 step 7 from (R)-5,6,7,8-tetrahydroquinolin-8-amine and 5-(1,4-dimethyl-1H-pyrazol-5-yl)picolinaldehyde. LC-MS (M+H)⁺=334.3.

### Step 3: (R)—N-((5-(1,4-dimethyl-1H-pyrazol-5-yl) pyridin-2-yl)methyl)-5-((diphenylmethylene)amino)-6-methyl-N-(5,6,7,8-tetrahydroquinolin-8-yl)-1-((2-(trimethylsilyl)ethoxy)methyl)-1H-pyrrolo[3,2-b]pyridine-2-carboxamide

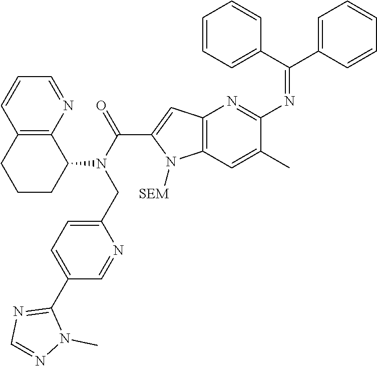 [SMILES: Cc1cc2c(cc(C(=O)N(Cc3ccc(-c4ncnn4C)cn3)[C@@H]3CCCc4cccnc43)n2COCC[Si](C)(C)C)nc1N=C(c1ccccc1)c1ccccc1]

The title compound (20 mg, 50%) was prepared in a manner similar to that in Example 1 step 8 from 5-((diphenylmethylene)amino)-6-methyl-1-((2-(trimethylsilyl)ethoxy)methyl)-1H-pyrrolo[3,2-b]pyridine-2-carboxylic acid and (R)—N-((5-(1,4-dimethyl-1H-pyrazol-5-yl) pyridin-2-yl)methyl)-5,6,7,8-tetrahydroquinolin-8-amine. LC-MS (M+H)⁺=801.6.

### Step 4: (R)-5-amino-N-((5-(1,4-dimethyl-1H-pyrazol-5-yl) pyridin-2-yl)methyl)-6-methyl-N-(5,6,7,8-tetrahydroquinolin-8-yl)-1H-pyrrolo[3,2-b]pyridine-2-carboxamide

Example 4 (2.5 mg, 20%) was prepared in a manner similar to that in Example 1 step 9 from (R)—N-((5-(1,4-dimethyl-1H-pyrazol-5-yl) pyridin-2-yl)methyl)-5-((diphenylmethylene)amino)-6-methyl-N-(5,6,7,8-tetrahydroquinolin-8-yl)-1-((2-(trimethylsilyl)ethoxy)methyl)-1H-pyrrolo[3,2-b]pyridine-2-carboxamide. ¹H NMR (500 MHz, DMSO-d6) δ 11.56-11.08 (m, 1H), 8.73-8.46 (m, 1H), 8.46-8.29 (m, 1H), 7.99-7.45 (m, 3H), 7.43-7.27 (m, 2H), 7.27-7.07 (m, 1H), 6.76-6.19 (m, 1H), 6.02-5.33 (m, 1H), 5.30 (s, 2H), 5.23-4.74 (m, 1H), 4.02-3.58 (m, 4H), 2.93-2.66 (m, 2H), 2.49-2.42 (m, 1H), 2.26-1.69 (m, 9H). LC-MS (M+H)⁺=507.4.

### Example A5: (R)-5-amino-3-bromo-N-((5-(2,6-difluorophenyl)pyridin-2-yl)methyl)-6-methyl-N-(5,6,7,8-tetrahydroquinolin-8-yl)-1H-pyrrolo[3,2-b]pyridine-2-carboxamide

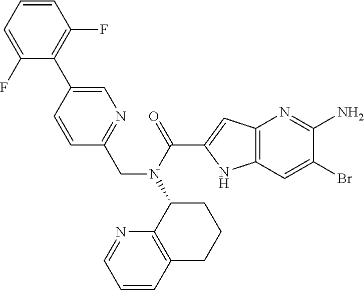 [SMILES: Nc1nc2cc(C(=O)N(Cc3ccc(-c4c(F)cccc4F)cn3)[C@@H]3CCCc4cccnc43)[nH]c2cc1Br]

### Step 1: 3-bromo-5-((diphenylmethylene)amino)-6-methyl-1-((2-(trimethylsilyl)ethoxy)methyl)-1H-pyrrolo[3,2-b]pyridine-2-carboxylic Acid

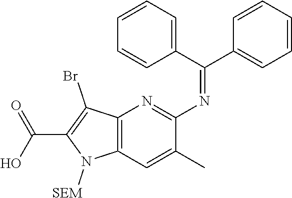 [SMILES: Cc1cc2c(nc1N=C(c1ccccc1)c1ccccc1)c(Br)c(C(=O)O)n2COCC[Si](C)(C)C]

To a solution of 5-((diphenylmethylene)amino)-6-methyl-1-((2-(trimethylsilyl)ethoxy)methyl)-1H-pyrrolo[3,2-b]pyridine-2-carboxylic acid (150 mg, 0.31 mmol) in DMF (2 mL) was added NBS (66 mg, 0.37 mmol). The mixture was stirred at room temperature for 1 h. Water (10 mL) was added and the mixture was extracted with EtOAc (20 mL). The organic layer was dried over Na₂SO₄, filtered and concentrated in vacuo. The residue was purified by prep-TLC (DCM:MeOH=15:1) to give the title compound (130 mg, 75%). LC-MS (M+H)⁺=564.1.

### Step 2: (R)-3-bromo-N-((5-(2,6-difluorophenyl)pyridin-2-yl)methyl)-5-((diphenylmethylene)amino)-6-methyl-N-(5,6,7,8-tetrahydroquinolin-8-yl)-1-((2-(trimethylsilyl)ethoxy)methyl)-1H-pyrrolo[3,2-b]pyridine-2-carboxamide

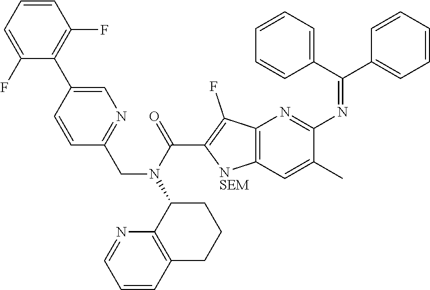 [SMILES: Cc1cc2c(nc1N=C(c1ccccc1)c1ccccc1)c(F)c(C(=O)N(Cc1ccc(-c3c(F)cccc3F)cn1)[C@@H]1CCCc3cccnc31)n2COCC[Si](C)(C)C]

The title compound (20 mg, 20%) was prepared in a manner similar to that in Example 1 step 8 from 3-bromo-5-((diphenylmethylene)amino)-6-methyl-1-((2-(trimethylsilyl)ethoxy)methyl)-1H-pyrrolo[3,2-b]pyridine-2-carboxylic acid and (R)—N-((5-(2,6-difluorophenyl)pyridin-2-yl)methyl)-5,6,7,8-tetrahydroquinolin-8-amine. LC-MS (M+H)⁺=897.2.

### Step 3: (R)-5-amino-3-bromo-N-((5-(2,6-difluorophenyl)pyridin-2-yl)methyl)-6-methyl-N-(5,6,7,8-tetrahydroquinolin-8-yl)-1H-pyrrolo[3,2-b]pyridine-2-carboxamide

Example 5 (10 mg, 77%) was prepared in a manner similar to that in Example 1 step 9 from (R)-3-bromo-N-((5-(2,6-difluorophenyl)pyridin-2-yl)methyl)-5-((diphenylmethylene)amino)-6-methyl-N-(5,6,7,8-tetrahydroquinolin-8-yl)-1-((2-(trimethylsilyl)ethoxy)methyl)-1H-pyrrolo[3,2-b]pyridine-2-carboxamide. ¹H NMR (500 MHz, DMSO-d6) δ 11.70 (s, 1H), 8.56 (s, 1H), 8.44 (d, J=3.9 Hz, 1H), 7.94 (d, J=7.9 Hz, 1H), 7.73 (d, J=8.1 Hz, 1H), 7.59-7.47 (m, 2H), 7.33 (s, 1H), 7.28 (t, J=7.9 Hz, 2H), 7.21 (dd, J=7.3, 4.9 Hz, 1H), 5.61 (s, 2H), 5.23-5.20 (m, 1H), 4.97-4.94 (m, 1H), 3.94-3.91 (m, 1H), 2.85-2.80 (m, 1H), 2.68-2.64 (m, 1H), 2.57 (s, 1H), 2.14 (s, 3H), 2.06-1.90 (m, 2H), 1.75-1.60 (m, 1H). LC-MS (M+H)⁺=603.1.

### Example A6: 5-amino-N-(4-cyclopropyl-5,6,7,8-tetrahydroquinolin-8-yl)-N-((5-(2,6-difluorophenyl)pyridin-2-yl)methyl)-6-methyl-1H-pyrrolo[3,2-b]pyridine-2-carboxamide

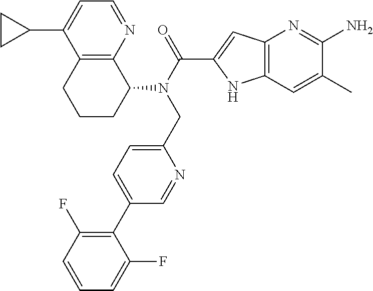 [SMILES: Cc1cc2[nH]c(C(=O)N(Cc3ccc(-c4c(F)cccc4F)cn3)[C@@H]3CCCc4c(C5CC5)ccnc43)cc2nc1N]

### Step 1: tert-butyl ((5-(2,6-difluorophenyl)pyridin-2-yl)methyl)carbamate

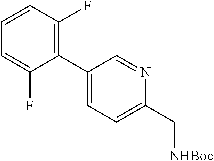 [SMILES: CC(C)(C)OC(=O)NCc1ccc(-c2c(F)cccc2F)cn1]

To a solution of tert-butyl ((5-bromopyridin-2-yl)methyl)carbamate (5.0 g, 17.4 mmol) in dioxane (50 mL) was added BPD (5.31 g, 20.9 mmol), Pd(dppf)Cl₂ (1.27 g, 1.74 mmol) and KOAc (4.27 g, 43.5 mmol). The mixture was stirred at 85° C. for 12 h. The mixture was cooled to room temperature, filtered and concentrated under reduced pressure. The crude was re-dissolved in dioxane (25 mL), toluene (12 mL) and water (12 mL) followed by addition of 1,3-difluoro-2-iodobenzene (4.28 g, 17.8 mmol), K₃PO₄ (6.32 g, 29.8 mmol) and Pd(dppf)Cl₂ (871 mg, 1.19 mmol). The mixture was stirred at 85° C. for 3 h. The mixture was cooled to room temperature, diluted with water (30 mL) and extracted with EtOAc (20 mL×3). The combined organic layer was washed with brine (10 mL×2), dried over Na₂SO₄, filtered and concentrated under reduced pressure. The residue was purified by silica gel chromatograph (PE:EtOAc=1:0 to 5:1) to give the title compound (3.0 g, 54%). ¹H NMR (400 MHZ, CDCl₃) δ 8.64 (s, 1H), 7.78 (d, J=8.4 Hz, 1H), 7.39 (d, J=8.4 Hz, 1H), 7.36-7.28 (m, 1H), 7.06-6.96 (m, 1H), 5.62 (br s, 1H), 4.50 (d, J=5.6 Hz, 2H), 7.47 (s, 9H). LC-MS (M+H)⁺=321.1.

### Step 2: (5-(2,6-difluorophenyl)pyridin-2-yl)methanamine

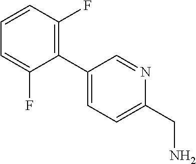 [SMILES: NCc1ccc(-c2c(F)cccc2F)cn1]

A mixture of tert-butyl ((5-(2,6-difluorophenyl)pyridin-2-yl)methyl)carbamate (3.0 g, 9.37 mmol) in methanolic HCl (4 M, 50 mL) was stirred at 25° C. for 2 h, then the mixture was neutralized with methanolic NaOH (1 M) to pH 7. The mixture was concentrated to dryness under reduced pressure, and the residue was triturated with DCM/MeOH (10/1, 10 mL). The filtrate was collected by filtration and concentrated under vacuum to give the title compound (1.5 g, 73%). ¹H NMR (400 MHZ, CD₃OD) δ 8.72 (s, 1H), 7.97 (d, J=6.0 Hz, 1H), 7.60 (d, J=6.0 Hz, 1H), 7.54-7.45 (m, 1H), 7.20-7.10 (m, 1H), 4.37 (s, 2H). LC-MS (M+H)⁺=221.1.

### Step 3: 4-methoxy-5,6,7,8-tetrahydroquinoline

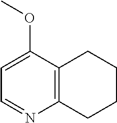

To a solution of PtO₂ (3.71 g, 16.3 mmol) in TFA (200 mL) was added 4-methoxyquinoline (20 g, 126 mmol) under nitrogen. The suspension was degassed and purged with hydrogen for 3 times. The mixture was stirred under hydrogen (50 psi) at 50° C. for 3 h. The filtrate was collected by filtration, concentration to about 50 mL and diluted with water (100 mL). The pH of the mixture was adjusted to 9 with aqueous NaOH (1 M). The mixture was extracted with DCM (50 mL×3). The combined organic layer was washed with brine (50 mL×2), dried over Na₂SO₄, filtered and concentrated under reduced pressure to give the title compound (20 g, 98%). ¹H NMR (400 MHz, CD₃OD) δ 8.40 (d, J=6.4 Hz, 1H), 7.30 (d, J=6.4 Hz, 1H), 4.11 (s, 3H), 2.96 (t, J=6.2 Hz, 2H), 2.71 (t, J=6.2 Hz, 2H), 1.96-1.83 (m, 4H). LC-MS (M+H)⁺=164.2.

### Step 4: 4-bromo-5,6,7,8-tetrahydroquinoline

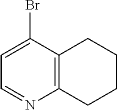 [SMILES: Brc1ccnc2c1CCCC2]

To a solution of 4-methoxy-5,6,7,8-tetrahydroquinoline (17 g, 104 mmol) in DMF (170 mL) at 0° C. was added POBr₃ (89.6 g, 312 mmol). The mixture was heated to 100° C. and stirred for 12 h. The reaction mixture was cooled to 0° C. and slowly added to iced water (300 mL). The mixture was extracted with EtOAc (100 mL×3). The combined organic layer was washed with brine (50 mL×2), dried over Na₂SO₄, filtered and concentrated under reduced pressure. The residue was purified by silica gel chromatograph (PE:EtOAc=1:0 to 5:1) to give the title compound (15 g). LC-MS (M+H)⁺=212.1.

### Step 5: 4-bromo-5,6,7,8-tetrahydroquinoline 1-oxide

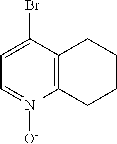 [SMILES: [O-][n+]1ccc(Br)c2c1CCCC2]

To a solution of 4-bromo-5,6,7,8-tetrahydroquinoline (15 g, 70.7 mmol) in DCM (195 mL) was added m-CPBA (80%, 30.5 g, 141 mmol). The mixture was stirred at 42° C. for 2 h then cooled to room temperature. The mixture was washed with water (100 mL) and the aqueous phase was extracted with DCM (50 mL×3). The combined organic layer was washed with brine, (50 mL×2), dried over Na₂SO₄, filtered and concentrated under reduced pressure. The residue was purified by silica gel chromatograph (PE:EtOAc=1:0 to 3:1) to give the title compound (14 g, 87%). LC-MS (M+H)⁺=228.2.

### Step 6:4-bromo-5,6,7,8-tetrahydroquinolin-8-yl acetate

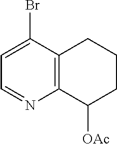 [SMILES: CC(=O)OC1CCCc2c(Br)ccnc21]

A mixture of 4-bromo-5,6,7,8-tetrahydroquinoline 1-oxide (14 g, 61.4 mmol) in Ac₂O (70 mL) was stirred at 55° C. for 2 h and cooled to room temperature. The filtrate was collected by filtration and concentrated under reduced pressure to give the title compound (14 g, 84%). LC-MS (M+H)⁺=270.1.

### Step 7: 4-bromo-5,6,7,8-tetrahydroquinolin-8-ol

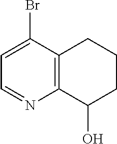 [SMILES: OC1CCCc2c(Br)ccnc21]

Four identical reactions as described below were set up in parallel. To a solution of 4-bromo-5,6,7,8-tetrahydroquinolin-8-yl acetate (3.0 g, 11.1 mmol) in MeOH (15 mL) was added K₂CO₃ (5.8 g, 42.2 mmol). The mixture was stirred at 25° C. for 2 h.

The four batches of reaction mixture were combined. The filtrate was collected by filtration and concentrated under reduced pressure. The residue was purified by silica gel chromatograph (PE:EtOAc=1:0 to 1:1) to give the title compound (5.2 g, 51%). LC-MS (M+H)⁺=228.2.

### Step 8: 4-bromo-6,7-dihydroquinolin-8(5H)-one

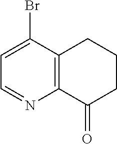 [SMILES: O=C1CCCc2c(Br)ccnc21]

To a solution of 4-bromo-5,6,7,8-tetrahydroquinolin-8-ol (5.0 g, 21.9 mmol) in DCM (260 mL) was added DMP (16.7 g, 39.5 mmol). The mixture was stirred at 25° C. for 8 h. The mixture was filtered and the filtrate was concentrated under reduced pressure. The residue was purified by silica gel chromatograph (PE:EtOAc=1:0 to 1:1) to give the title compound (4.0 g, 81%). LC-MS (M+H)⁺=226.1.

### Step 9: 4-cyclopropyl-6,7-dihydroquinolin-8 (5H)-one

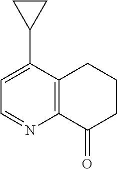 [SMILES: O=C1CCCc2c(C3CC3)ccnc21]

To a solution of 4-bromo-6,7-dihydroquinolin-8 (5H)-one (1.0 g, 4.42 mmol) and cyclopropylboronic acid (950 mg, 11.1 mmol) in water (1 mL) and toluene (9 mL) was added SPhos (145 mg, 0.35 mmol), Pd(OAc)₂ (99 mg, 0.44 mmol) and K₃PO₄ (2.35 g, 11.1 mmol). The mixture was stirred at 90° C. for 12 h and cooled to room temperature. The mixture was diluted with water (10 mL) and extracted with EtOAc (15 mL×3). The combined organic layer was washed with brine (15 mL×2), dried over Na₂SO₄, filtered and concentrated under reduced pressure. The residue was purified by silica gel chromatograph (PE:EtOAc=1:0 to 1:1) to give the title compound (0.20 g, 24%). LC-MS (M+H)⁺=188.3.

### Step 10: 4-cyclopropyl-N-((5-(2,6-difluorophenyl)pyridin-2-yl)methyl)-5,6,7,8-tetrahydroquinolin-8-amine

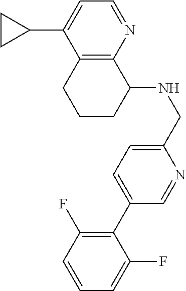 [SMILES: Fc1cccc(F)c1-c1ccc(CNC2CCCc3c(C4CC4)ccnc32)nc1]

The title compound (0.30 g, 29%) was prepared in a manner similar to that in Example 10 step 3 from 4-cyclopropyl-6,7-dihydroquinolin-8(5H)-one and (5-(2,6-difluorophenyl)pyridin-2-yl)methanamine. LC-MS (M+H)⁺=392.2.

### Step 11: N-(4-cyclopropyl-5,6,7,8-tetrahydroquinolin-8-yl)-N-((5-(2,6-difluorophenyl)pyridin-2-yl)methyl)-5-((diphenylmethylene)amino)-6-methyl-1-((2-(trimethylsilyl)ethoxy)methyl)-1H-pyrrolo[3,2-b]pyridine-2-carboxamide

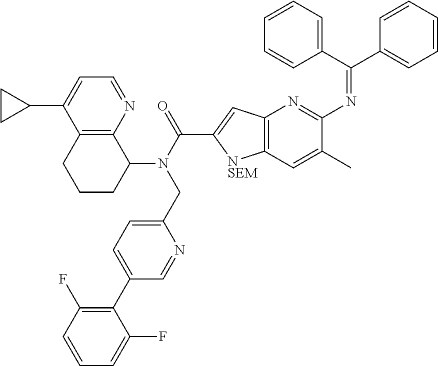 [SMILES: Cc1cc2c(cc(C(=O)N(Cc3ccc(-c4c(F)cccc4F)cn3)C3CCCc4c(C5CC5)ccnc43)n2COCC[Si](C)(C)C)nc1N=C(c1ccccc1)c1ccccc1]

The title compound (20 mg, 42%) was prepared in a manner similar to that in Example 1 step 8 from 5-((diphenylmethylene)amino)-6-methyl-1-((2-(trimethylsilyl)ethoxy)methyl)-1H-pyrrolo[3,2-b]pyridine-2-carboxylic acid and 4-cyclopropyl-N-((5-(2,6-difluorophenyl)pyridin-2-yl)methyl)-5,6,7,8-tetrahydroquinolin-8-amine. LC-MS (M+H)⁺=859.3.

### Step 12: 5-amino-N-(4-cyclopropyl-5,6,7,8-tetrahydroquinolin-8-yl)-N-((5-(2,6-difluorophenyl)pyridin-2-yl)methyl)-6-methyl-1H-pyrrolo[3,2-b]pyridine-2-carboxamide

Example 6 (5 mg, 38%) was prepared in a manner similar to that in Example 1 step 9 from N-(4-cyclopropyl-5,6,7,8-tetrahydroquinolin-8-yl)-N-((5-(2,6-difluorophenyl)pyridin-2-yl)methyl)-5-((diphenylmethylene)amino)-6-methyl-1-((2-(trimethylsilyl)ethoxy)methyl)-1H-pyrrolo[3,2-b]pyridine-2-carboxamide. ¹H NMR (500 MHz, DMSO-d6) δ 11.29-11.23 (m, 1H), 8.63-8.46 (m, 1H), 8.24-8.15 (m, 1H), 7.92-7.69 (m, 1H), 7.47 (d, J=6.9 Hz, 2H), 7.28-7.20 (m, 3H), 6.80-6.46 (m, 1H), 6.07-5.84 (m, 1H), 5.22 (s, 1H), 4.76-4.72 (m, 1H), 3.74-3.71 (m, 1H), 2.90-2.87 (m, 1H), 2.75 (s, 1H), 2.36 (s, 1H), 2.15-2.05 (m, 3H), 2.0-1.85 (m, 3H), 1.72 (s, 1H), 1.02-0.87 (m, 2H), 0.73-0.58 (m, 2H). LC-MS (M+H)⁺=565.2.

### Example A7: 5-amino-N-((5-(2,6-difluorophenyl)pyridin-2-yl)methyl)-N-(4-(2-hydroxypropan-2-yl)-5,6,7,8-tetrahydroquinolin-8-yl)-6-methyl-1H-pyrrolo[3,2-b]pyridine-2-carboxamide

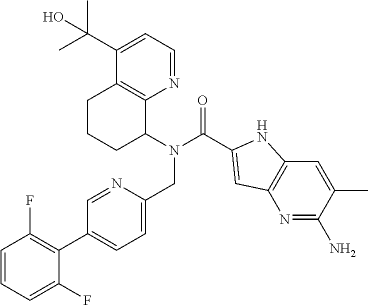 [SMILES: Cc1cc2[nH]c(C(=O)N(Cc3ccc(-c4c(F)cccc4F)cn3)C3CCCc4c(C(C)(C)O)ccnc43)cc2nc1N]

### Step 1: methyl 5,6,7,8-tetrahydroquinoline-4-carboxylate

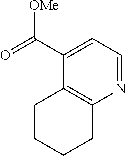 [SMILES: COC(=O)c1ccnc2c1CCCC2]

To a solution of 4-bromo-5,6,7,8-tetrahydroquinoline (2.0 g, 9.43 mmol) in MeOH (40 mL) was added Et₃N (2.86 g, 28.3 mmol) and Pd(dppf)Cl₂ DCM (770 mg, 943 μmol) under nitrogen. The mixture was degassed and purged with CO for 3 times, and then the mixture was stirred at 50° C. for 12 h under CO (50 psi). The mixture was filtered and the filtrate was concentrated in vacuum. The residue was purified by silica gel chromatography (PE/EtOAc=1/0 to 3/1) the title compound (0.76 g, 42%). LC-MS (M+H)⁺=192.3.

### Step 2: 2-(5,6,7,8-tetrahydroquinolin-4-yl) propan-2-ol

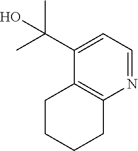

To a solution of MeMgBr (3 M in THF, 3.1 mL, 9.3 mmol) in THF (6 mL) was added a solution of methyl 5,6,7,8-tetrahydroquinoline-4-carboxylate (0.60 g, 3.14 mmol) in THF (3 mL) dropwise at 0° C. under nitrogen. The mixture was warmed to room temperature and stirred for 2 h. Aqueous HCl (0.05 M, 10 mL) was slowly added to the reaction mixture, and the mixture was extracted with EtOAc (2 mL×3). The combined organic layer was washed with brine (2 mL), dried over Na₂SO₄, filtered and concentrated in vacuum. The residue was purified by silica gel chromatograph (PE/EtOAc=1/0 to 1/1) to give the title compound (0.40 g, 67%). ¹H NMR (400 MHZ, DMSO-d6) δ 8.19 (d, J=5.2 Hz, 1H), 7.20 (d, J=5.2 Hz, 1H), 5.08 (s, 1H), 3.02 (t, J=6.0 Hz, 1H), 2.84 (t, J=6.6 Hz, 1H), 1.83-1.65 (m, 4H), 1.48 (s, 6H). LC-MS (M+H)⁺=192.3.

### Step 3: 4-(2-hydroxypropan-2-yl)-5,6,7,8-tetrahydroquinoline 1-oxide

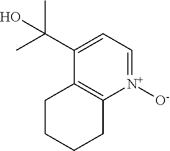

The title compound (0.30 g, 92%) was prepared in a manner similar to that in Example 6 step 5 from 2-(5,6,7,8-tetrahydroquinolin-4-yl) propan-2-ol. ¹H NMR (400 MHZ, DMSO-d6) δ 8.05 (d, J=6.8 Hz, 1H), 7.27 (d, J=6.8 Hz, 1H), 5.17 (s, 1H), 3.02 (t, J=6.0 Hz, 1H), 2.85 (t, J=6.6 Hz, 1H), 1.82-1.60 (m, 4H), 1.48 (s, 6H). LC-MS (M+H)⁺=208.3.

### Step 4: 4-(2-hydroxypropan-2-yl)-5,6,7,8-tetrahydroquinolin-8-yl acetate

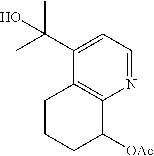 [SMILES: CC(=O)OC1CCCc2c(C(C)(C)O)ccnc21]

The title compound (0.48 g, 100%) was prepared in a manner similar to that in Example 6 step 6 from 4-(2-hydroxypropan-2-yl)-5,6,7,8-tetrahydroquinoline 1-oxide. LC-MS (M+H)⁺=250.3.

### Step 5: 4-(2-hydroxypropan-2-yl)-5,6,7,8-tetrahydroquinolin-8-ol

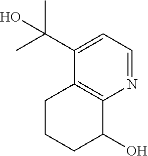

The title compound (0.48 g, 100%) was prepared in a manner similar to that in Example 6 step 7 from 4-(2-hydroxypropan-2-yl)-5,6,7,8-tetrahydroquinolin-8-yl acetate. ¹H NMR (400 MHZ, DMSO-d6) δ 8.31 (d, J=5.2 Hz, 1H), 7.31 (d, J=5.2 Hz, 1H), 5.13 (s, 1H), 4.98 (d, J=3.6 Hz, 1H), 4.59-4.53 (m, 1H), 3.22-3.10 (m, 1H), 2.97-2.85 (m, 1H), 1.98-1.60 (m, 4H), 1.48 (s, 6H). LC-MS (M+H)⁺=208.3.

### Step 6: 4-(2-hydroxypropan-2-yl)-6,7-dihydroquinolin-8(5H)-one

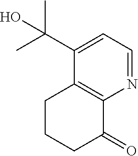

To a solution of 4-(2-hydroxypropan-2-yl)-5,6,7,8-tetrahydroquinolin-8-ol (0.26 g, 1.25 mmol) in DCM (10 mL) was added MnO₂ (1.09 g, 12.5 mmol). The mixture was stirred at 40° C. for 12 h and cooled to room temperature. Solid was filtered off and the filtrate was concentrated in vacuum. The residue was purified by silica gel chromatograph (PE/EtOAc=1/0 to 0/1) to give the title compound (0.20 g, 78%). ¹H NMR (400 MHz, DMSO-d6) δ 8.53 (d, J=4.8 Hz, 1H), 7.58 (d, J=4.8 Hz, 1H), 5.33 (s, 1H), 3.29-3.22 (m, 2H), 2.68 (t, J=6.4 Hz, 2H), 2.06-1.99 (m, 2H), 1.53 (s, 6H). LC-MS (M+H)⁺=206.1.

### Step 7: 2-(8-(((5-(2,6-difluorophenyl)pyridin-2-yl)methyl)amino)-5,6,7,8-tetrahydroquinolin-4-yl) propan-2-ol

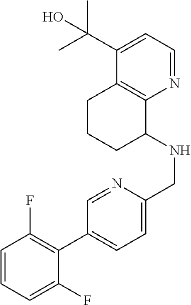 [SMILES: CC(C)(O)c1ccnc2c1CCCC2NCc1ccc(-c2c(F)cccc2F)cn1]

The title compound (0.10 g, 33%) was prepared in a manner similar to that in Example 10 step 3 from 4-(2-hydroxypropan-2-yl)-6,7-dihydroquinolin-8 (5H)-one and (5-(2,6-difluorophenyl)pyridin-2-yl)methanamine. LC-MS (M+H)⁺=410.2.

### Step 8: N-((5-(2,6-difluorophenyl)pyridin-2-yl)methyl)-5-((diphenylmethylene)amino)-N-(4-(2-hydroxypropan-2-yl)-5,6,7,8-tetrahydroquinolin-8-yl)-6-methyl-1-((2-(trimethylsilyl)ethoxy)methyl)-1H-pyrrolo[3,2-b]pyridine-2-carboxamide

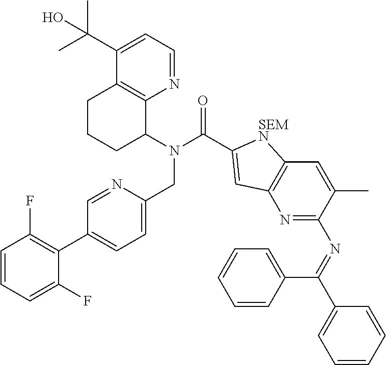 [SMILES: Cc1cc2c(cc(C(=O)N(Cc3ccc(-c4c(F)cccc4F)cn3)C3CCCc4c(C(C)(C)O)ccnc43)n2COCC[Si](C)(C)C)nc1N=C(c1ccccc1)c1ccccc1]

The title compound (10 mg, 28%) was prepared in a manner similar to that in Example 1 step 8 from 5-((diphenylmethylene)amino)-6-methyl-1-((2-(trimethylsilyl)ethoxy)methyl)-1H-pyrrolo[3,2-b]pyridine-2-carboxylic acid and 2-(8-(((5-(2,6-difluorophenyl)pyridin-2-yl)methyl)amino)-5,6,7,8-tetrahydroquinolin-4-yl) propan-2-ol. LC-MS (M+H)⁺=877.4.

### Step 9: 5-amino-N-((5-(2,6-difluorophenyl)pyridin-2-yl)methyl)-N-(4-(2-hydroxypropan-2-yl)-5,6,7,8-tetrahydroquinolin-8-yl)-6-methyl-1H-pyrrolo[3,2-b]pyridine-2-carboxamide

Example 7 (2 mg, 30%) was prepared in a manner similar to that in Example 1 step 9 from N-((5-(2,6-difluorophenyl)pyridin-2-yl)methyl)-5-((diphenylmethylene)amino)-N-(4-(2-hydroxypropan-2-yl)-5,6,7,8-tetrahydroquinolin-8-yl)-6-methyl-1-((2-(trimethylsilyl)ethoxy)methyl)-1H-pyrrolo[3,2-b]pyridine-2-carboxamide. ¹H NMR (500 MHz, DMSO-d6) δ 11.50-11.23 (m, 1H), 8.77-8.50 (m, 1H), 8.43-8.18 (m, 1H), 8.01-7.23 (m, 7H), 6.66-6.09 (m, 1H), 6.00-5.35 (m, 1H), 5.33-4.70 (m, 5H), 3.95-3.34 (m, 1H), 3.05-2.81 (m, 1H), 2.47-2.38 (m, 1H), 2.21-1.60 (m, 7H), 1.50 (s, 6H). LC-MS (M+H)⁺=583.4.

### Example A8: (R)-5-amino-N-((4-chloro-5-phenylpyridin-2-yl)methyl)-6-methyl-N-(5,6,7,8-tetrahydroquinolin-8-yl)-1H-pyrrolo[3,2-b]pyridine-2-carboxamide

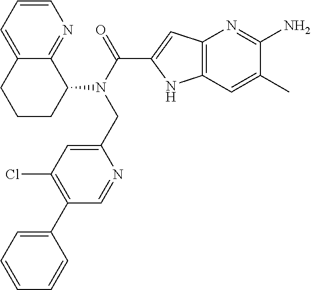 [SMILES: Cc1cc2[nH]c(C(=O)N(Cc3cc(Cl)c(-c4ccccc4)cn3)[C@@H]3CCCc4cccnc43)cc2nc1N]

### Step 1: methyl 4-amino-5-phenylpicolinate

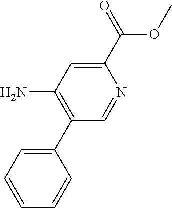 [SMILES: COC(=O)c1cc(N)c(-c2ccccc2)cn1]

To a solution of methyl 4-amino-5-iodopicolinate (2.0 g, 7.2 mmol) and phenylboronic acid (1.05 g, 8.6 mmol) in dioxane (16 mL) and water (4 mL) was added Pd(dppf)Cl₂ (526 mg, 0.72 mmol) and K₃PO₄ (3.05 g, 14.4 mmol). The mixture was stirred at 100° C. for 2 h and cooled to room temperature. The mixture was diluted with water (10 mL) and extracted with EtOAc (8 mL×2). The combined organic layer was washed with brine (5.0 mL), dried with Na₂SO₄, filtered and concentrated under vacuum. The residue was purified by silica gel chromatograph (PE:EtOAc=50:1 to 5:1) to give the title compound (400 mg, 24%). LC-MS (M+H)⁺=229.2.

### Step 2: methyl 4-chloro-5-phenylpicolinate

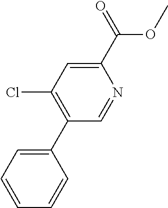 [SMILES: COC(=O)c1cc(Cl)c(-c2ccccc2)cn1]

To a mixture of CuCl₂ (509 mg, 3.79 mmol) in MeCN (2 mL) was added tert-butyl nitrite (488 mg, 4.73 mmol). The mixture was stirred at room temperature for 15 min, then cooled to 0° C. A solution of methyl 4-amino-5-phenylpicolinate (360 mg, 1.58 mmol) in MeCN (2 mL) was added dropwise. Upon completion of addition, the mixture was stirred at 0° C. for 1 h and room temperature for 16 h. The mixture was concentrated in vacuum and the residue was purified by silica gel chromatograph (PE:EtOAc=50:1 to 3:1) to give the title compound (160 mg, 41%). ¹H NMR (400 MHZ, DMSO-d6) δ 8.79 (s, 1H), 8.27 (s, 1H), 7.68-7.53 (m, 5H), 3.99 (s, 3H). LC-MS (M+H)⁺=248.2.

### Step 3: 4-chloro-5-phenylpicolinaldehyde

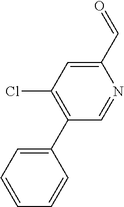 [SMILES: O=Cc1cc(Cl)c(-c2ccccc2)cn1]

To a solution of methyl 4-chloro-5-phenylpicolinate (160 mg, 0.65 mmol) in THF (2 mL) was added DIBAL-H (1 M in toluene, 840 μL) at −70° C. The mixture was stirred at −70° C. for 1 h. The mixture was poured into water (10.0 mL), the mixture was extracted with EtOAc (8 mL×2). The combined organic layer was washed with brine (5.0 mL), dried with Na₂SO₄, filtered and concentrated in vacuum. The residue was purified by silica gel chromatograph (PE:EtOAc=50:1 to 5:1) to give the title compound (130 mg, 92%). ¹H NMR (400 MHZ, DMSO-d6) δ 10.09 (s, 1H), 8.91 (s, 1H), 8.18 (s, 1H), 7.70-7.52 (m, 5H). LC-MS (M+H)⁺=218.2.

### Step 4: (R)—N-((4-chloro-5-phenylpyridin-2-yl)methyl)-5,6,7,8-tetrahydroquinolin-8-amine

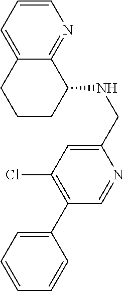 [SMILES: Clc1cc(CN[C@@H]2CCCc3cccnc32)ncc1-c1ccccc1]

The title compound (150 mg, 75%) was prepared in a manner similar to that in Example 1 step 7 from (R)-5,6,7,8-tetrahydroquinolin-8-amine and 4-chloro-5-phenylpicolinaldehyde. LC-MS (M+H)⁺=350.2.

### Step 5: (R)—N-((4-chloro-5-phenylpyridin-2-yl)methyl)-5-((diphenylmethylene)amino)-6-methyl-N-(5,6,7,8-tetrahydroquinolin-8-yl)-1-((2-(trimethylsilyl)ethoxy)methyl)-1H-pyrrolo[3,2-b]pyridine-2-carboxamide

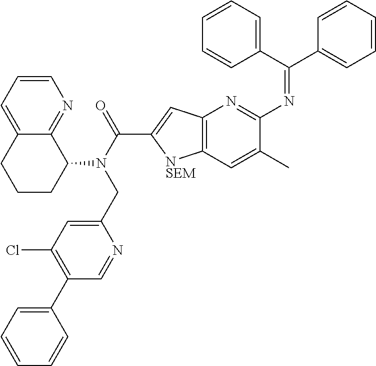 [SMILES: Cc1cc2c(cc(C(=O)N(Cc3cc(Cl)c(-c4ccccc4)cn3)[C@@H]3CCCc4cccnc43)n2COCC[Si](C)(C)C)nc1N=C(c1ccccc1)c1ccccc1]

The title compound (10 mg, 30%) was prepared in a manner similar to that in Example 1 step 8 from 5-((diphenylmethylene)amino)-6-methyl-1-((2-(trimethylsilyl)ethoxy)methyl)-1H-pyrrolo[3,2-b]pyridine-2-carboxylic acid and (R)—N-((4-chloro-5-phenylpyridin-2-yl)methyl)-5,6,7,8-tetrahydroquinolin-8-amine. LC-MS (M+H)⁺=817.4.

### Step 6: (R)-5-amino-N-((4-chloro-5-phenylpyridin-2-yl)methyl)-6-methyl-N-(5,6,7,8-tetrahydroquinolin-8-yl)-1H-pyrrolo[3,2-b]pyridine-2-carboxamide

Example 8 (1 mg, 16%) was prepared in a manner similar to that in Example 1 step 9 from (R)—N-((4-chloro-5-phenylpyridin-2-yl)methyl)-5-((diphenylmethylene)amino)-6-methyl-N-(5,6,7,8-tetrahydroquinolin-8-yl)-1-((2-(trimethylsilyl)ethoxy)methyl)-1H-pyrrolo[3,2-b]pyridine-2-carboxamide. ¹H NMR (500 MHZ, DMSO-d6) δ 11.30 (s, 1H), 8.47-8.30 (m, 2H), 7.63-7.44 (m, 8H), 7.37-7.29 (m, 1H), 7.26-7.13 (m, 1H), 5.29 (s, 2H), 4.79-3.92 (m, 1H), 2.92-2.81 (m, 2H), 2.78-2.71 (m, 2H), 2.14 (s, 3H), 2.03-1.93 (m, 2H), 1.85-1.72 (m, 2H). LC-MS (M+H)⁺=523.3.

### Example A9: 5-amino-6-methyl-N-((6-methyl-5-(1H-pyrazol-4-yl) pyridin-2-yl)methyl)-N-(5,6,7,8-tetrahydroquinolin-8-yl)-1H-pyrrolo[3,2-b]pyridine-2-carboxamide

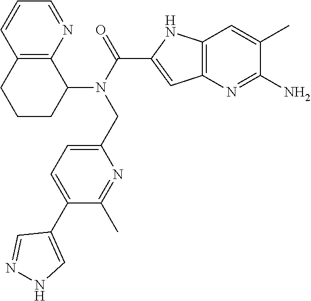 [SMILES: Cc1cc2[nH]c(C(=O)N(Cc3ccc(-c4cn[nH]c4)c(C)n3)C3CCCc4cccnc43)cc2nc1N]

### Step 1: tert-butyl ((5-bromo-6-methylpyridin-2-yl)methyl)carbamate

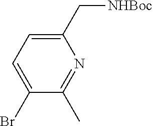 [SMILES: Cc1nc(CNC(=O)OC(C)(C)C)ccc1Br]

To a solution of 5-bromo-6-methylpicolinonitrile (5.0 g, 25.4 mmol), Boc₂O (11.1 g, 50.8 mmol) and NiCl₂ (987 mg, 7.61 mmol) in MeOH (75 mL) was added NaBH₄ (3.84 g, 102 mmol) at 0° C. the mixture was warmed to room temperature and stirred for 2 h. The mixture was slowly added to iced water (100 mL). Upon cease of bubbling, the mixture was extracted with EtOAc (50 mL×3). The combined organic layer was washed with brine (20 ml×3), dried over Na₂SO₄, filtered and concentrated in vacuum. The residue was purified by silica gel chromatograph (PE:EtOAc=1:0 to 3:1) to give the title compound (1.3 g, 17%). LC-MS (M+H)⁺=301.2.

### Step 2: (5-bromo-6-methylpyridin-2-yl)methanamine

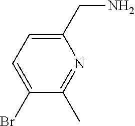

A mixture of tert-butyl ((5-bromo-6-methylpyridin-2-yl)methyl)carbamate (1.3 g, 4.32 mmol) in HCl (4 M in MeOH, 20 mL) was stirred at room temperature for 2 h. The mixture was neutralized with 1 M NaOH in MeOH to a pH of 7, then concentrated under vacuum. The solid was triturated with a mixture of DCM (10 mL) and MeOH (1 mL). The solid was filtered off and the filtrate was concentrated under reduced pressure to give the title compound (0.80 g, 92%). LC-MS (M+H)⁺=201.1.

### Step 3: N-((5-bromo-6-methylpyridin-2-yl)methyl)-5,6,7,8-tetrahydroquinolin-8-amine

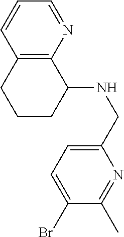 [SMILES: Cc1nc(CNC2CCCc3cccnc32)ccc1Br]

The title compound (0.50 g, 47%) was prepared in a manner similar to that in Example 10 step 3 from 6,7-dihydroquinolin-8 (5H)-one and (5-bromo-6-methylpyridin-2-yl)methanamine. LC-MS (M+H)⁺=332.2.

### Step 4: N-((6-methyl-5-(1-(tetrahydro-2H-pyran-2-yl)-1H-pyrazol-4-yl) pyridin-2-yl)methyl)-5,6,7,8-tetrahydroquinolin-8-amine

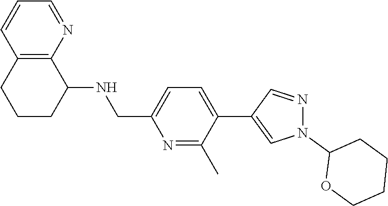 [SMILES: Cc1nc(CNC2CCCc3cccnc32)ccc1-c1cnn(C2CCCCO2)c1]

To a mixture of N-((5-bromo-6-methylpyridin-2-yl)methyl)-5,6,7,8-tetrahydroquinolin-8-amine (0.80 g, 2.4 mmol) and 1-(tetrahydro-2H-pyran-2-yl)-4-(4,4,5,5-tetramethyl-1,3,2-dioxaborolan-2-yl)-1H-pyrazole (1.67 g, 6.0 mmol) in dioxane (4 mL), toluene (2 mL) and water (2 mL) was added K₃PO₄ (1.28 g, 6.0 mmol) and Pd(dppf)Cl₂ (176 mg, 0.24 mmol). The mixture was stirred at 90° C. for 12 h then cooled to room temperature. The reaction was diluted with water (10 mL) and extracted with EtOAc (10 mL×3). The combined organic layer was washed with brine (10 mL×3), dried over Na₂SO₄, filtered and concentrated in vacuum. The residue was purified by silica gel chromatography (PE:EtOAc=1:0 to 1:1) to give the title compound (139 mg, 14% yield). LC-MS (M+H)⁺=404.2.

### Step 5: 5-((diphenylmethylene)amino)-6-methyl-N-((6-methyl-5-(1-(tetrahydro-2H-pyran-2-yl)-1H-pyrazol-4-yl) pyridin-2-yl)methyl)-N-(5,6,7,8-tetrahydroquinolin-8-yl)-1-((2-(trimethylsilyl)ethoxy)methyl)-1H-pyrrolo[3,2-b]pyridine-2-carboxamide

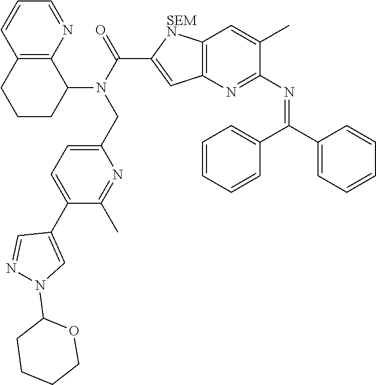 [SMILES: Cc1cc2c(cc(C(=O)N(Cc3ccc(-c4cnn(C5CCCCO5)c4)c(C)n3)C3CCCc4cccnc43)n2COCC[Si](C)(C)C)nc1N=C(c1ccccc1)c1ccccc1]

The title compound (30 mg, 42%) was prepared in a manner similar to that in Example 1 step 8 from 5-((diphenylmethylene)amino)-6-methyl-1-((2-(trimethylsilyl)ethoxy)methyl)-1H-pyrrolo[3,2-b]pyridine-2-carboxylic acid and N-((6-methyl-5-(1-(tetrahydro-2H-pyran-2-yl)-1H-pyrazol-4-yl) pyridin-2-yl)methyl)-5,6,7,8-tetrahydroquinolin-8-amine. LC-MS (M+H)⁺=871.4.

### Step 6: 5-amino-6-methyl-N-((6-methyl-5-(1H-pyrazol-4-yl) pyridin-2-yl)methyl)-N-(5,6,7,8-tetrahydroquinolin-8-yl)-1H-pyrrolo[3,2-b]pyridine-2-carboxamide

Example 9 (6 mg, 26%) was prepared in a manner similar to that in Example 1 step 9 from 5-((diphenylmethylene)amino)-6-methyl-N-((6-methyl-5-(1-(tetrahydro-2H-pyran-2-yl)-1H-pyrazol-4-yl) pyridin-2-yl)methyl)-N-(5,6,7,8-tetrahydroquinolin-8-yl)-1-((2-(trimethylsilyl)ethoxy)methyl)-1H-pyrrolo[3,2-b]pyridine-2-carboxamide. ¹H NMR (500 MHz, DMSO-d6) δ 12.37 (s, 1H), 11.38 (s, 1H), 8.46-8.31 (m, 1H), 8.00-7.83 (m, 2H), 7.82-7.67 (m, 2H), 7.61-7.53 (m, 1H), 7.52-7.14 (m, 3H), 6.67-6.58 (m, 0.5H), 6.20-5.86 (m, 1H), 5.31-5.04 (m, 2H), 4.83-3.65 (m, 1H), 2.88-2.67 (m, 2H), 2.65-2.58 (m, 2H), 2.43-2.34 (m, 1H), 2.17-2.09 (m, 4H), 1.97-1.90 (m, 2H), 1.80-1.69 (m, 1H). LC-MS (M+H)⁺=493.3.

### Example A10: 5-amino-N-((5-(2,6-dichlorophenyl)pyridin-2-yl)methyl)-6-methyl-N-(5,6,7,8-tetrahydroquinolin-8-yl)-1H-pyrrolo[3,2-b]pyridine-2-carboxamide

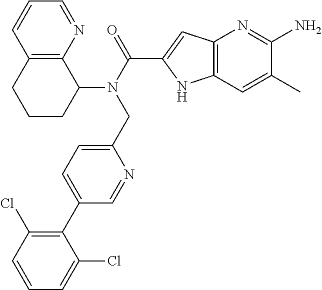 [SMILES: Cc1cc2[nH]c(C(=O)N(Cc3ccc(-c4c(Cl)cccc4Cl)cn3)C3CCCc4cccnc43)cc2nc1N]

### Step 1: tert-butyl ((5-(2,6-dichlorophenyl)pyridin-2-yl)methyl)carbamate

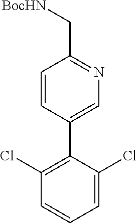 [SMILES: CC(C)(C)OC(=O)NCc1ccc(-c2c(Cl)cccc2Cl)cn1]

To a solution of tert-butyl ((5-bromopyridin-2-yl)methyl)carbamate (18.0 g, 62.7 mmol) and (2,6-dichlorophenyl) boronic acid (23.9 g, 125 mmol) in dioxane (80 mL), toluene (40 mL) and water (40 mL) was added K₃PO₄ (33.3 g, 157 mmol) and Pd(dppf)Cl₂ (4.59 g, 6.27 mmol) under nitrogen. The mixture was stirred at 85° C. for 12 h and cooled to room temperature. The mixture was diluted with water (150 mL) and extracted with EtOAc (80 mL×3). The combined organic layer was washed with brine (50 mL×2), dried over Na₂SO₄, filtered and concentrated under reduced pressure. The residue was purified by silica gel chromatography (PE:EtOAc=1:0 to 5:1) to give the title compound (9.0 g, 41%). LC-MS (M+H)⁺=353.1.

### Step 2: (5-(2,6-dichlorophenyl)pyridin-2-yl)methanamine

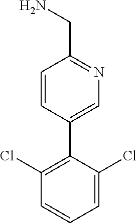 [SMILES: NCc1ccc(-c2c(Cl)cccc2Cl)cn1]

A mixture of tert-butyl ((5-(2,6-dichlorophenyl)pyridin-2-yl)methyl)carbamate (9.0 g, 25.5 mmol) in methanolic HCl (4 M, 90 mL) was stirred at 25° C. for 1 h, then the mixture was neutralized with methanolic NaOH (1 M) to pH 7. The mixture was concentrated to dryness under reduced pressure, and the residue was triturated with DCM/MeOH (10/1, 10 mL). The filtrate was collected by filtration and concentrated under vacuum to give the title compound (6.0 g, 93%). ¹H NMR (400 MHz, CDCl₃) δ 8.45 (s, 1H), 7.66-7.58 (m, 1H), 7.54 (d, J=8.0 Hz, 1H), 7.38 (d, J=8.0 Hz, 2H), 7.30-7.24 (m, 1H), 4.58 (s, 2H).

### Step 3: N-((5-(2,6-dichlorophenyl)pyridin-2-yl)methyl)-5,6,7,8-tetrahydroquinolin-8-amine

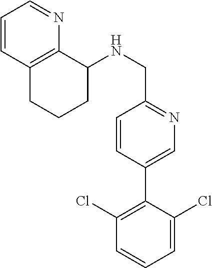 [SMILES: Clc1cccc(Cl)c1-c1ccc(CNC2CCCc3cccnc32)nc1]

To a solution of (5-(2,6-dichlorophenyl)pyridin-2-yl)methanamine (10.8 g, 42.8 mmol) and 6,7-dihydroquinolin-8 (5H)-one (7.0 g, 47.6 mmol) in DCM (140 mL) and MeOH (1.4 mL) was added NaBH(OAc)₃ (18.1 g, 85.6 mmol). The mixture was stirred at 25° C. for 2 h. The mixture was poured into saturated NaHCO₃ (100 mL). The mixture was extracted with DCM (50 mL×3). The combined organic layer was washed with brine (30 mL), dried with Na₂SO₄, filtered and concentrated under vacuum. The residue was purified by silica gel chromatography (PE:EtOAc=1:0 to 0:1) to give the title compound (5.5 g, 30%). ¹H NMR (400 MHZ, CD₃OD) δ 8.43-8.35 (m, 2H), 7.78-7.70 (m, 1H), 7.66 (d, J=8.0 Hz, 1H), 7.58-7.50 (m, 3H), 7.44-7.36 (m, 1H), 7.25-7.17 (m, 1H), 4.73-4.56 (m, 2H), 3.90 (dd, J=7.6, 6.4 Hz, 1H), 2.95-2.76 (m, 2H), 2.33-2.23 (m, 1H), 2.12-2.03 (m, 2H), 1.93-1.72 (m, 2H). LC-MS (M+H)⁺=384.0.

### Step 4: N-((5-(2,6-dichlorophenyl)pyridin-2-yl)methyl)-5-((diphenylmethylene)amino)-6-methyl-N-(5,6,7,8-tetrahydroquinolin-8-yl)-1-((2-(trimethylsilyl)ethoxy)methyl)-1H-pyrrolo[3,2-b]pyridine-2-carboxamide

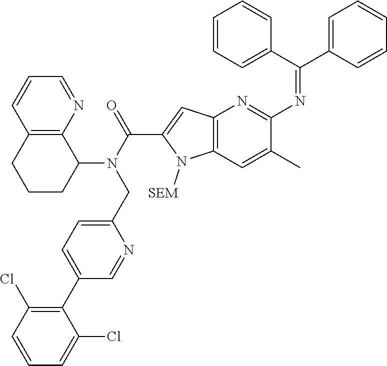 [SMILES: Cc1cc2c(cc(C(=O)N(Cc3ccc(-c4c(Cl)cccc4Cl)cn3)C3CCCc4cccnc43)n2COCC[Si](C)(C)C)nc1N=C(c1ccccc1)c1ccccc1]

To a solution of 5-((diphenylmethylene)amino)-6-methyl-1-((2-(trimethylsilyl)ethoxy)methyl)-1H-pyrrolo[3,2-b]pyridine-2-carboxylic acid (430 mg, 0.88 mmol) in DMF (15 mL) was added HATU (370 mg, 0.97 mmol), N-((5-(2,6-dichlorophenyl)pyridin-2-yl)methyl)-5,6,7,8-tetrahydroquinolin-8-amine (408 mg, 1.06 mmol) and DIPEA (228 mg, 1.77 mmol). The solution was stirred for 16 h at 55° C. The mixture was cooled to room temperature, diluted into water (50 mL) and then extracted with EtOAc (50 mL). The combined organic layer was washed with brine (50 mL×2), dried over Na₂SO₄, filtered and concentrated under vacuum. The residue was purified by silica gel chromatograph (DCM:MeOH=20:1) to give the title compound (600 mg, 80%). LC-MS (M+H)⁺=851.2.

### Step 5: 5-amino-N-((5-(2,6-dichlorophenyl)pyridin-2-yl)methyl)-6-methyl-N-(5,6,7,8-tetrahydroquinolin-8-yl)-1H-pyrrolo[3,2-b]pyridine-2-carboxamide

To a solution of N-((5-(2,6-dichlorophenyl)pyridin-2-yl)methyl)-5-((diphenylmethylene)amino)-6-methyl-N-(5,6,7,8-tetrahydroquinolin-8-yl)-1-((2-(trimethylsilyl)ethoxy)methyl)-1H-pyrrolo[3,2-b]pyridine-2-carboxamide (600 mg, 0.71 mmol) in DCM (8 mL) was added TFA (8 mL). The mixture was stirred for 2 h at room temperature. The reaction solution was concentrated under vacuum, then HCl (4M in MeOH, 5 mL) was added. The mixture was stirred for 2 h at room temperature. The mixture was concentrated under vacuum, then K₂CO₃ (390 mg, 2.82 mmol), water (3 mL) and MeOH (15 mL) was added. The mixture was stirred for 3 h at 60° C. The mixture was cooled to room temperature and concentrated under vacuum. The residue was purified by silica gel chromatograph (DCM:MeOH=20:1) to give the title compound (277 mg, 70.4%). ¹H NMR (500 MHz, DMSO-d6) δ 11.64-11.19 (m, 1H), 8.67-8.19 (m, 2H), 8.06-7.06 (m, 8H), 6.91-6.12 (m, 1H), 6.05-5.07 (m, 3H), 5.07-3.71 (m, 2H), 3.02-2.65 (m, 2H), 2.49-2.40 (m, 1H), 2.31-1.89 (m, 5H), 1.86-1.64 (m, 1H). LC-MS (M+H)⁺=557.3.

### Example A11: (R)-5-amino-6-methyl-N-((5-(perfluorophenyl)pyridin-2-yl)methyl)-N-(5,6,7,8-tetrahydroquinolin-8-yl)-1H-pyrrolo[3,2-b]pyridine-2-carboxamide

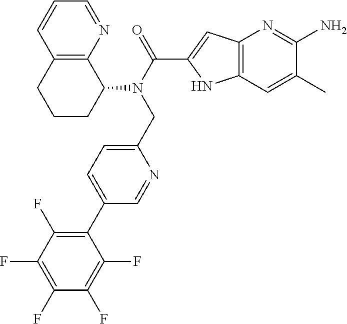 [SMILES: Cc1cc2[nH]c(C(=O)N(Cc3ccc(-c4c(F)c(F)c(F)c(F)c4F)cn3)[C@@H]3CCCc4cccnc43)cc2nc1N]

### Step 1: 5-(perfluorophenyl)picolinaldehyde

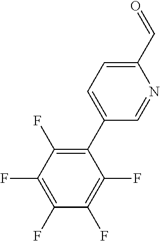 [SMILES: O=Cc1ccc(-c2c(F)c(F)c(F)c(F)c2F)cn1]

To a mixture of 5-(4,4,5,5-tetramethyl-1,3,2-dioxaborolan-2-yl)picolinaldehyde (4.76 g, 20.4 mmol) and 1,2,3,4,5-pentafluoro-6-iodobenzene (2.0 g, 6.80 mmol) in dioxane (10 mL), toluene (5 mL) and water (5 mL) was added Pd(dppf)Cl₂ (249 mg, 0.34 mmol) and K₃PO₄ (3.61 g, 17.0 mmol). The mixture was stirred at 85° C. for 16 h then cooled to room temperature. The mixture was diluted with water (10 mL), extracted with EtOAc (8 mL×2). The combined organic layer was washed with brine (5 mL), dried with Na₂SO₄, filtered and concentrated in vacuum. The residue was purified by silica gel chromatograph (PE:EtOAc=50:1 to 5:1) to give the title compound (0.80 mg, 43%).

### Step 2: (R)—N-((5-(perfluorophenyl)pyridin-2-yl)methyl)-5,6,7,8-tetrahydroquinolin-8-amine

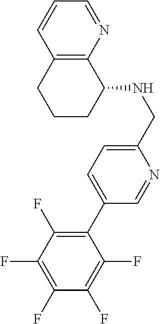 [SMILES: Fc1c(F)c(F)c(-c2ccc(CN[C@@H]3CCCc4cccnc43)nc2)c(F)c1F]

The title compound (0.20 g, 45%) was prepared in a manner similar to that in Example 1 step 7 from (R)-5,6,7,8-tetrahydroquinolin-8-amine and 5-(perfluorophenyl)picolinaldehyde. LC-MS (M+H)⁺=406.1.

### Step 3: (R)-5-((diphenylmethylene)amino)-6-methyl-N-((5-(perfluorophenyl)pyridin-2-yl)methyl)-N-(5,6,7,8-tetrahydroquinolin-8-yl)-1-((2-(trimethylsilyl)ethoxy)methyl)-1H-pyrrolo[3,2-b]pyridine-2-carboxamide

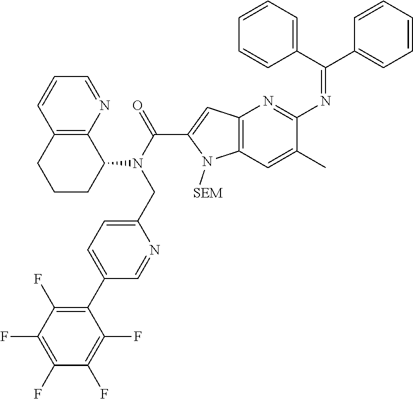 [SMILES: Cc1cc2c(cc(C(=O)N(Cc3ccc(-c4c(F)c(F)c(F)c(F)c4F)cn3)[C@@H]3CCCc4cccnc43)n2COCC[Si](C)(C)C)nc1N=C(c1ccccc1)c1ccccc1]

The title compound (44 mg, 62%) was prepared in a manner similar to that in Example 1 step 8 from 5-((diphenylmethylene)amino)-6-methyl-1-((2-(trimethylsilyl)ethoxy)methyl)-1H-pyrrolo[3,2-b]pyridine-2-carboxylic acid and (R)—N-((5-(perfluorophenyl)pyridin-2-yl)methyl)-5,6,7,8-tetrahydroquinolin-8-amine. LC-MS (M+H)⁺=873.4.

### Step 4: (R)-5-amino-6-methyl-N-((5-(perfluorophenyl)pyridin-2-yl)methyl)-N-(5,6,7,8-tetrahydroquinolin-8-yl)-1H-pyrrolo[3,2-b]pyridine-2-carboxamide

Example 11 (2 mg, 8%) was prepared in a manner similar to that in Example 1 step 9 from (R)-5-((diphenylmethylene)amino)-6-methyl-N-((5-(perfluorophenyl)pyridin-2-yl)methyl)-N-(5,6,7,8-tetrahydroquinolin-8-yl)-1-((2-(trimethylsilyl)ethoxy)methyl)-1H-pyrrolo[3,2-b]pyridine-2-carboxamide. ¹H NMR (500 MHz, DMSO-d6) δ 11.54-11.20 (m, 1H), 8.82-8.53 (m, 1H), 8.49-8.31 (m, 1H), 8.12-7.44 (m, 3H), 7.39-7.27 (m, 1H), 7.27-7.12 (m, 1H), 6.72-6.07 (m, 1H), 6.00-5.35 (m, 1H), 5.36-5.26 (m, 2H), 5.26-4.76 (m, 1H), 4.00-3.35 (m, 1H), 2.98-2.69 (m, 2H), 2.28-1.69 (m, 7H). LC-MS (M+H)⁺=579.4.

### Example A12: (R)-5-amino-6-bromo-N-((5-(2,6-difluorophenyl)pyridin-2-yl)methyl)-N-(5,6,7,8-tetrahydroquinolin-8-yl)-1H-pyrrolo[3,2-b]pyridine-2-carboxamide

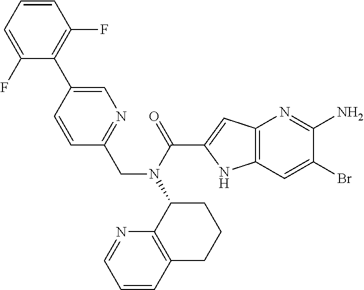 [SMILES: Nc1nc2cc(C(=O)N(Cc3ccc(-c4c(F)cccc4F)cn3)[C@@H]3CCCc4cccnc43)[nH]c2cc1Br]

### Step 1: tert-butyl (3-bromo-6-methyl-5-nitropyridin-2-yl)(tert-butoxycarbonyl)carbamate

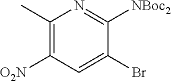

To a solution of 3-bromo-6-methyl-5-nitropyridin-2-amine (55 g, 237 mmol) and DMAP (2.90 g, 23.7 mmol) in THF (500 mL) was added Boc₂O (129 g, 593 mmol) at room temperature. After 12 h, the mixture was diluted with water (2.5 L) and extracted with EtOAc (400 mL×3). The combined organic layer was washed with brine (100 ml×4), dried over Na₂SO₄, filtered and concentrated in vacuum. The residue was purified by silica gel chromatograph (PE/EtOAc=3/1) to give the title compound (80 g, 78%). ¹H NMR (400 MHz, DMSO-d6) δ 8.87 (s, 1H), 2.70 (s, 3H), 1.38 (s, 18H).

### Step 2: ethyl 3-(5-bromo-6-((tert-butoxycarbonyl)amino)-3-nitropyridin-2-yl)-2-hydroxyacrylate

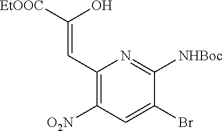 [SMILES: CCOC(=O)/C(O)=C/c1nc(NC(=O)OC(C)(C)C)c(Br)cc1[N+](=O)[O-]]

EtONa (20% in EtOH, 23.6 g, 69.4 mmol) was dissolved in THF (150 mL) at 0° C., and to this mixture was add diethyl oxalate (15.2 g, 104 mmol). The mixture was stirred at 0° C. for 0.5 h followed by addition of tert-butyl (3-bromo-6-methyl-5-nitropyridin-2-yl)(tert-butoxycarbonyl)carbamate (15 g, 34.7 mmol) in THF (100 mL) dropwise. The mixture was warmed to room temperature and stirred for 24 h. The mixture was diluted with water (2 L) and extracted with EtOAc (300 mL×4). The combined organic layer was washed with brine (100 mL×4), dried over Na₂SO₄, filtered and concentrated in vacuum. The residue was purified by silica gel chromatograph (PE/EtOAc=3/1) to give the title compound (5.0 g, 33% yield). ¹H NMR (400 MHZ, DMSO-d6) δ 13.15 (s, 1H), 9.57 (s, 1H), 8.79 (s, 1H), 7.26 (s, 1H), 4.29 (q, J=7.0 Hz, 2H), 1.52 (s, 9H), 1.31 (t, J=7.0 Hz, 3H).

### Step 3: 1-(tert-butyl) 2-ethyl 5-(bis(tert-butoxycarbonyl)amino)-6-bromo-1H-pyrrolo[3,2-b]pyridine-1,2-dicarboxylate

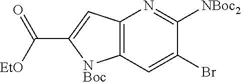

To a mixture of ethyl 3-(5-bromo-6-((tert-butoxycarbonyl)amino)-3-nitropyridin-2-yl)-2-hydroxyacrylate (2.5 g, 5.78 mmol) in AcOH (30 mL) was added iron powder (1.94 g, 34.7 mmol) and the mixture was stirred at 80° C. for 12 h. The mixture was cooled to room temperature and filtered. The filter cake was rinsed with THF (20 mL×2). The filtrate was concentrated under reduced pressure. The residue was diluted with water (10 mL) and neutralized with saturated NaHCO₃ until pH reached 7. The mixture was extracted with EtOAc (50 mL×4). The combined organic layer was washed with brine (10 mL×2), dried over Na₂SO₄, filtered and concentrated in vacuum. The residue (0.50 g) was re-dissolved in THF (5 mL) then DIPEA (1.14 g, 8.80 mmol) and DMAP (21.5 mg, 0.18 mmol) was added followed by Boc₂O (1.34 g, 6.16 mmol). The mixture was stirred at room temperature for 2 h. The mixture was diluted with water (10 mL) and extracted with EtOAc (10 mL×3). The combined organic layer was washed with brine (5 mL×3), dried over Na₂SO₄, filtered and concentrated in vacuum. The residue was purified by silica gel chromatograph (PE/EtOAc=3/1) to give the title compound (0.55 g, 16%). ¹H NMR (400 MHZ, DMSO-d6) δ 8.58 (s, 1H), 7.39 (s, 1H), 4.37 (q, J=7.0 Hz, 2H), 1.59 (s, 9H), 1.40-1.25 (m, 21H).

### Step 4: 6-bromo-1-(tert-butoxycarbonyl)-5-((tert-butoxycarbonyl)amino)-1H-pyrrolo[3,2-b]pyridine-2-carboxylic Acid

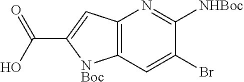 [SMILES: CC(C)(C)OC(=O)Nc1nc2cc(C(=O)O)n(C(=O)OC(C)(C)C)c2cc1Br]

To a mixture of 1-(tert-butyl) 2-ethyl 5-(bis(tert-butoxycarbonyl)amino)-6-bromo-1H-pyrrolo[3,2-b]pyridine-1,2-dicarboxylate (100 mg, 0.17 mmol) in THF (1 mL), MeOH (0.3 mL) and water (0.1 mL) was added LiOH·H₂O (11 mg, 0.26 mmol). The mixture was stirred at 40° C. for 0.5 h then cooled to room temperature. The pH of the mixture was carefully adjusted to ˜4 with methanolic HCl (1 M). The mixture was concentrated in vacuum. The residue was triturated with a mixture of DCM (10 mL) and MeOH (2 mL). Solid was filtered off and the filtrate was concentrated under reduced pressure to give the title compound (78 mg, 100%). LC-MS (M−H)⁻=454.3.

### Step 5: tert-butyl (R)-6-bromo-5-((tert-butoxycarbonyl)amino)-2-(((5-(2,6-difluorophenyl)pyridin-2-yl)methyl)(5,6,7,8-tetrahydroquinolin-8-yl) carbamoyl)-1H-pyrrolo[3,2-b]pyridine-1-carboxylate

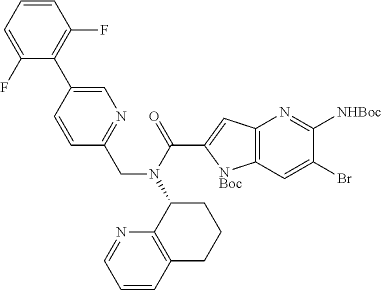 [SMILES: CC(C)(C)OC(=O)Nc1nc2cc(C(=O)N(Cc3ccc(-c4c(F)cccc4F)cn3)[C@@H]3CCCc4cccnc43)n(C(=O)OC(C)(C)C)c2cc1Br]

The title compound (40 mg, 18%) was prepared in a manner similar to that in Example 1 step 8 from 6-bromo-1-(tert-butoxycarbonyl)-5-((tert-butoxycarbonyl)amino)-1H-pyrrolo[3,2-b]pyridine-2-carboxylic acid and (R)—N-((5-(2,6-difluorophenyl)pyridin-2-yl)methyl)-5,6,7,8-tetrahydroquinolin-8-amine. LC-MS (M+H)⁺=789.2.

### Step 6: (R)-5-amino-6-bromo-N-((5-(2,6-difluorophenyl)pyridin-2-yl)methyl)-N-(5,6,7,8-tetrahydroquinolin-8-yl)-1H-pyrrolo[3,2-b]pyridine-2-carboxamide

A mixture of tert-butyl (R)-6-bromo-5-((tert-butoxycarbonyl)amino)-2-(((5-(2,6-difluorophenyl)pyridin-2-yl)methyl)(5,6,7,8-tetrahydroquinolin-8-yl) carbamoyl)-1H-pyrrolo[3,2-b]pyridine-1-carboxylate (40 mg, 51 μmol) and HCl (1 M in MeOH, 5 mL) was stirred for 2 h at room temperature. The mixture was concentrated under reduced pressure and the residue was purified by prep-HPLC to give Example 12 (3 mg, 10%). ¹H NMR (500 MHz, DMSO-d6) δ 11.72-11.61 (m, 1H), 8.78-8.52 (m, 1H), 8.49-8.31 (m, 1H), 8.00-7.73 (m, 2H), 7.59-7.50 (m, 2H), 7.34-7.13 (m, 3H), 6.73-6.20 (m, 1H), 5.88-5.38 (m, 3H), 5.25-4.76 (m, 1H), 3.96-3.30 (m, 1H), 2.93-2.64 (m, 2H), 2.49-1.77 (m, 4H). LC-MS (M+H)⁺=589.1.

### Example A13: (R)-5-amino-6-methyl-N-((5-(1-methyl-1H-1,2,4-triazol-5-yl) pyridin-2-yl)methyl)-N-(5,6,7,8-tetrahydroquinolin-8-yl)-1H-pyrrolo[3,2-b]pyridine-2-carboxamide

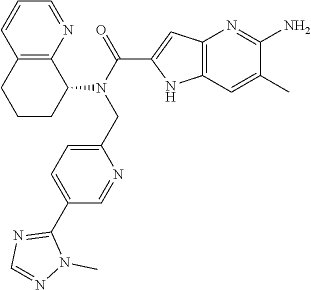 [SMILES: Cc1cc2[nH]c(C(=O)N(Cc3ccc(-c4ncnn4C)cn3)[C@@H]3CCCc4cccnc43)cc2nc1N]

### Step 1: 2-(1,3-dioxolan-2-yl)-5-(1-methyl-1H-1,2,4-triazol-5-yl)pyridine

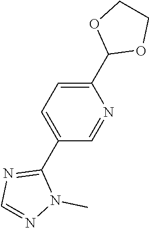 [SMILES: Cn1ncnc1-c1ccc(C2OCCO2)nc1]

To a mixture of (6-(1,3-dioxolan-2-yl) pyridin-3-yl) boronic acid (2.0 g, 10.3 mmol) in dioxane (20 mL), toluene (10 mL) and water (10 mL) was added 5-bromo-1-methyl-1H-1,2,4-triazole (1.66 g, 10.3 mmol), K₃PO₄ (6.53 g, 30.8 mmol) and Pd(dppf)Cl₂ (751 mg, 1.0 mmol). The mixture was stirred at 80° C. for 12 h and cooled to room temperature. Solid was filtered off and the filtrate was concentrated under reduced pressure. The residue was purified by silica gel chromatograph (PE:EtOAc=5:1 to 0:1) to give the title compound (1.3 g, 55%). LC-MS (M+H)⁺=233.3.

### Step 2: 5-(1-methyl-1H-1,2,4-triazol-5-yl)picolinaldehyde

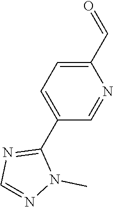

To a mixture of 2-(1,3-dioxolan-2-yl)-5-(1-methyl-1H-1,2,4-triazol-5-yl)pyridine (1.2 g, 5.17 mmol) in water (6 mL) and dioxane (12 mL) was added concentrated HCl (6.5 mL) and the mixture was stirred at 60° C. for 3 h. The mixture was cooled to room temperature and neutralized with saturated NaHCO₃ until pH reached 7˜8. The mixture was diluted with water (100 mL) and extracted with EtOAc (100 mL×3). The combined organic layer was dried with Na₂SO₄, filtered and concentrated in vacuum to give the title compound (0.60 g, 62%). LC-MS (M+H)⁺=189.3.

### Step 3: (R)—N-((5-(1-methyl-1H-1,2,4-triazol-5-yl) pyridin-2-yl)methyl)-5,6,7,8-tetrahydroquinolin-8-amine

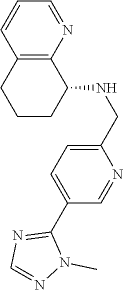 [SMILES: Cn1ncnc1-c1ccc(CN[C@@H]2CCCc3cccnc32)nc1]

The title compound (0.28 g, 25%) was prepared in a manner similar to that in Example 1 step 7 from (R)-5,6,7,8-tetrahydroquinolin-8-amine and 5-(1-methyl-1H-1,2,4-triazol-5-yl)picolinaldehyde. LC-MS (M+H)⁺=321.1.

### Step 4: (R)-5-((diphenylmethylene)amino)-6-methyl-N-((5-(1-methyl-1H-1,2,4-triazol-5-yl) pyridin-2-yl)methyl)-N-(5,6,7,8-tetrahydroquinolin-8-yl)-1-((2-(trimethylsilyl)ethoxy)methyl)-1H-pyrrolo[3,2-b]pyridine-2-carboxamide

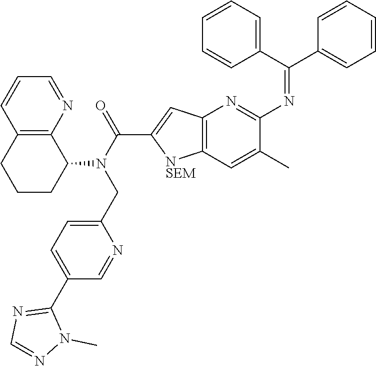 [SMILES: Cc1cc2c(cc(C(=O)N(Cc3ccc(-c4ncnn4C)cn3)[C@@H]3CCCc4cccnc43)n2COCC[Si](C)(C)C)nc1N=C(c1ccccc1)c1ccccc1]

The title compound (30 mg, 46%) was prepared in a manner similar to that in Example 1 step 8 from 5-((diphenylmethylene)amino)-6-methyl-1-((2-(trimethylsilyl)ethoxy)methyl)-1H-pyrrolo[3,2-b]pyridine-2-carboxylic acid and (R)—N-((5-(1-methyl-1H-1,2,4-triazol-5-yl) pyridin-2-yl)methyl)-5,6,7,8-tetrahydroquinolin-8-amine. LC-MS (M+H)⁺=788.4.

### Step 5: (R)-5-amino-6-methyl-N-((5-(1-methyl-1H-1,2,4-triazol-5-yl) pyridin-2-yl)methyl)-N-(5,6,7,8-tetrahydroquinolin-8-yl)-1H-pyrrolo[3,2-b]pyridine-2-carboxamide

Example 13 (12 mg, 64%) was prepared in a manner similar to that in Example 1 step 9 from (R)-5-((diphenylmethylene)amino)-6-methyl-N-((5-(1-methyl-1H-1,2,4-triazol-5-yl) pyridin-2-yl)methyl)-N-(5,6,7,8-tetrahydroquinolin-8-yl)-1-((2-(trimethylsilyl)ethoxy)methyl)-1H-pyrrolo[3,2-b]pyridine-2-carboxamide. ¹H NMR (500 MHz, DMSO-d6) δ 11.54-11.10 (m, 1H), 9.13-8.73 (m, 1H), 8.49-8.32 (m, 1H), 8.29-8.09 (m, 1H), 8.07-7.44 (m, 3H), 7.38-7.09 (m, 2H), 6.74-6.11 (m, 1H), 6.01-5.35 (m, 1H), 5.32-5.23 (m, 2H), 4.92-4.70 (m, 1H), 4.06-3.80 (m, 4H), 2.93-2.67 (m, 2H), 2.26-1.69 (m, 7H). LC-MS (M+H)⁺=494.4.

### Example A14: 5-amino-N-((5-(2-chloro-6-fluorophenyl)pyridin-2-yl)methyl)-6-methyl-N-(5,6,7,8-tetrahydroquinolin-8-yl)-1H-pyrrolo[3,2-b]pyridine-2-carboxamide

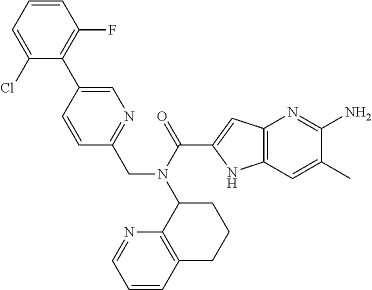 [SMILES: Cc1cc2[nH]c(C(=O)N(Cc3ccc(-c4c(F)cccc4Cl)cn3)C3CCCc4cccnc43)cc2nc1N]

### Step 1: tert-butyl ((5-(2-chloro-6-fluorophenyl)pyridin-2-yl)methyl)carbamate

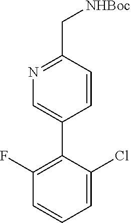 [SMILES: CC(C)(C)OC(=O)NCc1ccc(-c2c(F)cccc2Cl)cn1]

The title compound (180 mg, 69%) was prepared in a manner similar to that in Example 6 step 1 from tert-butyl ((5-bromopyridin-2-yl)methyl)carbamate and 1-chloro-3-fluoro-2-iodobenzene. LC-MS (M+H)⁺=337.2.

### Step 2: (5-(2-chloro-6-fluorophenyl)pyridin-2-yl)methanamine

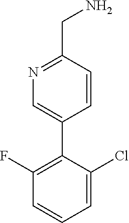 [SMILES: NCc1ccc(-c2c(F)cccc2Cl)cn1]

The title compound (140 mg, 99%) was prepared in a manner similar to that in Example 6 step 2 from tert-butyl ((5-(2-chloro-6-fluorophenyl)pyridin-2-yl)methyl)carbamate. LC-MS (M+H)⁺=237.1.

### Step 3: N-((5-(2-chloro-6-fluorophenyl)pyridin-2-yl)methyl)-5,6,7,8-tetrahydroquinolin-8-amine

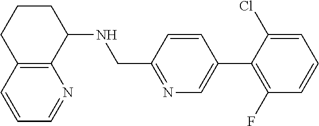 [SMILES: Fc1cccc(Cl)c1-c1ccc(CNC2CCCc3cccnc32)nc1]

The title compound (78 mg, 29%) was prepared in a manner similar to that in Example 10 step 3 from (5-(2-chloro-6-fluorophenyl)pyridin-2-yl)methanamine and 6,7-dihydroquinolin-8 (5H)-one. LC-MS (M+H)⁺=368.1.

### Step 4: 5-amino-6-methyl-1H-pyrrolo[3,2-b]pyridine-2-carboxylic acid

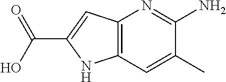

A mixture of 5-((diphenylmethylene)amino)-6-methyl-1-((2-(trimethylsilyl)ethoxy)methyl)-1H-pyrrolo[3,2-b]pyridine-2-carboxylic acid (1.7 g, 3.5 mmol) in TFA (10 mL) and DCM (10 mL) was stirred at 30° C. for 4 h. The mixture was concentrated under reduced pressure and treated with of aqueous HCl (1 M, 15 mL). The mixture was stirred at room temperature for 1 h and concentrated under reduced pressure. The residue was treated with aqueous K₂CO₃ (1 M, 10 mL) and MeOH (10 mL), and the mixture was stirred at 50° C. for 2 h. The mixture was concentrated under reduced pressure, and the crude was purification by C18 chromatograph (10% MeCN in water with 0.03% formic acid) to give the title compound (0.60 g, 90%). LCMS (M+H)⁺=192.2.

### Step 5: 5-amino-N-((5-(2-chloro-6-fluorophenyl)pyridin-2-yl)methyl)-6-methyl-N-(5,6,7,8-tetrahydroquinolin-8-yl)-1H-pyrrolo[3,2-b]pyridine-2-carboxamide

To a solution of 5-amino-6-methyl-1H-pyrrolo[3,2-b]pyridine-2-carboxylic acid (20 mg, 0.10 mmol) in DMF (4 mL) was added HATU (44 mg, 0.11 mmol), N-((5-(2-chloro-6-fluorophenyl)pyridin-2-yl)methyl)-5,6,7,8-tetrahydroquinolin-8-amine (42 mg, 0.12 mmol) and DIPEA (27 mg, 0.21 mmol). The mixture was stirred for 3 h at room temperature, then aqueous NaOH (1 M, 3 mL) was added and stirred for 1 h at room temperature. The mixture was diluted with water (50 mL) and extracted with EtOAc (50 mL). The organic layer was washed with brine (50 mL×2), dried over Na₂SO₄, filtered and concentrated under vacuum. The residue was purified by prep-HPLC to give Example 14 (3 mg, 5%). ¹H NMR (500 MHz, DMSO-d6) δ 11.51-11.16 (m, 1H), 8.72-8.26 (m, 2H), 8.05-6.56 (m, 9H), 6.31-5.77 (m, 1H), 5.57-3.66 (m, 4H), 2.97-2.67 (m, 2H), 2.45-1.85 (m, 6H), 1.86-1.67 (m, 1H). LC-MS (M+H)⁺=541.3.

### Example A15: 5-amino-N-((5-(2-cyano-6-fluorophenyl)pyridin-2-yl)methyl)-6-methyl-N-(5,6,7,8-tetrahydroquinolin-8-yl)-1H-pyrrolo[3,2-b]pyridine-2-carboxamide

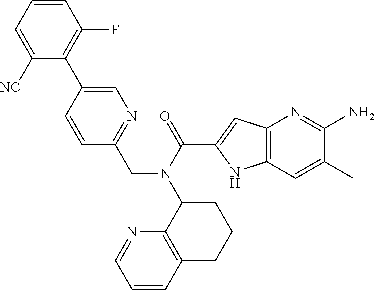 [SMILES: Cc1cc2[nH]c(C(=O)N(Cc3ccc(-c4c(F)cccc4C#N)cn3)C3CCCc4cccnc43)cc2nc1N]

### Step 1: tert-butyl ((5-(2-cyano-6-fluorophenyl)pyridin-2-yl)methyl)carbamate

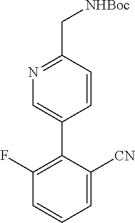 [SMILES: CC(C)(C)OC(=O)NCc1ccc(-c2c(F)cccc2C#N)cn1]

The title compound (308 mg, 77%) was prepared in a manner similar to that in Example 6 step 1 from tert-butyl ((5-bromopyridin-2-yl)methyl)carbamate and 3-fluoro-2-iodobenzonitrile. ¹H NMR (400 MHZ, DMSO-d6) δ 8.65 (s, 1H), 8.00 (d, J=8.0 Hz, 1H), 7.89 (d, J=6.8 Hz, 1H), 7.82-7.73 (m, 1H), 7.73-7.66 (m, 1H), 7.56-7.50 (m, 1H), 7.44 (d, J=8.0 Hz, 1H), 4.36-4.28 (m, 2H), 4.50 (d, J=5.6 Hz, 2H), 1.40 (s, 9H).

### Step 2: 2-(6-(aminomethyl)pyridin-3-yl)-3-fluorobenzonitrile

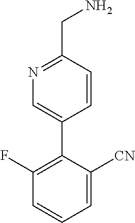 [SMILES: N#Cc1cccc(F)c1-c1ccc(CN)nc1]

The title compound (0.20 g, 96%) was prepared in a manner similar to that in Example 6 step 2 from tert-butyl ((5-(2-cyano-6-fluorophenyl)pyridin-2-yl)methyl)carbamate.

### Step 3: 3-fluoro-2-(6-(((5,6,7,8-tetrahydroquinolin-8-yl)amino)methyl)pyridin-3-yl)benzonitrile

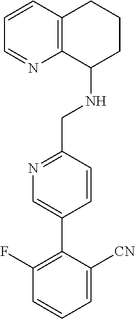 [SMILES: N#Cc1cccc(F)c1-c1ccc(CNC2CCCc3cccnc32)nc1]

The title compound (212 mg, 34%) was prepared in a manner similar to that in Example 10 step 3 from 2-(6-(aminomethyl)pyridin-3-yl)-3-fluorobenzonitrile and 6,7-dihydroquinolin-8 (5H)-one. LC-MS (M+H)⁺=359.1.

### Step 4: 5-amino-N-((5-(2-cyano-6-fluorophenyl)pyridin-2-yl)methyl)-6-methyl-N-(5,6,7,8-tetrahydroquinolin-8-yl)-1H-pyrrolo[3,2-b]pyridine-2-carboxamide

Example 15 (6 mg, 11%) was prepared in a manner similar to that in Example 14 step 5 from 5-amino-6-methyl-1H-pyrrolo[3,2-b]pyridine-2-carboxylic acid and 3-fluoro-2-(6-(((5,6,7,8-tetrahydroquinolin-8-yl)amino)methyl)pyridin-3-yl)benzonitrile. ¹H NMR (500 MHz, DMSO-d6) δ 11.44-11.20 (m, 1H), 8.90-8.52 (m, 1H), 8.49-8.27 (m, 1H), 8.14-6.56 (m, 9H), 6.31-5.58 (m, 1H), 5.56-3.77 (m, 4H), 2.97-2.68 (m, 2H), 2.47-1.88 (m, 6H), 1.87-1.66 (m, 1H). LC-MS (M+H)⁺=532.4.

### Example A16: N-([2,3′-bipyridin]-6′-ylmethyl)-5-amino-6-methyl-N-(5,6,7,8-tetrahydroquinolin-8-yl)-1H-pyrrolo[3,2-b]pyridine-2-carboxamide

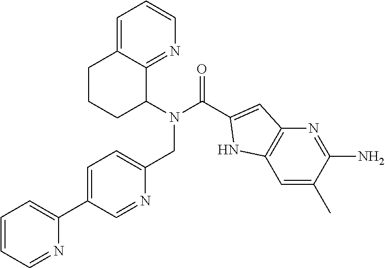 [SMILES: Cc1cc2[nH]c(C(=O)N(Cc3ccc(-c4ccccn4)cn3)C3CCCc4cccnc43)cc2nc1N]

### Step 1: tert-butyl ([2,3′-bipyridin]-6′-ylmethyl)carbamate

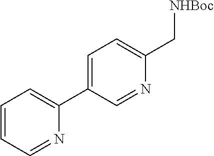 [SMILES: CC(C)(C)OC(=O)NCc1ccc(-c2ccccn2)cn1]

The title compound (0.40 g, 59%) was prepared in a manner similar to that in Example 6 step 1 from tert-butyl ((5-bromopyridin-2-yl)methyl)carbamate and 2-bromopyridine. LC-MS (M+H)⁺=286.3.

### Step 2: [2,3′-bipyridin]-6′-ylmethanamine

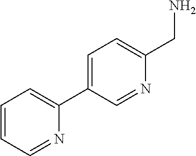 [SMILES: NCc1ccc(-c2ccccn2)cn1]

To a solution of tert-butyl ([2,3′-bipyridin]-6′-ylmethyl)carbamate (400 mg, 1.40 mmol) in DCM (4 mL) was added TFA (2 mL). The mixture was stirred at room temperature for 1 h. The mixture was neutralized with aqueous NaHCO₃ until pH reached 8. The mixture was diluted with water (10 mL) and extracted with DCM (10 mL×2). The combined organic layer was washed with brine (10 mL), dried with Na₂SO₄, filtered and concentrated in vacuum to give the title compound (200 mg, 77%). LC-MS (M+H)⁺=186.3.

### Step 3: N-([2,3′-bipyridin]-6′-ylmethyl)-5,6,7,8-tetrahydroquinolin-8-amine

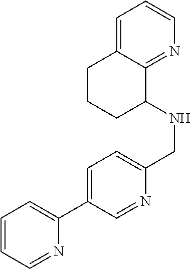 [SMILES: c1ccc(-c2ccc(CNC3CCCc4cccnc43)nc2)nc1]

The title compound (0.30 g, 29%) was prepared in a manner similar to that in Example 10 step 3 from 6,7-dihydroquinolin-8 (5H)-one and [2,3′-bipyridin]-6′-ylmethanamine. LC-MS (M+H)⁺=317.1.

### Step 4: N-([2,3′-bipyridin]-6′-ylmethyl)-5-((diphenylmethylene)amino)-6-methyl-N-(5,6,7,8-tetrahydroquinolin-8-yl)-1-((2-(trimethylsilyl)ethoxy)methyl)-1H-pyrrolo[3,2-b]pyridine-2-carboxamide

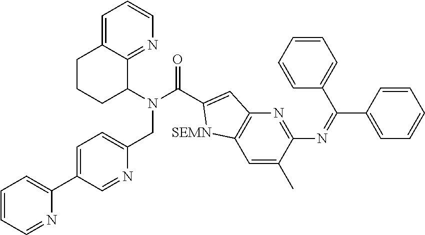 [SMILES: Cc1cc2c(cc(C(=O)N(Cc3ccc(-c4ccccn4)cn3)C3CCCc4cccnc43)n2COCC[Si](C)(C)C)nc1N=C(c1ccccc1)c1ccccc1]

The title compound (22 mg, 43%) was prepared in a manner similar to that in Example 1 step 8 from 5-((diphenylmethylene)amino)-6-methyl-1-((2-(trimethylsilyl)ethoxy)methyl)-1H-pyrrolo[3,2-b]pyridine-2-carboxylic acid and N-([2,3′-bipyridin]-6′-ylmethyl)-5,6,7,8-tetrahydroquinolin-8-amine. LC-MS (M+H)⁺=784.2.

### Step 5: N-([2,3′-bipyridin]-6′-ylmethyl)-5-amino-6-methyl-N-(5,6,7,8-tetrahydroquinolin-8-yl)-1H-pyrrolo[3,2-b]pyridine-2-carboxamide

Example 16 (7 mg, 51%) was prepared in a manner similar to that in Example 1 step 9 from N-([2,3′-bipyridin]-6′-ylmethyl)-5-((diphenylmethylene)amino)-6-methyl-N-(5,6,7,8-tetrahydroquinolin-8-yl)-1-((2-(trimethylsilyl)ethoxy)methyl)-1H-pyrrolo[3,2-b]pyridine-2-carboxamide. ¹H NMR (500 MHZ, CD₃OD) δ 9.27-8.93 (m, 1H), 8.73-8.23 (m, 3H), 8.02-7.84 (m, 2H), 7.81-7.47 (m, 3H), 7.46-7.11 (m, 2H), 6.89-6.44 (m, 1H), 6.08-5.43 (m, 1H), 5.28-4.96 (m, 1H), 4.22-3.34 (m, 1H), 2.97-2.75 (m, 2H), 2.49-1.93 (m, 6H), 1.86-1.73 (m, 1H). LC-MS (M+H)⁺=490.3.

### Example A17: (R)-5-amino-N-((5-(2,6-difluorophenyl)pyridin-2-yl)methyl)-3-fluoro-6-methyl-N-(5,6,7,8-tetrahydroquinolin-8-yl)-1H-pyrrolo[3,2-b]pyridine-2-carboxamide

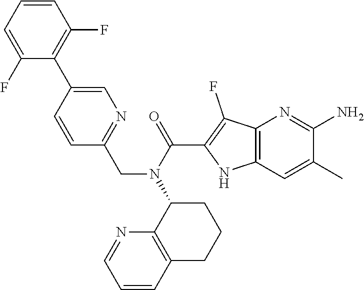 [SMILES: Cc1cc2[nH]c(C(=O)N(Cc3ccc(-c4c(F)cccc4F)cn3)[C@@H]3CCCc4cccnc43)c(F)c2nc1N]

### Step 1: methyl 5-((diphenylmethylene)amino)-3-fluoro-6-methyl-1-((2-(trimethylsilyl)ethoxy)methyl)-1H-pyrrolo[3,2-b]pyridine-2-carboxylate

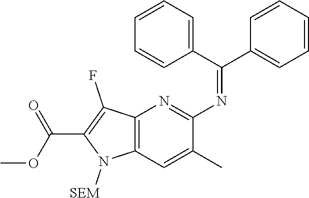 [SMILES: COC(=O)c1c(F)c2nc(N=C(c3ccccc3)c3ccccc3)c(C)cc2n1COCC[Si](C)(C)C]

To a solution of methyl 5-((diphenylmethylene)amino)-6-methyl-1-((2-(trimethylsilyl)ethoxy)methyl)-1H-pyrrolo[3,2-b]pyridine-2-carboxylate (500 mg, 1.0 mmol) in MeCN (5 mL) was added NaHCO₃ (168 mg, 2.0 mmol) and Selectfluor® (886 mg, 2.50 mmol). The mixture was stirred at room temperature for 16 h, diluted with water (5 mL) and extracted with EtOAc (5 mL×3). The combined organic layer was washed with brine (5 mL×2), dried over Na₂SO₄, filtered and concentrated under reduced pressure. The residue was purified by prep-HPLC to give the title compound (15 mg, 3%). LC-MS (M+H)⁺=518.3.

### Step 2: 5-((diphenylmethylene)amino)-3-fluoro-6-methyl-1-((2-(trimethylsilyl)ethoxy)methyl)-1H-pyrrolo[3,2-b]pyridine-2-carboxylic Acid

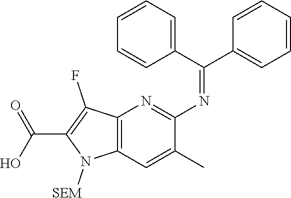 [SMILES: Cc1cc2c(nc1N=C(c1ccccc1)c1ccccc1)c(F)c(C(=O)O)n2COCC[Si](C)(C)C]

The title compound (13 mg, 89%) was prepared in a manner similar to that in Example 1 step 5 from methyl 5-((diphenylmethylene)amino)-3-fluoro-6-methyl-1-((2-(trimethylsilyl)ethoxy)methyl)-1H-pyrrolo[3,2-b]pyridine-2-carboxylate. LC-MS (M+H)⁺=504.3.

### Step 3: (R)—N-((5-(2,6-difluorophenyl)pyridin-2-yl)methyl)-5-((diphenylmethylene)amino)-3-fluoro-6-methyl-N-(5,6,7,8-tetrahydroquinolin-8-yl)-1-((2-(trimethylsilyl)ethoxy)methyl)-1H-pyrrolo[3,2-b]pyridine-2-carboxamide

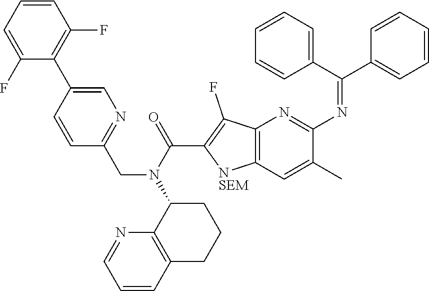 [SMILES: Cc1cc2c(nc1N=C(c1ccccc1)c1ccccc1)c(F)c(C(=O)N(Cc1ccc(-c3c(F)cccc3F)cn1)[C@@H]1CCCc3cccnc31)n2COCC[Si](C)(C)C]

To a solution of (R)—N-((5-(2,6-difluorophenyl)pyridin-2-yl)methyl)-5,6,7,8-tetrahydroquinolin-8-amine (6 mg, 18 μmol) and 5-((diphenylmethylene)amino)-3-fluoro-6-methyl-1-((2-(trimethylsilyl)ethoxy)methyl)-1H-pyrrolo[3,2-b]pyridine-2-carboxylic acid (9 mg, 18 μmol) in THF (1 mL) was added PyBroP® (9 mg, 20 μmol) and DIPEA (6 mg, 45 μmol). The mixture was stirred at room temperature for 12 h. The mixture was diluted with water (3 mL) and extracted with DCM (3 mL×3). The combined organic layer was washed with brine (10 mL), dried over Na₂SO₄, filtered and concentrated under reduced pressure. The residue was purified by prep-TLC (PE:EtOAc=1:1) to give the title compound (10 mg, 67%). LC-MS (M+H)⁺=837.5.

### Step 4: (R)-5-amino-N-((5-(2,6-difluorophenyl)pyridin-2-yl)methyl)-3-fluoro-6-methyl-N-(5,6,7,8-tetrahydroquinolin-8-yl)-1H-pyrrolo[3,2-b]pyridine-2-carboxamide

Example 17 (1.5 mg, 23%) was prepared in a manner similar to that in Example 1 step 9 from (R)—N-((5-(2,6-difluorophenyl)pyridin-2-yl)methyl)-5-((diphenylmethylene)amino)-3-fluoro-6-methyl-N-(5,6,7,8-tetrahydroquinolin-8-yl)-1-((2-(trimethylsilyl)ethoxy)methyl)-1H-pyrrolo[3,2-b]pyridine-2-carboxamide. ¹H NMR (400 MHZ, CDCl₃) δ 9.76 (s, 1H), 8.76-8.23 (m, 2H), 7.79-7.68 (m, 1H), 7.63-7.31 (m, 3H), 7.18-6.94 (m, 4H), 5.68-5.47 (m, 1H), 5.16-5.04 (m, 1H), 4.46 (s, 2H), 4.28-4.01 (m, 1H), 2.97-2.80 (m, 1H), 2.80-2.63 (m, 1H), 2.50-2.33 (m, 1H), 2.26 (s, 3H), 2.16-1.97 (m, 2H), 1.89-1.70 (m, 1H). LC-MS (M+H)⁺=543.1.

### Example A18: 5-amino-N-(4-(1,4-dimethyl-1H-pyrazol-5-yl)-2-fluorobenzyl)-6-methyl-N-(5,6,7,8-tetrahydroquinolin-8-yl)-1H-pyrrolo[3,2-b]pyridine-2-carboxamide

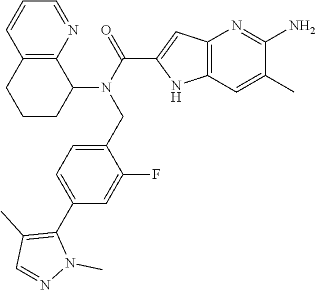

### Step 1: 4-(1,4-dimethyl-1H-pyrazol-5-yl)-2-fluorobenzaldehyde

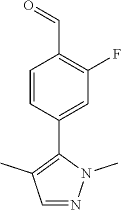

A mixture of (3-fluoro-4-formylphenyl) boronic acid (435 mg, 2.6 mmol), 5-bromo-1,4-dimethyl-1H-pyrazole (450 mg, 2.6 mmol), Pd(PPh₃)₄ (300 mg, 0.10 mmol), K₂CO₃ (900 mg, 6.5 mmol) was added in dioxane (10 mL) and water (1 mL) under nitrogen. The mixture was stirred for overnight at 90° C. The mixture was cooled to room temperature and diluted with EtOAc (50 mL). The mixture was successively washed with water (30 mL) and brine (20 mL), dried over Na₂SO₄ and concentrated under reduced pressure. The residue was purified by prep-TLC (PE:EtOAc=2:1) to give the title compound (400 mg, 73%). LC-MS (M+H)⁺=219.2.

### Step 2: N-(4-(1,4-dimethyl-1H-pyrazol-5-yl)-2-fluorobenzyl)-5,6,7,8-tetrahydroquinolin-8-amine

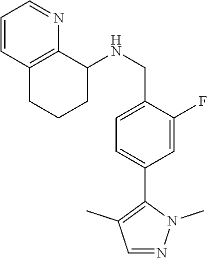 [SMILES: Cc1cnn(C)c1-c1ccc(CNC2CCCc3cccnc32)c(F)c1]

The title compound (120 mg, 48%) was prepared in a manner similar to that in Example 1 step 7 from 5,6,7,8-tetrahydroquinolin-8-amine and 4-(1,4-dimethyl-1H-pyrazol-5-yl)-2-fluorobenzaldehyde. LC-MS (M+H)⁺=351.3.

### Step 3: N-(4-(1,4-dimethyl-1H-pyrazol-5-yl)-2-fluorobenzyl)-5-((diphenylmethylene)amino)-6-methyl-N-(5,6,7,8-tetrahydroquinolin-8-yl)-1-((2-(trimethylsilyl)ethoxy)methyl)-1H-pyrrolo[3,2-b]pyridine-2-carboxamide

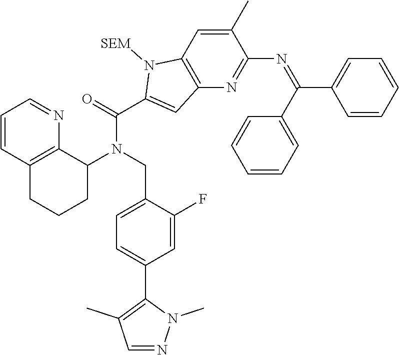 [SMILES: Cc1cc2c(cc(C(=O)N(Cc3ccc(-c4c(C)cnn4C)cc3F)C3CCCc4cccnc43)n2COCC[Si](C)(C)C)nc1N=C(c1ccccc1)c1ccccc1]

The title compound (40 mg, 48%) was prepared in a manner similar to that in Example 1 step 8 from 5-((diphenylmethylene)amino)-6-methyl-1-((2-(trimethylsilyl)ethoxy)methyl)-1H-pyrrolo[3,2-b]pyridine-2-carboxylic acid and N-(4-(1,4-dimethyl-1H-pyrazol-5-yl)-2-fluorobenzyl)-5,6,7,8-tetrahydroquinolin-8-amine. LC-MS (M+H)⁺=818.5.

### Step 4: 5-amino-N-(4-(1,4-dimethyl-1H-pyrazol-5-yl)-2-fluorobenzyl)-6-methyl-N-(5,6,7,8-tetrahydroquinolin-8-yl)-1H-pyrrolo[3,2-b]pyridine-2-carboxamide

Example 18 (15 mg, 59%) was prepared in a manner similar to that in Example 1 step 9 from N-(4-(1,4-dimethyl-1H-pyrazol-5-yl)-2-fluorobenzyl)-5-((diphenylmethylene)amino)-6-methyl-N-(5,6,7,8-tetrahydroquinolin-8-yl)-1-((2-(trimethylsilyl)ethoxy)methyl)-1H-pyrrolo[3,2-b]pyridine-2-carboxamide. ¹H NMR (500 MHz, DMSO-d6) δ 11.37 (s, 1H), 8.41 (s, 1H), 8.02-7.47 (m, 2H), 7.40-7.10 (m, 5H), 6.66 (s, 1H), 6.23-5.85 (m, 1H), 5.29 (s, 2H), 4.83-4.68 (m, 1H), 3.90-3.79 (m, 1H), 3.70 (s, 3H), 2.92-2.79 (m, 1H), 2.78-2.68 (m, 1H), 2.15 (s, 3H), 2.09-2.01 (m, 1H), 1.97 (s, 3H), 1.84-1.70 (m, 1H). LC-MS (M+H)⁺=524.3.

### Example A19: (R)-5-amino-N-((5-(2,6-difluorophenyl)pyridin-2-yl)methyl)-3-iodo-6-methyl-N-(5,6,7,8-tetrahydroquinolin-8-yl)-1H-pyrrolo[3,2-b]pyridine-2-carboxamide

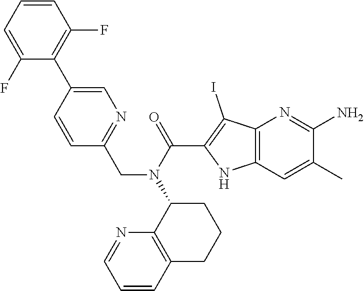 [SMILES: Cc1cc2[nH]c(C(=O)N(Cc3ccc(-c4c(F)cccc4F)cn3)[C@@H]3CCCc4cccnc43)c(I)c2nc1N]

### Step 1: 5-((diphenylmethylene)amino)-3-iodo-6-methyl-1-((2-(trimethylsilyl)ethoxy)methyl)-1H-pyrrolo[3,2-b]pyridine-2-carboxylic acid

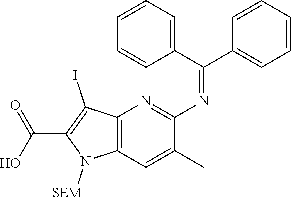 [SMILES: Cc1cc2c(nc1N=C(c1ccccc1)c1ccccc1)c(I)c(C(=O)O)n2COCC[Si](C)(C)C]

To a solution of 5-((diphenylmethylene)amino)-6-methyl-1-((2-(trimethylsilyl)ethoxy)methyl)-1H-pyrrolo[3,2-b]pyridine-2-carboxylic acid (200 mg, 0.41 mmol) in MeCN (10 mL) was added NIS (110 mg, 0.49 mmol). The mixture was stirred at room temperature for 4 h. The mixture was diluted with chloroform and isopropyl alcohol (3:1, 40 mL), washed with brine (20 mL), dried over Na₂SO₄, filtered and concentrated under reduce pressure to give the title compound (175 mg, 70%). LC-MS (M+H)⁺=612.1.

### Step 2: (R)—N-((5-(2,6-difluorophenyl)pyridin-2-yl)methyl)-5-((diphenylmethylene)amino)-3-iodo-6-methyl-N-(5,6,7,8-tetrahydroquinolin-8-yl)-1-((2-(trimethylsilyl)ethoxy)methyl)-1H-pyrrolo[3,2-b]pyridine-2-carboxamide

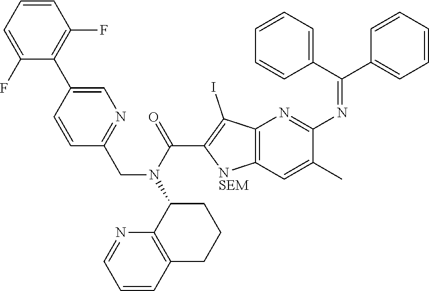 [SMILES: Cc1cc2c(nc1N=C(c1ccccc1)c1ccccc1)c(I)c(C(=O)N(Cc1ccc(-c3c(F)cccc3F)cn1)[C@@H]1CCCc3cccnc31)n2COCC[Si](C)(C)C]

The title compound (134 mg, 58%) was prepared in a manner similar to that in Example 1 step 8 from 5-((diphenylmethylene)amino)-3-iodo-6-methyl-1-((2-(trimethylsilyl)ethoxy)methyl)-1H-pyrrolo[3,2-b]pyridine-2-carboxylic acid and (R)—N-((5-(2,6-difluorophenyl)pyridin-2-yl)methyl)-5,6,7,8-tetrahydroquinolin-8-amine. LC-MS (M+H)⁺=945.3

### Step 3: (R)-5-amino-N-((5-(2,6-difluorophenyl)pyridin-2-yl)methyl)-3-iodo-6-methyl-N-(5,6,7,8-tetrahydroquinolin-8-yl)-1H-pyrrolo[3,2-b]pyridine-2-carboxamide

Example 19 (0.5 mg, 7%) was prepared in a manner similar to that in Example 1 step 9 from (R)—N-((5-(2,6-difluorophenyl)pyridin-2-yl)methyl)-5-((diphenylmethylene)amino)-3-iodo-6-methyl-N-(5,6,7,8-tetrahydroquinolin-8-yl)-1-((2-(trimethylsilyl)ethoxy)methyl)-1H-pyrrolo[3,2-b]pyridine-2-carboxamide. ¹H NMR (500 MHz, CD₃OD) δ 8.62-7.90 (m, 1H), 7.88-7.37 (m, 3H), 7.31-7.07 (m, 3H), 5.68-4.93 (m, 2H), 4.77-4.07 (m, 1H), 3.01-2.30 (m, 2H), 2.30-2.17 (m, 3H), 2.14-1.52 (m, 3H). LC-MS (M+H)⁺=651.3.

### Example B1: 5-amino-N-cyclobutyl-6-methyl-N-((5-phenylpyridin-2-yl)methyl)-1H-pyrrolo[3,2-b]pyridine-2-carboxamide

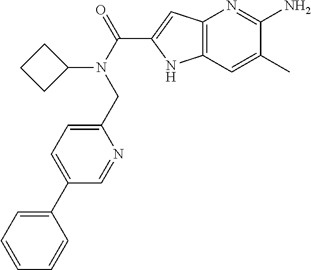 [SMILES: Cc1cc2[nH]c(C(=O)N(Cc3ccc(-c4ccccc4)cn3)C3CCC3)cc2nc1N]

### Step 1: 5-chloro-6-methyl-1H-pyrrolo[3,2-b]pyridine-2-carboxylic Acid

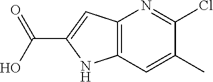

A mixture of 2,6-dichloro-5-methylpyridin-3-amine (10.0 g, 56.5 mmol), pyruvic acid (14.9 g, 169 mmol), Pd(OAc)₂ (0.94 g, 5.65 mmol), PPh₃ (11.8 g, 45.2 mmol) in DMF (100 mL) was stirred at 110° C. for 24 h. The mixture was cooled to room temperature and volatiles were removed in vacuo. Aqueous K₂CO₃ (24 g in 200 mL of water) and MTBE (300 mL) was added, and the mixture was stirred for 10 min. Solid was removed by filtration. The aqueous layer was separated, and the pH was adjusted to 3-4 with aqueous HCl (6 M). The mixture was extracted with EtOAc (500 mL×2). The combined organic layer was washed with brine (300 mL), dried over Na₂SO₄, filtered and concentrated under reduced pressure. The residue was purified by C18 chromatograph (MeCN: 0.03% aqueous formic acid, 0:1 to 1:1) to give the title compound (4.0 g, 34%). LC-MS (M+H)⁺=211.

### Step 2: methyl 5-chloro-6-methyl-1H-pyrrolo[3,2-b]pyridine-2-carboxylate

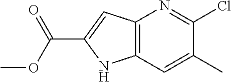

To a mixture of 5-chloro-6-methyl-1H-pyrrolo[3,2-b]pyridine-2-carboxylic acid (8.5 g, 40.3 mmol) in MeOH (100 mL) was added concentrated H₂SO₄ (10 mL) dropwise at 0° C. The mixture was stirred at reflux for overnight. The mixture was cooled to room temperature and most volatiles were removed in vacuo. The mixture was quenched with iced water (100 mL) and extracted with EtOAc (100 mL×2). The combined organic layer was washed with brine (100 mL), dried over Na₂SO₄, filtered and concentrated under reduced pressure. The residue was purified by silica gel chromatograph (PE/EtOAc=8/1) to give the title compound (7.6 g, 84%). LC-MS (M+H)⁺=225.0.

### Step 3: methyl 5-chloro-6-methyl-1-((2-(trimethylsilyl)ethoxy)methyl)-1H-pyrrolo[3,2-b]pyridine-2-carboxylate

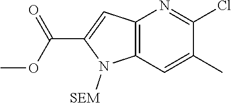

A mixture of methyl 5-chloro-6-methyl-1H-pyrrolo[3,2-b]pyridine-2-carboxylate (7.6 g, 33.9 mmol), SEMCl (11.3 g, 67.8 mmol), DIPEA (13.1 g, 101.7 mmol) in THF (120 mL) was stirred at 70° C. overnight under nitrogen. The mixture was cooled to room temperature and concentrated under reduced pressure. The crude was purified by silica gel chromatograph (PE/EtOAc=10/1) to give the title compound (6.8 g, 71%). LC-MS (M+H)⁺=355.1.

### Step 4: methyl 5-((diphenylmethylene)amino)-6-methyl-1-((2-(trimethylsilyl)ethoxy)methyl)-1H-pyrrolo[3,2-b]pyridine-2-carboxylate

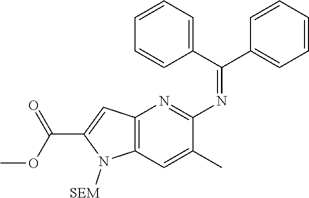 [SMILES: COC(=O)c1cc2nc(N=C(c3ccccc3)c3ccccc3)c(C)cc2n1COCC[Si](C)(C)C]

A mixture of methyl 5-chloro-6-methyl-1-((2-(trimethylsilyl)ethoxy)methyl)-1H-pyrrolo[3,2-b]pyridine-2-carboxylate (2.8 g, 7.89 mmol), diphenylmethanimine (2.14 g, 11.8 mmol), Pd₂(dba)₃ (361 mg, 0.39 mmol), BINAP (0.492 g, 0.79 mmol) and K₃PO₄ (5.0 g, 23.7 mmol) in dioxane (50 mL) was stirred at 100° C. under N₂ overnight. The mixture was cooled to room temperature, diluted with water (100 mL) and extracted with EtOAc (100 mL×2). The combined organic layer was washed with brine (100 mL) and concentrated under reduced pressure. The residue was purified by silica gel chromatograph (PE/EtOAc=4/1) to give the title compound (3.0 g, 76%). LC-MS (M+H)⁺=500.2.

### Step 5: 5-((diphenylmethylene)amino)-6-methyl-1-((2-(trimethylsilyl)ethoxy)methyl)-1H-pyrrolo[3,2-b]pyridine-2-carboxylic Acid

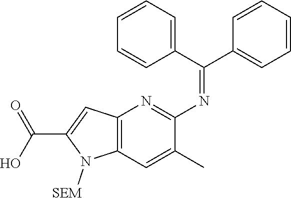 [SMILES: Cc1cc2c(cc(C(=O)O)n2COCC[Si](C)(C)C)nc1N=C(c1ccccc1)c1ccccc1]

To a mixture of methyl 5-((diphenylmethylene)amino)-6-methyl-1-((2-(trimethylsilyl)ethoxy)methyl)-1H-pyrrolo[3,2-b]pyridine-2-carboxylate (3.0 g, 6.0 mmol) in THF (50 mL) and water (50 mL) was added LiOH·H₂O (0.75 g, 18 mol). The mixture was stirred at 60° C. for 4 h. The mixture was cooled to room temperature and the pH was adjusted to 7-8 with aqueous HCl (1 M). The mixture was extracted with EtOAc (50 mL×3). The combined organic layer was washed with brine (50 mL), dried over Na₂SO₄, filtered and concentrated under reduced pressure to give the title compound (2.5 g, 86%). LC-MS (M+H)⁺=486.2.

### Step 6: N-((5-phenylpyridin-2-yl)methyl)cyclobutanamine

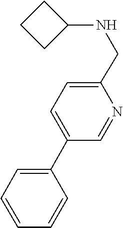 [SMILES: c1ccc(-c2ccc(CNC3CCC3)nc2)cc1]

To a solution of 5-phenylpicolinaldehyde (300 mg, 1.64 mmol) and cyclobutanamine (233 mg, 3.28 mmol) in DCM (2 mL) and MeOH (0.2 mL) was added NaBH(OAc)₃ (694 mg, 3.28 mmol). The mixture was stirred at room temperature for 1 h, then carefully added to water (100 mL) dropwise with vigorous stirring. The mixture was extracted with EtOAc (200 mL). The organic layer was separated and washed with brine (100 mL), dried over Na₂SO₄, filtered and concentrated under vacuum. The residue was purified by prep-HPLC to give the title compound (100 mg, 26%). LC-MS (M+H)⁺=239.2.

### Step 7: N-cyclobutyl-5-((diphenylmethylene)amino)-6-methyl-N-((5-phenylpyridin-2-yl)methyl)-1-((2-(trimethylsilyl)ethoxy)methyl)-1H-pyrrolo[3,2-b]pyridine-2-carboxamide

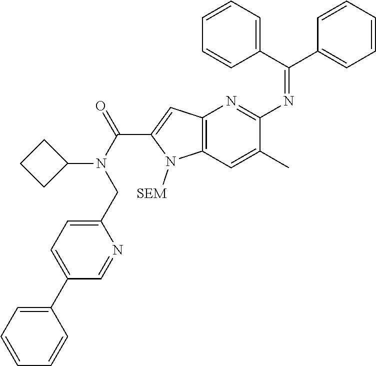 [SMILES: Cc1cc2c(cc(C(=O)N(Cc3ccc(-c4ccccc4)cn3)C3CCC3)n2COCC[Si](C)(C)C)nc1N=C(c1ccccc1)c1ccccc1]

A mixture of 5-((diphenylmethylene)amino)-6-methyl-1-((2-(trimethylsilyl)ethoxy)methyl)-1H-pyrrolo[3,2-b]pyridine-2-carboxylic acid (40 mg, 0.082 mmol), N-((5-phenylpyridin-2-yl)methyl)cyclobutanamine (20 mg, 0.082 mmol), HATU (31 mg, 0.082 mmol) and DIPEA (21 mg, 0.164 mmol) in DMF (1.5 mL) was stirred at 60° C. for overnight under nitrogen. The mixture was cooled to room temperature and concentrated under reduced pressure. The residue was purified by silica gel chromatograph (DCM/MeOH=20/1) to give the title compound (40 mg, 69%). LC-MS (M+H)⁺=706.6.

### Step 8: 5-amino-N-cyclobutyl-6-methyl-N-((5-phenylpyridin-2-yl)methyl)-1H-pyrrolo[3,2-b]pyridine-2-carboxamide

To a solution of N-cyclobutyl-5-((diphenylmethylene)amino)-6-methyl-N-((5-phenylpyridin-2-yl)methyl)-1-((2-(trimethylsilyl)ethoxy)methyl)-1H-pyrrolo[3,2-b]pyridine-2-carboxamide (40 mg, 0.057 mmol) in methanol (2 mL) was added aqueous HCl (6 M, 0.5 mL). The mixture was stirred at room temperature for 1 h then concentrated under reduced pressure. The residue was re-dissolved in DCM (2 mL) then TFA (1 mL) was added. The mixture was stirred at room temperature for 1 h then concentrated under reduced pressure. The residue was re-dissolved in MeOH (2 mL) and water (0.2 mL) followed by addition of K₂CO₃ (39 mg, 0.285 mmol). The mixture was stirred at room temperature for 1 h then concentrated under reduced pressure. The mixture was triturated with DCM (5 mL) and solid was filtered off. The filtrate was concentrated under reduced pressure and the residue was purified by prep-HPLC to give Example 1 (6 mg, 26%). ¹H NMR (500 MHz, DMSO-d6) δ 11.29 (s, 1H), 8.87 (s, 1H), 8.13-7.97 (m, 1H), 7.79-7.66 (m, 2H), 7.50 (t, J=7.6 Hz, 2H), 7.42 (t, J=7.3 Hz, 1H), 7.39-7.27 (m, 2H), 6.74-6.07 (m, 1H), 5.32 (s, 2H), 5.18-4.85 (m, 3H), 2.26-2.05 (m, 7H), 1.71-1.49 (m, 2H). LC-MS (M+H)⁺=412.3.

### Example B2: 5-amino-N-((5-(2,6-difluorophenyl)pyridin-2-yl)methyl)-N-(1,3-dimethoxypropan-2-yl)-6-methyl-1H-pyrrolo[3,2-b]pyridine-2-carboxamide

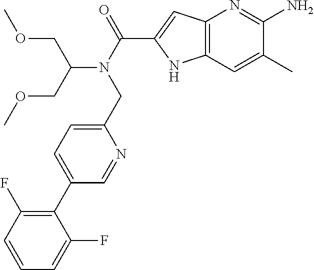 [SMILES: COCC(COC)N(Cc1ccc(-c2c(F)cccc2F)cn1)C(=O)c1cc2nc(N)c(C)cc2[nH]1]

### Step 1: tert-butyl ((5-(2,6-difluorophenyl)pyridin-2-yl)methyl)carbamate

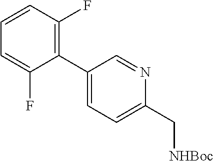 [SMILES: CC(C)(C)OC(=O)NCc1ccc(-c2c(F)cccc2F)cn1]

To a solution of tert-butyl ((5-bromopyridin-2-yl)methyl)carbamate (5.0 g, 17.4 mmol) in dioxane (50 mL) was added BPD (5.31 g, 20.9 mmol), Pd(dppf)Cl₂ (1.27 g, 1.74 mmol) and KOAc (4.27 g, 43.5 mmol). The mixture was stirred at 85° C. for 12 h. The mixture was cooled to room temperature, filtered and concentrated under reduced pressure. The crude was re-dissolved in dioxane (25 mL), toluene (12 mL) and water (12 mL) followed by addition of 1,3-difluoro-2-iodobenzene (4.28 g, 17.8 mmol), K₃PO₄ (6.32 g, 29.8 mmol) and Pd(dppf)Cl₂ (871 mg, 1.19 mmol). The mixture was stirred at 85° C. for 3 h. The mixture was cooled to room temperature, diluted with water (30 mL) and extracted with EtOAc (20 mL×3). The combined organic layer was washed with brine (10 mL×2), dried over Na₂SO₄, filtered and concentrated under reduced pressure. The residue was purified by silica gel chromatograph (PE:EtOAc=1:0 to 5:1) to give the title compound (3.0 g, 54%). ¹H NMR (400 MHZ, CDCl₃) δ 8.64 (s, 1H), 7.78 (d, J=8.4 Hz, 1H), 7.39 (d, J=8.4 Hz, 1H), 7.36-7.28 (m, 1H), 7.06-6.96 (m, 1H), 5.62 (br s, 1H), 4.50 (d, J=5.6 Hz, 2H), 7.47 (s, 9H). LC-MS (M+H)⁺=321.1.

### Step 2: (5-(2,6-difluorophenyl)pyridin-2-yl)methanamine

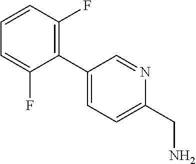 [SMILES: NCc1ccc(-c2c(F)cccc2F)cn1]

A mixture of tert-butyl ((5-(2,6-difluorophenyl)pyridin-2-yl)methyl)carbamate (3.0 g, 9.37 mmol) in methanolic HCl (4 M, 50 mL) was stirred at 25° C. for 2 h, then the mixture was neutralized with methanolic NaOH (1 M) to pH 7. The mixture was concentrated to dryness under reduced pressure, and the residue was triturated with DCM/MeOH (10/1, 10 mL). The filtrate was collected by filtration and concentrated under vacuum to give the title compound (1.5 g, 73%). ¹H NMR (400 MHZ, CD₃OD) δ 8.72 (s, 1H), 7.97 (d, J=6.0 Hz, 1H), 7.60 (d, J=6.0 Hz, 1H), 7.54-7.45 (m, 1H), 7.20-7.10 (m, 1H), 4.37 (s, 2H). LC-MS (M+H)⁺=221.1.

### Step 3: N-((5-(2,6-difluorophenyl)pyridin-2-yl)methyl)-1,3-dimethoxypropan-2-amine

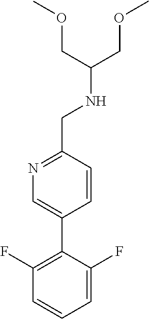 [SMILES: COCC(COC)NCc1ccc(-c2c(F)cccc2F)cn1]

The title compound (0.20 g, 76%) was prepared in a manner similar to that in Example 1 step 6 from 1,3-dimethoxypropan-2-one and (5-(2,6-difluorophenyl)pyridin-2-yl)methanamine. ¹H NMR (400 MHZ, DMSO-d6) δ 8.57 (s, 1H), 7.87 (d, J=8.4 Hz, 1H), 7.60-7.48 (m, 2H), 7.32-7.22 (m, 2H), 3.94 (s, 2H), 3.34 (d, J=5.6 Hz, 4H), 3.24 (s, 6H), 2.89 (quin, J=5.6 Hz, 1H). LC-MS (M+H)⁺=323.2.

### Step 4: N-((5-(2,6-difluorophenyl)pyridin-2-yl)methyl)-N-(1,3-dimethoxypropan-2-yl)-5-((diphenylmethylene)amino)-6-methyl-1-((2-(trimethylsilyl)ethoxy)methyl)-1H-pyrrolo[3,2-b]pyridine-2-carboxamide

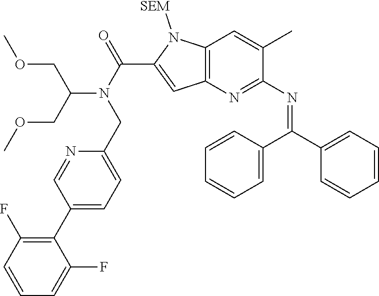 [SMILES: COCC(COC)N(Cc1ccc(-c2c(F)cccc2F)cn1)C(=O)c1cc2nc(N=C(c3ccccc3)c3ccccc3)c(C)cc2n1COCC[Si](C)(C)C]

The title compound (30 mg, 76%) was prepared in a manner similar to that in Example 1 step 7 from 5-((diphenylmethylene)amino)-6-methyl-1-((2-(trimethylsilyl)ethoxy)methyl)-1H-pyrrolo[3,2-b]pyridine-2-carboxylic acid and N-((5-(2,6-difluorophenyl)pyridin-2-yl)methyl)-1,3-dimethoxypropan-2-amine. LC-MS (M+H)⁺=790.6.

### Step 5: 5-amino-N-((5-(2,6-difluorophenyl)pyridin-2-yl)methyl)-N-(1,3-dimethoxypropan-2-yl)-6-methyl-1H-pyrrolo[3,2-b]pyridine-2-carboxamide

Example 2 (9 mg, 47%) was prepared in a manner similar to that in Example 1 step 8 from N-((5-(2,6-difluorophenyl)pyridin-2-yl)methyl)-N-(1,3-dimethoxypropan-2-yl)-5-((diphenylmethylene)amino)-6-methyl-1-((2-(trimethylsilyl)ethoxy)methyl)-1H-pyrrolo[3,2-b]pyridine-2-carboxamide. ¹H NMR (500 MHZ, DMSO-d6) δ 11.22 (s, 1H), 8.59 (s, 1H), 7.88 (s, 1H), 7.75-7.43 (m, 2H), 7.35-7.21 (m, 3H), 6.71 (s, 1H), 5.32 (s, 2H), 5.21-4.65 (m, 3H), 3.68-3.55 (m, 2H), 3.53-3.45 (m, 2H), 3.16 (s, 6H), 2.14 (s, 3H). LC-MS (M+H)⁺=496.3.

### Example B3: 5-amino-N-(3,3-difluorocyclobutyl)-N-((5-(2,6-difluorophenyl)pyridin-2-yl)methyl)-6-methyl-1H-pyrrolo[3,2-b]pyridine-2-carboxamide

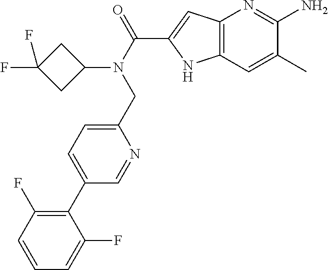 [SMILES: Cc1cc2[nH]c(C(=O)N(Cc3ccc(-c4c(F)cccc4F)cn3)C3CC(F)(F)C3)cc2nc1N]

### Step 1: 5-(2,6-difluorophenyl)picolinaldehyde

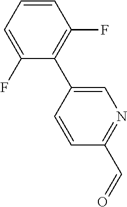 [SMILES: O=Cc1ccc(-c2c(F)cccc2F)cn1]

To a mixture of 5-(4,4,5,5-tetramethyl-1,3,2-dioxaborolan-2-yl)picolinaldehyde (62 g, 266 mmol) and 1,3-difluoro-2-iodobenzene (95.8 g, 399 mmol) in dioxane (600 mL), toluene (300 mL) and water (300 mL) was added Pd(dppf)Cl₂ (9.73 g, 13.3 mmol) and K₃PO₄ (141 g, 665 mmol). The mixture was stirred at 85° C. for 16 h, cooled to room temperature and poured into water (1000 mL). The organic layer was separated. The aqueous phase was extracted with EtOAc (800 mL×2). The combined organic layer was washed with brine (500 mL), dried with Na₂SO₄, filtered and concentrated under vacuum. The residue was purified by silica gel chromatography (PE:EtOAc=50:1 to 5:1) to give the title compound (30 g, 51%). LC-MS (M+H)⁺=220.2.

### Step 2: N-((5-(2,6-difluorophenyl)pyridin-2-yl)methyl)-3,3-difluorocyclobutan-1-amine

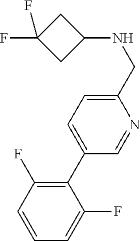 [SMILES: Fc1cccc(F)c1-c1ccc(CNC2CC(F)(F)C2)nc1]

The title compound (50 mg, 35%) was prepared in a manner similar to that in Example 1 step 6 from 5-(2,6-difluorophenyl)picolinaldehyde and 3,3-difluorocyclobutan-1-amine. LC-MS (M+H)⁺=311.1.

### Step 3: N-(3,3-difluorocyclobutyl)-N-((5-(2,6-difluorophenyl)pyridin-2-yl)methyl)-5-((diphenylmethylene)amino)-6-methyl-1-((2-(trimethylsilyl)ethoxy)methyl)-1H-pyrrolo[3,2-b]pyridine-2-carboxamide

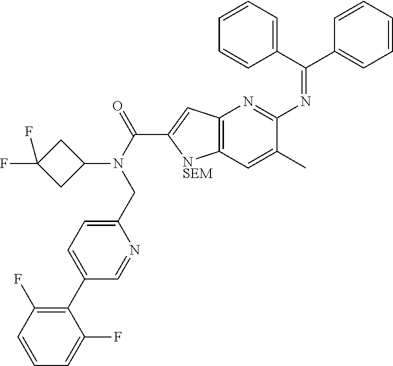 [SMILES: Cc1cc2c(cc(C(=O)N(Cc3ccc(-c4c(F)cccc4F)cn3)C3CC(F)(F)C3)n2COCC[Si](C)(C)C)nc1N=C(c1ccccc1)c1ccccc1]

The title compound (35 mg, 55%) was prepared in a manner similar to that in Example 1 step 7 from 5-((diphenylmethylene)amino)-6-methyl-1-((2-(trimethylsilyl)ethoxy)methyl)-1H-pyrrolo[3,2-b]pyridine-2-carboxylic acid and N-((5-(2,6-difluorophenyl)pyridin-2-yl)methyl)-3,3-difluorocyclobutan-1-amine. LC-MS (M+H)⁺=778.1.

### Step 4: 5-amino-N-(3,3-difluorocyclobutyl)-N-((5-(2,6-difluorophenyl)pyridin-2-yl)methyl)-6-methyl-1H-pyrrolo[3,2-b]pyridine-2-carboxamide

To a solution of N-(3,3-difluorocyclobutyl)-N-((5-(2,6-difluorophenyl)pyridin-2-yl)methyl)-5-((diphenylmethylene)amino)-6-methyl-1-((2-(trimethylsilyl)ethoxy)methyl)-1H-pyrrolo[3,2-b]pyridine-2-carboxamide (35 mg, 0.04 mmol) in DCM (6 mL) was added TFA (6 mL). The mixture was stirred for 2 h at room temperature then concentrated under reduced pressure. The mixture was then treated with HCl (4 M in methanol, 4 mL) and the mixture was stirred for 2 h at room temperature. The mixture was concentrated under vacuum, and the residue was re-dissolved in MeOH (10 mL) and water (2 mL), followed by addition of K₂CO₃ (31 mg, 0.20 mmol). The mixture was stirred for 2 h at 50° C. The mixture was cooled to room temperature and concentrated under vacuum. The residue was purified by prep-HPLC to give Example 3 (3 mg, 14%); ¹H NMR (500 MHz, DMSO-d6) δ 11.34 (s, 1H), 8.66 (s, 1H), 8.04-7.85 (m, 1H), 7.52 (s, 2H), 7.37-7.21 (m, 3H), 6.37 (s, 1H), 5.35 (s, 2H), 5.20-4.44 (m, 3H), 3.04-2.84 (m, 4H), 2.14 (s, 3H). LC-MS (M+H)⁺=484.3.

### Example B4: 5-amino-N-cyclopropyl-6-methyl-N-((5-phenylpyridin-2-yl)methyl)-1H-pyrrolo[3,2-b]pyridine-2-carboxamide

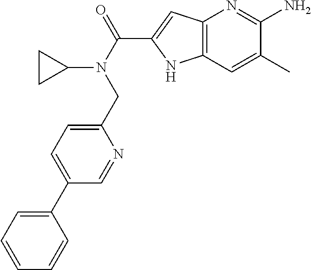 [SMILES: Cc1cc2[nH]c(C(=O)N(Cc3ccc(-c4ccccc4)cn3)C3CC3)cc2nc1N]

### Step 1: N-((5-phenylpyridin-2-yl)methyl)cyclopropanamine

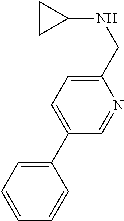 [SMILES: c1ccc(-c2ccc(CNC3CC3)nc2)cc1]

The title compound (150 mg, 61%) was prepared in a manner similar to that in Example 1 step 6 from 5-phenylpicolinaldehyde and cyclopropylamine. LC-MS (M+H)⁺=225.1.

### Step 2: N-cyclopropyl-5-((diphenylmethylene)amino)-6-methyl-N-((5-phenylpyridin-2-yl)methyl)-1-((2-(trimethylsilyl)ethoxy)methyl)-1H-pyrrolo[3,2-b]pyridine-2-carboxamide

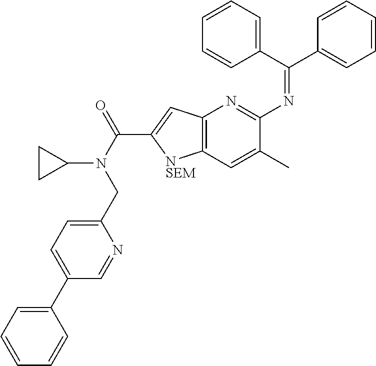 [SMILES: Cc1cc2c(cc(C(=O)N(Cc3ccc(-c4ccccc4)cn3)C3CC3)n2COCC[Si](C)(C)C)nc1N=C(c1ccccc1)c1ccccc1]

The title compound (20 mg, 70%) was prepared in a manner similar to that in Example 1 step 7 from 5-((diphenylmethylene)amino)-6-methyl-1-((2-(trimethylsilyl)ethoxy)methyl)-1H-pyrrolo[3,2-b]pyridine-2-carboxylic acid and N-((5-phenylpyridin-2-yl)methyl)cyclopropanamine. LC-MS (M+H)⁺=692.3.

### Step 3: 5-amino-N-cyclopropyl-6-methyl-N-((5-phenylpyridin-2-yl)methyl)-1H-pyrrolo[3,2-b]pyridine-2-carboxamide

Example 4 (1.6 mg, 14%) was prepared in a manner similar to that in Example 1 step 8 from N-cyclopropyl-5-((diphenylmethylene)amino)-6-methyl-N-((5-phenylpyridin-2-yl)methyl)-1-((2-(trimethylsilyl)ethoxy)methyl)-1H-pyrrolo[3,2-b]pyridine-2-carboxamide. ¹H NMR (500 MHZ, DMSO-d6) δ 11.17 (s, 1H), 8.83 (d, J=1.9 Hz, 1H), 8.05 (dd, J=8.2, 2.3 Hz, 1H), 7.72 (d, J=7.3 Hz, 2H), 7.50 (t, J=7.6 Hz, 2H), 7.45-7.33 (m, 3H), 6.93 (s, 1H), 5.34 (s, 2H), 4.88 (s, 2H), 2.16 (s, 3H), 2.04-1.91 (m, 1H), 1.33-1.23 (m, 2H), 0.95-0.82 (m, 2H). LC-MS (M+H)⁺=398.3.

### Example B5: 5-amino-N-(cyclopropylmethyl)-N-((5-(2,6-difluorophenyl)pyridin-2-yl)methyl)-6-methyl-1H-pyrrolo[3,2-b]pyridine-2-carboxamide

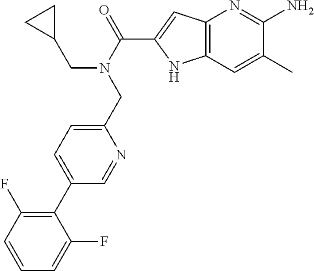 [SMILES: Cc1cc2[nH]c(C(=O)N(Cc3ccc(-c4c(F)cccc4F)cn3)CC3CC3)cc2nc1N]

### Step 1: 1-cyclopropyl-N-((5-(2,6-difluorophenyl)pyridin-2-yl)methyl)methanamine

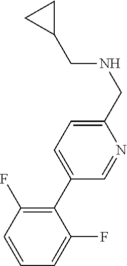 [SMILES: Fc1cccc(F)c1-c1ccc(CNCC2CC2)nc1]

The title compound (36 mg, 14%) was prepared in a manner similar to that in Example 1 step 6 from 5-(2,6-difluorophenyl)picolinaldehyde and cyclopropylmethanamine. LC-MS (M+H)⁺=275.1.

### Step 2: N-(cyclopropylmethyl)-N-((5-(2,6-difluorophenyl)pyridin-2-yl)methyl)-5-((diphenylmethylene)amino)-6-methyl-1-((2-(trimethylsilyl)ethoxy)methyl)-1H-pyrrolo[3,2-b]pyridine-2-carboxamide

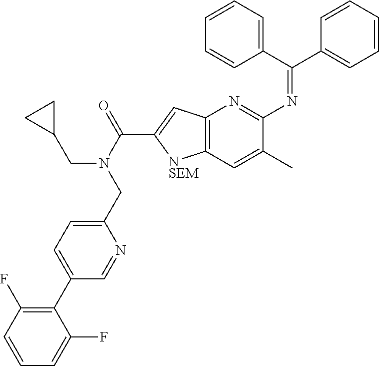 [SMILES: Cc1cc2c(cc(C(=O)N(Cc3ccc(-c4c(F)cccc4F)cn3)CC3CC3)n2COCC[Si](C)(C)C)nc1N=C(c1ccccc1)c1ccccc1]

The title compound (13 mg, 24%) was prepared in a manner similar to that in Example 1 step 7 from 5-((diphenylmethylene)amino)-6-methyl-1-((2-(trimethylsilyl)ethoxy)methyl)-1H-pyrrolo[3,2-b]pyridine-2-carboxylic acid and 1-cyclopropyl-N-((5-(2,6-difluorophenyl)pyridin-2-yl)methyl)methanamine. LC-MS (M+H)⁺=742.3.

### Step 3: 5-amino-N-(cyclopropylmethyl)-N-((5-(2,6-difluorophenyl)pyridin-2-yl)methyl)-6-methyl-1H-pyrrolo[3,2-b]pyridine-2-carboxamide

Example 5 (1.6 mg, 14%) was prepared in a manner similar to that in Example 1 step 8 from N-(cyclopropylmethyl)-N-((5-(2,6-difluorophenyl)pyridin-2-yl)methyl)-5-((diphenylmethylene)amino)-6-methyl-1-((2-(trimethylsilyl)ethoxy)methyl)-1H-pyrrolo[3,2-b]pyridine-2-carboxamide. ¹H NMR (500 MHZ, CD₃OD) δ 8.82-8.50 (m, 1H), 7.95 (s, 1H), 7.58 (d, J=8.1 Hz, 1H), 7.52 (s, 1H), 7.47 (dt, J=14.9, 7.6 Hz, 1H), 7.14 (t, J=8.0 Hz, 2H), 6.59 (s, 1H), 5.39-4.98 (m, 2H), 3.97-3.44 (m, 2H), 2.25 (s, 3H), 1.22-1.07 (m, 1H), 0.54 (d, J=7.3 Hz, 2H), 0.26 (d, J=4.7 Hz, 2H). LC-MS (M+H)⁺=448.3.

### Example B6 & Example B7: 5-amino-N-((1r,3r)-3-methoxycyclobutyl)-6-methyl-N-((5-phenylpyridin-2-yl)methyl)-1H-pyrrolo[3,2-b]pyridine-2-carboxamide & 5-amino-N-((1s,3s)-3-methoxycyclobutyl)-6-methyl-N-((5-phenylpyridin-2-yl)methyl)-1H-pyrrolo[3,2-b]pyridine-2-carboxamide

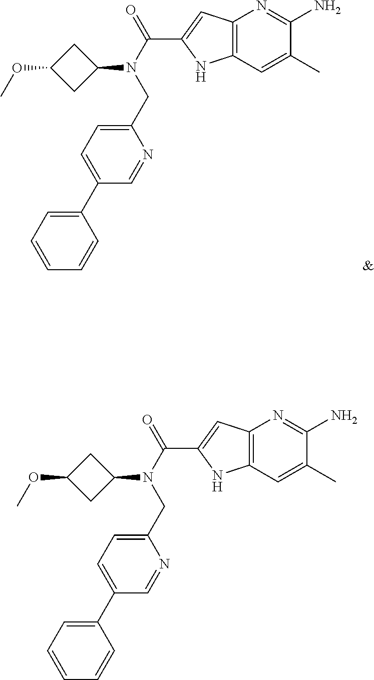

### Step 1: tert-butyl ((5-phenylpyridin-2-yl)methyl)carbamate

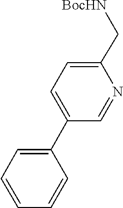 [SMILES: CC(C)(C)OC(=O)NCc1ccc(-c2ccccc2)cn1]

To a solution of 5-phenylpicolinonitrile (12.0 g, 66.6 mmol), Boc₂O (29.1 g, 133 mmol) and NiCl₂ (1.73 g, 13.3 mmol) in MeOH (150 mL) was slowly added NaBH₄ (15.1 g, 400 mmol) at 0° C. under nitrogen. The mixture was stirred at room temperature for 12 h, then slowly added to iced water (1 L). The mixture was extracted with EtOAc (500 mL×3). The combined organic layer was washed with brine (200 mL×3), dried over Na₂SO₄, filtered and concentrated under vacuum. The residue was purified by silica gel chromatograph (PE/EtOAc=10/1) to give the title compound (6.0 g, 32%). LC-MS (M+H)⁺=285.1.

### Step 2: (5-phenylpyridin-2-yl)methanamine

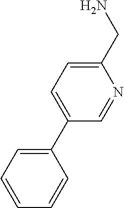 [SMILES: NCc1ccc(-c2ccccc2)cn1]

The title compound (1.93 g, 99%) was prepared in a manner similar to that in Example 2 step 2 from tert-butyl ((5-phenylpyridin-2-yl)methyl)carbamate. LC-MS (M+H)⁺=185.3.

### Step 3:3-methoxy-N-((5-phenylpyridin-2-yl)methyl)cyclobutan-1-amine

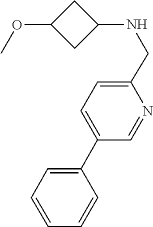 [SMILES: COC1CC(NCc2ccc(-c3ccccc3)cn2)C1]

The title compound (90 mg, 19%) was prepared in a manner similar to that in Example 1 step 6 from (5-phenylpyridin-2-yl)methanamine and 3-methoxycyclobutan-1-one. LC-MS (M+H)⁺=269.2.

### Step 4: 5-((diphenylmethylene)amino)-N-(3-methoxycyclobutyl)-6-methyl-N-((5-phenylpyridin-2-yl)methyl)-1-((2-(trimethylsilyl)ethoxy)methyl)-1H-pyrrolo[3,2-b]pyridine-2-carboxamide

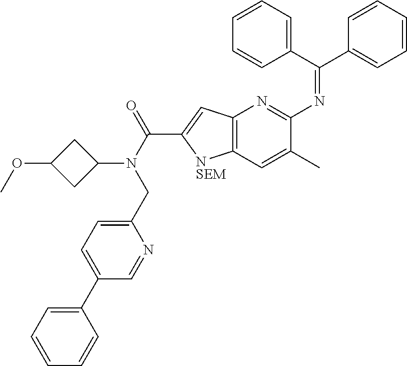 [SMILES: COC1CC(N(Cc2ccc(-c3ccccc3)cn2)C(=O)c2cc3nc(N=C(c4ccccc4)c4ccccc4)c(C)cc3n2COCC[Si](C)(C)C)C1]

The title compound (20 mg, 26%) was prepared in a manner similar to that in Example 1 step 7 from 5-((diphenylmethylene)amino)-6-methyl-1-((2-(trimethylsilyl)ethoxy)methyl)-1H-pyrrolo[3,2-b]pyridine-2-carboxylic acid and 3-methoxy-N-((5-phenylpyridin-2-yl)methyl)cyclobutan-1-amine. LC-MS (M+H)⁺=736.3.

### Step 5: 5-amino-N-((1r,3r)-3-methoxycyclobutyl)-6-methyl-N-((5-phenylpyridin-2-yl)methyl)-1H-pyrrolo[3,2-b]pyridine-2-carboxamide & 5-amino-N-((1s,3s)-3-methoxycyclobutyl)-6-methyl-N-((5-phenylpyridin-2-yl)methyl)-1H-pyrrolo[3,2-b]pyridine-2-carboxamide

Example 6 (2 mg, 17%) and Example 7 (1.6 mg, 13%) were prepared in a manner similar to that in Example 1 step 8 from 5-((diphenylmethylene)amino)-N-(3-methoxycyclobutyl)-6-methyl-N-((5-phenylpyridin-2-yl)methyl)-1-((2-(trimethylsilyl)ethoxy)methyl)-1H-pyrrolo[3,2-b]pyridine-2-carboxamide.

Example 6: ¹H NMR (500 MHz, DMSO-d6) δ 11.23 (s, 1H), 8.81 (s, 1H), 8.01 (d, J=7.6 Hz, 1H), 7.68 (d, J=7.4 Hz, 2H), 7.44 (t, J=7.6 Hz, 2H), 7.35 (t, J=7.3 Hz, 1H), 7.30 (d, J=7.7 Hz, 1H), 7.26 (s, 1H), 6.49-6.06 (m, 1H), 5.24 (s, 2H), 4.89 (s, 2H), 4.69-4.37 (m, 1H), 3.60-3.48 (m, 1H), 3.03 (s, 3H), 2.56-2.47 (m, 2H), 2.07 (s, 3H), 1.98-1.85 (m, 2H). LC-MS (M+H)⁺=442.3.

Example 7: ¹H NMR (500 MHz, DMSO-d6) δ 11.23 (s, 1H), 8.80 (s, 1H), 8.00 (d, J=7.0 Hz, 1H), 7.67 (d, J=7.2 Hz, 2H), 7.44 (t, J=7.5 Hz, 2H), 7.35 (t, J=7.3 Hz, 1H), 7.29 (d, J=8.0 Hz, 1H), 7.26 (s, 1H), 6.45-6.14 (m, 1H), 5.37-5.03 (m, 3H), 4.89 (s, 2H), 3.84-3.73 (m, 1H), 3.09-3.01 (m, 3H), 2.36-2.25 (m, 2H), 2.23-2.14 (m, 2H), 2.07 (s, 3H). LC-MS (M+H)⁺=442.4.

### Example B8: 5-amino-6-methyl-N-((5-phenylpyridin-2-yl)methyl)-N-(tetrahydro-2H-pyran-3-yl)-1H-pyrrolo[3,2-b]pyridine-2-carboxamide

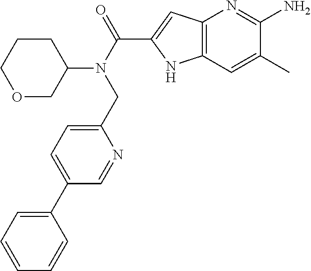 [SMILES: Cc1cc2[nH]c(C(=O)N(Cc3ccc(-c4ccccc4)cn3)C3CCCOC3)cc2nc1N]

### Step 1: N-((5-phenylpyridin-2-yl)methyl)tetrahydro-2H-pyran-3-amine

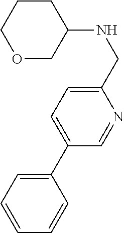 [SMILES: c1ccc(-c2ccc(CNC3CCCOC3)nc2)cc1]

The title compound (25 mg, 41%) was prepared in a manner similar to that in Example 1 step 6 from (5-phenylpyridin-2-yl)methanamine and dihydro-2H-pyran-3 (4H)-one. LC-MS (M+H)⁺=269.2.

### Step 2: 5-((diphenylmethylene)amino)-6-methyl-N-((5-phenylpyridin-2-yl)methyl)-N-(tetrahydro-2H-pyran-3-yl)-1-((2-(trimethylsilyl)ethoxy)methyl)-1H-pyrrolo[3,2-b]pyridine-2-carboxamide

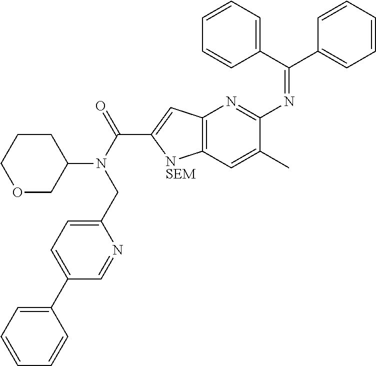 [SMILES: Cc1cc2c(cc(C(=O)N(Cc3ccc(-c4ccccc4)cn3)C3CCCOC3)n2COCC[Si](C)(C)C)nc1N=C(c1ccccc1)c1ccccc1]

The title compound (50 mg, 82%) was prepared in a manner similar to that in Example 1 step 7 from 5-((diphenylmethylene)amino)-6-methyl-1-((2-(trimethylsilyl)ethoxy)methyl)-1H-pyrrolo[3,2-b]pyridine-2-carboxylic acid and N-((5-phenylpyridin-2-yl)methyl)tetrahydro-2H-pyran-3-amine. LC-MS (M+H)⁺=736.6.

### Step 3: 5-amino-6-methyl-N-((5-phenylpyridin-2-yl)methyl)-N-(tetrahydro-2H-pyran-3-yl)-1H-pyrrolo[3,2-b]pyridine-2-carboxamide

Example 8 (13 mg, 44%) was prepared in a manner similar to that in Example 1 step 8 from 5-((diphenylmethylene)amino)-6-methyl-N-((5-phenylpyridin-2-yl)methyl)-N-(tetrahydro-2H-pyran-3-yl)-1-((2-(trimethylsilyl)ethoxy)methyl)-1H-pyrrolo[3,2-b]pyridine-2-carboxamide. ¹H NMR (500 MHZ, DMSO-d6) δ 11.30 (s, 1H), 8.88 (s, 1H), 8.07 (d, J=5.7 Hz, 1H), 7.75 (d, J=7.4 Hz, 2H), 7.52-7.41 (m, 5H), 7.34 (s, 1H), 5.30 (s, 2H), 4.93 (s, 2H), 4.51 (s, 1H), 3.87 (s, 1H), 3.74 (d, J=9.4 Hz, 1H), 3.44 (t, J=10.3 Hz, 1H), 3.24 (t, J=10.9 Hz, 1H), 2.14 (s, 3H), 2.01-1.77 (m, 2H), 1.65 (s, 2H). LC-MS (M+H)⁺=442.3.

### Example B9: 5-amino-N-((5-(2,6-difluorophenyl)pyridin-2-yl)methyl)-N-((3R,4R)-4-methoxytetrahydro-2H-pyran-3-yl)-6-methyl-1H-pyrrolo[3,2-b]pyridine-2-carboxamide

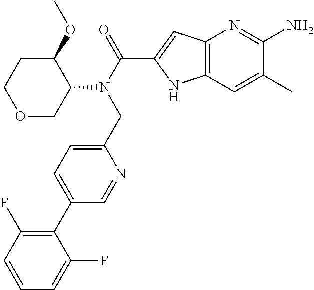 [SMILES: CO[C@@H]1CCOC[C@H]1N(Cc1ccc(-c2c(F)cccc2F)cn1)C(=O)c1cc2nc(N)c(C)cc2[nH]1]

### Step 1: (3R,4R)-3-(((5-(2,6-difluorophenyl)pyridin-2-yl)methyl)amino)tetrahydro-2H-pyran-4-ol

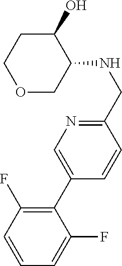 [SMILES: O[C@@H]1CCOC[C@H]1NCc1ccc(-c2c(F)cccc2F)cn1]

To a solution of (3R,4R)-3-aminotetrahydro-2H-pyran-4-ol (120 mg, 1.02 mmol) and 5-(2,6-difluorophenyl)picolinaldehyde (224 mg, 1.02 mmol) in DCM (10 mL) was added NaBH(OAc)₃ (430 mg, 2.03 mmol) at 0° C. The mixture was warmed to room temperature and stirred for 2 h. The mixture was concentrated under reduced pressure and the residue was purified by silica gel chromatograph (PE/EtOAc=1/1) to give the title compound (200 mg, 61%). LCMS (M+H)⁺=321.1.

### Step 2: tert-butyl ((5-(2,6-difluorophenyl)pyridin-2-yl)methyl)((3R,4R)-4-hydroxytetrahydro-2H-pyran-3-yl)carbamate

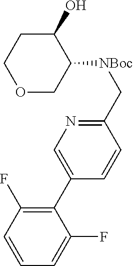

To a solution of of (3R,4R)-3-(((5-(2,6-difluorophenyl)pyridin-2-yl)methyl)amino)tetrahydro-2H-pyran-4-ol (200 mg, 0.62 mmol) in DCM (10 mL) was added Boc₂O (164 mg, 0.75 mmol) and Et₃N (188 mg, 1.86 mmol). The mixture was stirred at room temperature for overnight. The mixture was concentrated under reduced pressure and the residue was purified by silica gel chromatograph (PE/EtOAc=2/1) to give the title compound (230 mg, 88%). LCMS (M+H)⁺=421.1.

### Step 3: tert-butyl ((5-(2,6-difluorophenyl)pyridin-2-yl)methyl)((3R,4R)-4-methoxytetrahydro-2H-pyran-3-yl)carbamate

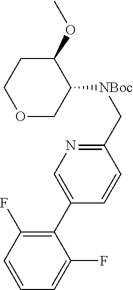

To a mixture of tert-butyl ((5-(2,6-difluorophenyl)pyridin-2-yl)methyl)((3R,4R)-4-hydroxytetrahydro-2H-pyran-3-yl)carbamate (230 mg, 0.55 mmol) and Mel (78 mg, 0.55 mmol) in DMF (10 mL) was added NaH (60%, 29 mg, 0.72 mmol) at 0° C. The mixture was stirred at room temperature under nitrogen for 5 h. The mixture was diluted with iced water (20 mL) and extracted with EtOAc (20 mL×2), The combined organic layer was washed with brine (20 mL), dried over Na₂SO₄, filtered and concentrated under reduced pressure. The residue was purified by silica gel chromatograph (PE/EtOAc=2/1) to give the title compound (230 mg, 96%). LCMS (M+H)⁺=435.1.

### Step 4: (3R,4R)—N-((5-(2,6-difluorophenyl)pyridin-2-yl)methyl)-4-methoxytetrahydro-2H-pyran-3-amine hydrochloride

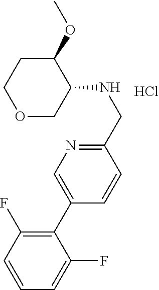 [SMILES: CO[C@@H]1CCOC[C@H]1NCc1ccc(-c2c(F)cccc2F)cn1 ; Cl]

A mixture of tert-butyl ((5-(2,6-difluorophenyl)pyridin-2-yl)methyl)((3R,4R)-4-methoxytetrahydro-2H-pyran-3-yl)carbamate (230 mg, 0.53 mmol) in HCl (4 M in MeOH, 5 mL) was stirred at room temperature for 2 h. The mixture was concentrated under reduced pressure to give the title compound (180 mg, 92%). LCMS (M+H)⁺=335.1.

### Step 5: 5-amino-6-methyl-1H-pyrrolo[3,2-b]pyridine-2-carboxylic Acid

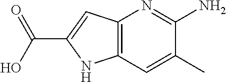

A mixture of 5-((diphenylmethylene)amino)-6-methyl-1-((2-(trimethylsilyl)ethoxy)methyl)-1H-pyrrolo[3,2-b]pyridine-2-carboxylic acid (1.7 g, 3.5 mmol) in TFA (10 mL) and DCM (10 mL) was stirred at 30° C. for 4 h. The mixture was concentrated under reduced pressure and treated with of aqueous HCl (1 M, 15 mL). The mixture was stirred at room temperature for 1 h and concentrated under reduced pressure. The residue was treated with aqueous K₂CO₃ (1 M, 10 mL) and MeOH (10 mL), and the mixture was stirred at 50° C. for 2 h. The mixture was concentrated under reduced pressure, and the crude was purification by C18 chromatograph (10% MeCN in water with 0.03% formic acid) to give the title compound (0.60 g, 90%). LCMS (M+H)⁺=192.2.

### Step 6: 5-amino-N-((5-(2,6-difluorophenyl)pyridin-2-yl)methyl)-N-((3R,4R)-4-methoxytetrahydro-2H-pyran-3-yl)-6-methyl-1H-pyrrolo[3,2-b]pyridine-2-carboxamide

A mixture of 5-amino-6-methyl-1H-pyrrolo[3,2-b]pyridine-2-carboxylic acid (50 mg, 0.26 mmol) in SOCl₂ (2 mL) was stirred at 60° C. under nitrogen for 2 h. The mixture was cooled to room temperature and concentrated under reduced pressure. The residue was re-dissolved in DCM (5 mL), cooled to room temperature and added (3R,4R)—N-((5-(2,6-difluorophenyl)pyridin-2-yl)methyl)-4-methoxytetrahydro-2H-pyran-3-amine hydrochloride (96 mg, 0.26 mmol) and Et₃N (131 mg, 1.3 mmol) under nitrogen. The mixture was warmed to room temperature and stirred for 3 h. The mixture was diluted with water (10 mL) and extracted with DCM (10 mL×2). The combined organic layer was washed with brine (10 mL) and concentrated under reduced pressure. The residue was purified by prep-HPLC to give the title compound (15 mg, 11%). ¹H NMR (500 MHz, DMSO-d6) δ 11.54-11.20 (m, 1H), 8.80-8.48 (m, 1H), 8.02-7.75 (m, 1H), 7.72-7.43 (m, 2H), 7.43-7.33 (m, 1H), 7.32-7.12 (m, 2H), 6.77-6.16 (m, 1H), 5.74-5.32 (m, 2H), 5.22-4.43 (m, 3H), 4.29-4.03 (m, 1H), 3.92-3.48 (m, 3H), 3.15-3.10 (m, 3H), 2.22-2.08 (m, 4H), 1.45-1.20 (m, 1H). LCMS (M+H)⁺=508.3.

### Example B10: 5-amino-N-((5-(2,6-difluorophenyl)pyridin-2-yl)methyl)-N-(1-methoxy-3,3-dimethylbutan-2-yl)-6-methyl-1H-pyrrolo[3,2-b]pyridine-2-carboxamide

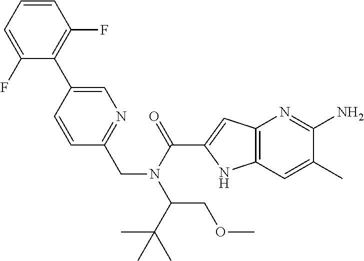 [SMILES: COCC(N(Cc1ccc(-c2c(F)cccc2F)cn1)C(=O)c1cc2nc(N)c(C)cc2[nH]1)C(C)(C)C]

### Step 1: tert-butyl (1-hydroxy-3,3-dimethylbutan-2-yl)carbamate

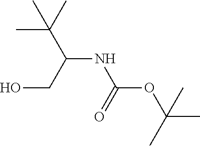

To a solution of 2-amino-3,3-dimethylbutan-1-ol (1.0 g, 8.55 mmol) in DCM (20 mL) was added Et₃N (1.72 g, 17.1 mmol) and Boc₂O (2.79 g, 12.8 mmol) at 0° C. The mixture was stirred for 16 h at room temperature. The mixture was diluted with water (50 mL) and then extracted with DCM (50 mL). The organic layer was washed with brine (50 mL), dried over Na₂SO₄, filtered and concentrated under vacuum. The residue was purified by silica gel chromatograph (PE:EtOAc=10:1) to give the title compound (1.6 g, 86%). LC-MS (M+H)⁺=218.2.

### Step 2: tert-butyl (1-methoxy-3,3-dimethylbutan-2-yl)carbamate

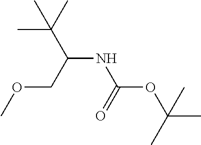 [SMILES: COCC(NC(=O)OC(C)(C)C)C(C)(C)C]

To a solution of tert-butyl (1-hydroxy-3,3-dimethylbutan-2-yl)carbamate (700 mg, 3.22 mmol) in MeCN (8 mL) was added Ag₂O (1.19 g, 5.16 mmol) and Mel (2.29 g, 16.1 mmol). The mixture was stirred for 16 h at 70° C. in a sealed tube. The reaction mixture was cooled to room temperature, diluted with water (50 mL) and then extracted with EtOAc (50 mL). The organic layers was washed with brine (50 mL×2), dried over Na₂SO₄, filtered and concentrated under vacuum. The residue was purified by silica gel chromatograph (PE:EtOAc=10:1) to give the title compound (600 mg, 80%).

### Step 3: 1-methoxy-3,3-dimethylbutan-2-amine hydrochloride

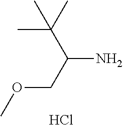

To tert-butyl (1-methoxy-3,3-dimethylbutan-2-yl)carbamate (600 mg, 2.60 mmol) was added HCl (4 M in dioxane), and the mixture was stirred for 3 h at room temperature. The mixture was concentrated under vacuum to give the title compound (440 mg, 100%). LC-MS (M+H)⁺=132.1.

### Step 4: N-((5-(2,6-difluorophenyl)pyridin-2-yl)methyl)-1-methoxy-3,3-dimethylbutan-2-amine

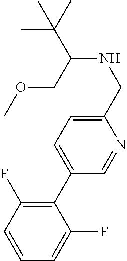 [SMILES: COCC(NCc1ccc(-c2c(F)cccc2F)cn1)C(C)(C)C]

To a mixture of 1-methoxy-3,3-dimethylbutan-2-amine hydrochloride (150 mg, 0.90 mmol) in DCM (5 mL) was added 5-(2,6-difluorophenyl)picolinaldehyde (198 mg, 0.90 mmol), Et₃N (91 mg, 0.90 mmol) and NaBH(OAc)₃ (383 mg, 1.81 mmol). The mixture was stirred for 16 h at room temperature. The mixture was carefully added to saturated NaHCO₃ (50 mL) and then extracted with DCM (50 mL). The organic layer was washed with brine (50 ml), dried over Na₂SO₄, filtered and concentrated under vacuum. The residue was purified by silica gel chromatograph (DCM:MeOH=10:1) to give the title compound (200 mg, 66%). LC-MS (M+H)⁺=335.3.

### Step 5: N-((5-(2,6-difluorophenyl)pyridin-2-yl)methyl)-5-((diphenylmethylene)amino)-N-(1-methoxy-3,3-dimethylbutan-2-yl)-6-methyl-1-((2-(trimethylsilyl)ethoxy)methyl)-1H-pyrrolo[3,2-b]pyridine-2-carboxamide

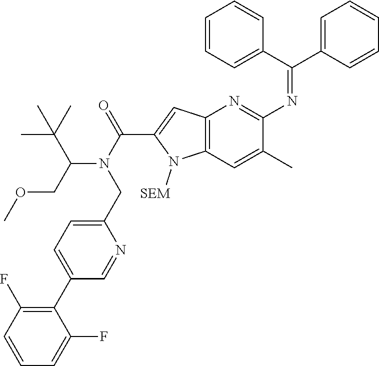 [SMILES: COCC(N(Cc1ccc(-c2c(F)cccc2F)cn1)C(=O)c1cc2nc(N=C(c3ccccc3)c3ccccc3)c(C)cc2n1COCC[Si](C)(C)C)C(C)(C)C]

To a solution of 5-((diphenylmethylene)amino)-6-methyl-1-((2-(trimethylsilyl)ethoxy)methyl)-1H-pyrrolo[3,2-b]pyridine-2-carboxylic acid (50 mg, 0.10 mmol) in DCM (6 mL) was added DMF (2 drops) and (COCl)₂ (25 mg, 0.20 mmol). The was stirred for 2 h at room temperature. The reaction mixture was transferred into a solution of N-((5-(2,6-difluorophenyl)pyridin-2-yl)methyl)-1-methoxy-3,3-dimethylbutan-2-amine (40 mg, 0.12 mmol) and DIPEA (25 mg, 0.2 mmol) in DCM (6 mL). The mixture was stirred for 2 h at room temperature, then concentrated under vacuum. The residue was purified by silica gel chromatograph (DCM:MeOH=20:1) to give the title compound (55 mg, 667%). LC-MS (M+H)⁺=802.2.

### Step 6: 5-amino-N-((5-(2,6-difluorophenyl)pyridin-2-yl)methyl)-N-(1-methoxy-3,3-dimethylbutan-2-yl)-6-methyl-1H-pyrrolo[3,2-b]pyridine-2-carboxamide

Example 10 (17 mg, 49%) was prepared in a manner similar to that in Example 1 step 8 from N-((5-(2,6-difluorophenyl)pyridin-2-yl)methyl)-5-((diphenylmethylene)amino)-N-(1-methoxy-3,3-dimethylbutan-2-yl)-6-methyl-1-((2-(trimethylsilyl)ethoxy)methyl)-1H-pyrrolo[3,2-b]pyridine-2-carboxamide; ¹H NMR (500 MHZ, DMSO-d6) δ 11.30-11.11 (m, 1H), 8.66-8.52 (m, 1H), 7.94-7.80 (m, 1H), 7.61-7.44 (m, 2H), 7.38-7.18 (m, 3H), 6.80-6.23 (m, 1H), 5.45-4.48 (m, 5H), 3.76-3.41 (m, 2H), 3.26-2.88 (m, 3H), 2.20-2.07 (m, 3H), 1.11-0.87 (m, 9H). LC-MS (M+H)⁺=508.4.

### Compounds Cell Killing in HCT116 Isogenic Pair

HCT116 cell line was obtained from ATCC (CCL-247). It has a mutation in codon 13 of the ras proto-oncogene and can be used as a positive control for PCR assays of mutation in this codon. HCT116-MTAP-KO was knocked-out-MTAP-gene based HCT116, and one single clone was passed for this assay. HCT116-mock-RNA-KO was knocked-out-mock-gene based HCT116 with MTAP wildtype genotype. The base medium for HCT116 isogenic pair is RPMI 1640, HEPES (Gibco, 22400105). To make the complete growth medium, add the following components to the base medium: fetal bovine serum to a final concentration of 10% (Gibco, 10099-141C). The cell line was grown in a humidified 5% CO₂ atmosphere at 37° C. and regularly tested for the presence of mycoplasma with MycoAlert™ PLUS Mycoplasma Detection Kit (Lonza, LT07-710).

### EXPERIMENTAL

Seed HCT116-mock-RNA-KO (400/well) or HCT116-MTAP-KO (400/well) cells into a 96-well plate (Greiner: 655090), 100 μL/well.
Incubate at 37° C., 5% CO₂, overnight.
Add 50 μL fresh growth-medium containing serial dilution of compounds for a final compound concentration of 0-10 μM to each well.
Incubate at 37° C., 5% CO₂, 6 days.
The cell viability detected by Cell Titer-Glo (Promega, G7573). Add 70 μL of CellTiter-Glo® reagent in each well.
Incubate at room temperature for 10 minutes to stabilize luminescent signal.
Analyze with Microplate Reader (TECAN, SPARK) and fit IC₅₀ using Prism® 9.

The compounds disclosed herein showed cell killing activity values as in Table 1

TABLE 1
Cell killing IC₅₀ (nM) for the compounds disclosed herein

| | | Cell killing | | | Cell killing |
|---|---|---|---|---|---|
| | Cell killing | IC₅₀ (nM) in | | Cell killing | IC₅₀ (nM) in |
| | IC₅₀ (nM) in | HCT116- | | IC₅₀ (nM) in | HCT116- |
| | HCT116- | mock-RNA- | | HCT116- | mock-RNA- |
| Example | MTAP-KO | KO | Example | MTAP-KO | KO |
| A1 | <0.5* | 155 | A2 | <0.5* | 486 |
| A3 | 0.008 | 49 | A4 | 30 | 2356 |
| A5 | 167 | 3344 | A6 | <4.6* | 1487 |
| A7 | <4.6* | 339 | A8 | <4.6* | 2036 |
| A9 | 86 | 4180 | A10 | 217 | 3477 |
| A11 | <4.6* | 1466 | A12 | <4.6* | 661 |
| A13 | 83 | >10000 | A14 | 2.9 | 1125 |
| A15 | 4.3 | 424 | A16 | 17 | 1645 |
| A17 | 66 | 2837 | A18 | 21 | 2579 |
| A19 | <4.6* | 288 | | | |
| B1 | 359 | >10000 | B2 | 5.5 | 1215 |
| B3 | 864 | >10000 | B4 | 7820 | >10000 |
| B5 | 606 | 3615 | B6 | 1727 | >10000 |
| B7 | 2054 | >10000 | B8 | 1353 | >10000 |

*More than 50% viability inhibition at tested concentration without a full curve for IC₅₀ determination

What is claimed is:

1. A compound of Formula (X): 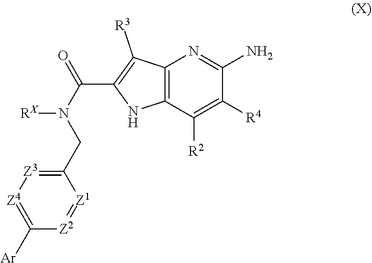
or a N-oxide thereof, or a pharmaceutically acceptable salt thereof, or a stereoisomer thereof, or a tautomer thereof, or a deuterated analog thereof, wherein:
Z¹, Z², Z³ and Z⁴ are each independently selected from N or CR^{Z};
at each of its occurrences, R^{Z} is independently selected from hydrogen, halogen, —C₁₋₄alkyl or —C₁₋₄alkoxy; wherein each of said —C₁₋₄alkyl or —C₁₋₄alkoxy is optionally substituted with at least one substituent selected from halogen;
Ar is selected from phenyl or 5- to 6-membered heteroaryl, each of said phenyl or 5- to 6-membered heteroaryl is optionally substituted with at least one substituent R^{Ar},
R^{Ar} is each independently selected from hydrogen, halogen, —C₁₋₄alkyl, —C₁₋₄alkoxy, —C₃-C₆cycloalkyl, —C₃-C₆cycloalkoxy or —CN; wherein each of said —C₁₋₄alkyl, —C₁₋₄alkoxy, —C₃-C₆cycloalkyl or —C₃-C₆cycloalkoxy is optionally substituted with at least one substituent selected from halogen;
R^{X} is R¹ or 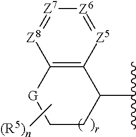
R¹ is each independently selected from —C₁₋₈alkyl, —C₃-C₈cycloalkyl (preferably, mono, bridged, fused or spiral cycloalkyl) or 3- to 8-membered saturated heterocyclyl (preferably, mono, bridged, fused or spiral heterocyclyl); wherein each of said —C₁₋₈alkyl, —C₃-C₈cycloalkyl (preferably, mono, bridged, fused or spiral cycloalkyl) or 3- to 8-membered saturated heterocyclyl (preferably, mono, bridged, fused or spiral heterocyclyl) is optionally substituted with at least one substituent R^{1a};
R^{1a} is each independently selected from halogen, —C₁₋₄alkyl, —C₃-C₆cycloalkyl, 3- to 6-membered saturated heterocyclyl, —C₁₋₄alkoxy, —C₃-C₆cycloalkoxy, —CN, —OH, —NH₂ or oxo; wherein each of said —C₁₋₄alkyl, —C₃-C₆cycloalkyl, 3- to 6-membered saturated heterocyclyl, —C₁₋₄alkoxy or —C₃-C₆cycloalkoxy is optionally substituted with at least one substituent selected from halogen, —C₁₋₄alkyl, —C₃-C₆cycloalkyl, 3- to 6-membered saturated heterocyclyl, —C₁₋₄alkoxy, —C₃-C₆cycloalkoxy, —CN, —OH, —NH₂ or oxo;
G is selected from CH₂, O, or NH, provided that when G is NH and NH is substituted with R⁵, R⁵ is not halogen;
Z⁵, Z⁶, Z⁷ and Z⁸ are each independently selected from N or CR¹, provided that at least one of Z⁵, Z⁶, Z⁷ and Z⁸ is N, and others are CR^{Z1}, and Z⁵ and Z⁸ are not N at the same time;
at each of its occurrences, R^{Z1} is independently selected from hydrogen, halogen, —C₁₋₄alkyl, —C₁₋₄alkoxy, —C₃-C₆cycloalkyl or —C₃-C₆cycloalkoxy; wherein each of said —C₁₋₄alkyl, —C₁₋₄alkoxy, —C₃-C₆cycloalkyl or —C₃-C₆cycloalkoxy is optionally substituted with at least one substituent select ed from halogen, —C₁₋₄alkoxy, —CN, —OH, —NH₂ or oxo;
at each of its occurrences, R⁵ is independently selected from hydrogen, halogen, —C₁₋₄alkyl or —C₃-C₆cycloalkyl, wherein —C₁₋₄alkyl or —C₃-C₆cycloalkyl is optionally substituted with at least one substituent selected from halogen, —C₁₋₄alkoxy, —CN, —OH, —NH₂ or oxo; or
two geminal R⁵ and the carbon atom which they attach to form a 3- to 5-membered carbocyclic ring;
wherein said ring is optionally substituted with at least one substituent selected from halogen, —C₁₋₄alkoxy, —CN, —OH, —NH₂ or oxo;
n is 0, 1, 2, 3 or 4;
r is 0 or 1;
R² is selected from hydrogen, halogen, —C₁₋₄alkyl, —C₁₋₄alkoxy, —C₃-C₆cycloalkyl, —C₃-C₆cycloalkoxy or —CN; wherein each of said —C₁₋₄alkyl, —C₁₋₄alkoxy, —C₃-C₆cycloalkyl or —C₃-C₆cycloalkoxy is optionally substituted with at least one substituent selected from halogen, —C₁₋₄alkoxy or —C₃-C₆cycloalkoxy;
R³ is selected from hydrogen, halogen, —C₁₋₄alkyl or —CN, wherein —C₁₋₄alkyl is optionally substituted with at least one substituent selected from hydrogen, halogen, —C₁₋₄alkoxy, —C₃-C₆cycloalkyl, —CN, —OH, —NH₂ or oxo;
R⁴ is selected from hydrogen, halogen, —C₁₋₄alkyl, —C₁₋₄alkoxy, —C₃-C₆cycloalkyl or —C₃-C₆cycloalkoxy, wherein each of said —C₁₋₄alkyl, —C₁₋₄alkoxy, —C₃-C₆cycloalkyl or —C₃-C₆cycloalkoxy is optionally substituted with at least one substituent selected from halogen, —C₁₋₄alkoxy, —C₃-C₆cycloalkoxy, —CN, —OH, —NH₂ or oxo.

2. The compound of claim 1, wherein the compound is Formula (AI): 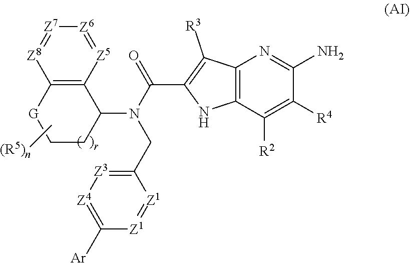
or a N-oxide thereof, or a pharmaceutically acceptable salt thereof, or a stereoisomer thereof, or a tautomer thereof, or a deuterated analog thereof, wherein:
Z¹, Z², Z³ and Z⁴ are each independently selected from N or CR^{Z};
at each of its occurrences, R^{Z} is independently selected from hydrogen, halogen, —C₁₋₄alkyl or —C₁₋₄alkoxy; wherein each of said —C₁₋₄alkyl or —C₁₋₄alkoxy is optionally substituted with at least one substituent selected from halogen;
Ar is selected from phenyl or 5- to 6-membered heteroaryl, each of said phenyl or 5- to 6-membered heteroaryl is optionally substituted with at least one substituent R^{Ar},
R^{Ar} is each independently selected from hydrogen, halogen, —C₁₋₄alkyl, —C₁₋₄alkoxy, —C₃-C₆cycloalkyl, —C₃-C₆cycloalkoxy or —CN; wherein each of said —C₁₋₄alkyl, —C₁₋₄alkoxy, —C₃-C₆cycloalkyl or —C₃-C₆cycloalkoxy is optionally substituted with at least one substituent selected from halogen;
G is selected from CH₂, O, or NH, provided that when G is NH and NH is substituted with R⁵, R⁵ is not halogen;
Z⁵, Z⁶, Z⁷ and Z⁸ are each independently selected from N or CR²¹, provided that at least one of Z⁵, Z⁶, Z⁷ and Z⁸ is N, and others are CR^{Z1};
at each of its occurrences, R^{Z1} is independently selected from hydrogen, halogen, —C₁₋₄alkyl, —C₁₋₄alkoxy, —C₃-C₆cycloalkyl or —C₃-C₆cycloalkoxy; wherein each of said —C₁₋₄alkyl, —C₁₋₄alkoxy, —C₃-C₆cycloalkyl or —C₃-C₆cycloalkoxy is optionally substituted with at least one substituent selected from halogen, —C₁₋₄alkoxy, —CN, —OH, —NH₂ or oxo;
R² is selected from hydrogen, halogen, —C₁₋₄alkyl, —C₁₋₄alkoxy, —C₃-C₆cycloalkyl, —C₃-C₆cycloalkoxy or —CN;
wherein each of said —C₁₋₄alkyl, —C₁₋₄alkoxy, —C₃-C₆cycloalkyl or —C₃-C₆cycloalkoxy is optionally substituted with at least one substituent selected from halogen, —C₁₋₄alkoxy or —C₃-C₆cycloalkoxy;
R³ is selected from hydrogen, halogen, —C₁₋₄alkyl or —CN, wherein —C₁₋₄alkyl is optionally substituted with at least one substituent selected from hydrogen, halogen, —C₁₋₄alkoxy, —C₃-C₆cycloalkyl, —CN, —OH, —NH² or oxo;
R⁴ is selected from hydrogen, halogen, —C₁₋₄alkyl, —C₁₋₄alkoxy, —C₃-C₆cycloalkyl or —C₃-C₆cycloalkoxy, wherein each of said —C₁₋₄alkyl, —C₁₋₄alkoxy, —C₃-C₆cycloalkyl or —C₃-C₆cycloalkoxy is optionally substituted with at least one substituent selected from halogen, —C₁₋₄alkoxy, —C₃-C₆cycloalkoxy, —CN, —OH, —NH₂ or oxo;
at each of its occurrences, R⁵ is independently selected from hydrogen, halogen, —C₁₋₄alkyl or —C₃-C₆cycloalkyl, wherein —C₁₋₄alkyl or —C₃-C₆cycloalkyl is optionally substituted with at least one substituent selected from halogen, —C₁₋₄alkoxy, —CN, —OH, —NH₂ or oxo; or
two geminal R⁵ and the carbon atom which they attach to form a 3- to 5-membered carbocyclic ring; wherein said ring is optionally substituted with at least one substituent selected from halogen, —C₁₋₄alkoxy, —CN, —OH, —NH₂ or oxo;
n is 0, 1, 2, 3 or 4;
r is 0 or 1.

3. The compound of anyone of claim 1, wherein the compound is of Formula (BI): 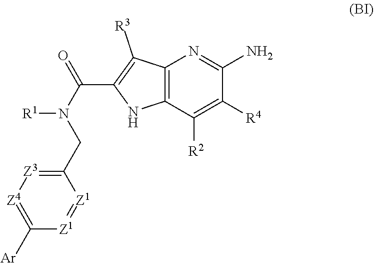
or a N-oxide thereof, or a pharmaceutically acceptable salt thereof, or a stereoisomer thereof, or a tautomer thereof, or a deuterated analog thereof, wherein:
Z¹, Z², Z³ and Z⁴ are each independently selected from N or CR^{Z};
at each of its occurrences, R^{Z} is each independently selected from hydrogen, halogen, —C₁₋₄alkyl or —C₁₋₄alkoxy; wherein each of said —C₁₋₄alkyl or —C₁₋₄alkoxy is optionally substituted with at least one substituent selected from halogen;
Ar is independently selected from phenyl or 5- to 6-membered heteroaryl, wherein each of said phenyl or 5- to 6-membered heteroaryl is optionally substituted with at least one substituent R^{Ar},
R^{Ar} is each independently selected from hydrogen, halogen, —C₁₋₄alkyl, —C₁₋₄alkoxy, —C₃-C₆cycloalkyl, —C₃-C₆cycloalkoxy or —CN; wherein each of said —C₁₋₄alkyl, —C₁₋₄alkoxy, —C₃-C₆cycloalkyl or —C₃-C₆cycloalkoxy is optionally substituted with at least one substituent selected from halogen;
R¹ is each independently selected from —C₁₋₈alkyl, —C₃-C₈cycloalkyl (preferably, mono, bridged, fused or spiral cycloalkyl) or 3- to 8-membered saturated heterocyclyl (preferably, mono, bridged, fused or spiral heterocyclyl); wherein each of said —C₁₋₈alkyl, —C₃-C₈cycloalkyl (preferably, mono, bridged, fused or spiral cycloalkyl) or 3- to 8-membered saturated heterocyclyl (preferably, mono, bridged, fused or spiral heterocyclyl) is optionally substituted with at least one substituent R^{1a};
R^{1a} is each independently selected from halogen, —C₁₋₄alkyl, —C₃-C₆cycloalkyl, 3- to 6-membered saturated heterocyclyl, —C₁₋₄alkoxy, —C₃-C₆cycloalkoxy, —CN, —OH, —NH₂ or oxo; wherein each of said —C₁₋₄alkyl, —C₃-C₆cycloalkyl, 3- to 6-membered saturated heterocyclyl, —C₁₋₄alkoxy or —C₃-C₆cycloalkoxy is optionally substituted with at least one substituent selected from halogen, —C₁₋₄alkyl, —C₃-C₆cycloalkyl, 3- to 6-membered saturated heterocyclyl, —C₁₋₄alkoxy, —C₃-C₆cycloalkoxy, —CN, —OH, —NH₂ or oxo;
R² is selected from hydrogen, halogen, —C₁₋₄alkyl, —C₁₋₄alkoxy, —C₃-C₆cycloalkyl, —C₃-C₆cycloalkoxy or —CN; wherein each of said —C₁₋₄alkyl, —C₁₋₄alkoxy, —C₃-C₆cycloalkyl or —C₃-C₆cycloalkoxy is optionally substituted with at least one substituent selected from halogen, —C₁₋₄alkoxy or —C₃-C₆cycloalkoxy;
R³ is selected from hydrogen, halogen, —C₁₋₄alkyl or —CN, wherein —C₁₋₄alkyl is optionally substituted with at least one substituent selected from hydrogen, halogen, —C₁₋₄alkoxy, —C₃-C₆cycloalkyl, —CN, —OH, —NH₂ or oxo;
R⁴ is selected from hydrogen, halogen, —C₁₋₄alkyl, —C₁₋₄alkoxy, —C₃-C₆cycloalkyl or —C₃-C₆cycloalkoxy, wherein each of said —C₁₋₄alkyl, —C₁₋₄alkoxy, —C₃-C₆cycloalkyl or —C₃-C₆cycloalkoxy is optionally substituted with at least one substituent selected from halogen, —C₁₋₄alkoxy, —C₃-C₆cycloalkoxy, —CN, —OH, —NH₂ or oxo.

4. The compound of claim 1, wherein the compound is selected from formula (AIIa), (AIIb), (AIIc) or (AIId): 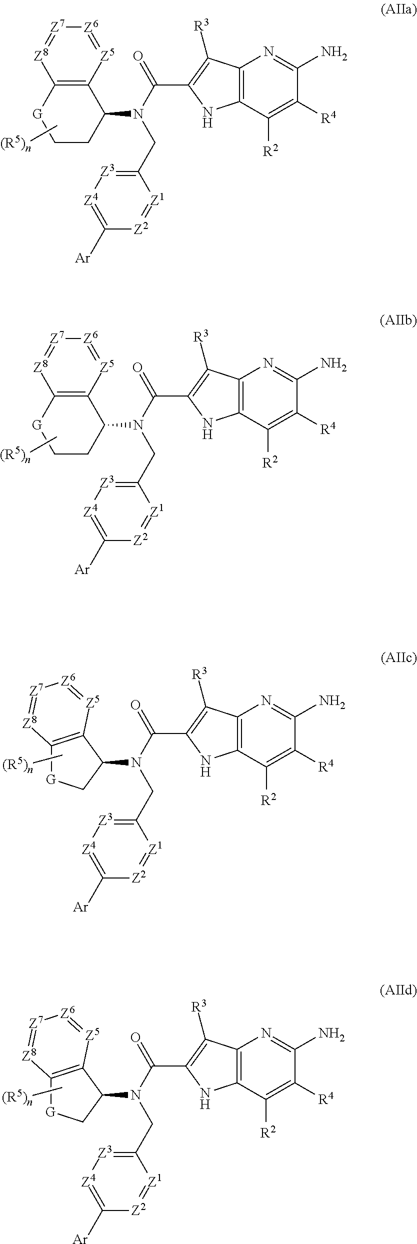
R², R³, R⁴, R⁵, Z¹, Z², Z³, Z⁴, Z⁵, Z⁶, Z⁷, Z⁸, Ar, G and n are as defined as claim 1.

5. The compound of claim 1, wherein the compound is selected from formula (AIIe), (AIIf), (AIIg), (AIIh), (AIIi), (AIIj), (AIIk), (AIIl), (AIIm) or (AIIn): 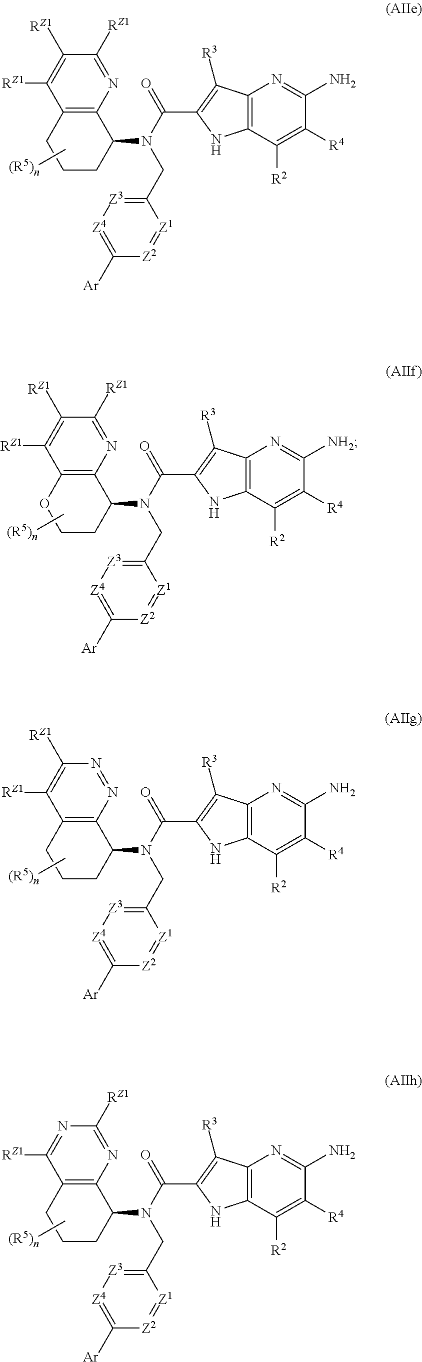 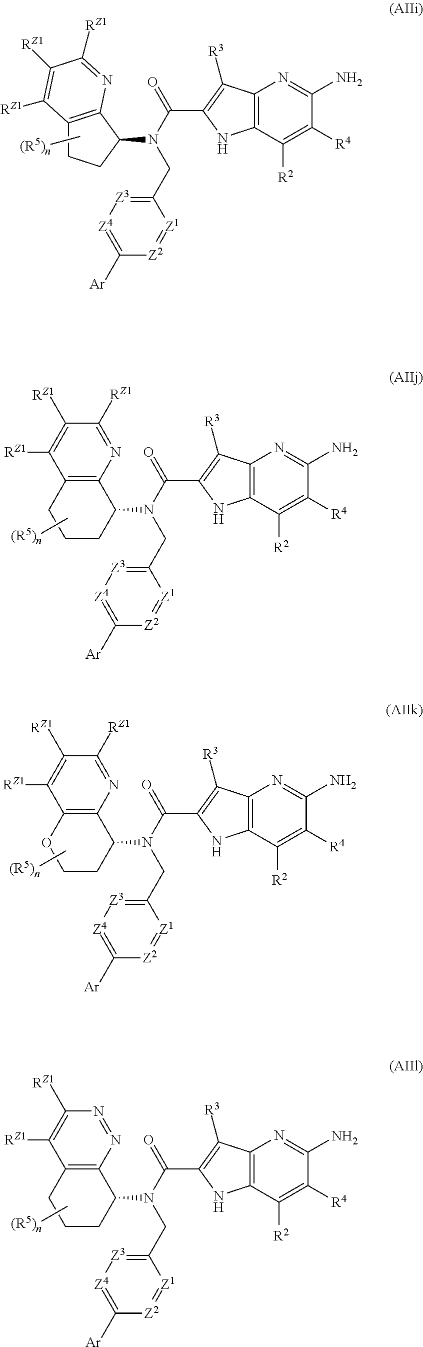 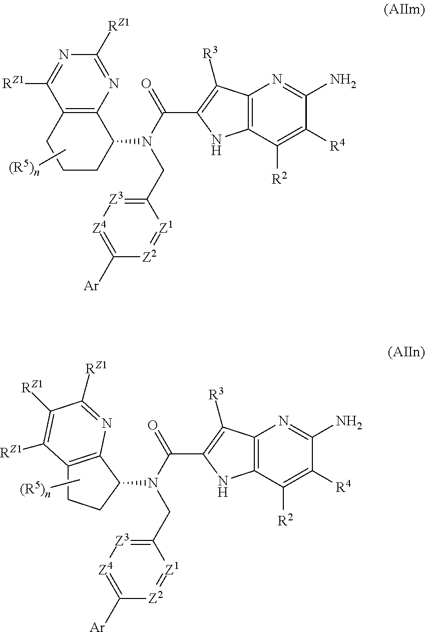
R^{Z1}, R², R³, R⁴, R⁵, Z¹, Z², Z³, Z⁴, Ar and n are as defined as claim 1.

6. The compound of claim 1, wherein the compound is formula (AIIIa), (AIIIb), (AIIIc), (AIIId), (AIIIe), (AIIIf) or (AIIIg): 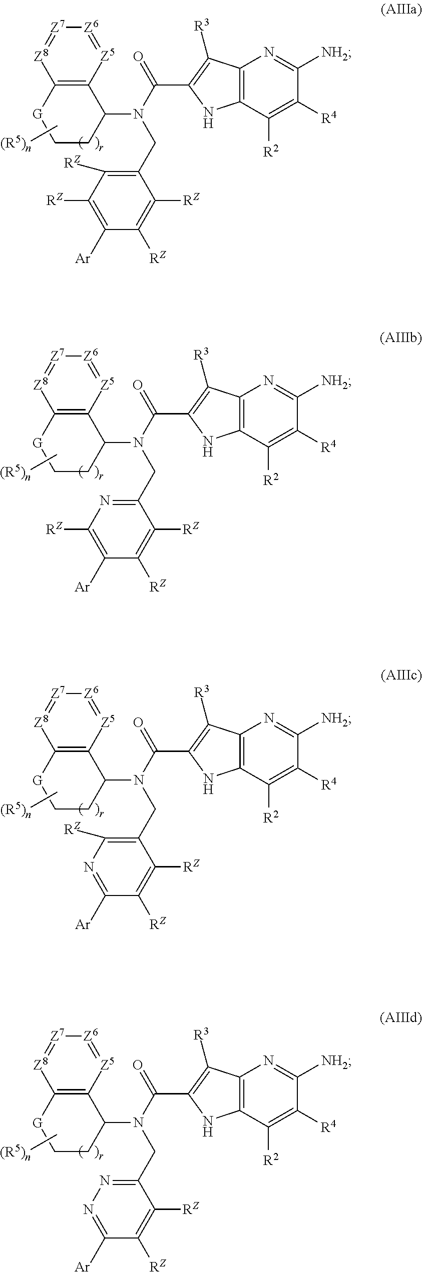 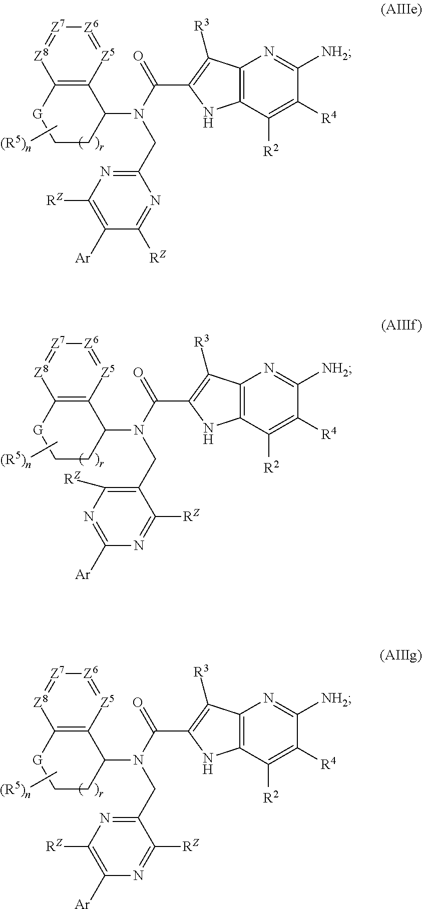
R², R³, R⁴, R⁵, R^{Z}, Z⁵, Z⁶, Z⁷, Z⁸, G, n and r are as defined as claim 1.

7. The compound of claim 1, wherein the compound is formula (AIVa), (AIVb), (AIVc): 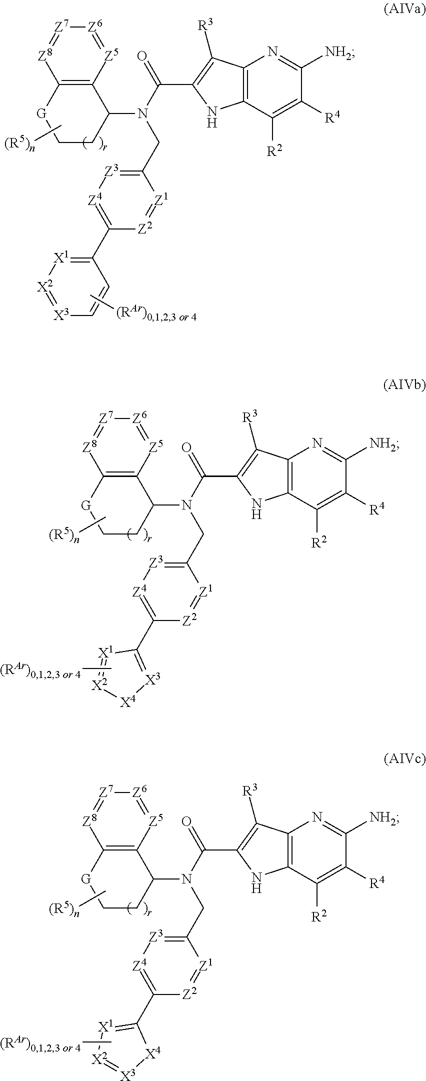
wherein, X¹, X² and X³ are each independently selected from N or CH, provided that when anyone of X¹, X² or X³ is CH, CH is optionally substituted with R^{Ar};
X⁴ is each independently selected from N, O or S;
R², R³, R⁴, R⁵, R^{Ar}, Z¹, Z², Z³, Z⁴, Z⁵, Z⁶, Z⁷, Z⁸, G and n and r are as defined as claim 1.

8. The compound of claim 1, wherein the compound is formula (BIIa), (BIIb), (BIIc), (BIId), (BIIe), (BIIf) or (BIIg): 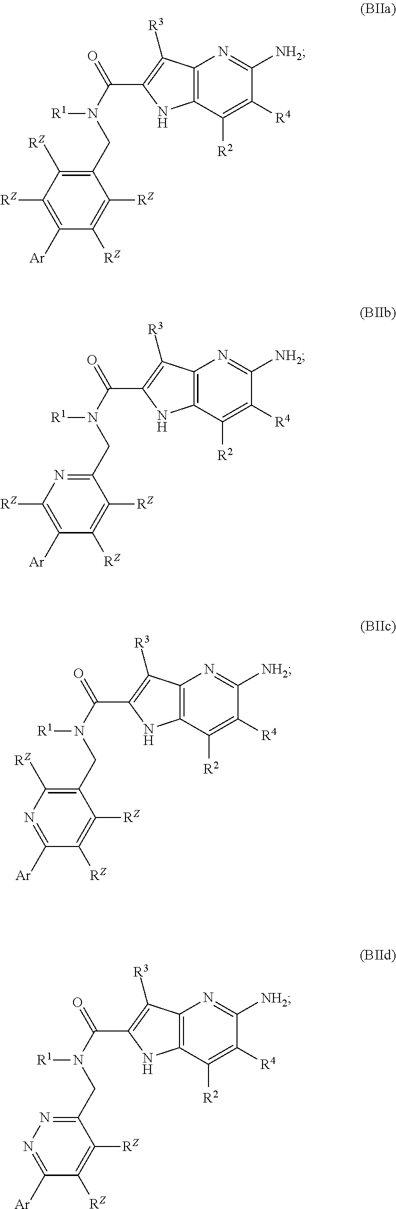 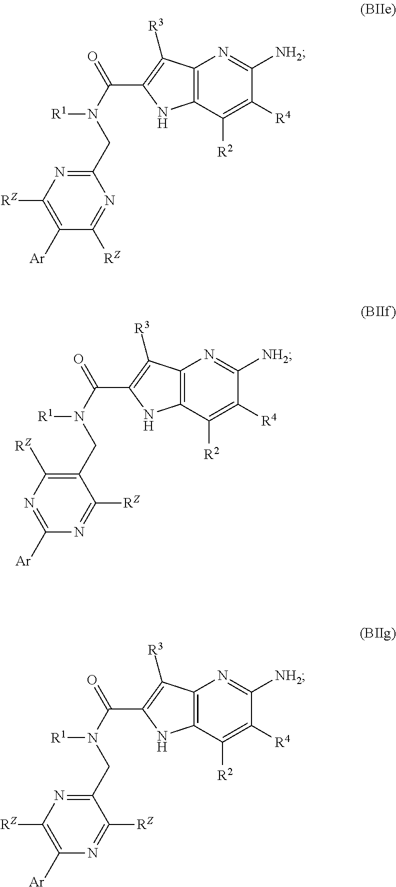
R¹, R², R³, R⁴, R^{Z}, Ar are as defined as claim 1.

9. The compound of claim 1, wherein the compound is formula (BIIIa), (BIIIb), (BIIIc): 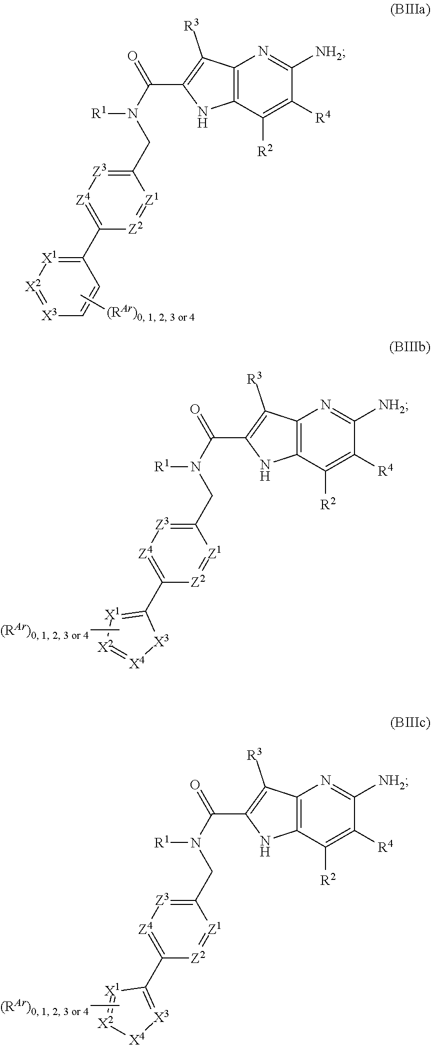
wherein, X¹, X² and X³ are each independently selected from N or CH; provided that when anyone of X¹, X² or X³ is CH, CH is optionally substituted with R^{Ar};
X⁴ is each independently selected from NH, O or S; provided that when anyone of X⁴ is NH, NH is optionally substituted with R^{Ar};
R¹, R², R³, R⁴, R^{Ar}, Z¹, Z², Z³ and Z⁴ are as defined as claim 1;

10. The compound of claim 1, wherein the compound is formula (BIIId), (BIIIe), (BIIIf), (BIIIg), (BIIIh) or (BIIIi): 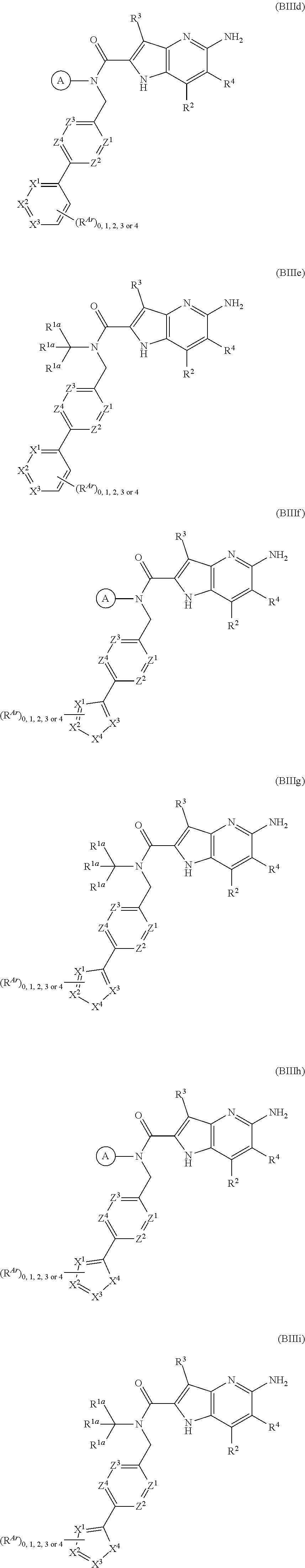
wherein, ring A is —C₃-C₈cycloalkyl (preferably, mono, bridged, fused or spiral cycloalkyl) or 3- to 8-membered saturated heterocyclyl (preferably, mono, bridged, fused or spiral heterocyclyl); wherein each of said —C₃-C₈cycloalkyl (preferably, mono, bridged, fused or spiral cycloalkyl) or 3- to 8-membered saturated heterocyclyl (preferably, mono, bridged, fused or spiral heterocyclyl) is optionally substituted with at least one substituent R^{1a};
X¹, X² and X³ are each independently selected from N or CH; provided that when anyone of X¹, X² or X³ is CH, CH is optionally substituted with R^{Ar};
X⁴ is each independently selected from NH, O or S; provided that when anyone of X⁴ is NH, NH is optionally substituted with R^{Ar};
R¹, R², R³, R⁴, R^{Ar}, Z¹, Z², Z³ and Z⁴ are as defined as claim 1.

11. The compound of any anyone of preceding claims, wherein R^{Z1} is selected from hydrogen, —F, —Cl, —Br, —I, methyl, ethyl, propyl (iso-propyl or n-propyl), butyl (n-butyl, sec-butyl, iso-butyl or tert-butyl), methoxy, ethoxy, propoxy, butoxy, cyclopropyl, cyclobutyl, cyclopentyl, cyclohexyl, cyclopropoxy, cyclobutoxy, cyclopentoxy or cyclohexoxy; wherein each of said methyl, ethyl, propyl (iso-propyl or n-propyl), butyl (n-butyl, sec-butyl, iso-butyl or tert-butyl), methoxy, ethoxy, propoxy, butoxy, cyclopropyl, cyclobutyl, cyclopentyl, cyclohexyl, cyclopropoxy, cyclobutoxy, cyclopentoxy or cyclohexoxy is optionally substituted with at least one substituent selected from —F, —Cl, —Br, —I, methoxy, ethoxy, propoxy, butoxy, —CN, —OH, —NH₂ or oxo.

12. The compound of any anyone of preceding claims, wherein R^{Z1} is selected from hydrogen, —F, —Cl, —Br, —I, methyl, ethyl, propyl (iso-propyl or n-propyl), butyl (n-butyl, sec-butyl, iso-butyl or tert-butyl), methoxy, ethoxy, propoxy, butoxy, cyclopropyl, cyclobutyl, cyclopentyl, cyclohexyl, cyclopropoxy, cyclobutoxy, cyclopentoxy, cyclohexoxy, hydroxymethyl, hydroxyethyl, hydroxypropyl, hydroxybutyl.

13. The compound of any anyone of preceding claims, wherein R^{Z1} is selected from hydrogen, —F, —Cl, —Br, —I, methyl, ethyl, propyl (iso-propyl or n-propyl), methoxy, ethoxy, cyclopropyl, cyclobutyl, cyclopropoxy or hydroxypropyl

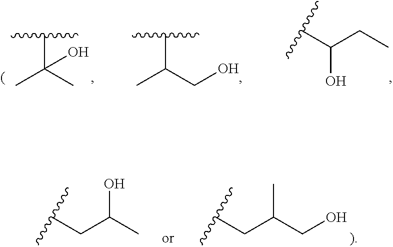

14. The compound of anyone of preceding claims, wherein R⁵ is selected from hydrogen, —F, —Cl, —Br, —I, methyl, ethyl, propyl (iso-propyl or n-propyl), butyl (n-butyl, sec-butyl, iso-butyl or tert-butyl), cyclopropyl, cyclobutyl, cyclopentyl or cyclohexyl, wherein methyl, ethyl, propyl (iso-propyl or n-propyl), butyl (n-butyl, sec-butyl, iso-butyl or tert-butyl), cyclopropyl, cyclobutyl, cyclopentyl or cyclohexyl is optionally substituted with at least one substituent selected from —F, —Cl, —Br, —I, methoxy, ethoxy, propoxy, butoxy, —CN, —OH, —NH₂ or oxo;

15. The compound of anyone of preceding claims, wherein R⁵ is selected from hydrogen, —F, —Cl, —Br, —I, methyl, ethyl, propyl (iso-propyl or n-propyl) or butyl (n-butyl, sec-butyl, iso-butyl or tert-butyl); more preferably, R⁵ is selected from hydrogen, —F, —Cl, —Br, methyl, ethyl.

16. The compound of anyone of preceding claims, wherein two geminal R⁵ and the carbon atom which they attach to form a 3-, 4- or 5-membered carbocyclic ring; wherein said ring is optionally substituted with at least one substituent selected from —F, —Cl, —Br, —I, methoxy, ethoxy, propoxy, butoxy, —CN, —OH, —NH₂ or oxo.

17. The compound of anyone of preceding claims, wherein two geminal R⁵ and the carbon atom which they attach to form a 3-, 4- or 5-membered carbocyclic ring.

18. The compound of anyone of preceding claims, wherein two geminal R⁵ and the carbon atom which they attach to form a 3-membered carbocyclic ring.

19. The compound of anyone of preceding claims, wherein G is selected from CH₂, O or NH; preferably, G is selected from CH₂ or O.

20. The compound of any anyone of preceding claims, wherein R¹ is selected from methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cyclopentyl, cyclohexyl, cycloheptyl, cyclooctyl, 3-, 4-, 5-, 6-, 7- or 8-membered saturated mono-heterocyclyl, 5-, 6-, 7- or 8-membered saturated bridged-heterocyclyl, 4-, 5-, 6-, 7- or 8-membered saturated fused-heterocyclyl, or 5-, 6-, 7- or 8-membered saturated spiral-heterocyclyl; wherein each of said methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cyclopentyl, cyclohexyl, cycloheptyl, cyclooctyl, 3-, 4-, 5-, 6-, 7- or 8-membered saturated mono-heterocyclyl, 5-, 6-, 7- or 8-membered saturated bridged-heterocyclyl, 4-, 5-, 6-, 7- or 8-membered saturated fused-heterocyclyl, or 5-, 6-, 7- or 8-membered saturated spiral-heterocyclyl is optionally substituted with at least one substituent R^{1a};
R^{1a} is each independently selected from —F, —Cl, —Br, —I, methyl, ethyl, propyl (iso-propyl or n-propyl), butyl (n-butyl, sec-butyl, iso-butyl or tert-butyl), cyclopropyl, cyclobutyl, cyclopentyl, cyclohexyl, 3-, 4-, 5- or 6-membered saturated heterocyclyl, methoxy, ethoxy, propoxy, butoxy, cyclopropoxy, cyclobutoxy, cyclopentoxy, cyclohexoxy, —CN, —OH, —NH₂ or oxo; wherein each of said methyl, ethyl, propyl (iso-propyl or n-propyl), butyl (n-butyl, sec-butyl, iso-butyl or tert-butyl), cyclopropyl, cyclobutyl, cyclopentyl, cyclohexyl, 3-, 4-, 5- or 6-membered saturated heterocyclyl, methoxy, ethoxy, propoxy, butoxy, cyclopropoxy, cyclobutoxy, cyclopentoxy or cyclohexoxy is optionally substituted with at least one substituent selected from —F, —Cl, —Br, —I, methyl, ethyl, propyl (iso-propyl or n-propyl), butyl (n-butyl, sec-butyl, iso-butyl or tert-butyl), cyclopropyl, cyclobutyl, cyclopentyl, cyclohexyl, 3-, 4-, 5- or 6-membered saturated heterocyclyl, methoxy, ethoxy, propoxy, butoxy, cyclopropoxy, cyclobutoxy, cyclopentoxy, cyclohexoxy, —CN, —OH, —NH₂ or oxo.

21. The compound of any anyone of preceding claims, wherein R¹ is selected from methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cyclopentyl, cyclohexyl, cycloheptyl, cyclooctyl, 3-, 4-, 5-, 6-, 7- or 8-membered saturated mono-heterocyclyl, 5-, 6-, 7- or 8-membered saturated bridged-heterocyclyl, 4-, 5-, 6-, 7- or 8-membered saturated fused-heterocyclyl, or 5-, 6-, 7- or 8-membered saturated spiral-heterocyclyl each of said is optionally substituted with at least one substituent R^{1a};
R^{1a} is each independently selected from —F, —Cl, —Br, —I, methyl, ethyl, propyl (iso-propyl or n-propyl), butyl (n-butyl, sec-butyl, iso-butyl or tert-butyl), cyclopropyl, cyclobutyl, cyclopentyl, cyclohexyl, 3-, 4-, 5- or 6-membered saturated heterocyclyl, methoxy, ethoxy, propoxy, butoxy, cyclopropoxy, cyclobutoxy, cyclopentoxy, cyclohexoxy, —CN, —OH, —NH₂ or oxo;

22. The compound of any anyone of preceding claims, wherein R¹ is selected from methyl, ethyl, propyl (iso-propyl or n-propyl), cyclopropyl, butyl (n-butyl, sec-butyl, iso-butyl or tert-butyl), cyclobutyl, cyclopentyl, cyclohexyl, tetrahydropyranyl, tetrahydrofuranyl, piperidinyl or pyrrolidinyl; wherein each of said methyl, ethyl, propyl (iso-propyl or n-propyl), cyclopropyl, butyl (n-butyl, sec-butyl, iso-butyl or tert-butyl), pentyl, hexyl, cyclobutyl, cyclopentyl, cyclohexyl, tetrahydropyranyl, tetrahydrofuranyl, piperidinyl or pyrrolidinyl is optionally substituted with at least one substituent R^{1a};
R^{1a} is each independently selected from —F, —Cl, —Br, —I, methyl, ethyl, propyl (iso-propyl or n-propyl), cyclopropyl, butyl (n-butyl, sec-butyl, iso-butyl or tert-butyl), pentyl, hexyl, cyclobutyl, cyclopentyl, cyclohexyl, tetrahydropyranyl, tetrahydrofuranyl, piperidinyl, pyrrolidinyl, methoxy, ethoxy, propoxy, butoxy, cyclopropoxy, cyclobutoxy, cyclopentoxy, cyclohexoxy, —CN, —OH, —NH₂ or oxo.

23. The compound of any anyone of preceding claims, wherein R¹ is selected from

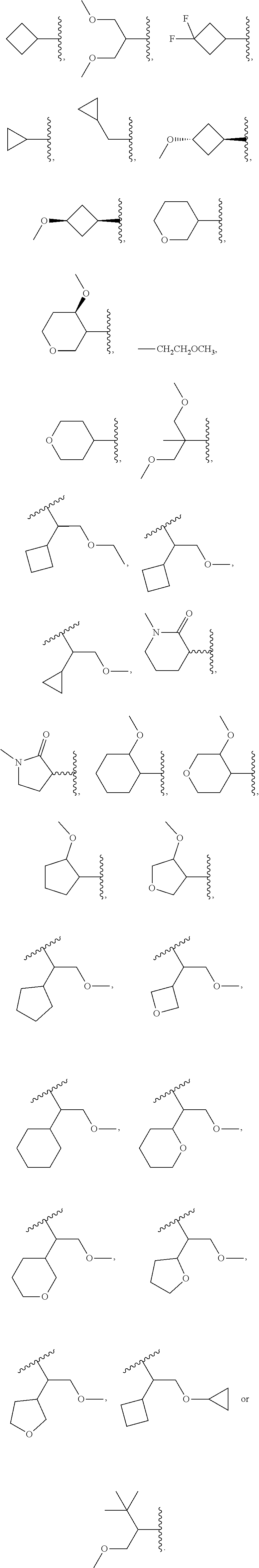

24. The compound of anyone of preceding claims, wherein R² is selected from hydrogen, —F, —Cl, —Br, —I, methyl, ethyl, propyl (iso-propyl or n-propyl), butyl (n-butyl, sec-butyl, iso-butyl or tert-butyl), methoxy, ethoxy, propoxy, butoxy, cyclopropyl, cyclobutyl, cyclopentyl, cyclohexyl, cyclopropoxy, cyclobutoxy, cyclopentoxy, cyclohexoxy or —CN; wherein methyl, ethyl, propyl (iso-propyl or n-propyl), butyl (n-butyl, sec-butyl, iso-butyl or tert-butyl), methoxy, ethoxy, propoxy, butoxy, cyclopropyl, cyclobutyl, cyclopentyl, cyclohexyl, cyclopropoxy, cyclobutoxy, cyclopentoxy or cyclohexoxy is optionally substituted with at least one substituent selected from —F, —Cl, —Br, —I, methoxy, ethoxy, propoxy, butoxy, cyclopropoxy, cyclobutoxy, cyclopentoxy, cyclohexoxy;

25. The compound of anyone of preceding claims, wherein R² is selected from hydrogen, methyl, ethyl, cyclopropyl, cyclobutyl, cyclopropoxy, cyclobutoxy or —CN; wherein methyl, ethyl, is optionally substituted with at least one substituent selected from —F, —Cl, —Br, —I, methoxy, ethoxy, propoxy, cyclopropoxy or cyclobutoxy;

26. The compound of anyone of preceding claims, wherein R² is selected from hydrogen, —F, —Cl, —Br, —I, methyl, ethyl, propyl (iso-propyl or n-propyl), butyl (n-butyl, sec-butyl, iso-butyl or tert-butyl), methoxy, ethoxy, propoxy, butoxy, cyclopropyl, cyclobutyl, cyclopentyl, cyclohexyl, cyclopropoxy, cyclobutoxy, cyclopentoxy, cyclohexoxy or —CN.

27. The compound of anyone of preceding claims, wherein R² is selected from hydrogen, methyl, ethyl or —CN.

28. The compound of anyone of preceding claims, wherein R³ is selected from hydrogen, —F, —Cl, —Br, —I, methyl, ethyl, propyl (iso-propyl or n-propyl), butyl (n-butyl, sec-butyl, iso-butyl or tert-butyl) or —CN, wherein methyl, ethyl, propyl (iso-propyl or n-propyl) or butyl (n-butyl, sec-butyl, iso-butyl or tert-butyl) is optionally substituted with at least one substituent selected from hydrogen, —F, —Cl, —Br, —I, methoxy, ethoxy, propoxy, butoxy, cyclopropyl, cyclobutyl, cyclopentyl, cyclohexyl, —CN, —OH, —NH₂ or oxo.

29. The compound of anyone of preceding claims, wherein R³ is selected from hydrogen, —F, —Cl, —Br, —I, methyl, ethyl, propyl (iso-propyl or n-propyl), butyl (n-butyl, sec-butyl, iso-butyl or tert-butyl) or —CN.

30. The compound of anyone of preceding claims, wherein R³ is selected from hydrogen, —F, —Cl, —Br, —I or —CN.

31. The compound of anyone of preceding claims, wherein R⁴ is selected from hydrogen, —F, —Cl, —Br, —I, methyl, ethyl, propyl (iso-propyl or n-propyl), butyl (n-butyl, sec-butyl, iso-butyl or tert-butyl), methoxy, ethoxy, propoxy, butoxy, cyclopropyl, cyclobutyl, cyclopentyl, cyclohexyl, cyclopropoxy, cyclobutoxy, cyclopentoxy or cyclohexoxy, wherein methyl, ethyl, propyl (iso-propyl or n-propyl) or butyl (n-butyl, sec-butyl, iso-butyl or tert-butyl), methoxy, ethoxy, propoxy, butoxy, cyclopropyl, cyclobutyl, cyclopentyl, cyclohexyl, cyclopropoxy, cyclobutoxy, cyclopentoxy or cyclohexoxy is optionally substituted with at least one substituent selected from —F, —Cl, —Br, —I, methoxy, ethoxy, propoxy, butoxy, cyclopropoxy, cyclobutoxy, cyclopentoxy, cyclohexoxy, —CN, —OH, —NH₂ or oxo;

32. The compound of anyone of preceding claims, wherein R⁴ is selected from hydrogen, —F, —Cl, —Br, methyl, ethyl, propyl (iso-propyl or n-propyl), methoxy, ethoxy, propoxy, cyclopropyl, cyclobutyl, cyclopropoxy or cyclobutoxy, wherein methyl, ethyl, propyl (iso-propyl or n-propyl), methoxy, ethoxy, propoxy, cyclopropyl, cyclobutyl, cyclopropoxy or cyclobutoxy is optionally substituted with at least one substituent selected from —F, —Cl, —Br, —I, methoxy, ethoxy, propoxy, cyclopropoxy, cyclobutoxy, —CN, —OH, —NH₂ or oxo;

33. The compound of anyone of preceding claims, wherein R⁴ is selected from hydrogen, —F, —Cl, —Br, —I, methyl, ethyl, propyl (iso-propyl or n-propyl) or butyl (n-butyl, sec-butyl, iso-butyl or tert-butyl).

34. The compound of anyone of preceding claims, wherein R⁴ is selected from hydrogen, —F, —Cl, —Br, —I, methyl, ethyl.

35. The compound of anyone of preceding claims, wherein at most two of Z¹, Z², Z³ and Z⁴ are N; R^{Z} is each independently selected from hydrogen, —F, —Cl, —Br, —I, methyl, ethyl, propyl (iso-propyl or n-propyl) or butyl (n-butyl, sec-butyl, iso-butyl or tert-butyl), methoxy, ethoxy, propoxy or butoxy; wherein each of said ethyl, propyl (iso-propyl or n-propyl) or butyl (n-butyl, sec-butyl, iso-butyl or tert-butyl), methoxy, ethoxy, propoxy or butoxy is optionally substituted with at least one substituent selected from —F, —Cl, —Br, —I;

36. The compound of anyone of preceding claims, wherein R^{Z} is each independently selected from hydrogen, —F, —Cl, —Br, —I, methyl, ethyl, propyl (iso-propyl or n-propyl), butyl (n-butyl, sec-butyl, iso-butyl or tert-butyl), methoxy, ethoxy, propoxy or butoxy;

37. The compound of anyone of preceding claims, wherein R^{Z} is each independently selected from hydrogen, —F, —Cl, —Br, methyl or ethyl.

38. The compound of anyone of preceding claims, wherein Ar is independently selected from phenyl, pyridinyl, pyridazinyl, pyrimidinyl, pyrazinyl, pyrrolyl, pyrazolyl, imidazolyl, 1,2,4-triazolyl, 1,2,3-triazolyl, oxazolyl, furanyl, thiazolyl or thiophenyl; each of said phenyl, pyridinyl, pyridazinyl, pyrimidinyl, pyrazinyl, pyrrolyl, pyrazolyl, imidazolyl, 1,2,4-triazolyl, 1,2,3-triazolyl, oxazolyl, furanyl, thiazolyl or thiophenyl is optionally substituted with at least one substituent R^{Ar};
R^{Ar} is each independently selected from hydrogen, —F, —Cl, —Br, —I, methyl, ethyl, propyl (iso-propyl or n-propyl), butyl (n-butyl, sec-butyl, iso-butyl or tert-butyl), methoxy, ethoxy, propoxy, butoxy, cyclopropyl, cyclobutyl, cyclopentyl, cyclohexyl, cyclopropoxy, cyclobutoxy, cyclopentoxy, cyclohexoxy or —CN; wherein each of said methyl, ethyl, propyl (iso-propyl or n-propyl), butyl (n-butyl, sec-butyl, iso-butyl or tert-butyl), methoxy, ethoxy, propoxy, butoxy, cyclopropyl, cyclobutyl, cyclopentyl, cyclohexyl, cyclopropoxy, cyclobutoxy, cyclopentoxy, cyclohexoxy is optionally substituted with at least one substituent selected from —F, —Cl, —Br or —I;

39. The compound of anyone of preceding claims, wherein Ar is independently selected from phenyl, pyridinyl, pyrimidinyl, pyrrolyl, pyrazolyl or 1,2,4-triazolyl; each of said phenyl, pyridinyl, pyrimidinyl, pyrrolyl, pyrazolyl or 1,2,4-triazolyl is optionally substituted with at least one substituent R^{Ar};
R^{Ar} is each independently selected from hydrogen, —F, —Cl, —Br, —I, methyl, ethyl, propyl (iso-propyl or n-propyl), butyl (n-butyl, sec-butyl, iso-butyl or tert-butyl), methoxy, ethoxy, propoxy, butoxy, cyclopropyl, cyclobutyl, cyclopentyl, cyclohexyl, cyclopropoxy, cyclobutoxy, cyclopentoxy, cyclohexoxy, —CF₃ or —CN;

40. The compound of anyone of preceding claims, Ar is independently selected from phenyl, pyridinyl, pyrimidinyl, pyrrolyl, pyrazolyl or 1,2,4-triazolyl; each of said phenyl, pyridinyl, pyrimidinyl, pyrrolyl, pyrazolyl or 1,2,4-triazolyl is optionally substituted with at least one substituent R^{Ar}; wherein R^{Ar} is each independently selected from hydrogen, —F, —Cl, —Br, methyl, ethyl, methoxy, ethoxy, —CF₃ or —CN.

41. The compound of any one of the preceding claims, wherein the compound is selected from

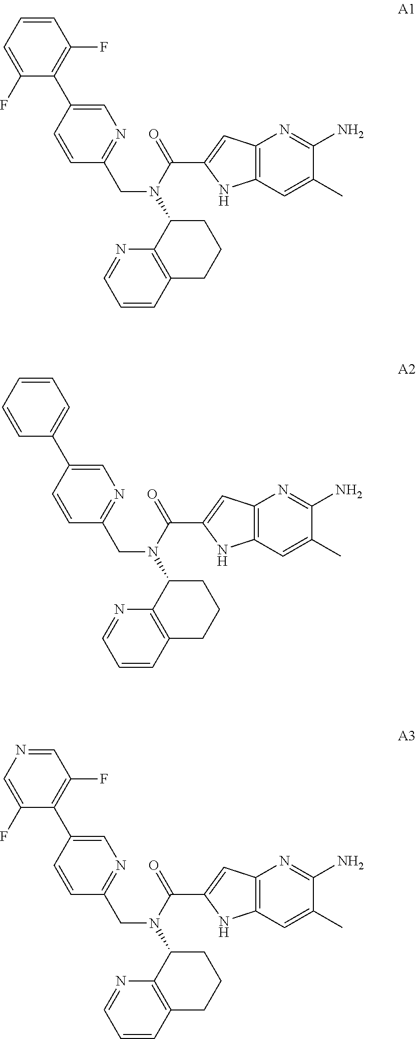 [SMILES: Cc1cc2[nH]c(C(=O)N(Cc3ccc(-c4c(F)cccc4F)cn3)[C@@H]3CCCc4cccnc43)cc2nc1N ; Cc1cc2[nH]c(C(=O)N(Cc3ccc(-c4ccccc4)cn3)[C@@H]3CCCc4cccnc43)cc2nc1N ; Cc1cc2[nH]c(C(=O)N(Cc3ccc(-c4c(F)cncc4F)cn3)[C@@H]3CCCc4cccnc43)cc2nc1N]

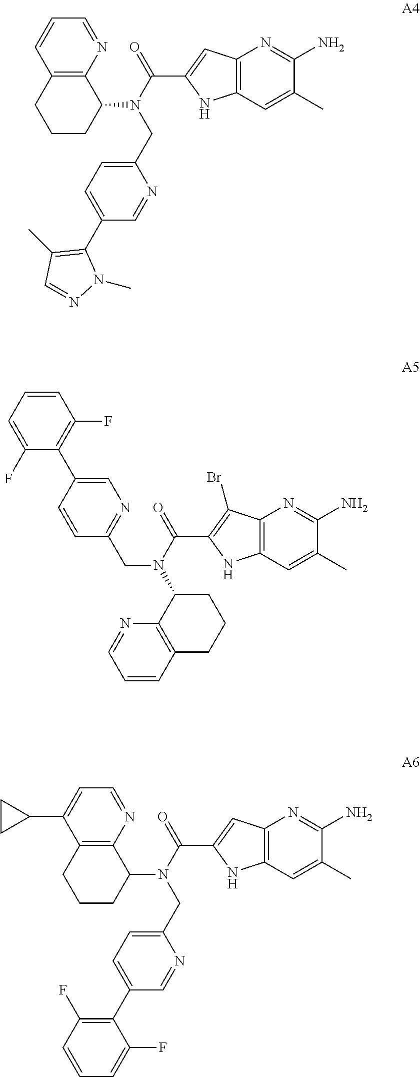

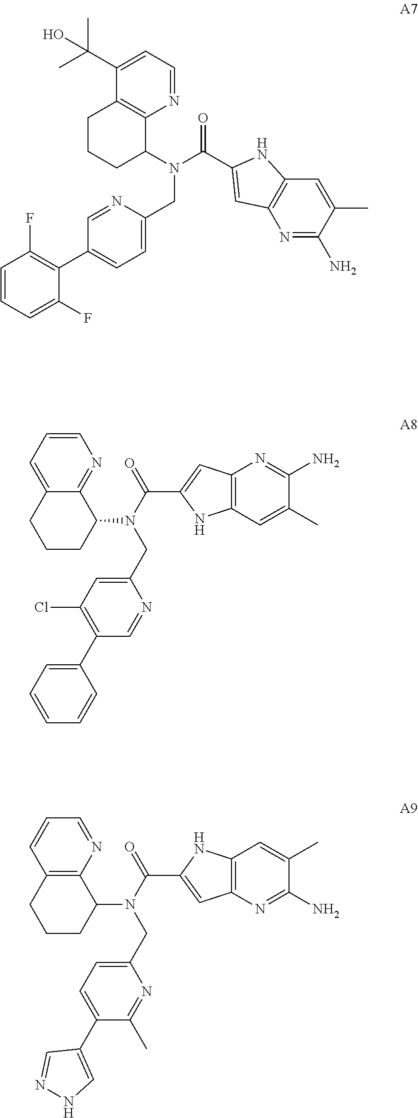 [SMILES: Cc1cc2[nH]c(C(=O)N(Cc3ccc(-c4c(F)cccc4F)cn3)C3CCCc4c(C(C)(C)O)ccnc43)cc2nc1N ; Cc1cc2[nH]c(C(=O)N(Cc3cc(Cl)c(-c4ccccc4)cn3)[C@@H]3CCCc4cccnc43)cc2nc1N ; Cc1cc2[nH]c(C(=O)N(Cc3ccc(-c4cn[nH]c4)c(C)n3)C3CCCc4cccnc43)cc2nc1N]

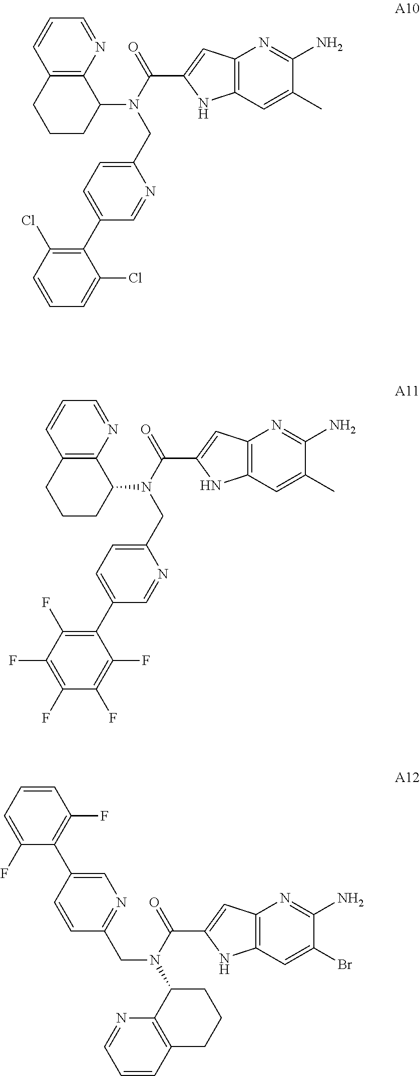 [SMILES: Cc1cc2[nH]c(C(=O)N(Cc3ccc(-c4c(Cl)cccc4Cl)cn3)C3CCCc4cccnc43)cc2nc1N ; Cc1cc2[nH]c(C(=O)N(Cc3ccc(-c4c(F)c(F)c(F)c(F)c4F)cn3)[C@@H]3CCCc4cccnc43)cc2nc1N ; Nc1nc2cc(C(=O)N(Cc3ccc(-c4c(F)cccc4F)cn3)[C@@H]3CCCc4cccnc43)[nH]c2cc1Br]

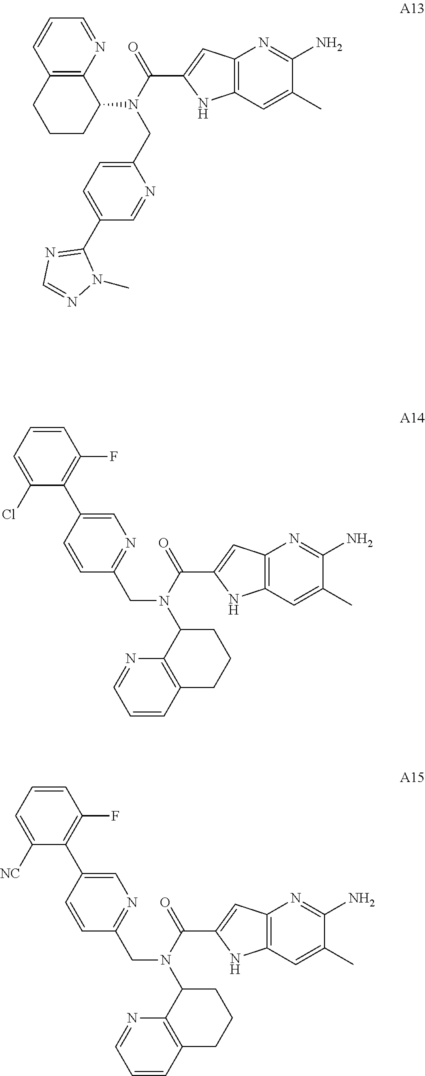 [SMILES: Cc1cc2[nH]c(C(=O)N(Cc3ccc(-c4ncnn4C)cn3)[C@@H]3CCCc4cccnc43)cc2nc1N ; Cc1cc2[nH]c(C(=O)N(Cc3ccc(-c4c(F)cccc4Cl)cn3)C3CCCc4cccnc43)cc2nc1N ; Cc1cc2[nH]c(C(=O)N(Cc3ccc(-c4c(F)cccc4C#N)cn3)C3CCCc4cccnc43)cc2nc1N]

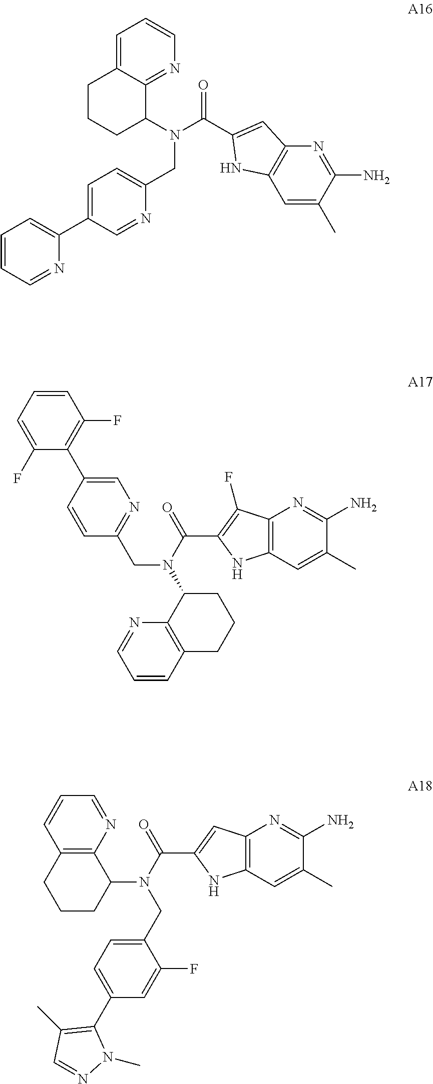

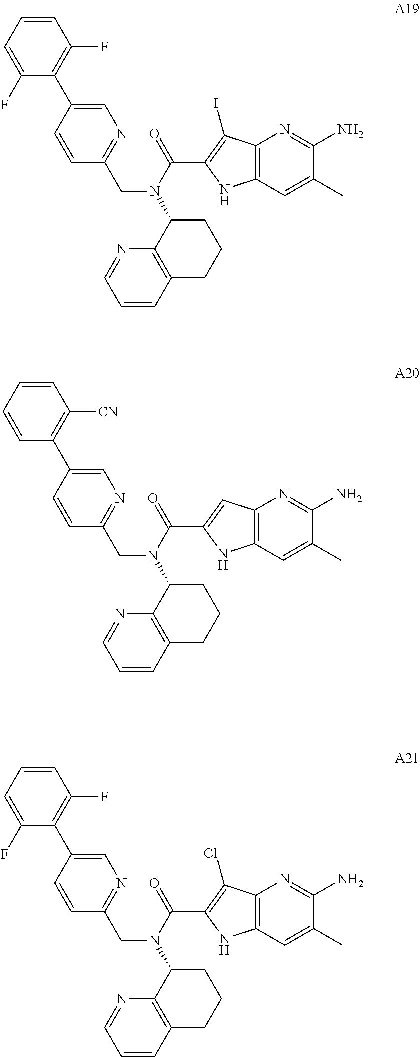 [SMILES: Cc1cc2[nH]c(C(=O)N(Cc3ccc(-c4c(F)cccc4F)cn3)[C@@H]3CCCc4cccnc43)c(I)c2nc1N ; Cc1cc2[nH]c(C(=O)N(Cc3ccc(-c4ccccc4C#N)cn3)[C@@H]3CCCc4cccnc43)cc2nc1N ; Cc1cc2[nH]c(C(=O)N(Cc3ccc(-c4c(F)cccc4F)cn3)[C@@H]3CCCc4cccnc43)c(Cl)c2nc1N]

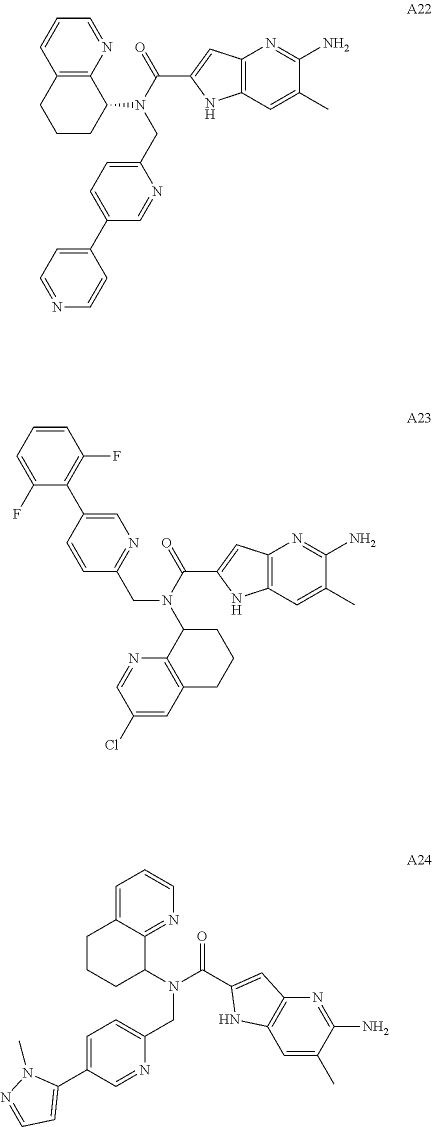 [SMILES: Cc1cc2[nH]c(C(=O)N(Cc3ccc(-c4ccncc4)cn3)[C@@H]3CCCc4cccnc43)cc2nc1N ; Cc1cc2[nH]c(C(=O)N(Cc3ccc(-c4c(F)cccc4F)cn3)C3CCCc4cc(Cl)cnc43)cc2nc1N ; Cc1cc2[nH]c(C(=O)N(Cc3ccc(-c4ccnn4C)cn3)C3CCCc4cccnc43)cc2nc1N]

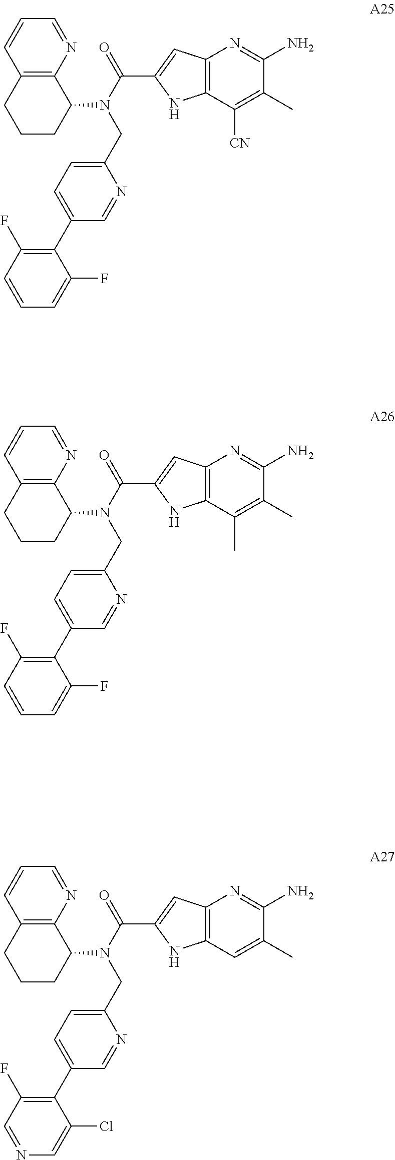 [SMILES: Cc1c(N)nc2cc(C(=O)N(Cc3ccc(-c4c(F)cccc4F)cn3)[C@@H]3CCCc4cccnc43)[nH]c2c1C#N ; Cc1c(N)nc2cc(C(=O)N(Cc3ccc(-c4c(F)cccc4F)cn3)[C@@H]3CCCc4cccnc43)[nH]c2c1C ; Cc1cc2[nH]c(C(=O)N(Cc3ccc(-c4c(F)cncc4Cl)cn3)[C@@H]3CCCc4cccnc43)cc2nc1N]

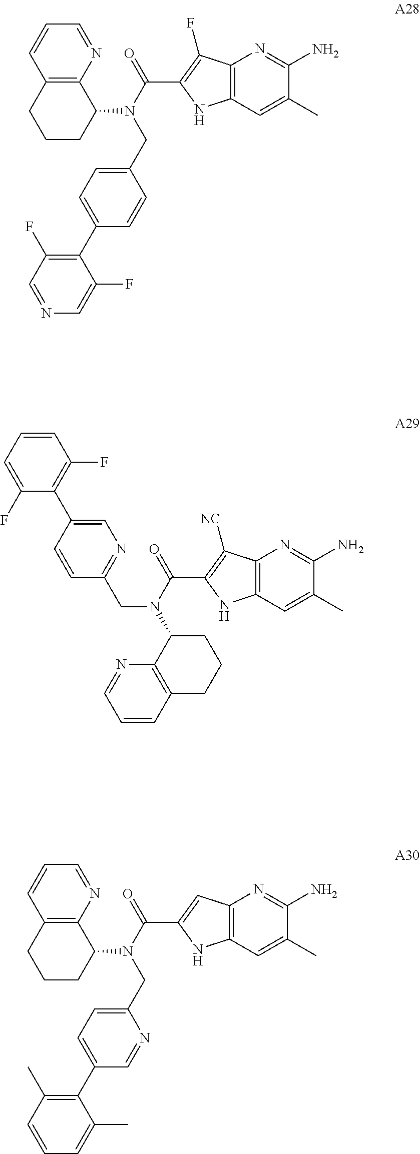 [SMILES: Cc1cc2[nH]c(C(=O)N(Cc3ccc(-c4c(F)cncc4F)cc3)[C@@H]3CCCc4cccnc43)c(F)c2nc1N ; Cc1cc2[nH]c(C(=O)N(Cc3ccc(-c4c(F)cccc4F)cn3)[C@@H]3CCCc4cccnc43)c(C#N)c2nc1N ; Cc1cc2[nH]c(C(=O)N(Cc3ccc(-c4c(C)cccc4C)cn3)[C@@H]3CCCc4cccnc43)cc2nc1N]

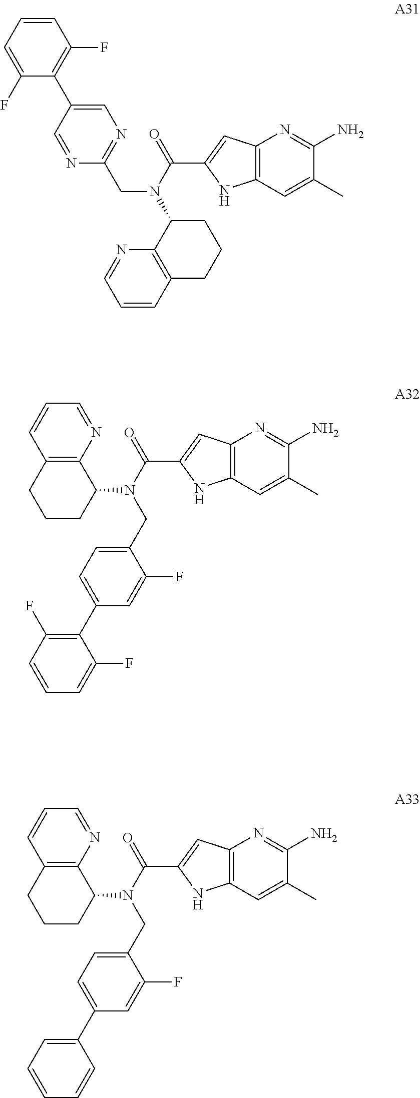 [SMILES: Cc1cc2[nH]c(C(=O)N(Cc3ncc(-c4c(F)cccc4F)cn3)[C@@H]3CCCc4cccnc43)cc2nc1N ; Cc1cc2[nH]c(C(=O)N(Cc3ccc(-c4c(F)cccc4F)cc3F)[C@@H]3CCCc4cccnc43)cc2nc1N ; Cc1cc2[nH]c(C(=O)N(Cc3ccc(-c4ccccc4)cc3F)[C@@H]3CCCc4cccnc43)cc2nc1N]

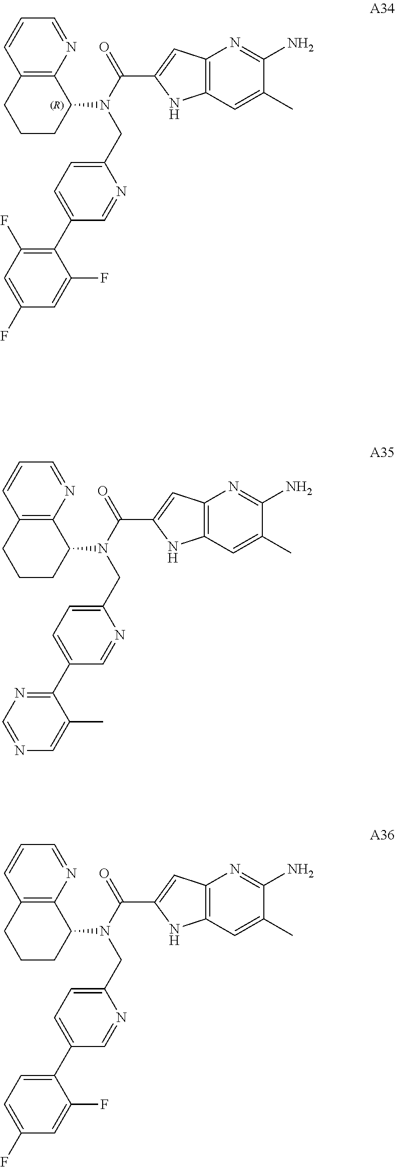

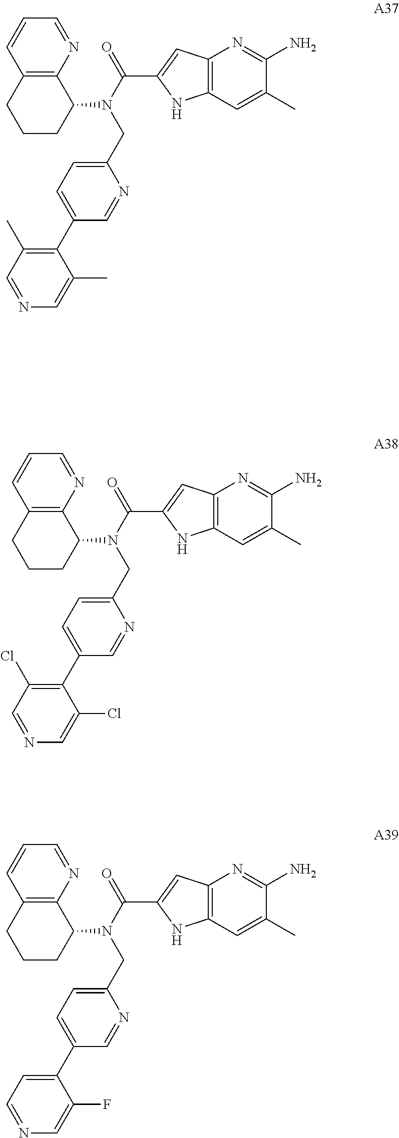 [SMILES: Cc1cc2[nH]c(C(=O)N(Cc3ccc(-c4c(C)cncc4C)cn3)[C@@H]3CCCc4cccnc43)cc2nc1N ; Cc1cc2[nH]c(C(=O)N(Cc3ccc(-c4c(Cl)cncc4Cl)cn3)[C@@H]3CCCc4cccnc43)cc2nc1N ; Cc1cc2[nH]c(C(=O)N(Cc3ccc(-c4ccncc4F)cn3)[C@@H]3CCCc4cccnc43)cc2nc1N]

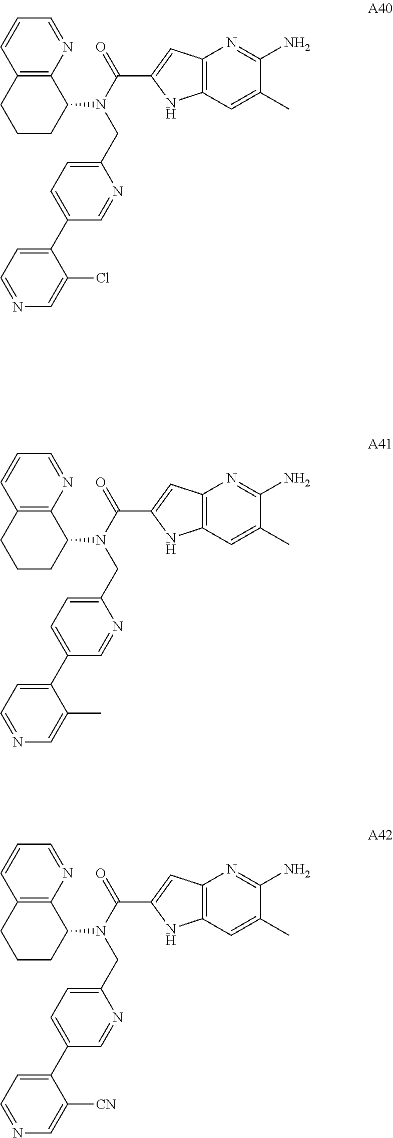 [SMILES: Cc1cc2[nH]c(C(=O)N(Cc3ccc(-c4ccncc4Cl)cn3)[C@@H]3CCCc4cccnc43)cc2nc1N ; Cc1cnccc1-c1ccc(CN(C(=O)c2cc3nc(N)c(C)cc3[nH]2)[C@@H]2CCCc3cccnc32)nc1 ; Cc1cc2[nH]c(C(=O)N(Cc3ccc(-c4ccncc4C#N)cn3)[C@@H]3CCCc4cccnc43)cc2nc1N]

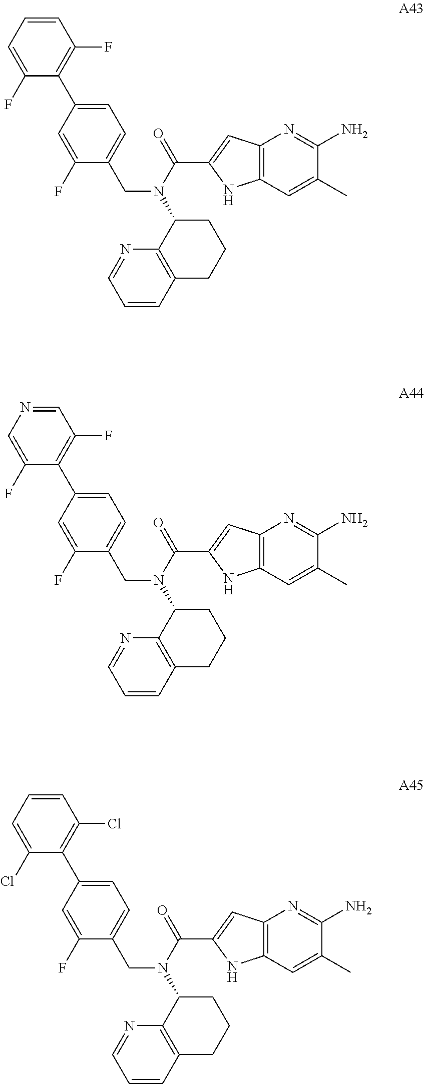 [SMILES: Cc1cc2[nH]c(C(=O)N(Cc3ccc(-c4c(F)cccc4F)cc3F)[C@@H]3CCCc4cccnc43)cc2nc1N ; Cc1cc2[nH]c(C(=O)N(Cc3ccc(-c4c(F)cncc4F)cc3F)[C@@H]3CCCc4cccnc43)cc2nc1N ; Cc1cc2[nH]c(C(=O)N(Cc3ccc(-c4c(Cl)cccc4Cl)cc3F)[C@@H]3CCCc4cccnc43)cc2nc1N]

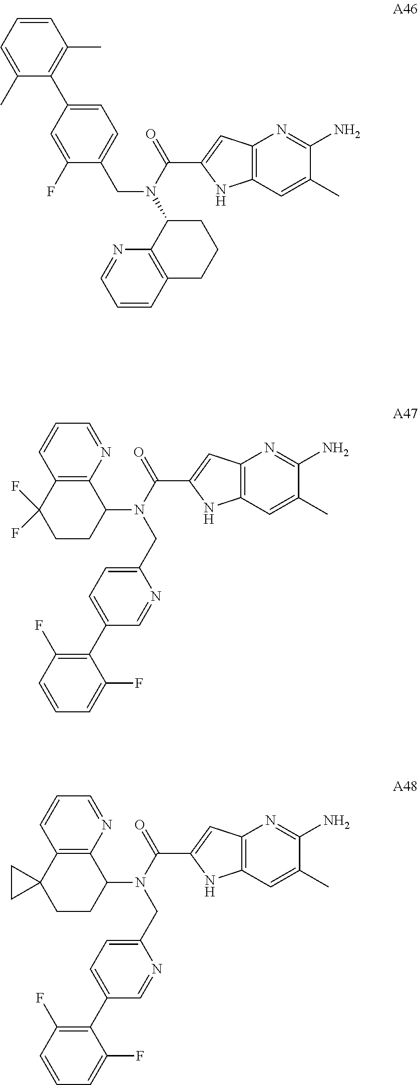 [SMILES: Cc1cc2[nH]c(C(=O)N(Cc3ccc(-c4c(C)cccc4C)cc3F)[C@@H]3CCCc4cccnc43)cc2nc1N ; Cc1cc2[nH]c(C(=O)N(Cc3ccc(-c4c(F)cccc4F)cn3)C3CCC(F)(F)c4cccnc43)cc2nc1N ; Cc1cc2[nH]c(C(=O)N(Cc3ccc(-c4c(F)cccc4F)cn3)C3CCC4(CC4)c4cccnc43)cc2nc1N]

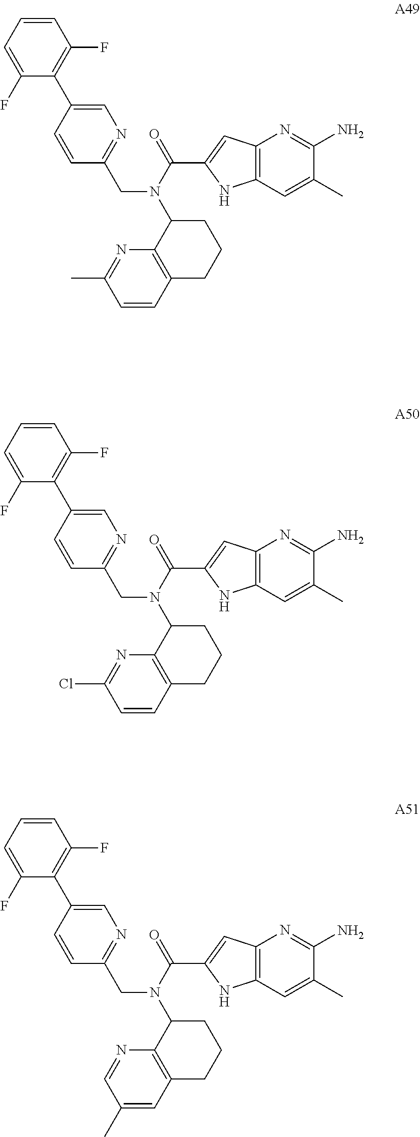 [SMILES: Cc1ccc2c(n1)C(N(Cc1ccc(-c3c(F)cccc3F)cn1)C(=O)c1cc3nc(N)c(C)cc3[nH]1)CCC2 ; Cc1cc2[nH]c(C(=O)N(Cc3ccc(-c4c(F)cccc4F)cn3)C3CCCc4ccc(Cl)nc43)cc2nc1N ; Cc1cnc2c(c1)CCCC2N(Cc1ccc(-c2c(F)cccc2F)cn1)C(=O)c1cc2nc(N)c(C)cc2[nH]1]

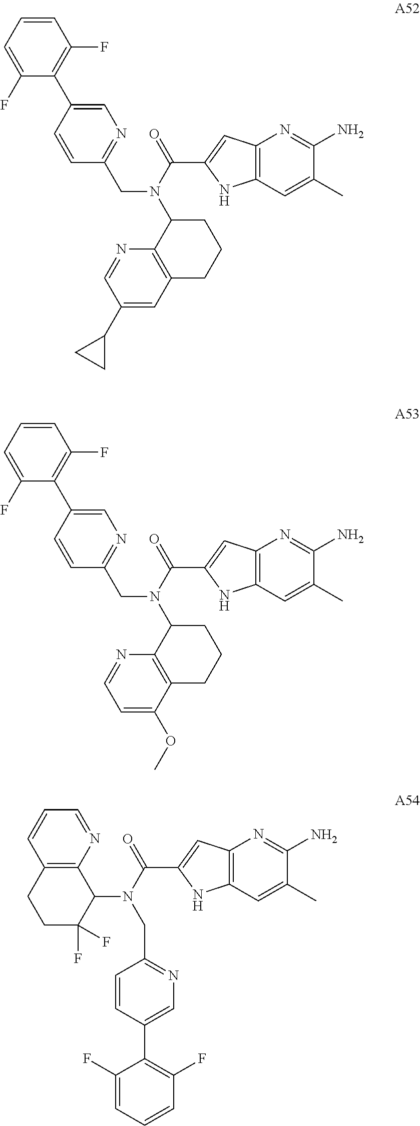 [SMILES: Cc1cc2[nH]c(C(=O)N(Cc3ccc(-c4c(F)cccc4F)cn3)C3CCCc4cc(C5CC5)cnc43)cc2nc1N ; COc1ccnc2c1CCCC2N(Cc1ccc(-c2c(F)cccc2F)cn1)C(=O)c1cc2nc(N)c(C)cc2[nH]1 ; Cc1cc2[nH]c(C(=O)N(Cc3ccc(-c4c(F)cccc4F)cn3)C3c4ncccc4CCC3(F)F)cc2nc1N]

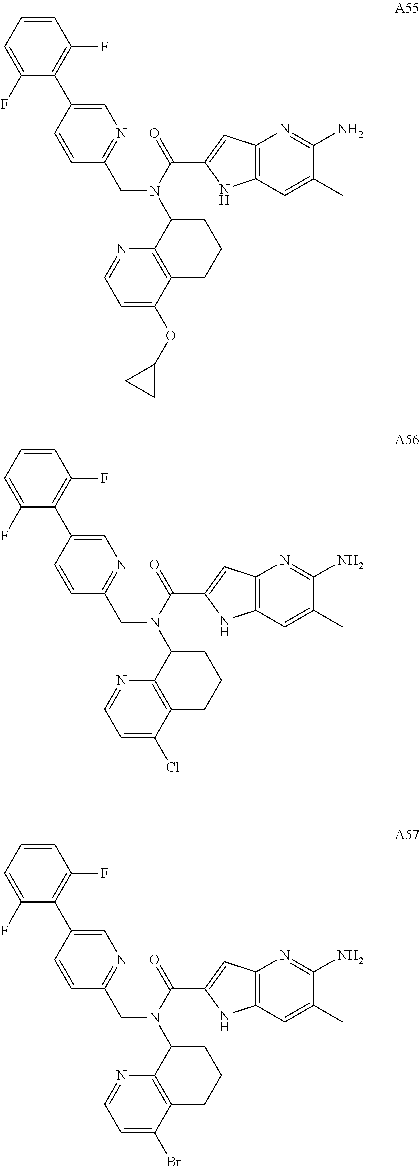 [SMILES: Cc1cc2[nH]c(C(=O)N(Cc3ccc(-c4c(F)cccc4F)cn3)C3CCCc4c(OC5CC5)ccnc43)cc2nc1N ; Cc1cc2[nH]c(C(=O)N(Cc3ccc(-c4c(F)cccc4F)cn3)C3CCCc4c(Cl)ccnc43)cc2nc1N ; Cc1cc2[nH]c(C(=O)N(Cc3ccc(-c4c(F)cccc4F)cn3)C3CCCc4c(Br)ccnc43)cc2nc1N]

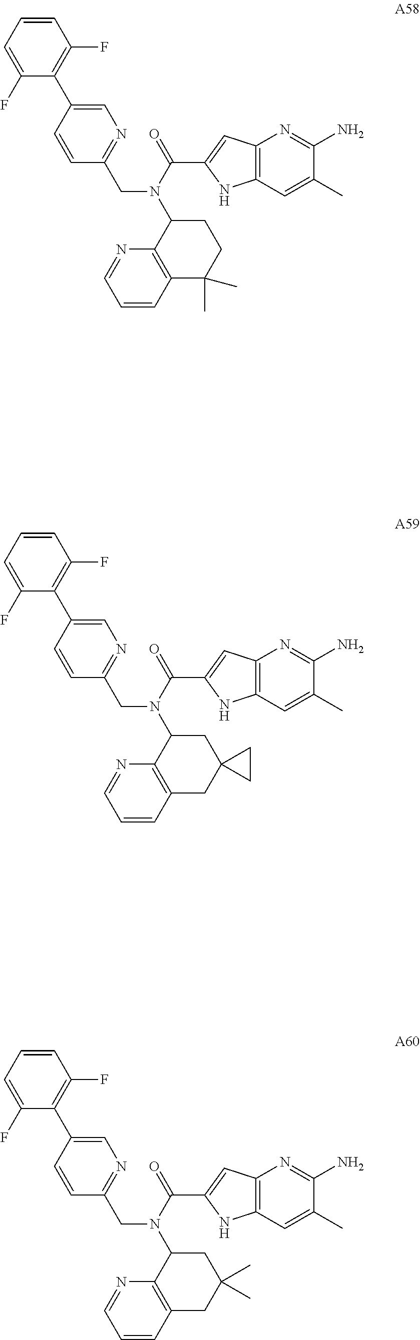 [SMILES: Cc1cc2[nH]c(C(=O)N(Cc3ccc(-c4c(F)cccc4F)cn3)C3CCC(C)(C)c4cccnc43)cc2nc1N ; Cc1cc2[nH]c(C(=O)N(Cc3ccc(-c4c(F)cccc4F)cn3)C3CC4(CC4)Cc4cccnc43)cc2nc1N ; Cc1cc2[nH]c(C(=O)N(Cc3ccc(-c4c(F)cccc4F)cn3)C3CC(C)(C)Cc4cccnc43)cc2nc1N]

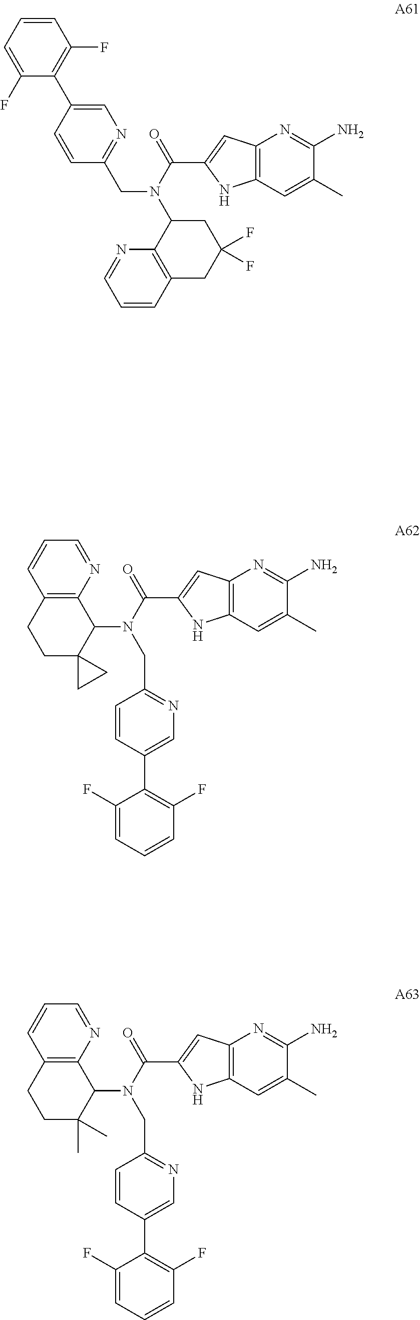 [SMILES: Cc1cc2[nH]c(C(=O)N(Cc3ccc(-c4c(F)cccc4F)cn3)C3CC(F)(F)Cc4cccnc43)cc2nc1N ; Cc1cc2[nH]c(C(=O)N(Cc3ccc(-c4c(F)cccc4F)cn3)C3c4ncccc4CCC34CC4)cc2nc1N ; Cc1cc2[nH]c(C(=O)N(Cc3ccc(-c4c(F)cccc4F)cn3)C3c4ncccc4CCC3(C)C)cc2nc1N]

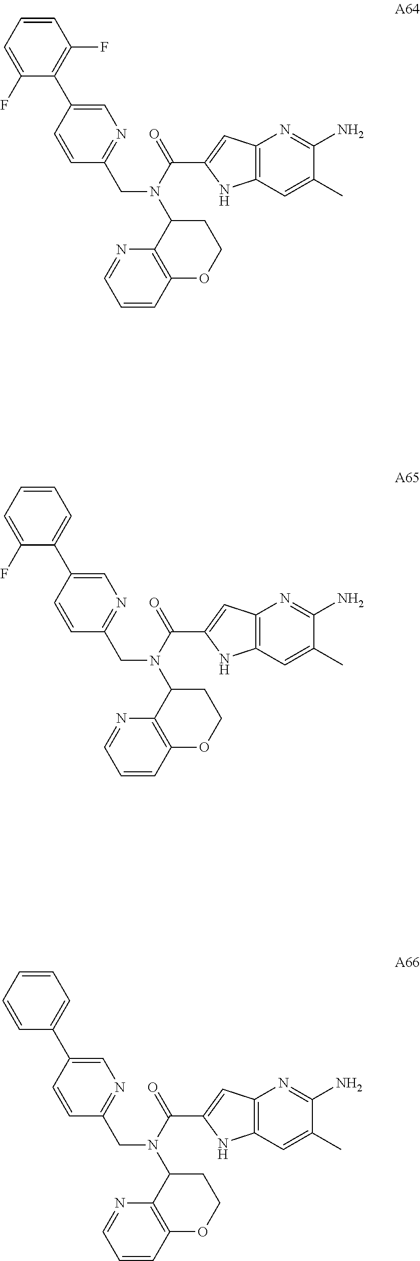 [SMILES: Cc1cc2[nH]c(C(=O)N(Cc3ccc(-c4c(F)cccc4F)cn3)C3CCOc4cccnc43)cc2nc1N ; Cc1cc2[nH]c(C(=O)N(Cc3ccc(-c4ccccc4F)cn3)C3CCOc4cccnc43)cc2nc1N ; Cc1cc2[nH]c(C(=O)N(Cc3ccc(-c4ccccc4)cn3)C3CCOc4cccnc43)cc2nc1N]

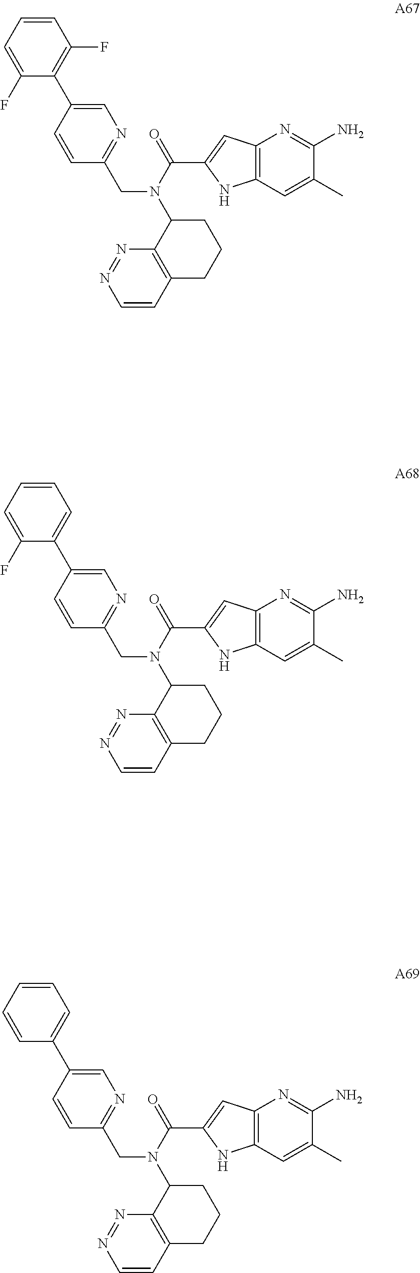 [SMILES: Cc1cc2[nH]c(C(=O)N(Cc3ccc(-c4c(F)cccc4F)cn3)C3CCCc4ccnnc43)cc2nc1N ; Cc1cc2[nH]c(C(=O)N(Cc3ccc(-c4ccccc4F)cn3)C3CCCc4ccnnc43)cc2nc1N ; Cc1cc2[nH]c(C(=O)N(Cc3ccc(-c4ccccc4)cn3)C3CCCc4ccnnc43)cc2nc1N]

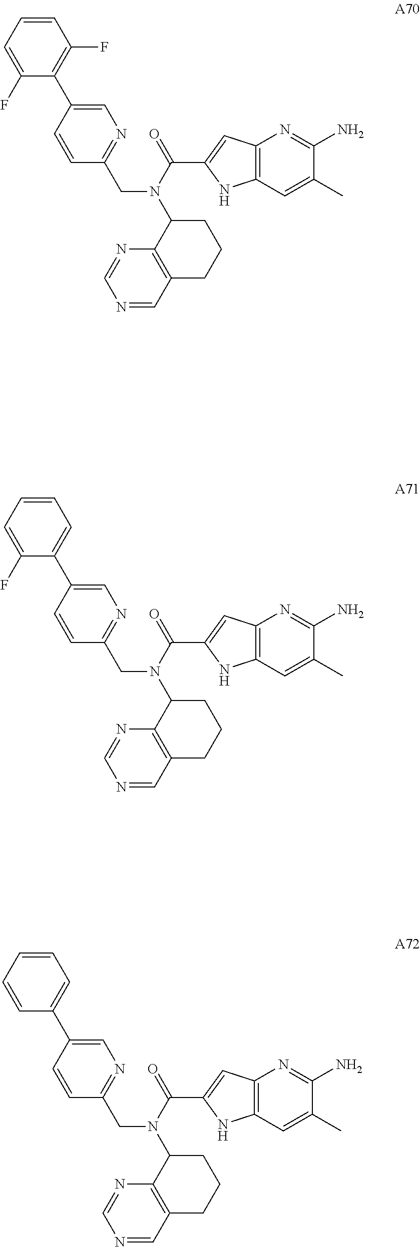 [SMILES: Cc1cc2[nH]c(C(=O)N(Cc3ccc(-c4c(F)cccc4F)cn3)C3CCCc4cncnc43)cc2nc1N ; Cc1cc2[nH]c(C(=O)N(Cc3ccc(-c4ccccc4F)cn3)C3CCCc4cncnc43)cc2nc1N ; Cc1cc2[nH]c(C(=O)N(Cc3ccc(-c4ccccc4)cn3)C3CCCc4cncnc43)cc2nc1N]

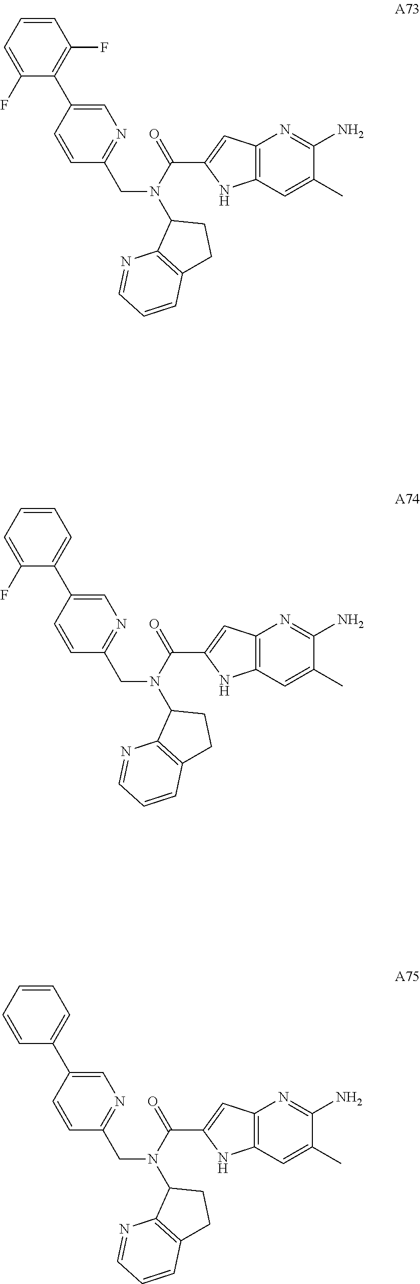 [SMILES: Cc1cc2[nH]c(C(=O)N(Cc3ccc(-c4c(F)cccc4F)cn3)C3CCc4cccnc43)cc2nc1N ; Cc1cc2[nH]c(C(=O)N(Cc3ccc(-c4ccccc4F)cn3)C3CCc4cccnc43)cc2nc1N ; Cc1cc2[nH]c(C(=O)N(Cc3ccc(-c4ccccc4)cn3)C3CCc4cccnc43)cc2nc1N]

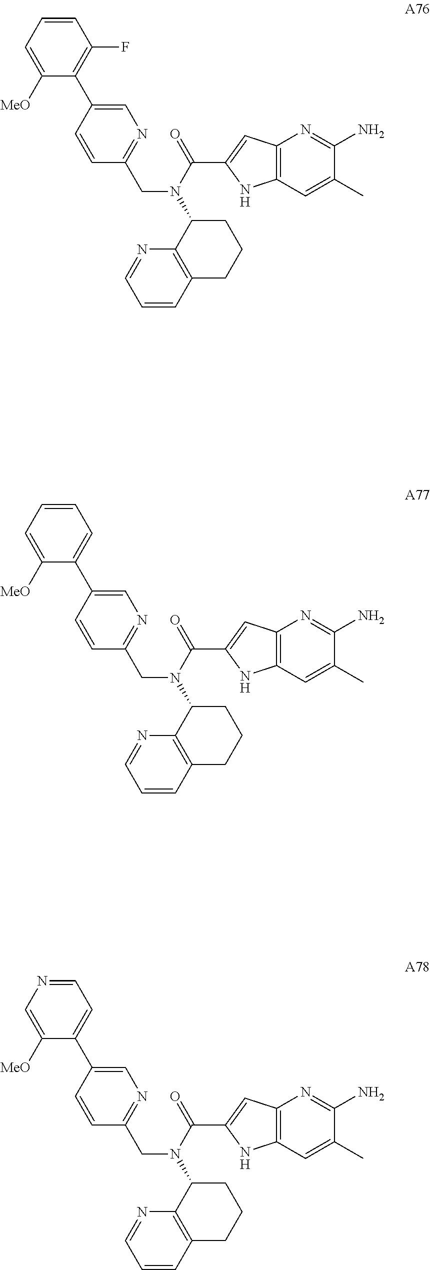 [SMILES: COc1cccc(F)c1-c1ccc(CN(C(=O)c2cc3nc(N)c(C)cc3[nH]2)[C@@H]2CCCc3cccnc32)nc1 ; COc1ccccc1-c1ccc(CN(C(=O)c2cc3nc(N)c(C)cc3[nH]2)[C@@H]2CCCc3cccnc32)nc1 ; COc1cnccc1-c1ccc(CN(C(=O)c2cc3nc(N)c(C)cc3[nH]2)[C@@H]2CCCc3cccnc32)nc1]

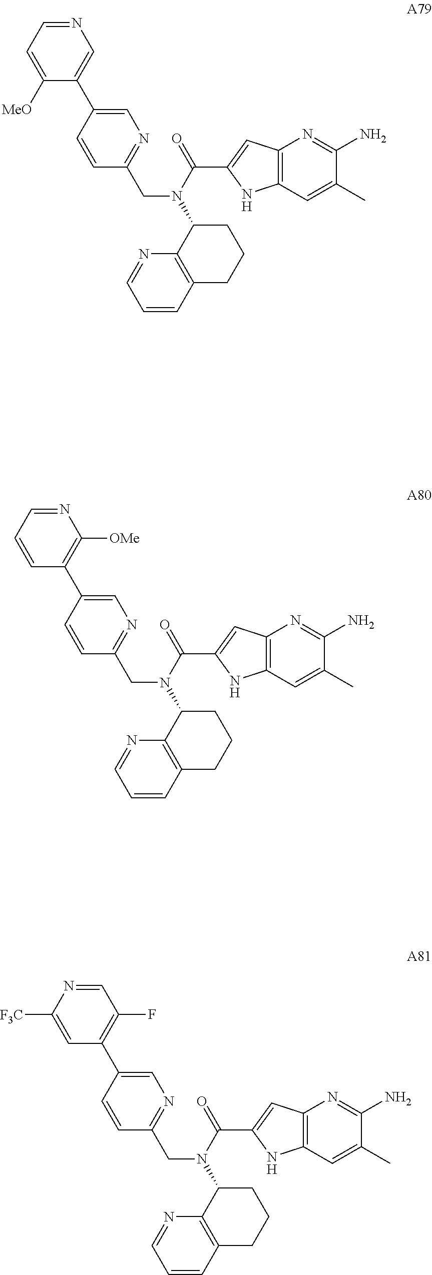 [SMILES: COc1ccncc1-c1ccc(CN(C(=O)c2cc3nc(N)c(C)cc3[nH]2)[C@@H]2CCCc3cccnc32)nc1 ; COc1ncccc1-c1ccc(CN(C(=O)c2cc3nc(N)c(C)cc3[nH]2)[C@@H]2CCCc3cccnc32)nc1 ; Cc1cc2[nH]c(C(=O)N(Cc3ccc(-c4cc(C(F)(F)F)ncc4F)cn3)[C@@H]3CCCc4cccnc43)cc2nc1N]

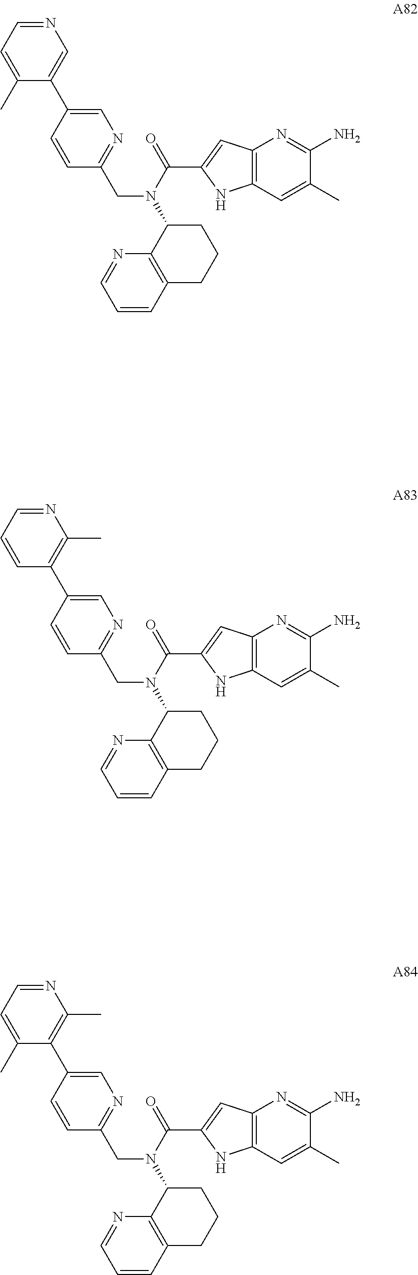 [SMILES: Cc1ccncc1-c1ccc(CN(C(=O)c2cc3nc(N)c(C)cc3[nH]2)[C@@H]2CCCc3cccnc32)nc1 ; Cc1cc2[nH]c(C(=O)N(Cc3ccc(-c4cccnc4C)cn3)[C@@H]3CCCc4cccnc43)cc2nc1N ; Cc1cc2[nH]c(C(=O)N(Cc3ccc(-c4c(C)ccnc4C)cn3)[C@@H]3CCCc4cccnc43)cc2nc1N]

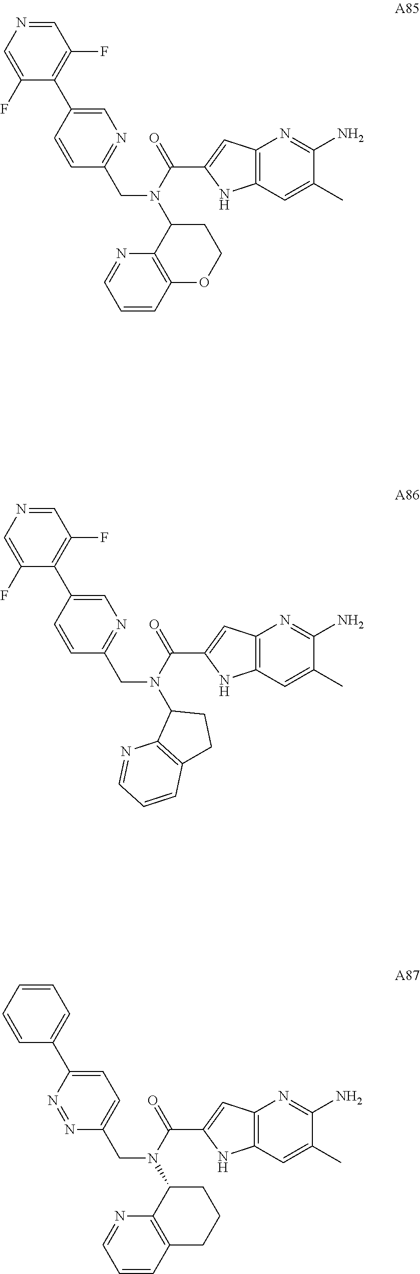 [SMILES: Cc1cc2[nH]c(C(=O)N(Cc3ccc(-c4c(F)cncc4F)cn3)C3CCOc4cccnc43)cc2nc1N ; Cc1cc2[nH]c(C(=O)N(Cc3ccc(-c4c(F)cncc4F)cn3)C3CCc4cccnc43)cc2nc1N ; Cc1cc2[nH]c(C(=O)N(Cc3ccc(-c4ccccc4)nn3)[C@@H]3CCCc4cccnc43)cc2nc1N]

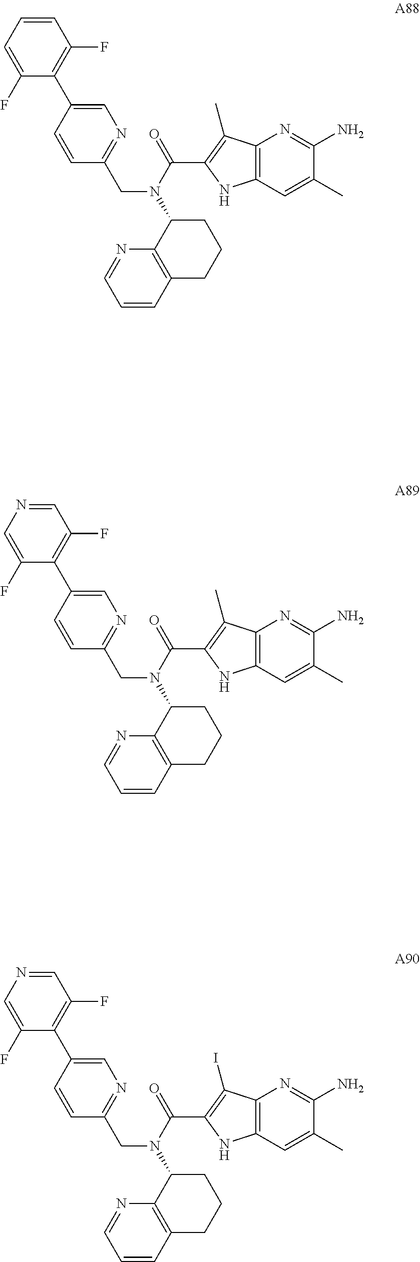 [SMILES: Cc1cc2[nH]c(C(=O)N(Cc3ccc(-c4c(F)cccc4F)cn3)[C@@H]3CCCc4cccnc43)c(C)c2nc1N ; Cc1cc2[nH]c(C(=O)N(Cc3ccc(-c4c(F)cncc4F)cn3)[C@@H]3CCCc4cccnc43)c(C)c2nc1N ; Cc1cc2[nH]c(C(=O)N(Cc3ccc(-c4c(F)cncc4F)cn3)[C@@H]3CCCc4cccnc43)c(I)c2nc1N]

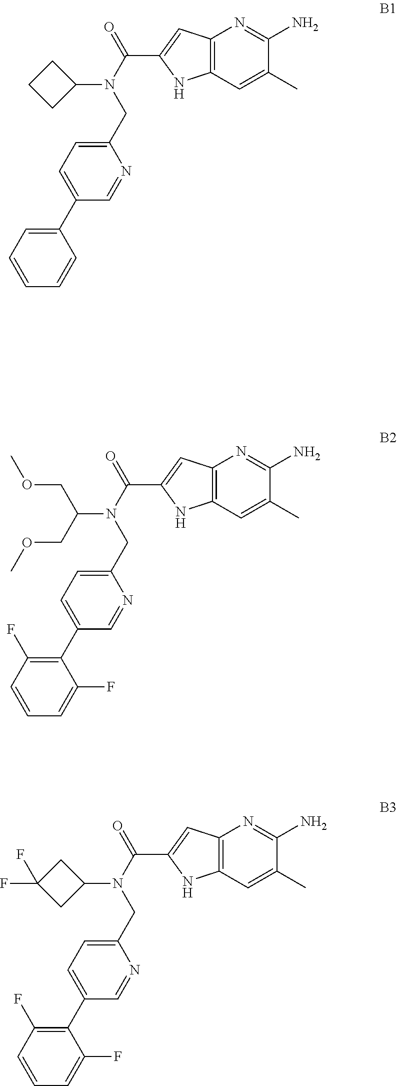 [SMILES: Cc1cc2[nH]c(C(=O)N(Cc3ccc(-c4ccccc4)cn3)C3CCC3)cc2nc1N ; COCC(COC)N(Cc1ccc(-c2c(F)cccc2F)cn1)C(=O)c1cc2nc(N)c(C)cc2[nH]1 ; Cc1cc2[nH]c(C(=O)N(Cc3ccc(-c4c(F)cccc4F)cn3)C3CC(F)(F)C3)cc2nc1N]

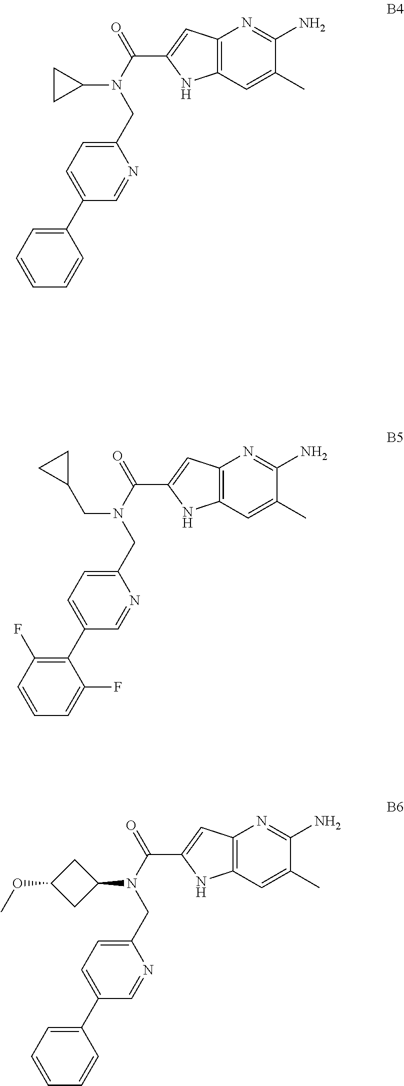

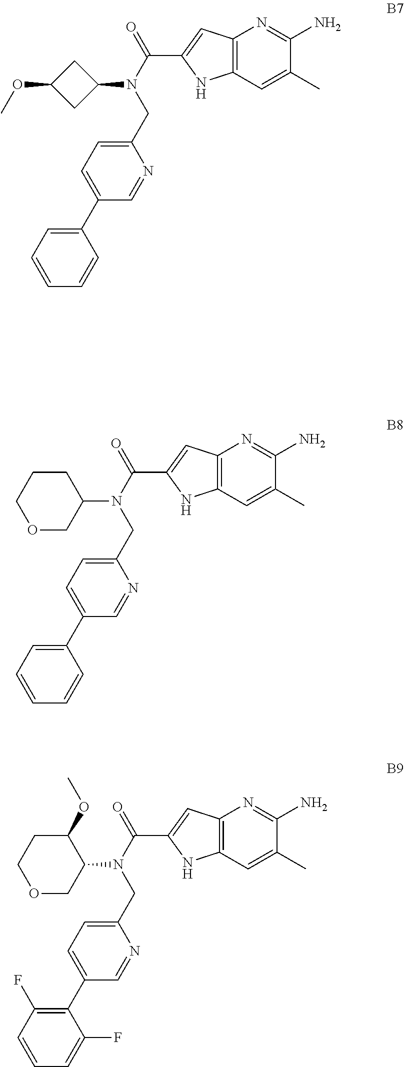

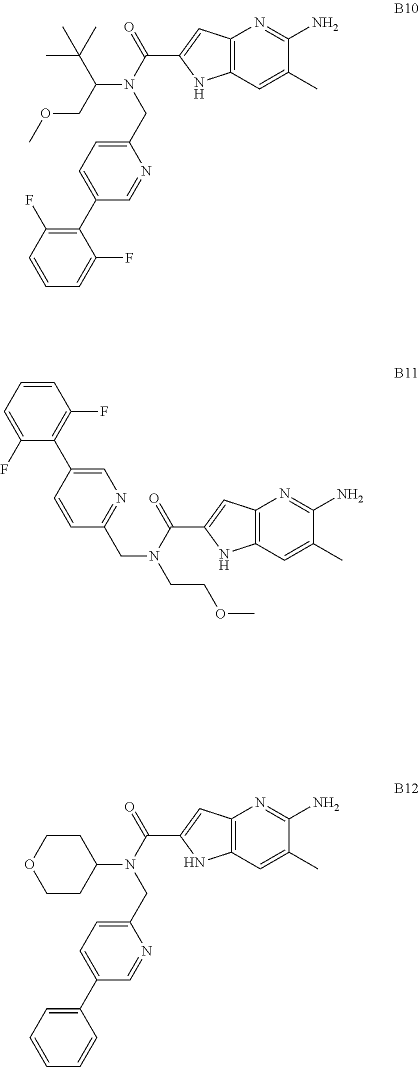 [SMILES: COCC(N(Cc1ccc(-c2c(F)cccc2F)cn1)C(=O)c1cc2nc(N)c(C)cc2[nH]1)C(C)(C)C ; COCCN(Cc1ccc(-c2c(F)cccc2F)cn1)C(=O)c1cc2nc(N)c(C)cc2[nH]1 ; Cc1cc2[nH]c(C(=O)N(Cc3ccc(-c4ccccc4)cn3)C3CCOCC3)cc2nc1N]

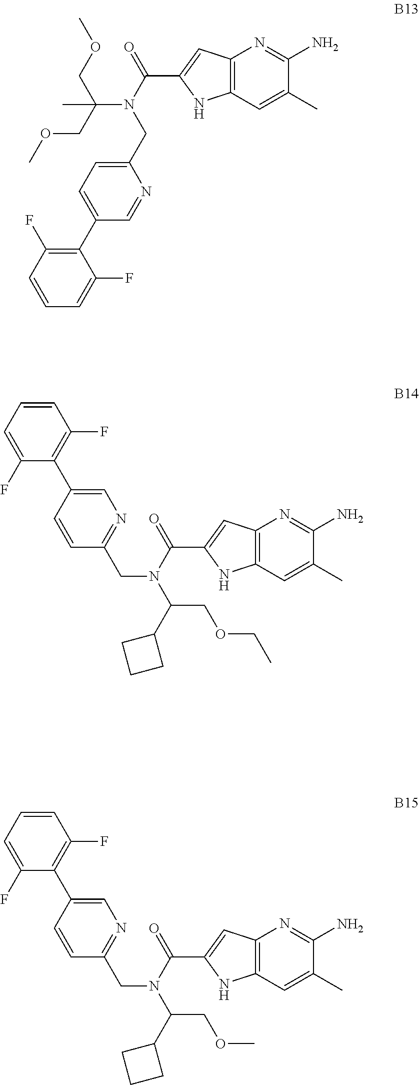 [SMILES: COCC(C)(COC)N(Cc1ccc(-c2c(F)cccc2F)cn1)C(=O)c1cc2nc(N)c(C)cc2[nH]1 ; CCOCC(C1CCC1)N(Cc1ccc(-c2c(F)cccc2F)cn1)C(=O)c1cc2nc(N)c(C)cc2[nH]1 ; COCC(C1CCC1)N(Cc1ccc(-c2c(F)cccc2F)cn1)C(=O)c1cc2nc(N)c(C)cc2[nH]1]

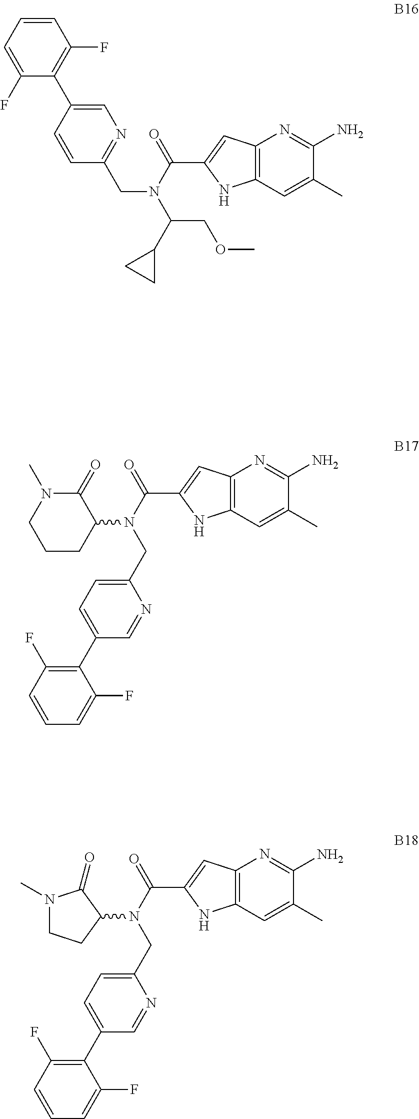

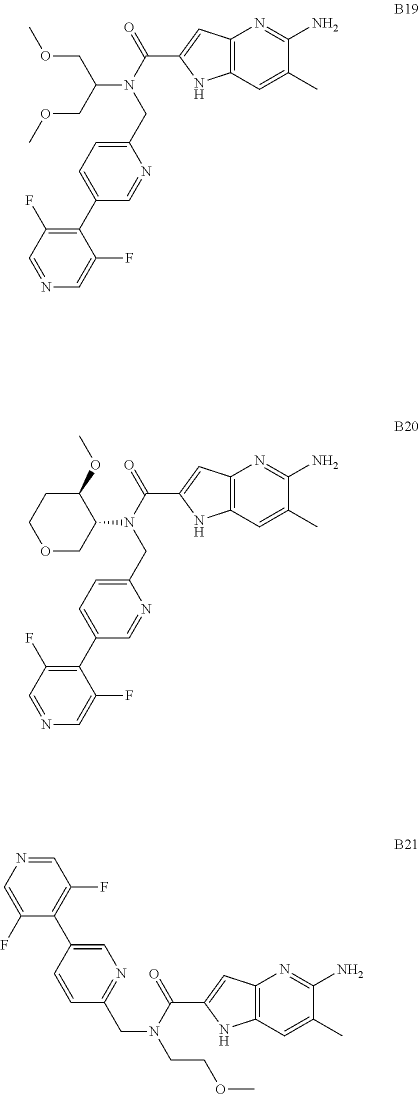 [SMILES: COCC(COC)N(Cc1ccc(-c2c(F)cncc2F)cn1)C(=O)c1cc2nc(N)c(C)cc2[nH]1 ; CO[C@@H]1CCOC[C@H]1N(Cc1ccc(-c2c(F)cncc2F)cn1)C(=O)c1cc2nc(N)c(C)cc2[nH]1 ; COCCN(Cc1ccc(-c2c(F)cncc2F)cn1)C(=O)c1cc2nc(N)c(C)cc2[nH]1]

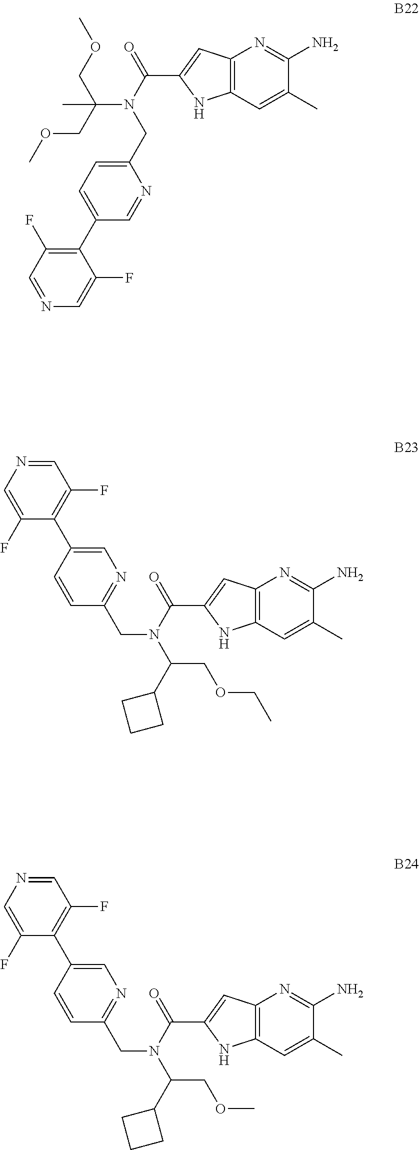 [SMILES: COCC(C)(COC)N(Cc1ccc(-c2c(F)cncc2F)cn1)C(=O)c1cc2nc(N)c(C)cc2[nH]1 ; CCOCC(C1CCC1)N(Cc1ccc(-c2c(F)cncc2F)cn1)C(=O)c1cc2nc(N)c(C)cc2[nH]1 ; COCC(C1CCC1)N(Cc1ccc(-c2c(F)cncc2F)cn1)C(=O)c1cc2nc(N)c(C)cc2[nH]1]

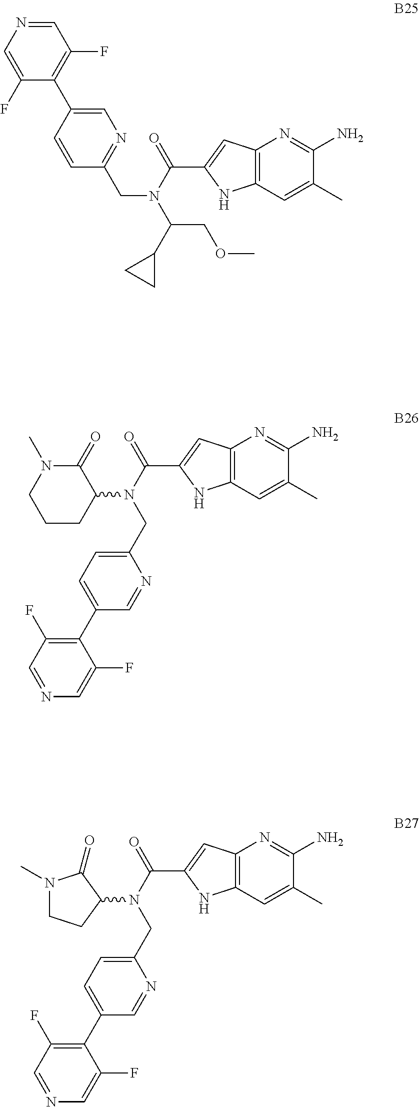

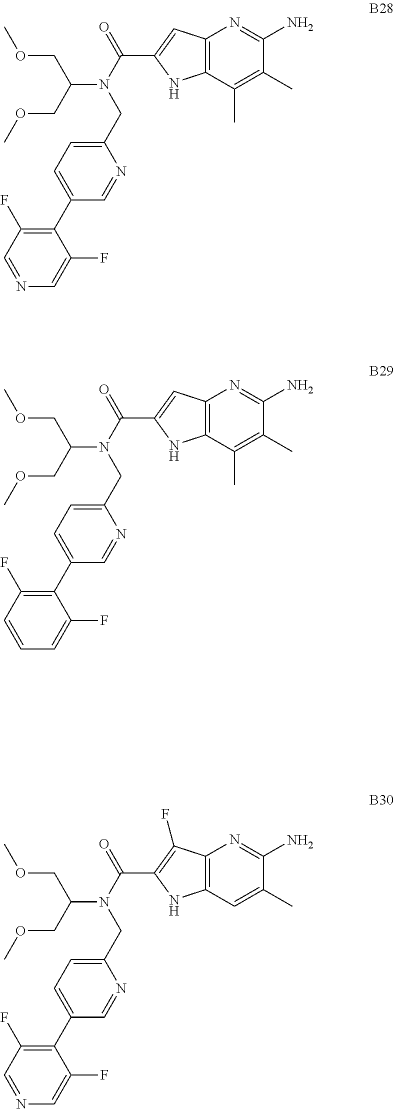 [SMILES: COCC(COC)N(Cc1ccc(-c2c(F)cncc2F)cn1)C(=O)c1cc2nc(N)c(C)c(C)c2[nH]1 ; COCC(COC)N(Cc1ccc(-c2c(F)cccc2F)cn1)C(=O)c1cc2nc(N)c(C)c(C)c2[nH]1 ; COCC(COC)N(Cc1ccc(-c2c(F)cncc2F)cn1)C(=O)c1[nH]c2cc(C)c(N)nc2c1F]

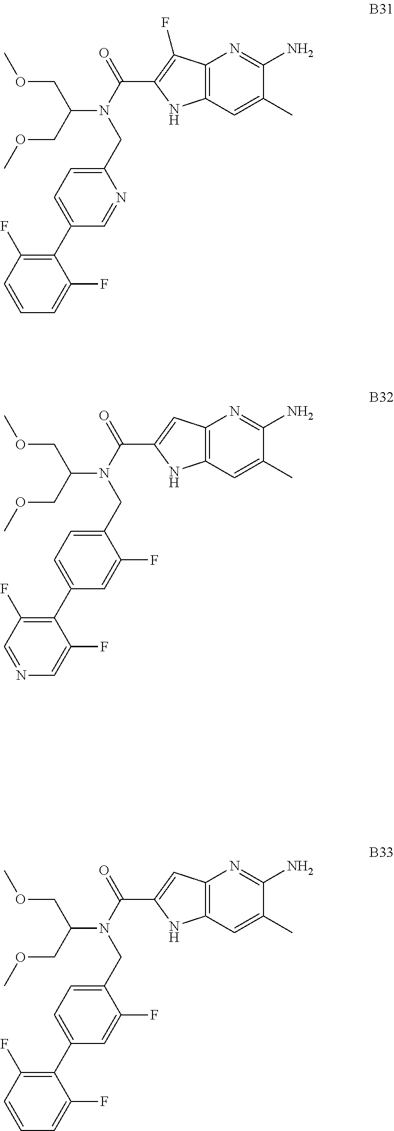 [SMILES: COCC(COC)N(Cc1ccc(-c2c(F)cccc2F)cn1)C(=O)c1[nH]c2cc(C)c(N)nc2c1F ; COCC(COC)N(Cc1ccc(-c2c(F)cncc2F)cc1F)C(=O)c1cc2nc(N)c(C)cc2[nH]1 ; COCC(COC)N(Cc1ccc(-c2c(F)cccc2F)cc1F)C(=O)c1cc2nc(N)c(C)cc2[nH]1]

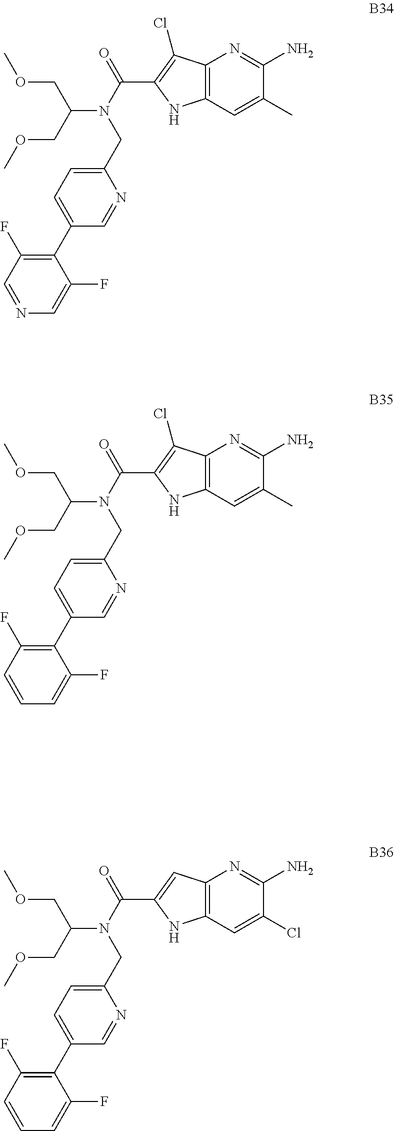 [SMILES: COCC(COC)N(Cc1ccc(-c2c(F)cncc2F)cn1)C(=O)c1[nH]c2cc(C)c(N)nc2c1Cl ; COCC(COC)N(Cc1ccc(-c2c(F)cccc2F)cn1)C(=O)c1[nH]c2cc(C)c(N)nc2c1Cl ; COCC(COC)N(Cc1ccc(-c2c(F)cccc2F)cn1)C(=O)c1cc2nc(N)c(Cl)cc2[nH]1]

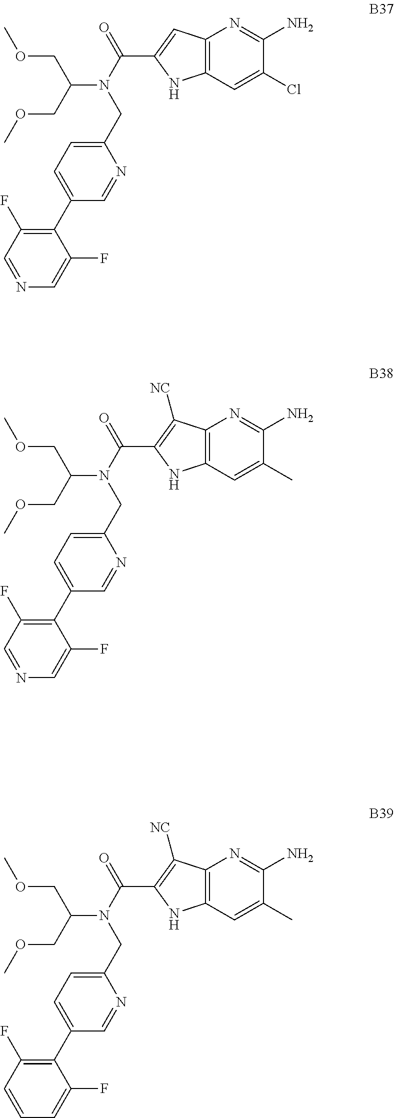 [SMILES: COCC(COC)N(Cc1ccc(-c2c(F)cncc2F)cn1)C(=O)c1cc2nc(N)c(Cl)cc2[nH]1 ; COCC(COC)N(Cc1ccc(-c2c(F)cncc2F)cn1)C(=O)c1[nH]c2cc(C)c(N)nc2c1C#N ; COCC(COC)N(Cc1ccc(-c2c(F)cccc2F)cn1)C(=O)c1[nH]c2cc(C)c(N)nc2c1C#N]

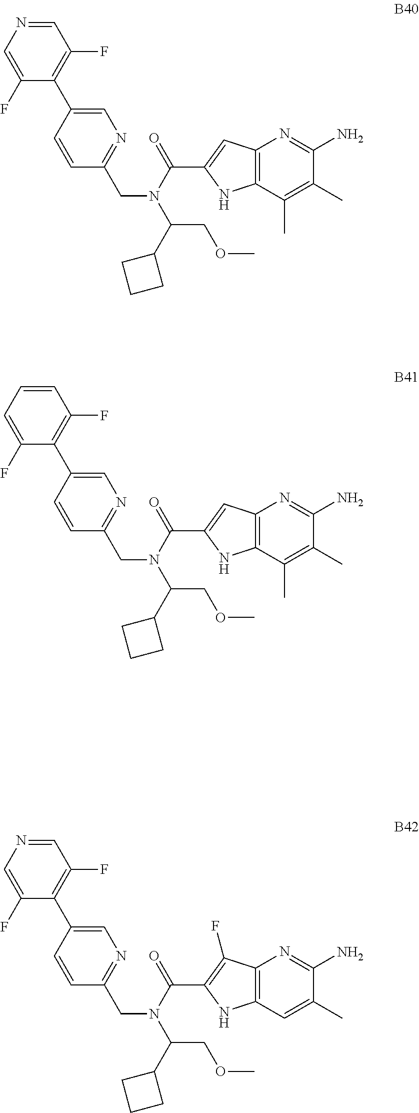 [SMILES: COCC(C1CCC1)N(Cc1ccc(-c2c(F)cncc2F)cn1)C(=O)c1cc2nc(N)c(C)c(C)c2[nH]1 ; COCC(C1CCC1)N(Cc1ccc(-c2c(F)cccc2F)cn1)C(=O)c1cc2nc(N)c(C)c(C)c2[nH]1 ; COCC(C1CCC1)N(Cc1ccc(-c2c(F)cncc2F)cn1)C(=O)c1[nH]c2cc(C)c(N)nc2c1F]

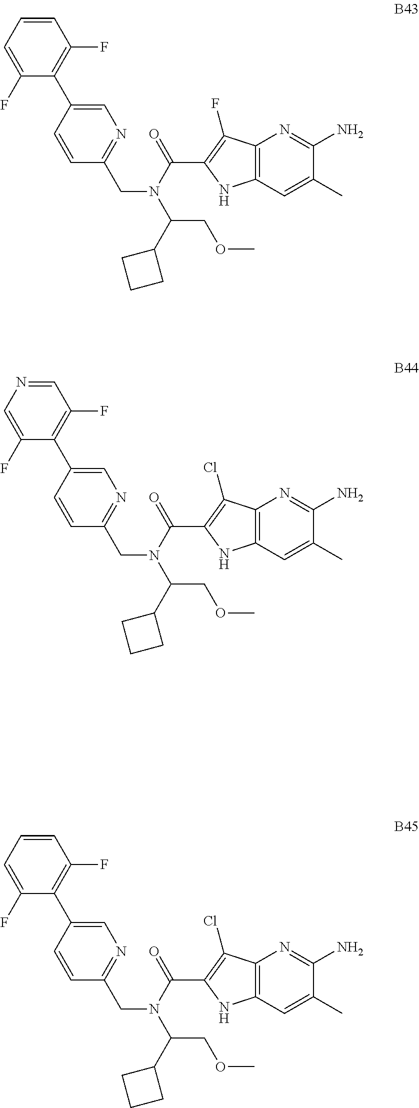 [SMILES: COCC(C1CCC1)N(Cc1ccc(-c2c(F)cccc2F)cn1)C(=O)c1[nH]c2cc(C)c(N)nc2c1F ; COCC(C1CCC1)N(Cc1ccc(-c2c(F)cncc2F)cn1)C(=O)c1[nH]c2cc(C)c(N)nc2c1Cl ; COCC(C1CCC1)N(Cc1ccc(-c2c(F)cccc2F)cn1)C(=O)c1[nH]c2cc(C)c(N)nc2c1Cl]

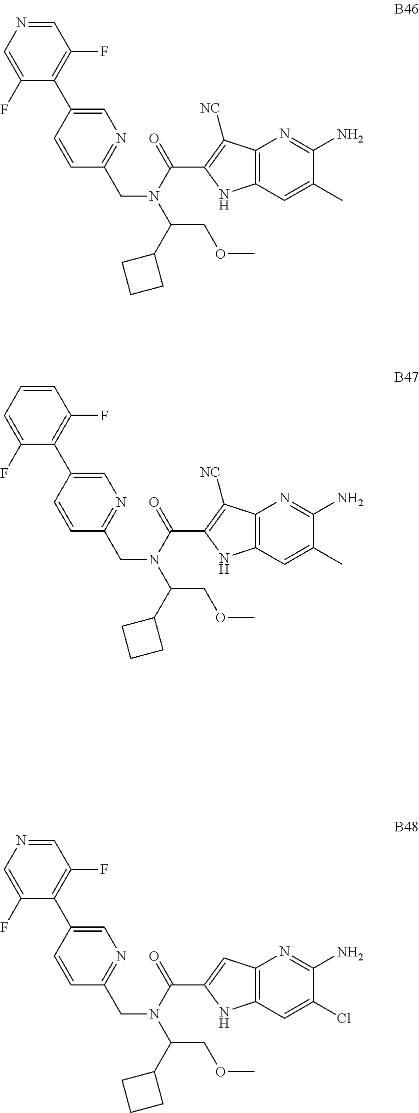 [SMILES: COCC(C1CCC1)N(Cc1ccc(-c2c(F)cncc2F)cn1)C(=O)c1[nH]c2cc(C)c(N)nc2c1C#N ; COCC(C1CCC1)N(Cc1ccc(-c2c(F)cccc2F)cn1)C(=O)c1[nH]c2cc(C)c(N)nc2c1C#N ; COCC(C1CCC1)N(Cc1ccc(-c2c(F)cncc2F)cn1)C(=O)c1cc2nc(N)c(Cl)cc2[nH]1]

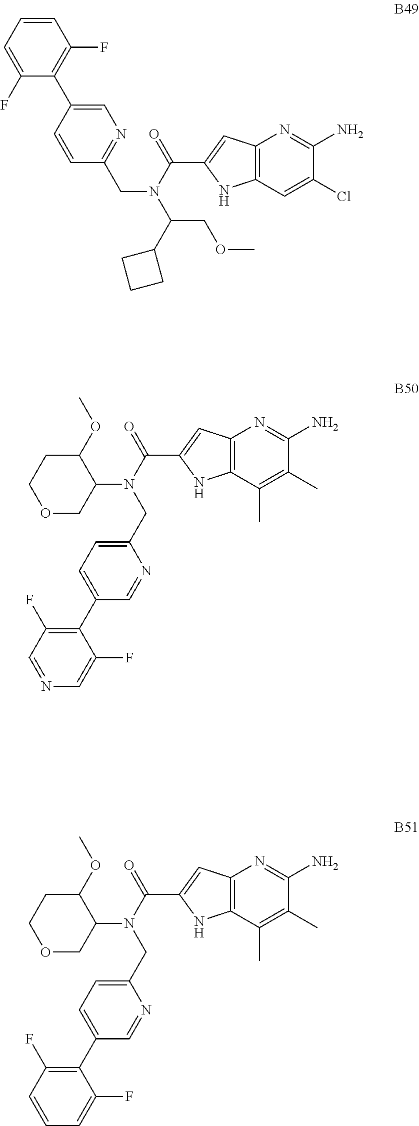 [SMILES: COCC(C1CCC1)N(Cc1ccc(-c2c(F)cccc2F)cn1)C(=O)c1cc2nc(N)c(Cl)cc2[nH]1 ; COC1CCOCC1N(Cc1ccc(-c2c(F)cncc2F)cn1)C(=O)c1cc2nc(N)c(C)c(C)c2[nH]1 ; COC1CCOCC1N(Cc1ccc(-c2c(F)cccc2F)cn1)C(=O)c1cc2nc(N)c(C)c(C)c2[nH]1]

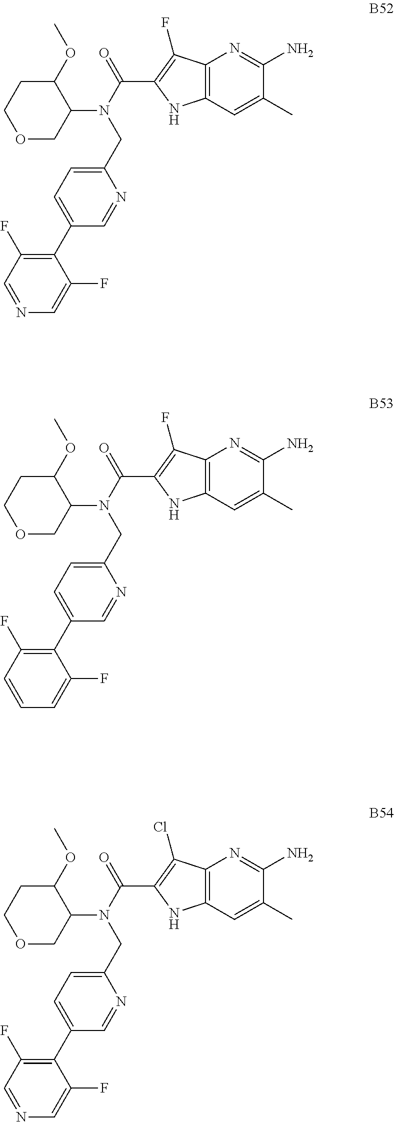 [SMILES: COC1CCOCC1N(Cc1ccc(-c2c(F)cncc2F)cn1)C(=O)c1[nH]c2cc(C)c(N)nc2c1F ; COC1CCOCC1N(Cc1ccc(-c2c(F)cccc2F)cn1)C(=O)c1[nH]c2cc(C)c(N)nc2c1F ; COC1CCOCC1N(Cc1ccc(-c2c(F)cncc2F)cn1)C(=O)c1[nH]c2cc(C)c(N)nc2c1Cl]

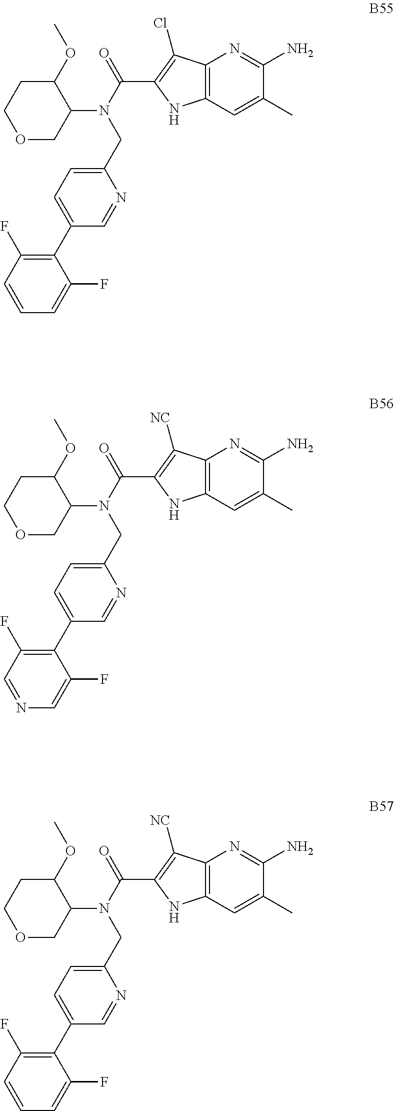 [SMILES: COC1CCOCC1N(Cc1ccc(-c2c(F)cccc2F)cn1)C(=O)c1[nH]c2cc(C)c(N)nc2c1Cl ; COC1CCOCC1N(Cc1ccc(-c2c(F)cncc2F)cn1)C(=O)c1[nH]c2cc(C)c(N)nc2c1C#N ; COC1CCOCC1N(Cc1ccc(-c2c(F)cccc2F)cn1)C(=O)c1[nH]c2cc(C)c(N)nc2c1C#N]

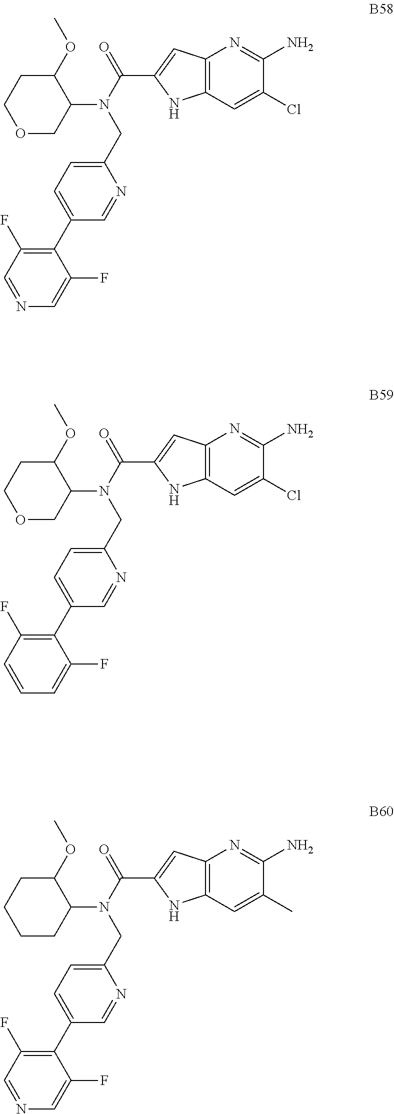 [SMILES: COC1CCOCC1N(Cc1ccc(-c2c(F)cncc2F)cn1)C(=O)c1cc2nc(N)c(Cl)cc2[nH]1 ; COC1CCOCC1N(Cc1ccc(-c2c(F)cccc2F)cn1)C(=O)c1cc2nc(N)c(Cl)cc2[nH]1 ; COC1CCCCC1N(Cc1ccc(-c2c(F)cncc2F)cn1)C(=O)c1cc2nc(N)c(C)cc2[nH]1]

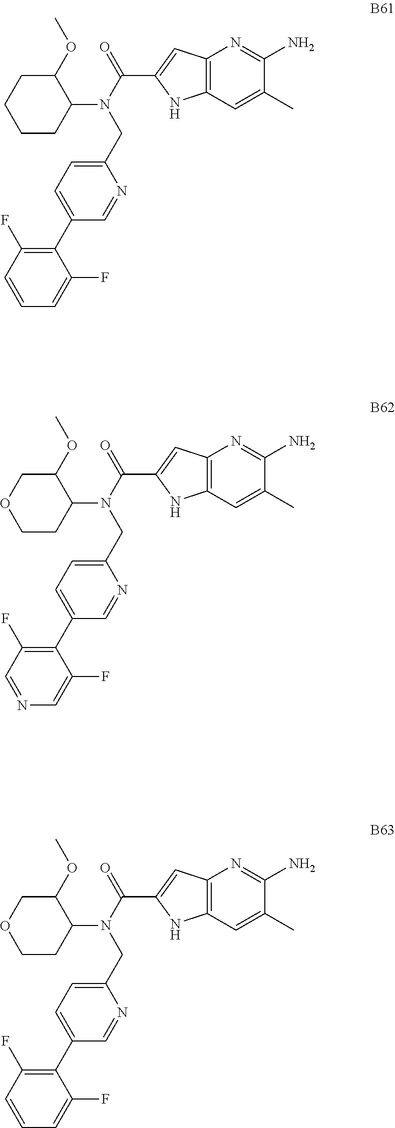 [SMILES: COC1CCCCC1N(Cc1ccc(-c2c(F)cccc2F)cn1)C(=O)c1cc2nc(N)c(C)cc2[nH]1 ; COC1COCCC1N(Cc1ccc(-c2c(F)cncc2F)cn1)C(=O)c1cc2nc(N)c(C)cc2[nH]1 ; COC1COCCC1N(Cc1ccc(-c2c(F)cccc2F)cn1)C(=O)c1cc2nc(N)c(C)cc2[nH]1]

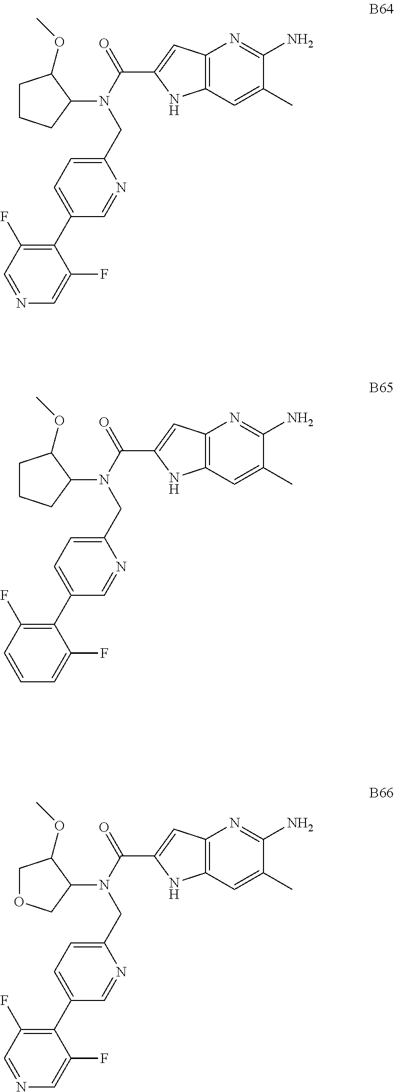 [SMILES: COC1CCCC1N(Cc1ccc(-c2c(F)cncc2F)cn1)C(=O)c1cc2nc(N)c(C)cc2[nH]1 ; COC1CCCC1N(Cc1ccc(-c2c(F)cccc2F)cn1)C(=O)c1cc2nc(N)c(C)cc2[nH]1 ; COC1COCC1N(Cc1ccc(-c2c(F)cncc2F)cn1)C(=O)c1cc2nc(N)c(C)cc2[nH]1]

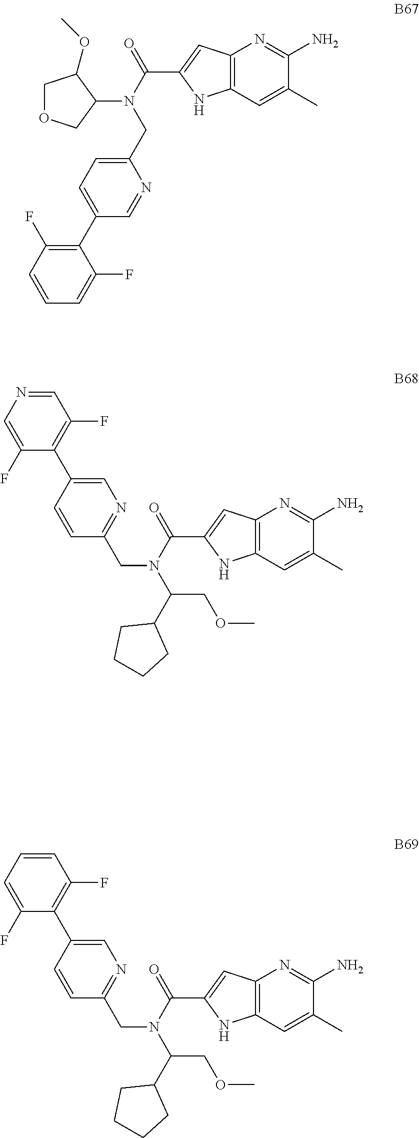 [SMILES: COC1COCC1N(Cc1ccc(-c2c(F)cccc2F)cn1)C(=O)c1cc2nc(N)c(C)cc2[nH]1 ; COCC(C1CCCC1)N(Cc1ccc(-c2c(F)cncc2F)cn1)C(=O)c1cc2nc(N)c(C)cc2[nH]1 ; COCC(C1CCCC1)N(Cc1ccc(-c2c(F)cccc2F)cn1)C(=O)c1cc2nc(N)c(C)cc2[nH]1]

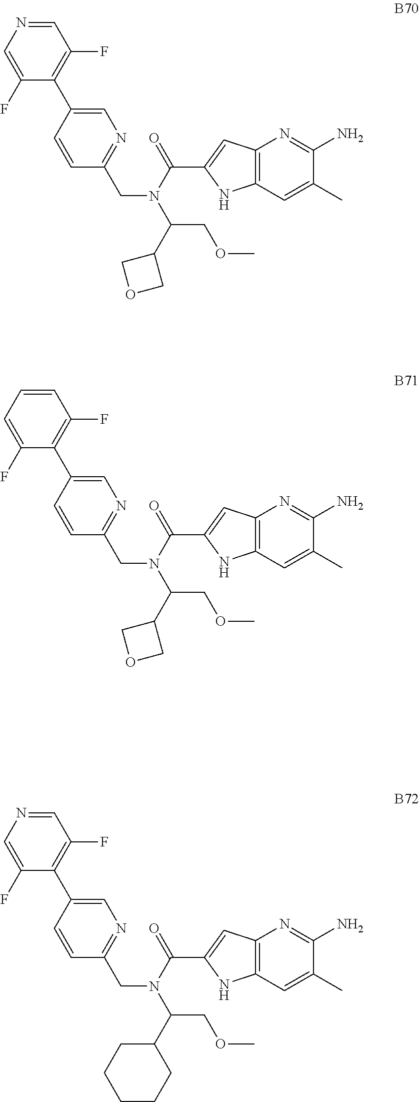 [SMILES: COCC(C1COC1)N(Cc1ccc(-c2c(F)cncc2F)cn1)C(=O)c1cc2nc(N)c(C)cc2[nH]1 ; COCC(C1COC1)N(Cc1ccc(-c2c(F)cccc2F)cn1)C(=O)c1cc2nc(N)c(C)cc2[nH]1 ; COCC(C1CCCCC1)N(Cc1ccc(-c2c(F)cncc2F)cn1)C(=O)c1cc2nc(N)c(C)cc2[nH]1]

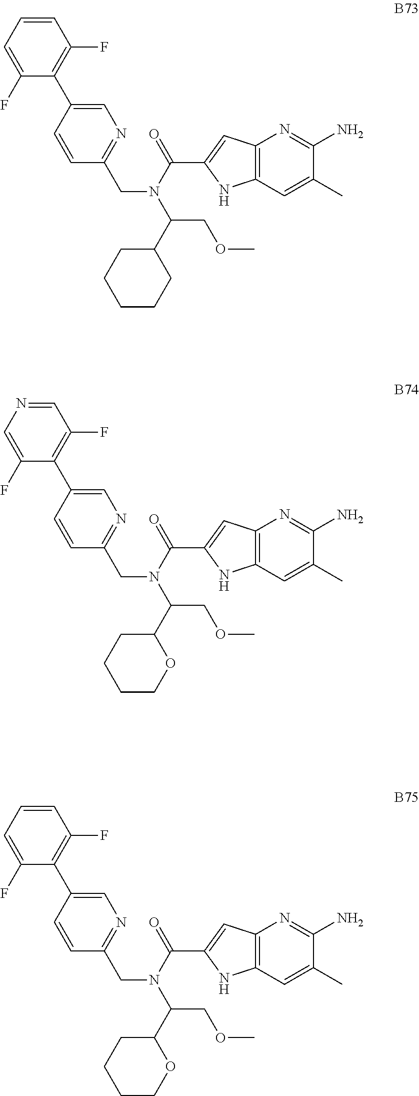 [SMILES: COCC(C1CCCCC1)N(Cc1ccc(-c2c(F)cccc2F)cn1)C(=O)c1cc2nc(N)c(C)cc2[nH]1 ; COCC(C1CCCCO1)N(Cc1ccc(-c2c(F)cncc2F)cn1)C(=O)c1cc2nc(N)c(C)cc2[nH]1 ; COCC(C1CCCCO1)N(Cc1ccc(-c2c(F)cccc2F)cn1)C(=O)c1cc2nc(N)c(C)cc2[nH]1]

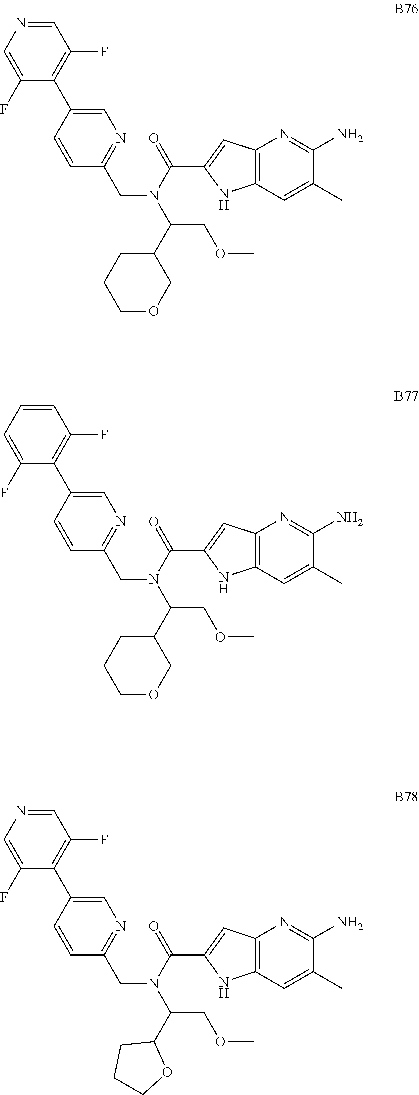 [SMILES: COCC(C1CCCOC1)N(Cc1ccc(-c2c(F)cncc2F)cn1)C(=O)c1cc2nc(N)c(C)cc2[nH]1 ; COCC(C1CCCOC1)N(Cc1ccc(-c2c(F)cccc2F)cn1)C(=O)c1cc2nc(N)c(C)cc2[nH]1 ; COCC(C1CCCO1)N(Cc1ccc(-c2c(F)cncc2F)cn1)C(=O)c1cc2nc(N)c(C)cc2[nH]1]

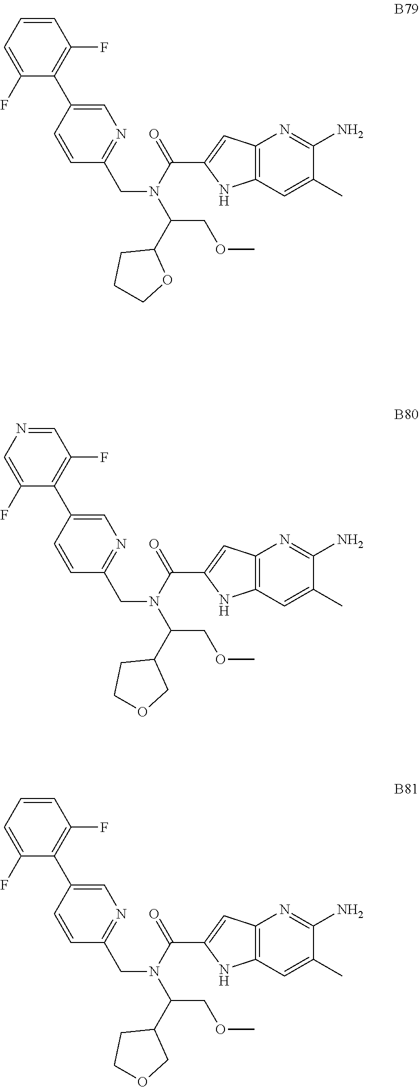 [SMILES: COCC(C1CCCO1)N(Cc1ccc(-c2c(F)cccc2F)cn1)C(=O)c1cc2nc(N)c(C)cc2[nH]1 ; COCC(C1CCOC1)N(Cc1ccc(-c2c(F)cncc2F)cn1)C(=O)c1cc2nc(N)c(C)cc2[nH]1 ; COCC(C1CCOC1)N(Cc1ccc(-c2c(F)cccc2F)cn1)C(=O)c1cc2nc(N)c(C)cc2[nH]1]

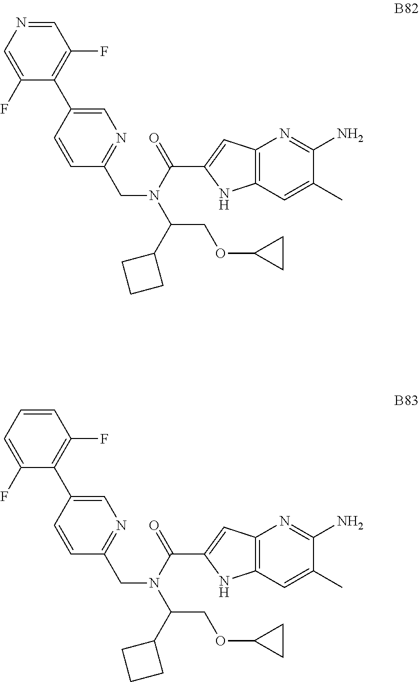 [SMILES: Cc1cc2[nH]c(C(=O)N(Cc3ccc(-c4c(F)cncc4F)cn3)C(COC3CC3)C3CCC3)cc2nc1N ; Cc1cc2[nH]c(C(=O)N(Cc3ccc(-c4c(F)cccc4F)cn3)C(COC3CC3)C3CCC3)cc2nc1N]

42. A pharmaceutical composition comprising a compound of any one of claims 1-41 or a pharmaceutically acceptable salt, stereoisomer, tautomer or prodrug thereof, together with a pharmaceutically acceptable excipient.

43. A method of decreasing PRMT5 activity by inhibition, which comprises administering to an individual the compound according to any one of claims 1-41, or a pharmaceutically acceptable salt thereof, including the compound of formula (I) or the specific compounds exemplified herein.

44. The method of claim 43, wherein the disease is selected from cancer.

45. Use of a compound of any one of claims 1-41 or a pharmaceutically acceptable salt, stereoisomer, tautomer or prodrug thereof in the preparation of a medicament for treating a disease that is modulated by PRMT5.

46. The use of claim 45, wherein the disease is cancer.

47. The use of claim 46, wherein the disease is MTAP-null solid tumor, including but not limited to lung cancer, bladder cancer, melanoma, pancreatic cancer, esophageal cancer, gastric adenocarcinoma, breast cancer or glioblastoma.
